(19) United States
(12) Patent Application Publication
(10) Pub. No.: US 2003/0100550 A1
(43) Pub. Date: May 29, 2003

(54) Carboxamide diazepin derivatives, preparation method, use as medicines, pharmaceutical compositions and use thereof

(76) Inventors: Neerja Bhatnagar, Savigny Sur Orge (FR); Jacques Mauger, Paris (FR)

Correspondence Address: MUSERLIAN AND LUCAS AND MERCANTI, LLP, 600 THIRD AVENUE, NEW YORK, NY, 10016, US

(21) Appl. No.: 10/168,116

(22) PCT Filed: Dec. 21, 2000
(86) PCT No.: PCT/FR00/03622

(51) Int. Cl. A61K 31/5513; C07D 487/04

(52) U.S. Cl. 514/221; 540/500

(57) ABSTRACT

The invention concerns products of formula (I) wherein: R₁ represents in particular —C(O)—R5, —SO2—R5 or —C(O)—NR6R5, R2 and R7 are such that either R7 represents a hydrogen atom and R2 is such that the group (a) represents the radical of a natural or non-natural amino acid, or R2 and R7 form together a cycle with the nitrogen and carbon atom whereto they are bound, R3 represents in particular the radical —CH═N2 or —CH2-L—R4, R4 represents in particular a linear or branched alkyl radical, and their additive salts with mineral or organic acids or with mineral or organic bases of said products of formula (I). The invention also concerns the method for preparing said products and their use as medicines.

A subject of the invention is therefore new 9(S)-amino-6,10-dioxo-1,2,3,4,7,8,9,10-octahydro-6H-pyridazino[1,2-a][1,2]diazepine 1(S)-carboxamide derivatives, their preparation process, their use as medicaments, the pharmaceutical compositions containing them and the new use of such derivatives.

A particular subject of the present invention is the preparation of a library of enzyme inhibitors using 9(S)-amino-6,10-dioxo-1,2,3,4,7,8,9,10-octahydro-6H-pyridazino[1,2-a][1,2]diazepine 1(S)-carboxamide. Metabolic enzymes such as proteases or kinases are enzymes which are widely distributed in the animal kingdom. By way of non-exhaustive examples, as bibliographical references for the proteases, the document: ‘Methods in Enzymology XLII (1975)’ and ‘Journal of Medicinal Chemistry’ vol. 43 No. 3 (‘D. Leung, G. Abbenante and D. P. Fairlie’) and for the kinases, the document: ‘Methods in Enzymology, Vol 80(1981)(Academic Press Inc.)’ can be mentioned.

Among the proteases capable of selectively catalysing the hydrolysis of the polypeptide bonds, the four main classes can be mentioned: aspartic, serine, cysteine protease and metallo-protease.

As aspartic protease, HIV-1 protease, renin, plasmepsins, cathepsin D can in particular be mentioned.

As serine protease thrombin, factor Xa, elastase, tryptase, “complements of convertases”, hepatitis C protease NS3 can in particular be mentioned.

Among the cysteine-proteases, there are three structurally distinct groups, the papain group and the cathepsins, the ICE group (the caspases) and the picorna-viral group (similar to the serine-proteases in which the serine is replaced by a cysteine).

Cathepsin K, cathepsin B, cathepsin L, cathepsin S, caspases, rhinovirus 3C protease and calpains can therefore in particular be mentioned.

As metalloprotease, angiotensin converting enzyme, neutral endopeptidase and the mixture of the two, metalloprotease matrix as well as the tumour-necrosis factor-α-converting enzyme can in particular be mentioned.

Cathepsin K, cathepsin B, cathepsin S, cathepsin L and papain and more particularly cathepsin K, cathepsin B and papain can be particularly mentioned.

These kinase or protease enzymes are involved in the processes of catabolization and of inter and intracellular communication: they play an important role in a large number of diseases in different domains such as in particular the cardiovascular domain, oncology, the central nervous system, inflammation, osteoporosis and also infectious, parasitic, fungal or viral diseases. That is why these proteins are targets of great interest to pharmaceutical research. Within the scope of the present invention, starting from a peptidomimetic intermediate, a chemical library directed against potential inhibitors of these enzymes was designed and constructed: a peptidomimetic intermediate corresponding here to the product of formula (II) as defined hereafter. The products of the present invention as defined above and hereafter possess inhibitory properties of metabolic enzymes such as that defined above in particular of kinases or proteases such as in particular cysteine proteases or serine proteases.

Therefore the products of the present invention can in particular be of use in the prevention or the treatment of diseases in which such metabolic enzymes are involved such as certain cardiovascular diseases, diseases of the central nervous system, inflammatory diseases, bone diseases such as for example osteoporosis, infectious diseases requiring in particular anti-infective agents in their therapy or also certain cancers.

A subject of the present invention is therefore the products of formula (I):

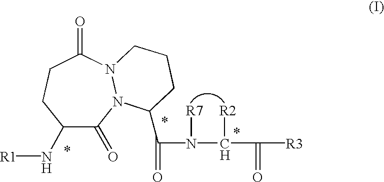

in which:

R₁ represents the —C(O)—R5, —SO2-R5 or —C(O)—NR6R5 radical in which R6 represents a hydrogen atom or a linear or branched alkyl radical containing at most 4 carbon atoms and R5 represents a linear or branched alkyl radical containing at most 6 carbon atoms, cycloalkyl radical containing at most 6 carbon atoms, phenyl, naphthyl or a monocyclic heterocyclic radical saturated or unsaturated with 5 or 6 members containing one or more identical or different heteroatoms chosen from O, N or S,

the alkyl, phenyl and heterocyclic radicals as defined above for R1, being optionally substituted by one or more radicals chosen from the halogen atoms and the linear or branched alkyl or alkoxy radicals containing at most 4 carbon atoms, hydroxyl, acyl radicals containing at most 7 carbon atoms, trifluoromethyl, cyano, thienyl, phenyl, phenoxy, and isoxazolyl radicals;

R2 and R7 are such that

either R7 represents a hydrogen atom and

R2 is such that the

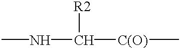

group in the products of formula (I) represents a so-called natural or non-natural amino acid remainder of

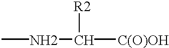

structure with the exception of aspartic acid,

or R2 and R7 together with the nitrogen and carbon atoms to which they are linked form a ring in such a way that the group thus formed in the products of formula (I):

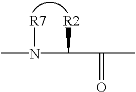

represents a so-called natural or non-natural amino acid remainder of:

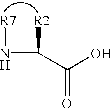

structure

R3 represents the —CH═N2 radical or the —CH2—L—R4 radical in which L represents a single bond or a divalent radical chosen from —O—, —O—C(O)—, —NH— and —S—(CH2)n-, with n representing an integer from 0 to 6,

and R4 represents a linear or branched alkyl radical containing at most 6 carbon atoms, a monocyclic or bicyclic carbocyclic or heterocyclic radical containing from 5 to 10 members, saturated or unsaturated, containing one or more identical or different heteroatoms chosen from O, N, NH or S and able to contain a —C(O) member,

the carbocyclic and heterocyclic alkyl radicals as defined above for R4, being optionally substituted by one or more radicals chosen from the halogen atoms; the hydroxyl radicals; free, salified or esterified carboxy radicals; —C(O)—NH2, —C(O)—NH(alkyl), —C(O)—N(alkyl)(alkyl), —NH—C(O)-(alkyl), —N(alkyl)-C(O)-(alkyl); thienyl; phenyl; alkylphenyl; alkyl and alkoxy radicals themselves optionally substituted by one or more radicals chosen from the acyl radicals containing at most 7 carbon atoms, cyano, —NH2, —NH(alkyl), —N(alkyl)(alkyl) and phenyl radicals, it being understood that in the above radicals, the alkyl and alkoxy radicals are linear or branched and contain at most 4 carbon atoms,

the * sign indicating the carbon atoms which can be in S or R configuration,

said products of formula (I) being in all possible racemic, enantiomeric and diastereoisomeric isomer forms, as well as the addition salts with mineral and organic acids or mineral and organic bases of said products of formula (I).

In the products of formula (I) and in the following:

the term linear or branched alkyl radical designates the methyl, ethyl, propyl, isopropyl, butyl, isobutyl, sec-butyl, tert-butyl, pentyl, isopentyl, hexyl, isohexyl and also heptyl, octyl, nonyl and decyl radicals as well as their linear or branched position isomers: among these alkyl radicals, the alkyl radicals which contain at most 6 carbon atoms or those which contain at most 4 carbon atoms can preferably be chosen

the term linear or branched alkoxy radical designates the methoxy, ethoxy, propoxy, isopropoxy, linear, secondary or tertiary butoxy radicals, pentoxy or hexoxy radicals as well as their linear or branched position isomers: among these alkoxy radicals, the alkoxy radicals which contain at most 6 carbon atoms or those which contain at most 4 carbon atoms can preferably be chosen

the term halogen atom designates chlorine, bromine, iodine or fluorine atoms and preferably the chlorine, bromine or iodine atom

the term cycloalkyl radical designates the cyclopropyl, cyclobutyl, cyclopentyl and cyclohexyl radicals and more particularly the cyclopentyl and cyclohexyl radicals,

the term monocyclic heterocyclic radical designates a saturated or unsaturated radical constituted by 5 or 6 members such that one or more of the members represents an oxygen, sulphur or nitrogen atom: such a heterocyclic radical therefore designates a carbocyclic radical interrupted by one or more heteroatoms chosen from oxygen, nitrogen or sulphur atoms it being understood that the heterocyclic radicals can contain one or more heteroatoms chosen from oxygen, nitrogen or sulphur atoms and that when these heterocyclic radicals comprise more than one heteroatom, the heteroatoms of these heterocyclic radicals can be identical or different. The following radicals can in particular be mentioned: dioxolane, dioxane, dithiolane, thiooxolane, thiooxane, morpholinyl, piperazinyl, piperazinyl substituted by a linear or branched alkyl radical, containing at most 4 carbon atoms, piperidyl, thienyl such as 2-thienyl and 3-thienyl, furyl such as 2-furyl, pyrimidinyl, pyridyl such as 2-pyridyl, 3-pyridyl and 4-pyridyl pyrimidyl, pyrrolyl, thiazolyl, isothiazolyl, diazolyl, thiadiazolyl, triazolyl, free or salified tetrazolyl, thiadiazolyl, thiatriazolyl, oxazolyl, oxadiazolyl, 3- or 4-isoxazolyl. The morpholinyl, thienyl such as 2-thienyl and 3-thienyl, furyl such as 2-furyl, tetrahydrofuryl, thienyl, tetrahydrothienyl, pyrrolyl, pyrrolinyl, pyridyl and pyrrolidinyl radicals can more particularly be mentioned.

the term bicyclic heterocyclic radical designates a saturated or unsaturated radical constituted by 8 to 12 members such that one or more of the members represents an oxygen, sulphur or nitrogen atom and in particular condensed heterocyclic groups containing at least one heteroatom chosen from sulphur, nitrogen and oxygen, for example benzothienyl such as 3-benzothienyl, benzothiazolyl, quinolyl, tetralone, benzofuryl, benzopyrrolyl, benzimidazolyl, benzoxazolyl, thionaphthyl, indolyl or purinyl.

The term carbocyclic or heterocyclic monocyclic or bicyclic radical containing from 5 to 10 members, saturated or unsaturated, containing one or more identical or different heteroatoms chosen from O, N, NH or S, and able to contain a —C(O) member, brings together the definitions indicated above on one hand for the term monocyclic heterocyclic radical and on the other hand for the term bicyclic heterocyclic radical, all of these radicals being optionally substituted. It should be noted that a carbocyclic radical as defined above is in particular the phenyl radical and that a carbocyclic radical containing a —C(O) member is for example the tetralone radical.

the term alkylphenyl designates a phenyl radical substituted by one or more linear or branched alkyl radicals as defined above preferably containing at most 4 carbon atoms the terms NH(alk) and N(alk)(alk) designate an amino radical substituted respectively by one or two alkyl radicals, such alkyl radicals being linear or branched and containing preferably at most 4 carbon atoms the term acylamino designates the —C(O)—NH2, —C(O)—NH(alk) and —C(O)—N(alk)(alk) radicals in which the NH(alk) and N(alk)(alk) radicals have the meaning indicated above

the term acyl designates an R—C(O)— radical in which R represents a radical chosen from the hydrogen atom, linear or branched alkyl radicals containing at most 6 carbon atoms, a phenyl radical or a pyrrolidinyl radical: the term acyl therefore designates in particular the formyl radicals, the acetyl, propionyl, butanoyl, pentanoyl, hexanoyl, benzoyl and pyrrolidinylcarbonyl radicals the term alkenyl designates linear or branched radicals containing at most 6 carbon atoms: the vinyl, 1-propenyl, allyl, butenyl, 3-methyl-2-butenyl radicals can in particular be mentioned

the term alkylthio designates linear or branched radicals containing at most 6 carbon atoms such as in particular the methylthio, ethylthio, propylthio, isopropylthio, butylthio, isobutylthio, sec-butylthio, tert-butylthio, pentylthio, isopentylthio, hexylthio or also isohexylthio radicals as well as their linear or branched position isomers: among these alkylthio radicals, a choice is preferably made from those mentioned above, those which contain at most 4 carbon atoms

The so-called natural or non-natural amino acids are known to a person skilled in the art and can be found in standard documents such as for example the document ‘Amino Acids (1999), 16(3-4), 345-379.

The carboxy radical(s) of the products of formula (1) can be salified or esterified by the various groups known to a person skilled in the art among which can be mentioned, for example:

among the salification compounds, mineral bases such as, for example, an equivalent of sodium, potassium, lithium, calcium, magnesium or ammonium or organic bases such as, for example, methylamine, propylamine, trimethylamine, diethylamine, triethylamine, N,N-dimethylethanolamine, tris (hydroxymethyl)amino methane, ethanolamine, pyridine, picoline, dicyclohexylamine, morpholine, benzylamine, procaine, lysine, arginine, histidine, N-methyl-glucamine,

among the esterification compounds, the alkyl radicals in order to form alkoxy carbonyl groups such as, for example, methoxycarbonyl, ethoxycarbonyl, tert-butoxycarbonyl or benzyloxycarbonyl, these alkyl radicals being able to be substituted by radicals chosen for example from halogen atoms, the hydroxyl, alkoxy, acyl, acyloxy, alkylthio, amino or aryl radicals such as, for example; in the chloromethyl, hydroxypropyl, methoxymethyl, propionyloxymethyl, methylthiomethyl, dimethylaminoethyl, benzyl or phenethyl groups.

The addition salts with mineral or organic acids of the products of formula (1) can be, for example, the salts formed with the hydrochloric, hydrobromic, hydroiodic, nitric, sulphuric, phosphoric, propionic, acetic, trifluoroacetic, formic, benzoic, maleic, fumaric, succinic, tartaric, citric, oxalic, glyoxylic, aspartic, ascorbic acids, the alkylmonosulphonic acids such as for example methanesulphonic acid, ethanesulphonic acid, propanesulphonic acid, alkyldisulphonic acids such as for example methanedisulphonic acid, alpha, beta-ethanedisulphonic acid, arylmonosulphonic acid such as benzenesulphonic acid and aryldisulphonic acids. It should be remembered that stereoisomerism can be defined in its broadest sense as the isomerism of compounds having the same structural formulae, but the different groups of which are arranged differently in space, such as in particular in monosubstituted cyclohexanes the substituent of which can be in the axial or equatorial position, and the different possible rotational configurations of ethane derivatives. However, there is another type of stereoisomerism, due to the different spatial arrangements of fixed substituents, either on double bonds or on rings, which is often called geometric isomerism or cis-trans isomerism. The term stereoisomers is used in the present application in its broadest sense and therefore relates to all of the compounds indicated above.

It should be noted that the products of formula (I) as defined above in which the carbon atoms indicated by the * sign are under the stereochemical configuration S are preferred.

It should be noted that in the products of formula (I) as defined above, the third carbon atom indicated by the * sign in the formula of the products of formula (I), obviously does not have any particular R or S stereochemistry when R2 represents the hydrogen atom.

A more particular subject of the invention is therefore the products of formula (I) as defined above in which R1 and R3 have the meanings indicated above and R2 represents a so-called natural amino acid remainder with the exception of aspartic acid,

said products of formula (I) being in all possible racemic, enatiomeric and diastereoisomeric isomer forms, as well as the addition salts with mineral and organic acids or mineral and organic bases of said products of formula (I).

Therefore a more particular subject of the present invention is also the products of formula (I) as defined above in which R1 and R3 have the meanings indicated above and R2 and R7 are such that:

either R7 represents a hydrogen atom and R2 represents a hydrogen atom or a linear or branched alkyl radical containing at most 6 carbon atoms optionally substituted by one or more radicals chosen from the amino; acylamino; NH═C(NH2)—NH—; mercapto; linear or branched alkylthio radicals containing at most 4 carbon atoms; linear or branched hydroxyl; alkoxyl radicals containing at most 4 carbon atoms; imidazolyl; indolyl radicals; the phenyl radical itself optionally substituted by one or more radicals chosen from halogen atoms, the hydroxyl radical, a linear or branched alkoxy radical containing at most 4 carbon atoms and the phenoxy radical itself optionally substituted by one or more radicals chosen from iodine atoms and the hydroxyl radical; and the —C(O)—O—R8 radical in which R8 represents a hydrogen atom or a linear or branched alkyl or alkenyl radical containing at most 6 carbon atoms,

or R2 and R7 form together with the nitrogen and carbon atoms to which they are linked a ring comprising 5 to 7 members optionally substituted by one or more hydroxyl or linear or branched alkoxy radicals containing at most 4 carbon atoms, said products of formula (I) being in all possible racemic, enatiomeric and diastereoisomeric isomer forms, as well as the addition salts with mineral and organic acids or mineral and organic bases of said products of formula (I).

A particular subject of the present invention is the products of formula (I) as defined above in which R1 and R3 have the meanings indicated above and R2 and R7 are such that:

either R7 represents a hydrogen atom and R2 has the meaning indicated above

or R2 and R7 form together with the nitrogen and carbon atoms a pyrrolidinyl ring optionally substituted by one or more hydroxyl or linear or branched alkoxy radicals containing at most 4 carbon atoms,

said products of formula (I) being in all possible racemic, enatiomeric and diastereoisomeric isomer forms, as well as the addition salts with mineral and organic acids or mineral and organic bases of said products of formula (I).

The products of formula (I) as defined above can in particular be mentioned in which R2 and R7 form together with the nitrogen and carbon atoms to which they are linked a pyrrolidinyl ring, the amino acid remainder then being proline, or a pyrrolidinyl ring substituted by an optionally protected hydroxyl radical, the amino acid remainder then being hydroxyproline.

A particular subject of the present invention is the products of formula (I) as defined above corresponding to formula (Ia):

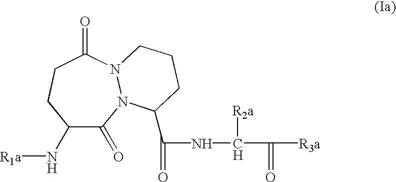

in which:

R₁ₐ represents the —C(O)—R5a, —SO2—R5a or —C(O)—NR6aR5a radical

in which R6a represents a hydrogen atom or a linear or branched alkyl radical containing at most 4 carbon atoms and R5a represents a radical chosen from the linear or branched alkyl radicals containing at most 6 carbon atoms; cycloalkyl containing at most 6 carbon atoms; phenyl; naphthyl; furyl; morpholinyl; thienyl; pyridyl,

the alkyl, phenyl, thienyl and pyridyl radicals being optionally substituted by one or more radicals chosen from the halogen atoms and the linear or branched alkyl or alkoxy radicals containing at most 4 carbon atoms, hydroxyl, acyl containing at most 7 carbon atoms, phenoxy, trifluoromethyl, cyano, thienyl, phenyl and isoxazolyl radicals;

R2a has the meaning indicated above when R7 represents a hydrogen atom,

R3a represents the —CH═N2 radical or the —CH2-La-R4a radical in which La represents a single bond or a divalent radical chosen from —O—, —O—C(O)— and —S—(CH2)na-, with na representing an integer from 0 to 3,

and R4a represents a radical chosen from the linear or branched alkyl radicals containing at most 6 carbon atoms; the phenyl; tetrazolyl; piperazinyl; piperidyl; pyridyl; benzothiazolyl; quinolyl; thiadiazolyl; pyrimidinyl; tetralone radicals;

the alkyl, phenyl, tetrazolyl, piperazinyl and piperidyl radicals as defined above for R4a being optionally substituted by one or more radicals chosen from the halogen atoms; the hydroxyl, free, salified or esterified carboxy radicals;

—C(O)—NH2; —C(O)—NH(alkyl); —C(O)—N(alkyl)(alkyl); —NH—C(O)-(alkyl); —N(alkyl)-C(O)-(alkyl); thienyl; phenyl; alkylphenyl; alkyl and alkoxy radicals themselves optionally substituted by one or more radicals chosen from the acyl radicals containing at most 7 carbon atoms, cyano, —NH2, —NH(alkyl), —N(alkyl)(alkyl) and phenyl radicals, it being understood that in the above radicals, the alkyl and alkoxy radicals are linear or branched and contain at most 4 carbon atoms,

said products of formula (Ia) being in all possible racemic, enatiomeric and diastereoisomeric isomer forms, as well as the addition salts with mineral and organic acids or mineral and organic bases of said products of formula (Ia).

A more particular subject of the present invention is the products of formula (I) as defined above corresponding to formula (Ib):

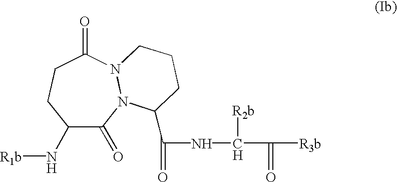

in which:

R_{1b} represents the —C(O)—R5b, —SO2—R5b or —C(O)—NR6bR5b radical

in which R6b represents a hydrogen atom or a methyl radical and R5b represents a radical chosen from the linear or branched alkyl radicals containing at most 4 carbon atoms optionally substituted by a thienyl; cyclohexyl; phenyl radical optionally substituted by a linear or branched alkoxy radical containing at most 4 carbon atoms, a phenoxy or trifluoromethyl; naphthyl; furyl; morpholinyl; thienyl optionally substituted by a radical isoxazolyl; pyridyl optionally substituted by a trifluoromethyl radical R2b represents a hydrogen atom or a linear or branched alkyl radical containing at most 6 carbon atoms optionally substituted either by the phenyl radical itself optionally substituted by the hydroxyl or linear or branched alkoxy radicals containing at most 4 carbon atoms, or by the —C(O)—O—R8b radical in which R8b represents a linear or branched alkyl or alkenyl radical containing at most 6 carbon atoms,

R3b represents the —CH═N2 radical or the —CH2-Lb-R4b radical in which Lb represents a single bond or a divalent radical chosen from —O—, —O—C(O)— and —S—(CH2)nb-, with nb representing the integer 0 or 1,

and R4b represents a radical chosen from the pyridyl; benzothiazolyl; quinolyl; thiadiazolyl; pyrimidinyl; tetralone radicals; the linear or branched alkyl radicals containing at most 4 carbon atoms optionally substituted by a free, salified or esterified carboxy or thienyl radical; phenyl optionally substituted by one or more radicals chosen from the halogen atoms, the free, salified or esterified carboxy radicals, the linear or branched alkyl and alkoxy radicals containing at most 4 carbon atoms, cyanoalkyl, alkylphenyl, —NH—C(O)—CH3, —C(O)—N(alkyl)(alkyl) radicals; tetrazolyl optionally substituted by a phenyl radical; piperazinyl optionally substituted on the second nitrogen atom by a phenyl radical or a linear or branched alkyl containing at most 4 carbon atoms itself optionally substituted by an acyl (pyrrolidinylcarbonyl) radical, one or two phenyl radicals or an —NH2, —NH(alkyl) or —N(alkyl)(alkyl); piperidyl radical optionally substituted by a benzyl or —C(O)—N(alkyl)(alkyl) radical, it being understood that in the above radicals, the alkyl and alkoxy radicals are linear or branched and contain at most 4 carbon atoms,

said products of formula (Ib) being in all possible racemic, enatiomeric and diastereoisomeric isomer forms, as well as the addition salts with mineral and organic acids or mineral and organic bases of said products of formula (Ib).

A subject of the present invention is especially the products of formula (I) as defined above in which R1 represents a radical chosen from the following radicals:

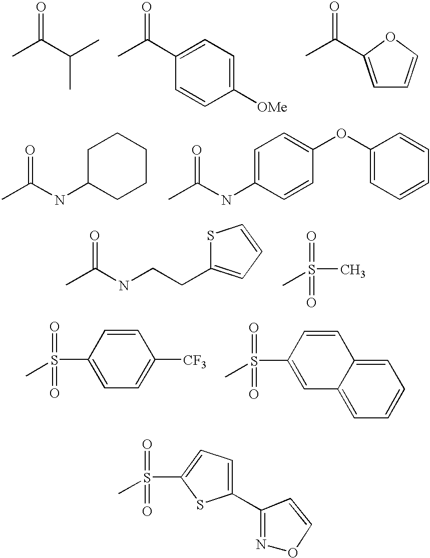

R2, R3 and R7 having the values indicated above, those in which R7 represents a hydrogen atom and R2 represents a radical chosen from the following radicals:

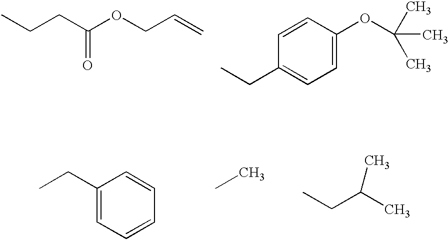

R1 and R3 having the values indicated above and those in which R3 represents a radical chosen from the following radicals:

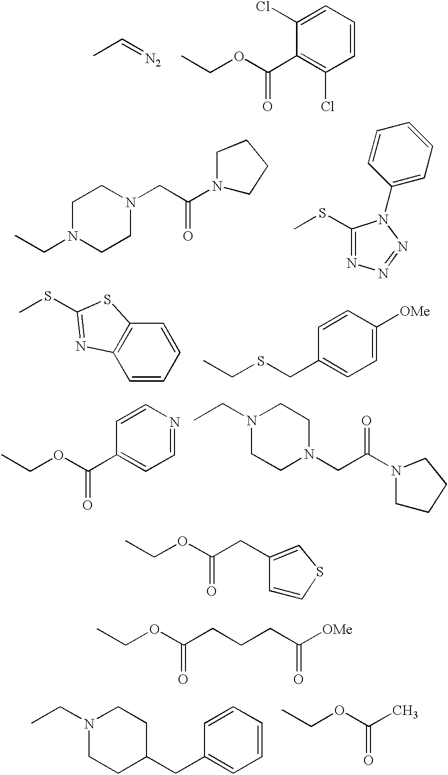

R1, R2 and R7 having the values indicated above, said products of formula (Ib) being in all possible racemic, enatiomeric and diastereoisomeric isomer forms, as well as the addition salts with mineral and organic acids or mineral and organic bases of said products of formula (Ib).

In the products of formula (I) of the present invention, R₂ in particular represents an amino acid remainder chosen from the natural amino acids: GLU, GLY, ALA, VAL, LEU, PHE, TYR or TYR(OtBU). TYR(OtBU) represents the amino acid TYR in which the hydroxyl radical is protected as the terbutoxy radical.

A quite particular subject of the present invention is the products of formula (I) as defined above, corresponding to the following formulae:

3-[9(S)-benzoylamino-6,10-dioxo-1,2,3,4,7,8,9,10-octahydro-6H-pyridazino[1,2-a][1,2]diazepine-1(S)-carboxamide]-5-methyl-1-benzoyloxy-hexane-2-one

3-[9(S)-(2-furanoyl)amino-6,10-dioxo-1,2,3,4,7,8,9,10-octahydro-6H-pyridazino[1,2-a][1,2]diazepine-1(S)-carboxamide]-5-methyl-1-(2,6-dichlorobenzoyloxy-hexane-2-one

3(S)-[9(S)-(2-furanoyl)amino-6,10-dioxo-1,2,3,4,7,8,9,10-octahydro-6H-pyridazino[1,2-a][1,2]diazepine-1(S)-carboxamide]-4-(4-hydroxyphenyl)-1-(2,6-dichlorobenzoyloxy)-butane-2-one

A quite particular subject of the present invention is also the products of formula (I) as defined above corresponding to the following formulae:

(2-propenyl)(4S)6-diazo-4-[[[(1S,9S)-9-[(2-methyl-1-oxo-propyl)amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[(3-methoxybenzoyl)amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[(2-furanyl)carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[(cyclohexylamino) carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[[2-(p-2-yl)-ethyl]amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[(methylsulphonyl)amino]-octahydro-6,10-dioxo-6H-pyridazino[1,2-a][l,2]diazepine-1-yl]carbonyl]amino]-5-oxo-hexanoate

(2-propenyl)(4S)6-diazo-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[(2-naphthalenyl) sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[5-(isoxazol-3-yl)-2-thienyl]sulphonyl]amino]-octahydro-6,10-dioxo-6H-pyridazino[1,2-a][1,2]diazepine-1-yl]carbonyl]amino]-5-oxo-hexanoate

9-[(9S)-[[(4-phenoxyphenyl)amino]carbonyl]amino]-N-[(2S)-4-diazo-1-[4-[(1,1-dimethyl)ethoxy]phenyl]-3-oxo-2-butyl]-6H-(1S)-pyridazino[1,2-a][1,2]diazepine-1-carboxamide

(2-propenyl)(4S)6-[(2,6-dichlorobenzoyl)oxy]-4-[[[(1S,9S)-9-[[(2-furanyl)carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-[4-[[2-oxo-2-(1-pyrrolidinyl)ethyl]piperazin-1-yl]-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-[4-[[2-oxo-2-(1-pyrrolidinyl)ethyl]piperazin-1-yl]-4-[[[(1S,9S)-9-[[(2-naphthalenyl)sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[(cyclohexylamino)carbonyl]amino]-octahydro-6,10-dioxo-o-6H-pyridazino[1,2-a][1,2]diazepine-1-yl]carbonyl]amino]-5-oxo-hexanoate

(2-propenyl)(4S)6-[(1-phenyl-1H-tetrazol-5-yl)thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(benzothiazol-2-yl)thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[[(4-methoxyphenyl)methyl]thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[[(4-pyridinyl)carbonyl]oxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)((4S)6-acetyloxy-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[1-oxo-2-(thien-3-yl)ethoxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(1-phenyl-1H-tetrazol-5-yl)thio]-4-[[[(1S,9S)-⁹-[[[[²-(thien-2-yl)-ethyl]amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[[[2-(thien-2-yl)-ethyl]amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[(methylsulphonyl)amino]-octahydro-6,10-dioxo-6H-pyridazino[1,2-a][1,2]diazepine-1-yl]carbonyl]amino]-5-oxo-hexanoate

9-[(9S)[[(4-phenoxyphenyl)amino]carbonyl]amino]-N-((2S)-4-diazo-1-phenyl-3-oxo-2-butyl)-octahydro-6,10-dioxo -6H-(1S)-pyridazino[1,2-a][1,2]diazepine-1-carboxamide

9-[(9S)-[[[2-(thien-2-yl)-ethyl]amino]carbonyl]amino]-N-((2S)-4-diazo-1-phenyl-3-oxo-2-butyl)-octahydro-6,10-dioxo-6H-(1S)-pyridazino[1,2-al[1,2]diazepine-1-carboxamide

9-[(9S)-[(2-naphthalenyl)sulphonyl]amino ]-N-( (2S)-4-diazo-1-phenyl-3-oxo-2-butyl)-octahydro-6,10-dioxo-6H-(1S)-pyridazino[1,2-a][1,2]diazepine-1-carboxamide

9-[(9S)-[[5-(isoxazol-3-yl)-2-thienyl]sulphonyl]amino]-N-((2S)-4-diazo-1-phenyl-3-oxo-2-butyl)-octahydro-6,10-dioxo-6H-(1S)-pyridazino[1,2-a][1,2]diazepine-1-carboxamide

9-[(9S)-[[(4-phenoxyphenyl)amino]carbonyl]amino]-N-((2S)-4-diazo-3-oxo-2-butyl)-octahydro-6,10-dioxo -6H-(1S)-pyridazino[1,2-a][1,2]diazepine-1-carboxamide

9-[(9S)-[[[3-(thien-2-yl)-ethyl]amino]carbonyl]amino]-N-((2S)-4-diazo-3-oxo-2-butyl)-octahydro-6,10-dioxo -6H-(1S)-pyridazino[1,2-a][1,2]diazepine-1-carboxamide

9-[(9S)-(3-methoxybenzoyl)amino]-N-((2S)-4-[(2,6-dichlorobenzoyl)oxy]-1-phenyl-3-oxo-2-butyl]-octahydro-6,10-dioxo-6H-(1S)-pyridazino[1,2-a][1,2]diazepine-1-carboxamide

9-[(9S)-[(2-furanyl)carbonyl]amino]-N-[(2S)-4-[(2,6-dichlorobenzoyl)oxy]-1-phenyl-3-oxo-2-butyl]-octahydro-6,10-dioxo-6H-(1S)-pyridazino[1,2-a][1,2]diazepine-1-carboxamide

9 L(9S)-[(cyclohexylamino)carbonyl]amino]-N-[(2S)-4-[(2,6-dichlorobenzoyl)oxy]-1-phenyl-3-oxo-2-butyl]-octahydro-6,10-dioxo-6H-(1S)-pyridazino[1,2-a][1,2]diazepine-1-carboxamide

9-[(9S)-[[(4-phenoxyphenyl)amino]carbonyl]amino]-N-[(2S)-1-phenyl-4-[(1-phenyl-1H-tetrazol-5-yl)thio]-3-oxo-2-butyl]-octahydro-6,10-dioxo-6H-(1S)-pyridazino[1,2-a][1,2]diazepine-1-carboxamide

9-[(9S)-[[(4-phenoxyphenyl)amino]carbonyl]amino]-N-[(2S)-1-phenyl-4-[1-oxo-2-(3-thienyl)ethoxy]-3-oxo-2-butyl]-octahydro-6,10-dioxo-6H-(1S)-pyridazino[1,2-a][1,2]diazepine-1-carboxamide

(methyl)[2-oxo-4-phenyl-3[(3S)-[[(4-phenoxyphenyl)amino]carbonyl]amino]-octahydro-6,10-dioxo-6H-(1S)-pyridazino[1,2-a][1,2]diazepine-1-yl]carbonyl]amino]butyl]pentanedioate

9-[(9S)-[[(4-phenoxyphenyl)amino]carbonyl]amino]-N-[(2S)-4-[(4-phenylmethyl)-1-piperidinyl ]-1-phenyl-3-oxo-2-butyl]-octahydro-6,10-dioxo-6H-(1S)-pyridazino[1,2-a][1,2]diazepine-1-carboxamide

9-[(9S)-[[[2-(thien-2-yl)-ethyl]amino]carbonyl]amino]-N-[(2S)-1-phenyl-4-[(1-phenyl-1H-tetrazol-5-yl)thio]-3-oxo-2-butyl]-octahydro-6,10-dioxo-6H-(1S)-pyridazino [1,2-a][1,2]diazepine-1-carboxamide

9-[(9S)-[[[2-(thien-2-yl)-ethyl]amino]carbonyl]amino]-N-[(2S)-1-phenyl-4-[(2-benzothiazolyl)thio]-3-oxo-2-butyl]-octahydro-6,10-dioxo-6H-(1S)-pyridazino[1,2-a][1,2]diazepine-1-carboxamide

9-[(9S)-[[[2-(thien-2-yl)-ethyl]amino]carbonyl]amino]-N-[(2S)-1-phenyl-4-[(-2,6-dichlorobenzoyl)oxy]-3-oxo-2-butyl]-octahydro-6,10-dioxo-6H-(1S)-pyridazino[1,2-a][1,2]diazepine-1-carboxamide

9-[(9S)-[[[2-(thien-2-yl)-ethyl]amino]carbonyl]amino]-N-[(2S)-1-phenyl-4-[4-[2-(1-pyrrolidinyl)-2-oxo-ethyl]piperazin-1-yl]]-3-oxo-2-butyl]-octahydro-6,10-dioxo-6H-(1S)-pyridazino[1,2-a][1,2]diazepine-1-carboxamide

9-[(9S)-(methylsulphonyl)amino]-N-[(2S)-1-phenyl-4-[(-2,6-dichlorobenzoyl)oxy]-3-oxo-2-butyl]-octahydro-6,10-dioxo-6H-(1S)-pyridazino[1,2-a][1,2]diazepine-1-carboxamide

9-[(9S)-[[[4-(trifluoromethyl)phenyl]sulphonyl]amino]-N-[(2S)-1-phenyl-4-[(1-phenyl-1H-tetrazol-5-yl)thio]-3-oxo-2-butyl]-octahydro-6,10-dioxo-6H-(1S)-pyridazino [1,2-a][1,2]diazepine-1-carboxamide

9-[(9S)-[[[4-(trifluoromethyl)phenyl]sulphonyl]amino]-N-[(2S)-1-phenyl-4-(2-benzothiazolyl)thio]-3-oxo-2-butyl]-octahydro-6,10-dioxo-6H-(1S)-pyridazino[1,2-a][1,2]diazepine-1-carboxamide

9-[(9S)-(methylsulphonyl)amino]-N-[(3S)-5-methyl-1-[(2,6-dichlorobenzoyl)oxy]-2-oxo-3-hexyl]-octahydro-6,10-dioxo-6H-(1S)-pyridazino[1,2-a][1,2]diazepine-1-carboxamide

9-[(9S)-[[[4-(trifluoromethyl)phenyl]sulphonyl]amino]]-N-[(3S)-5-methyl-1-[(2,6-dichlorobenzoyl)oxy]-2-oxo-3-hexyl]-octahydro-6,10-dioxo-6H-(1S)-pyridazino[1,2-a][1,2]diazepine-1-carboxamide

9-[(9S)-[(2-naphthalenyl)sulphonyl]amino ]-N-[(3S)-5-methyl-1-[(2,6-dichlorobenzoyl)oxy]-2-oxo-3-hexyl]-octahydro-6,10-dioxo-6H-(1S)-pyridazino[1,2-a][1,2]diazepine-1-carboxamide

9-[(9S)-(2-methyl-1-oxo-propyl)amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-(3-methoxybenzoyl)amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-[(2-furanyl)carbonyl]amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-[(cyclohexylamino)carbonyl]amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-[[(4-phenoxyphenyl)amino]carbonyl]amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-[[[2-(thien-2-yl)-ethyl]amino]carbonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide

9-[(9S)-(methylsulphonyl)amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-[[4-(trifluoromethyl)phenyl]sulphonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide

9-[(9S)-[(2-naphthalenyl)sulphonyl]amino]-N-((3S)-1-diazo-5-methyl-2-oxo-3-hexyl)-octahydro-6,10-dioxo -6H-(1S)-pyridazino[1,2-a][1,2]diazepine-1-carboxamide

9-[(9S)-(3-methoxybenzoyl)amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide

9-[(9S)-[(2-furanyl)carbonyl]amino]-N-[(3S)-1-[(benzothiazol-2-yl)thio]-4-methyl-2-oxo-3-pentyl]-octahydro-6,10-dioxo -6H-(1S)-pyridazino[1,2-a][1,2]diazepine-1-carboxamide

9-[(9S)-[(cyclohexylamino)carbonyl]amino]-N-[(3S)-1-[(benzothiazol-2-yl)thio]-4-methyl-2-oxo-3-pentyl]-octahydro-6,10-dioxo -6H-(1S)-pyridazino[1,2-a][1,2]diazepine-1-carboxamide

9-[(9S)-[[(4-phenoxyphenyl)amino]carbonyl]amino]-N-[(3S)-1-[(1-phenyl-1H-tetrazol-5-yl)thio]-4-methyl-2-oxo-3-pentyl]-octahydro-6,10-dioxo -6H-(1S)-pyridazino [1,2-a][1,2]diazepine-1-carboxamide

9-[(9S)-[[(4-phenoxyphenyl)amino]carbonyl]amino]-N-[3S)-1-[(benzothiazol-2-yl)thio]-4-methyl-2-oxo-3-pentyl]-octahydro-6,10-dioxo -6H-(1S)-pyridazino[1,2-a][1,2]diazepine-1-carboxamide

9-[(9S)-[[[2-(thien-2-yl)-ethyl]amino]carbonyl]amino]-N-[(3S)-1-[(1-phenyl-1H-tetrazol-5-yl)thio]-4-methyl-2-oxo-3-pentyl]-octahydro-6,10-dioxo -6H-(1S)-pyridazino [1,2-a][1,2]diazepine-1-carboxamide

9-[(9S)-[[[2-(thien-2-yl)-ethyl]amino]carbonyl]amino]-N-[(3S)-1-[(benzothiazol-2-yl)thio]-4-methyl-2-oxo-3-pentyl]-octahydro-6,10-dioxo -6H-(1S)-pyridazino[1,2-a][1,2]diazepine-1-carboxamide

9-[(9S)-[(cyclohexylamino)carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide

9-[(9S)-[(2-furanyl)carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide

9-[(9S)-[[(4-phenoxyphenyl)amino]carbonyl]amino]-N-[(2S)-4-[(benzothiazol-2-yl)thio]-3-oxo-2-butyl]-octahydro-6,10-dioxo -6H-(1S)-pyridazino[1,2-a][1,2]diazepine-1-carboxamide

9-[(9S)-[[(4-phenoxyphenyl)amino]carbonyl]amino]-N-[(2S)-4-[(2,6-dichlorobenzoyl)oxy]-3-oxo-2-butyl]-octahydro-6,10-dioxo -6H-(1S)-pyridazino[1,2-a][1,2]diazepine-1-carboxamide

9-[(9S)-(3-methoxybenzoyl)amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide

9-[(9S)-[[[2-(thien-2-yl)-ethyl]amino]carbonyl]amino]-N-[(2S)-4-[(2,6-dichlorobenzoyl)oxy]-3-oxo-2-butyl]-octahydro-6,10-dioxo -6H-(1S)-pyridazino[1,2-a][1,2]diazepine-1-carboxamide

9-[(9S)-[[4-(trifluoromethyl)phenyl]sulphonyl]amino]-N-[(2S)-4-[(2,6-dichlorobenzoyl)oxy]-3-oxo-2-butyl]-octahydro-6,10-dioxo -6H-(1S)-pyridazino[1,2-a][1,2]diazepine-1-carboxamide

9-[(9S)-[(cyclohexylamino)carbonyl]amino]-N-[(2S)-4-[(benzothiazol-2-yl)thio]-1-[4-[(1,1-dimethyl) ethoxy]phenyl]-3-oxo-2-butyl]-6H-(1S)-pyridazino[1,2-a][1,2]diazepine-1-carboxamide.

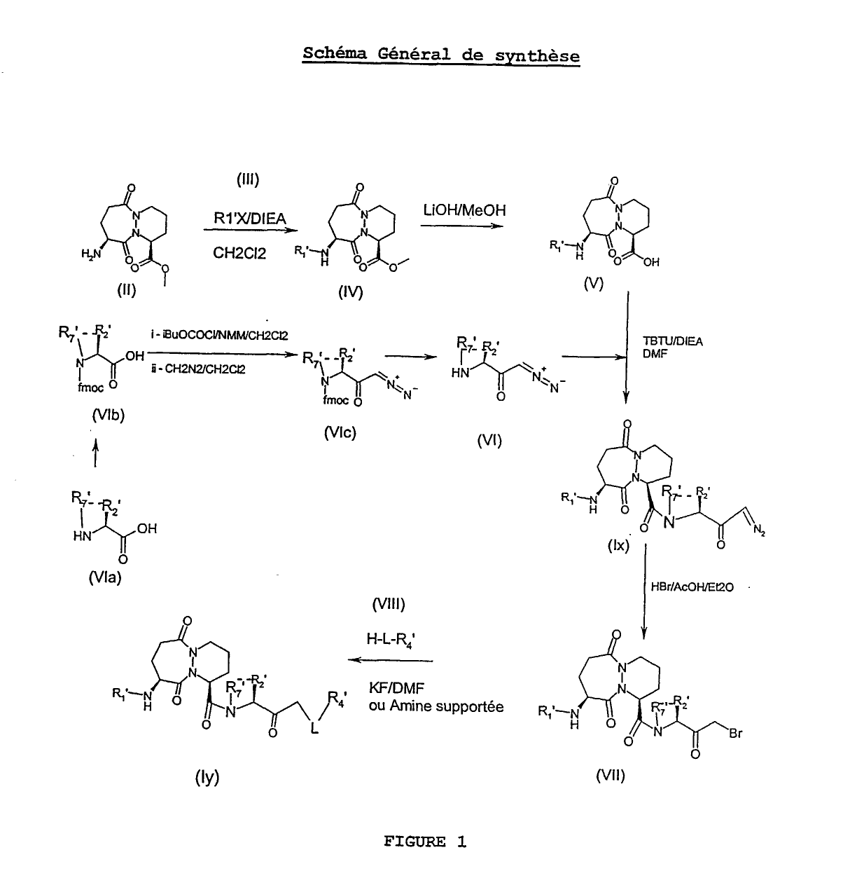

The products of formula (I) according to the present invention can be prepared according to the general synthesis diagram described in FIG. 1 of the present invention.

Therefore a subject of the present invention is also therefore a process for the preparation of the products of formula (I), as defined above, characterized in that the compound of formula (II):

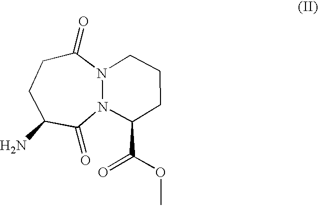

is subjected to a reaction with a compound of formula (III):

R1′-X (III)

in which X represents a halogen atom and R1′ has the meaning indicated above for R1, in which the optional reactive functions are optionally protected by protective groups, in order to obtain the product of formula (IV):

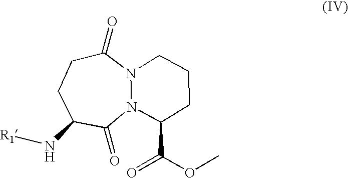

in which R1′ has the meaning indicated above, which product of formula (IV) is subjected to a saponification reaction in order to obtain the product of formula (V):

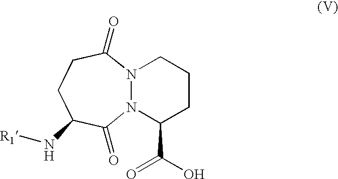

in which R1′ has the meaning indicated above, which product of formula (V) is subjected to a reaction with a product of formula (VI):

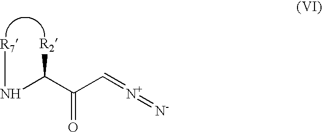

in which R2′ and R7′ have the meanings indicated above respectively for R2 and R7, in which the optional reactive functions are optionally protected by protective groups, in order to obtain a product of formula (Ix):

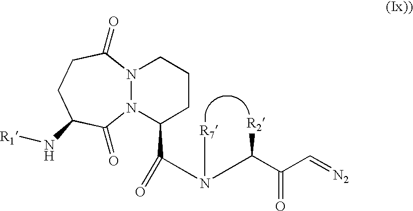

in which R1′, R2′ and R7′ have the meanings indicated above, which product of formula (Ix) can be subjected to a bromination reaction in order to obtain a product of formula (VII):

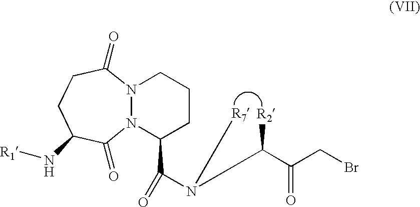

in which R1′, R2′ and R7′ have the meanings indicated above, which product of formula (VII) is subjected to a reaction with a product of formula (VIII):

H′—L—R4′ (VIII)

in which L has the meaning indicated above and R4′ has the meaning indicated above for R4, in which the optional reactive functions are optionally protected by protective groups,

in order to obtain a product of formula (Iy):

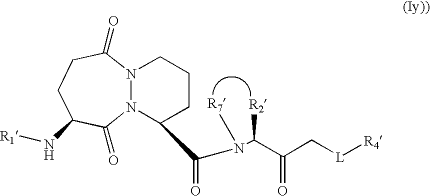

in which R1′, R2′, R4′ and R7′ have the meanings indicated above,

which products of formulae (Ix) and (Iy) can be products of formula (I) and which, in order to obtain other products of formula (I), can be subjected, if desired and if necessary, to one or more of the following conversion reactions, in any order:

a) an esterification reaction of the acid function,

b) a saponification reaction of the ester function to an acid function,

c) an oxidation reaction of the alkylthio group to a corresponding sulphoxide or sulphone,

d) a conversion reaction of the ketone function to an oxime function,

e) a reduction reaction of the free or esterified carboxy function to an alcohol function,

f) a conversion reaction of the alkoxy function to a hydroxyl function, or also of the hydroxyl function to an alkoxy function,

g) an oxidation reaction of the alcohol function to an aldehyde, acid or ketone function,

h) a conversion reaction of the nitrile radical to a tetrazolyl,

i) an elimination reaction of the protective groups which can be carried by the protected reactive functions,

j) a salification reaction by a mineral or organic acid or by a base in order to obtain the corresponding salt,

k) a resolution reaction of the racemic forms to resolved products, said products of formula (I) obtained in this way being in all possible racemic, enatiomeric and diastereoisomeric isomer forms.

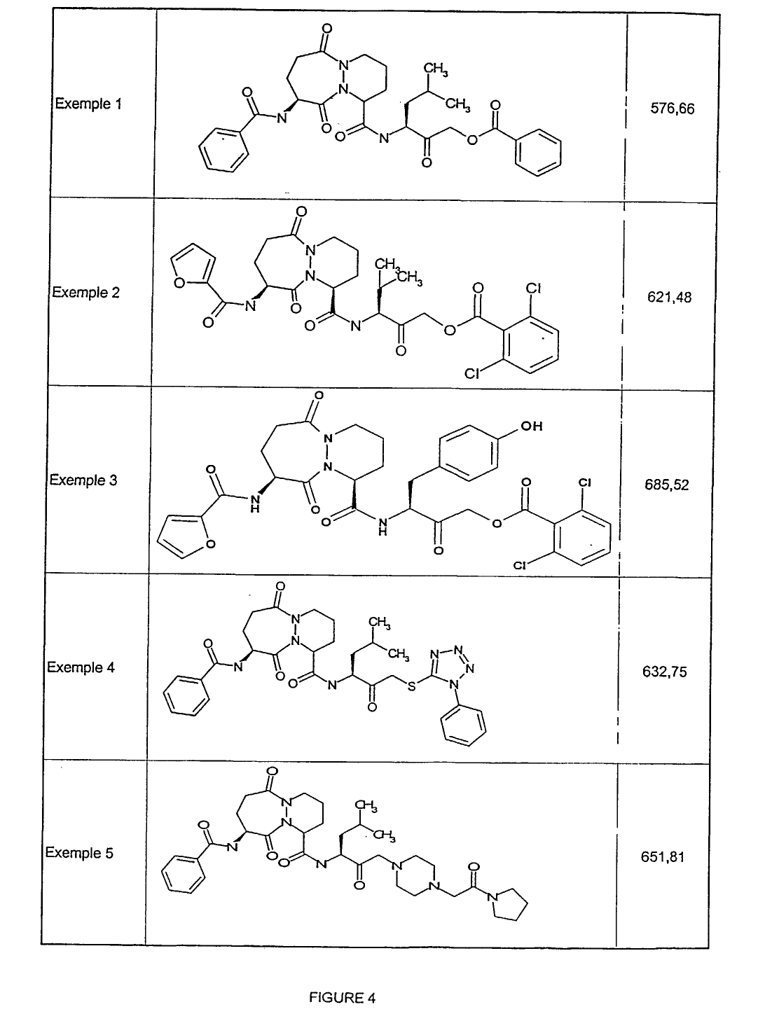

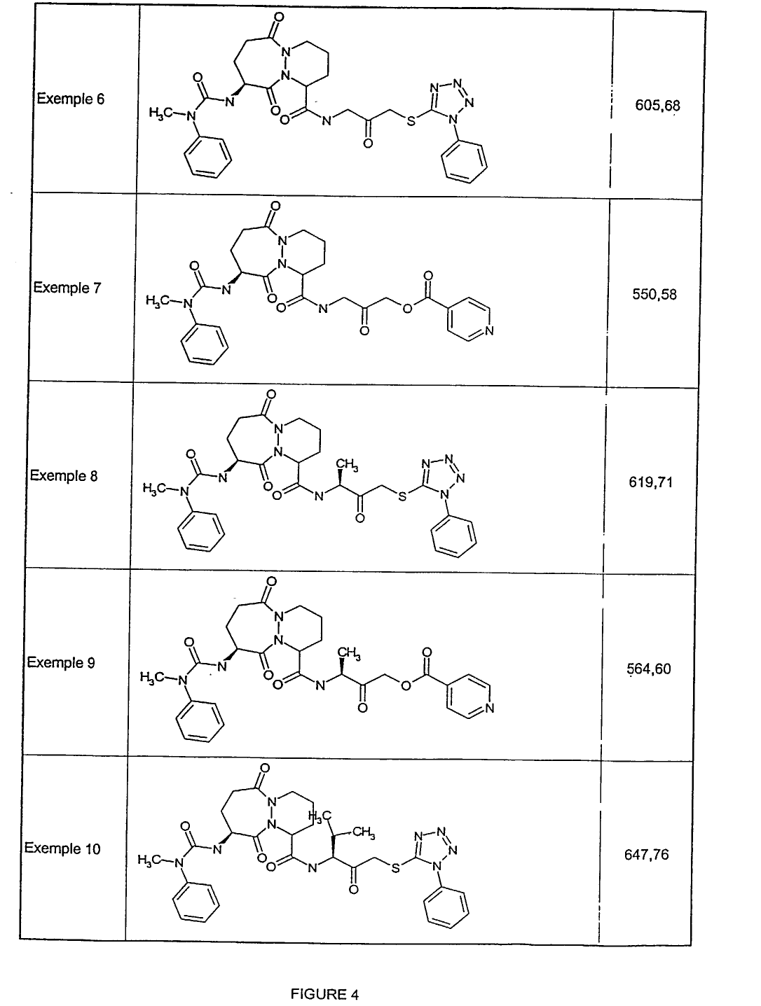

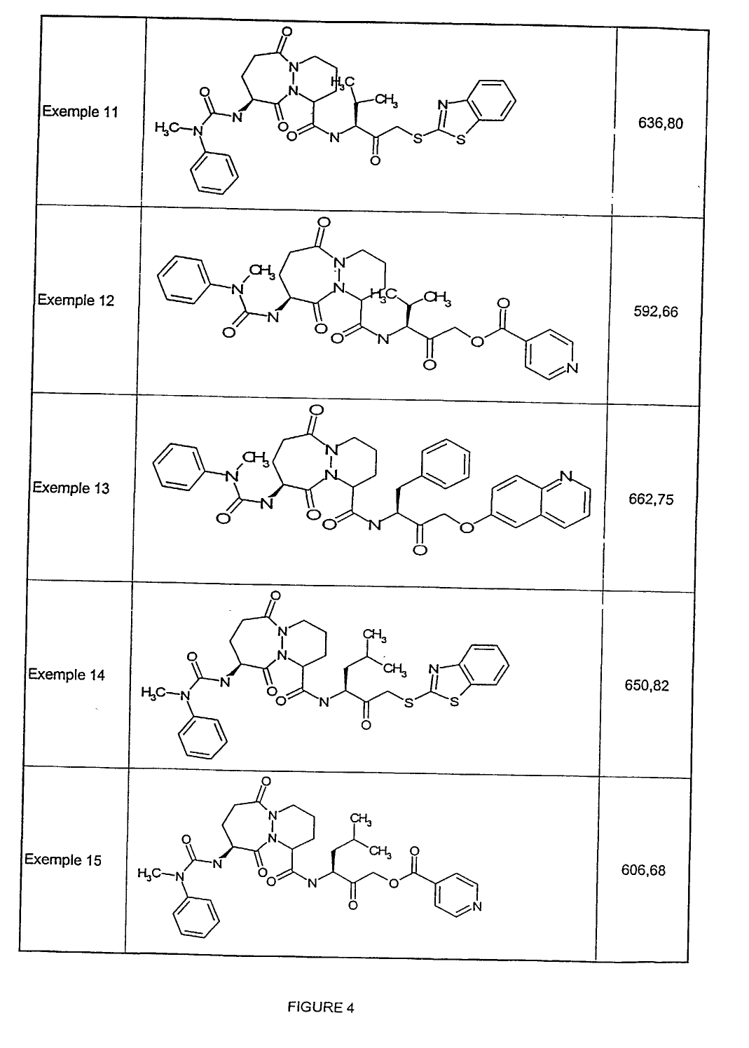

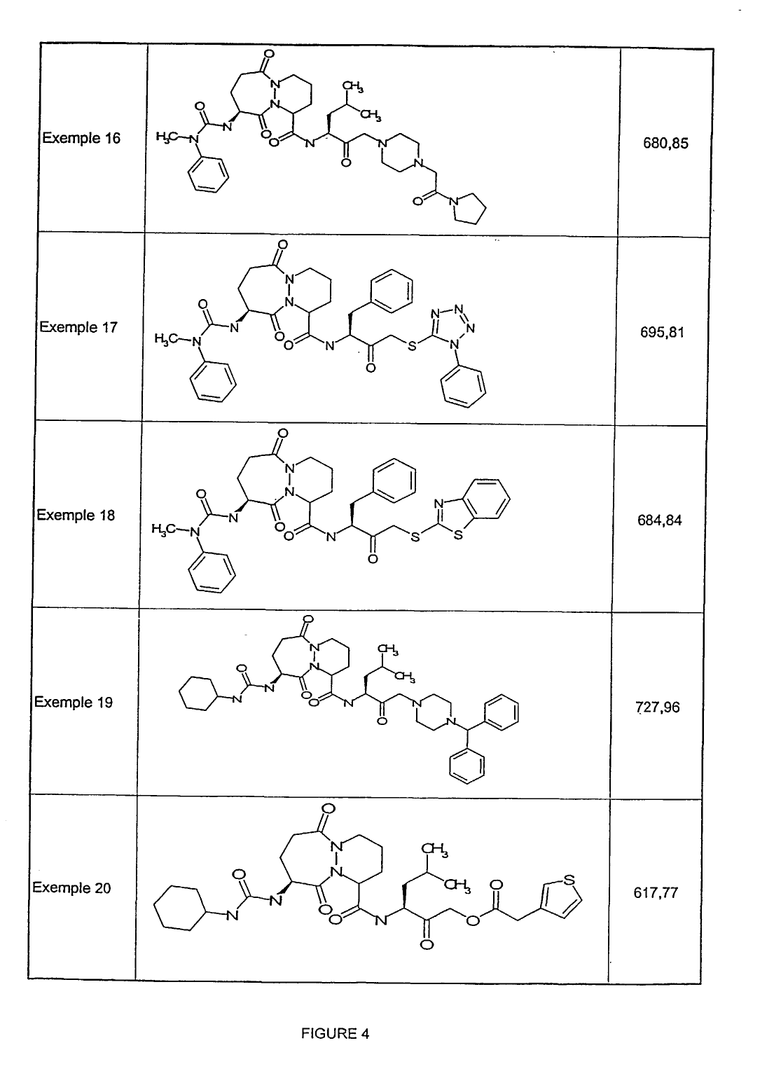

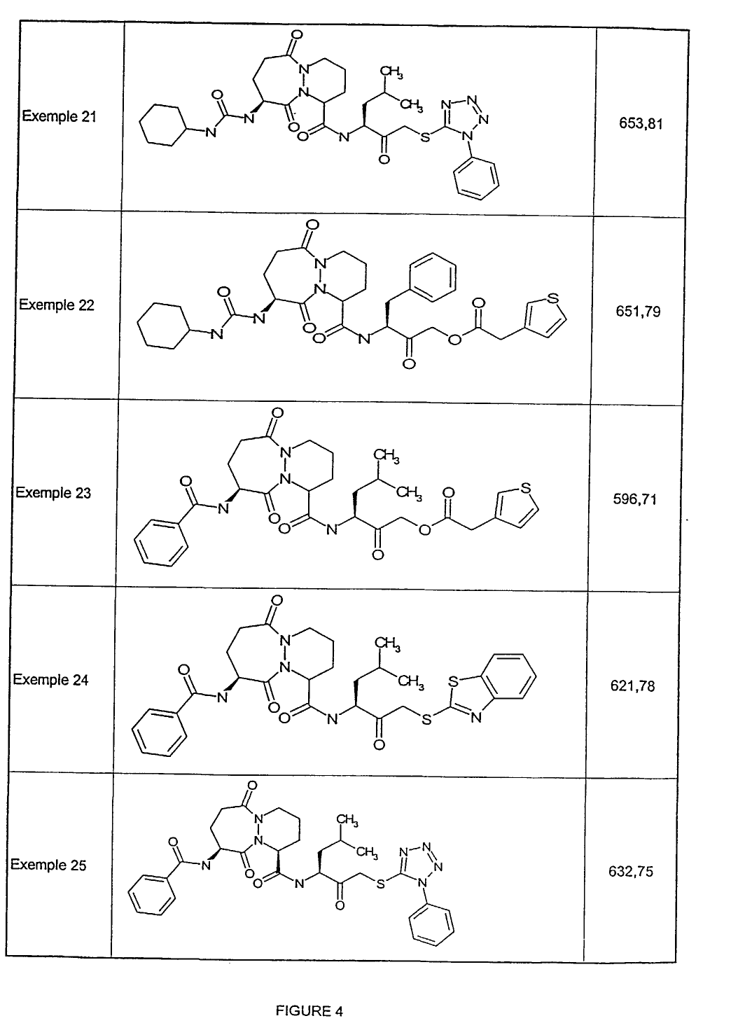

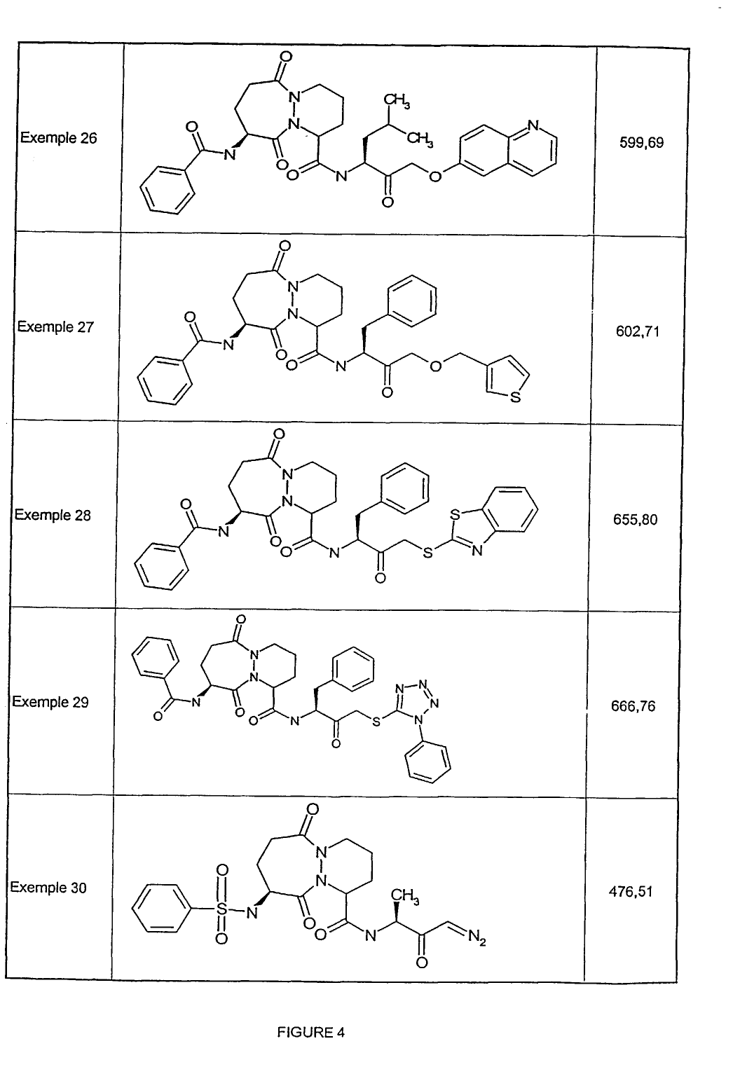

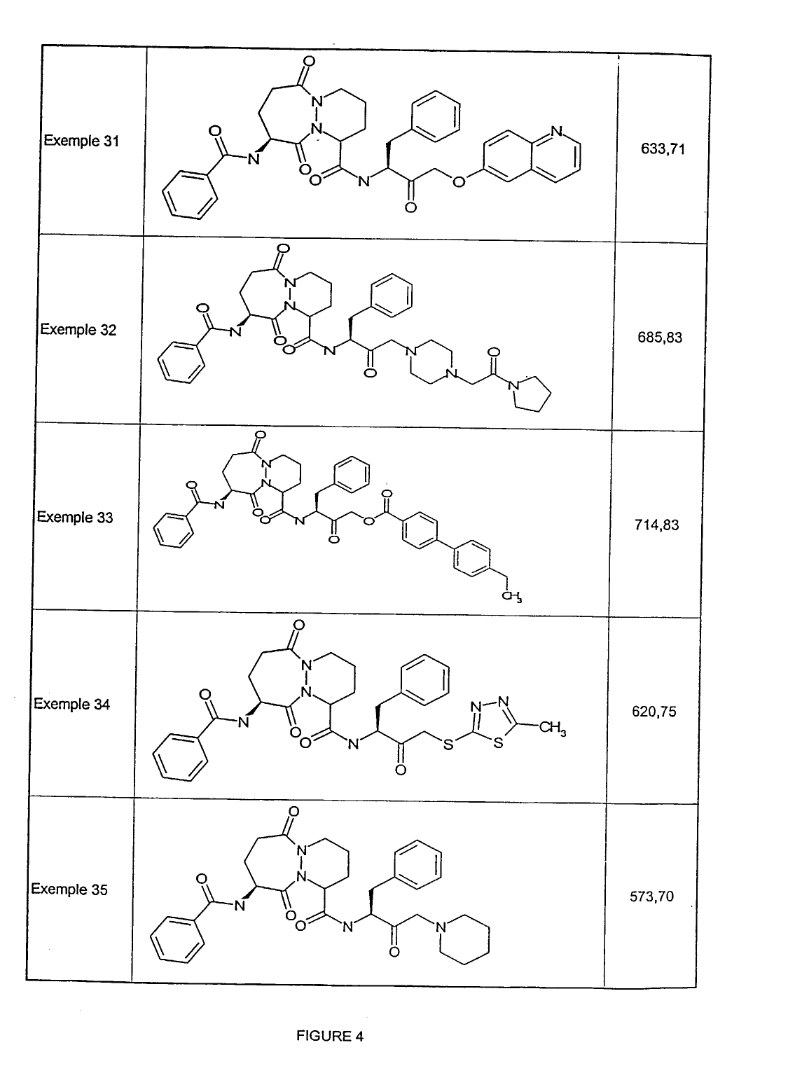

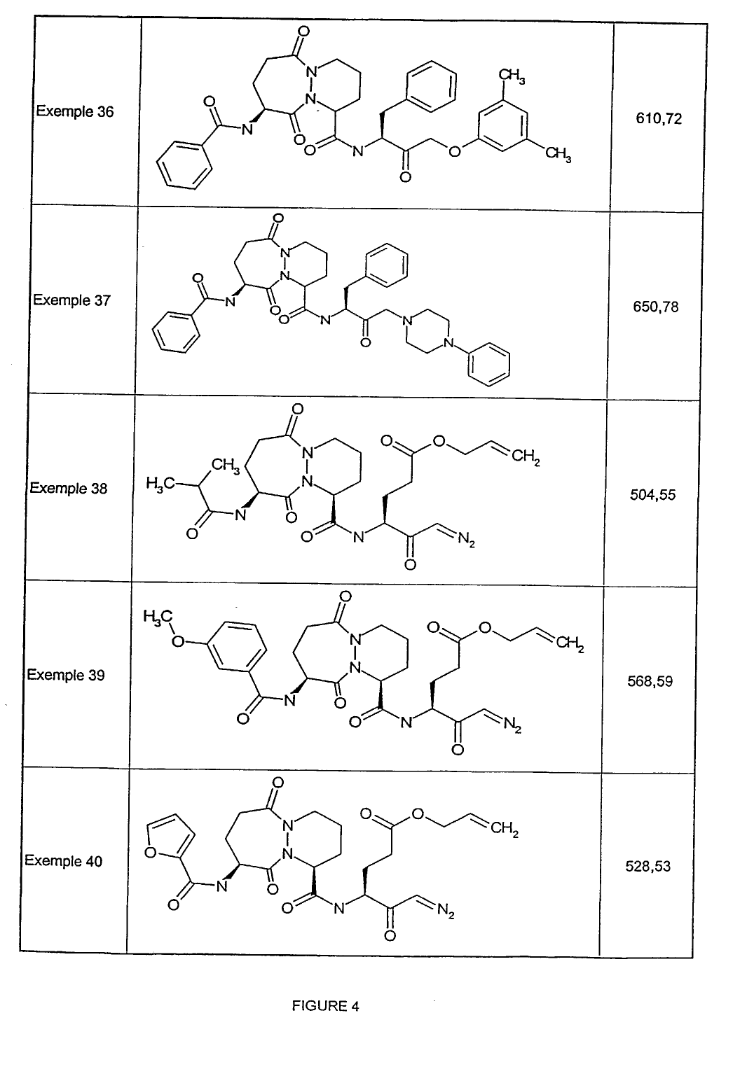

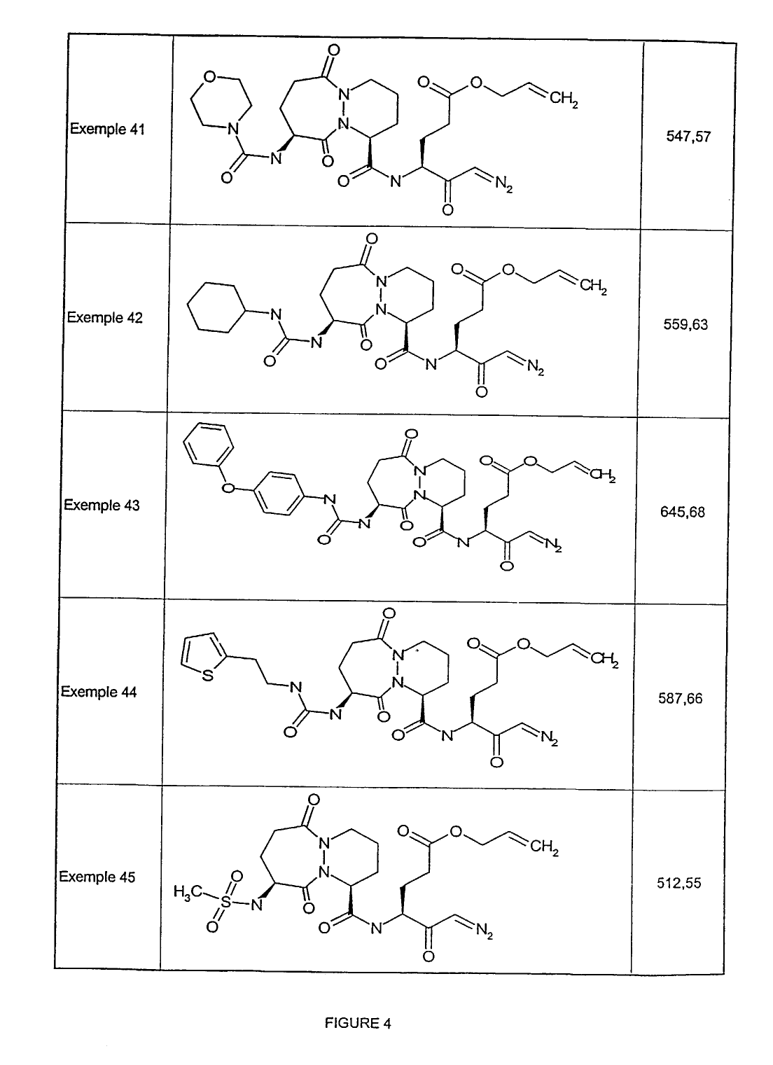

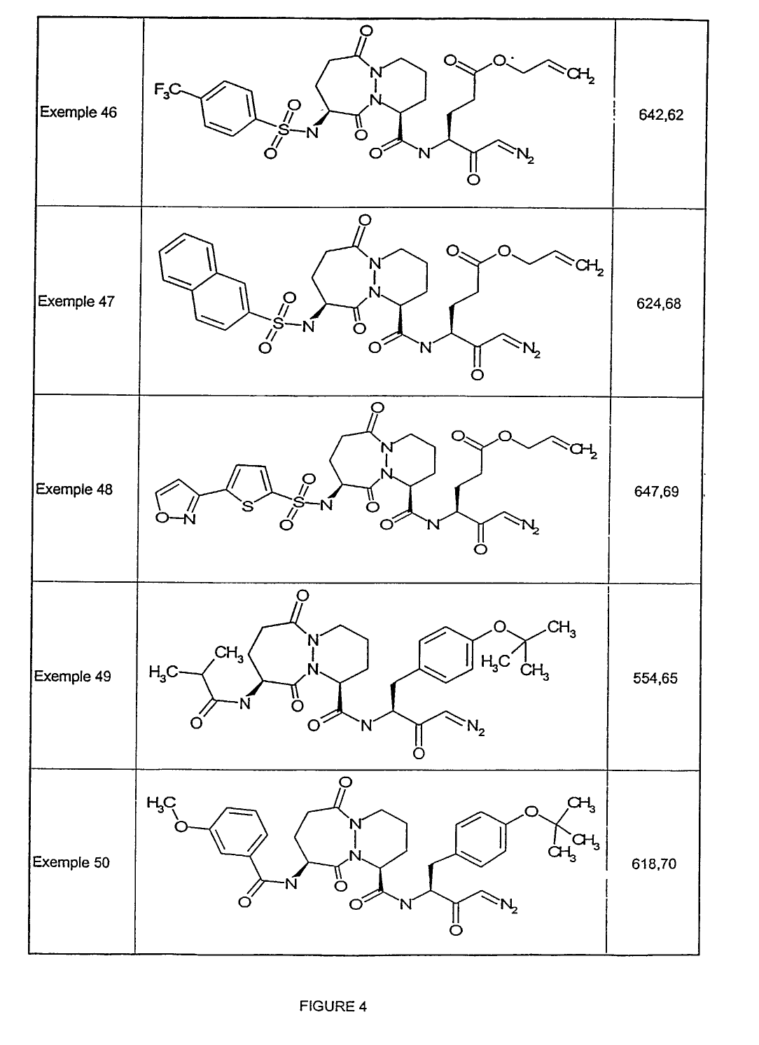

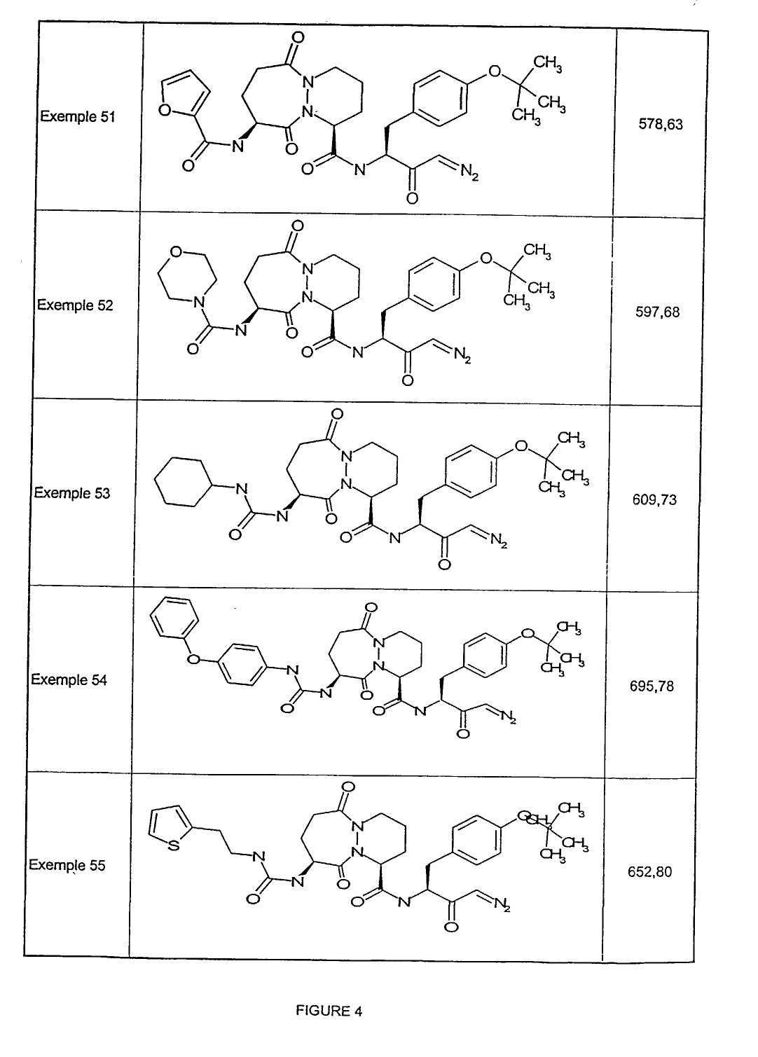

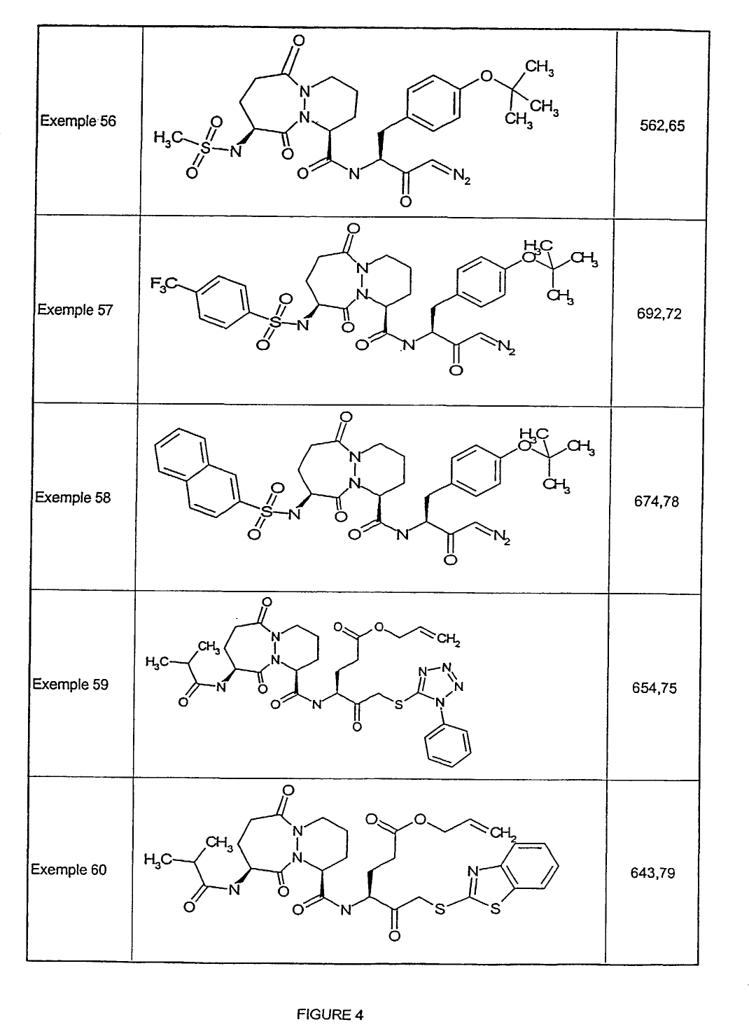

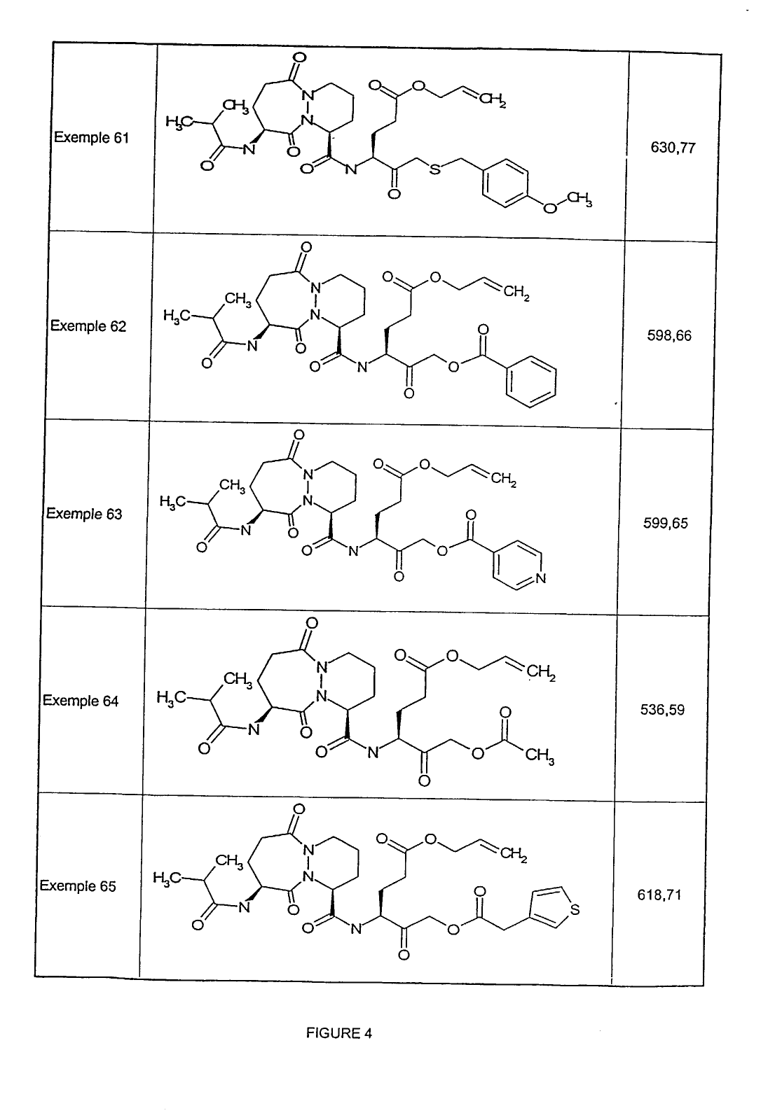

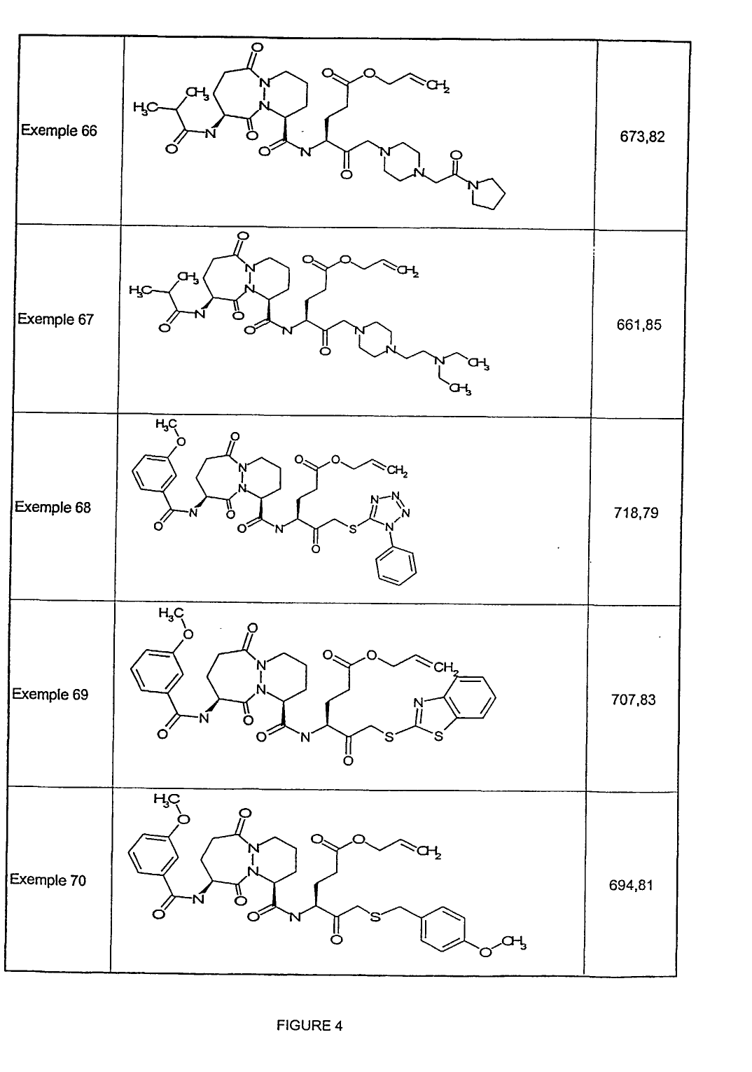

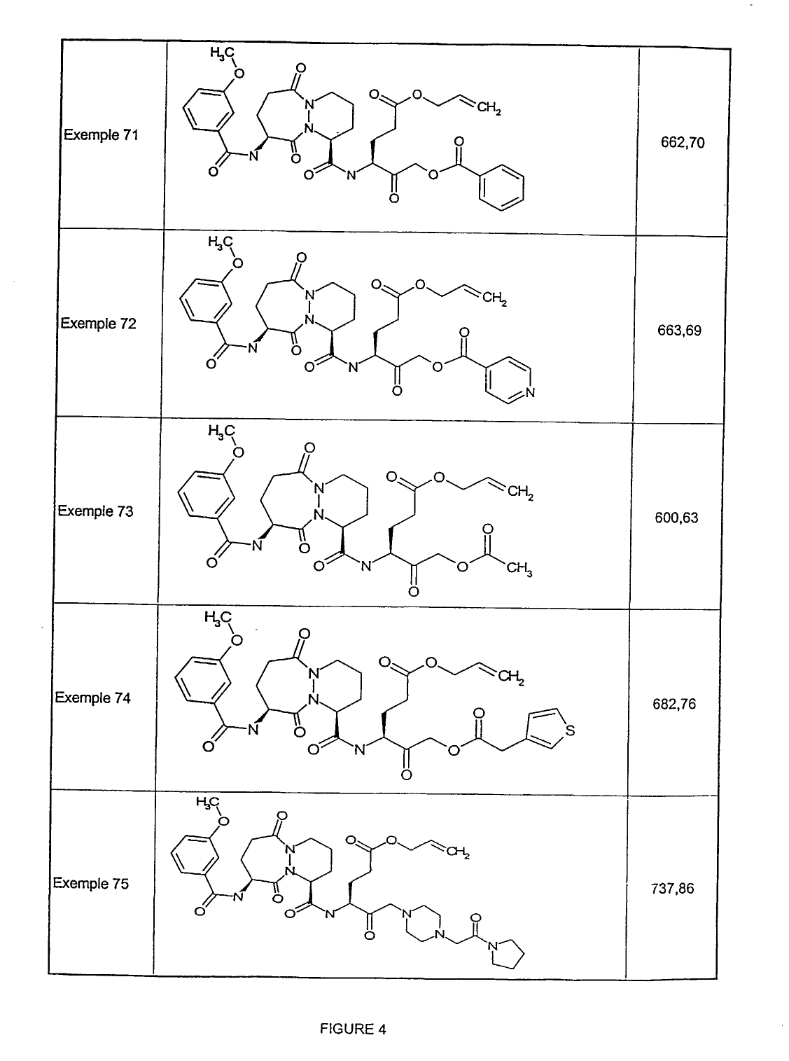

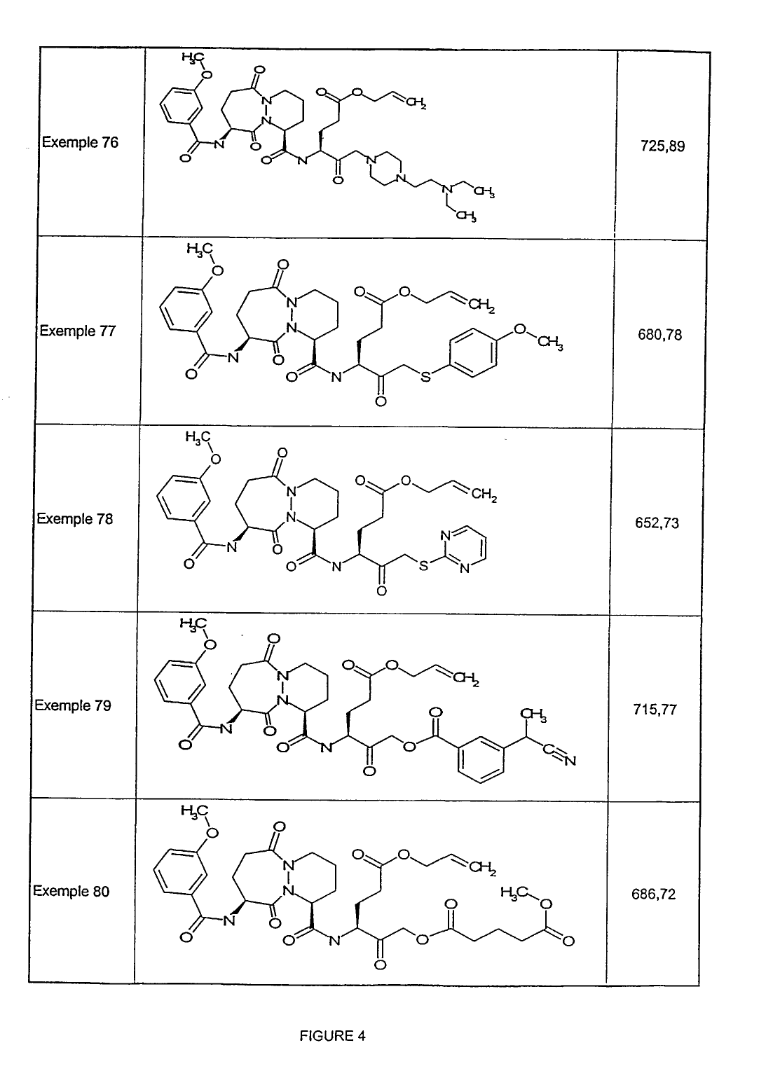

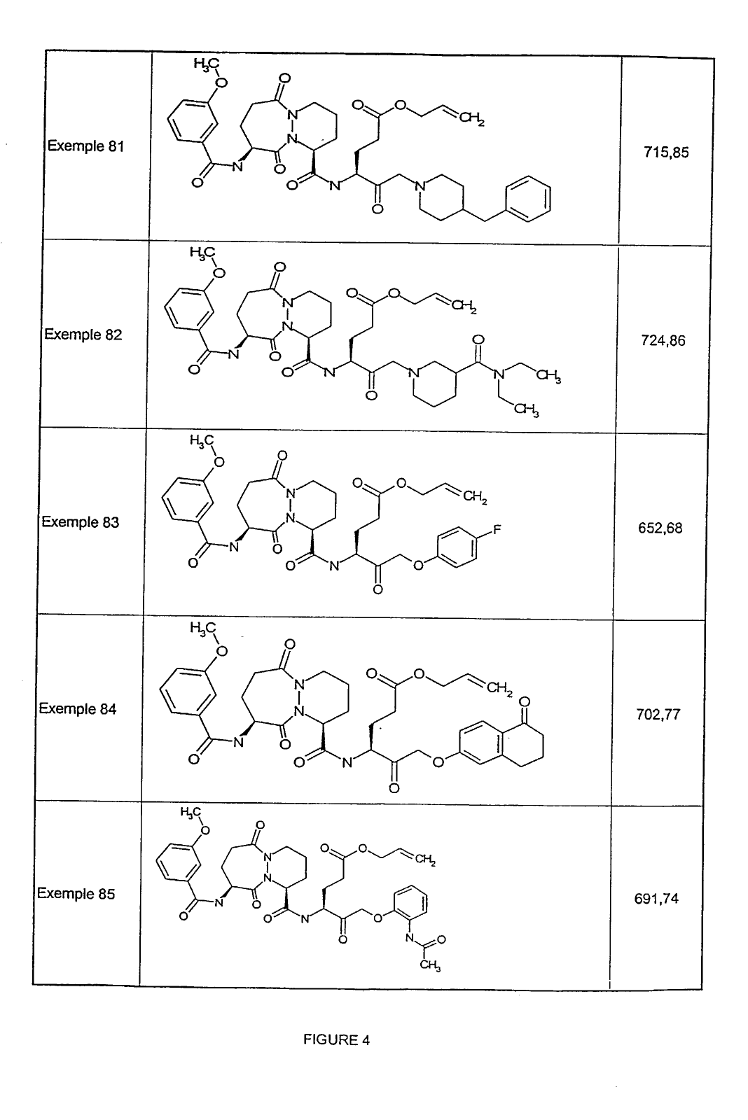

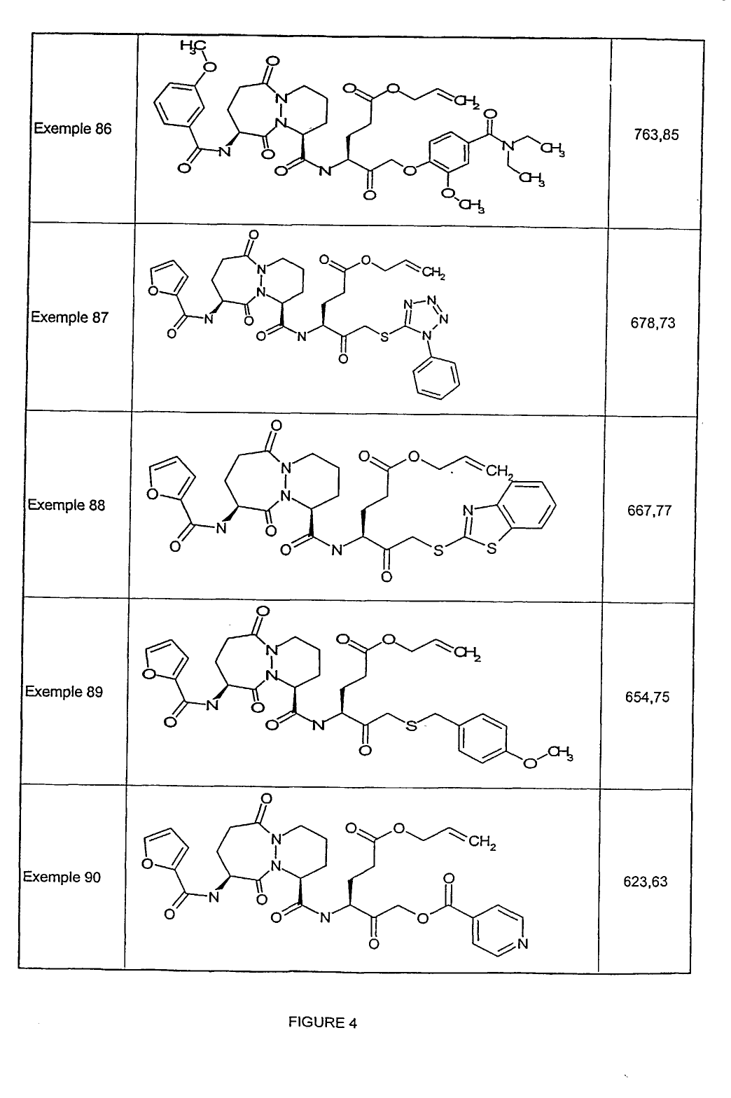

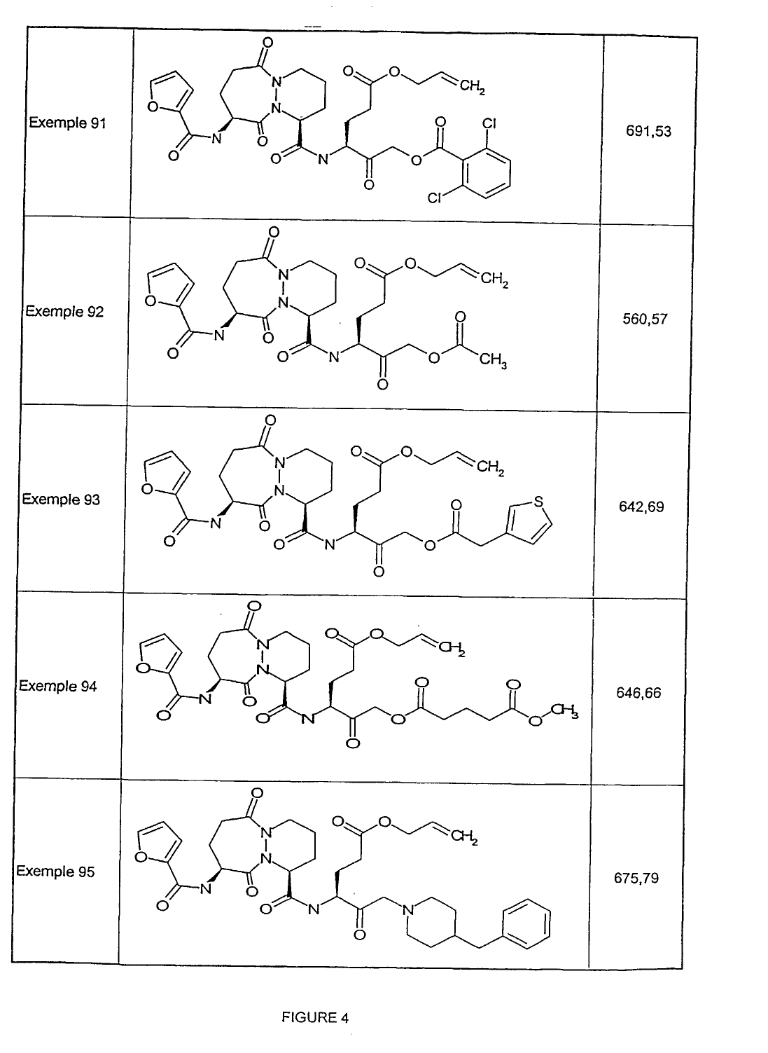

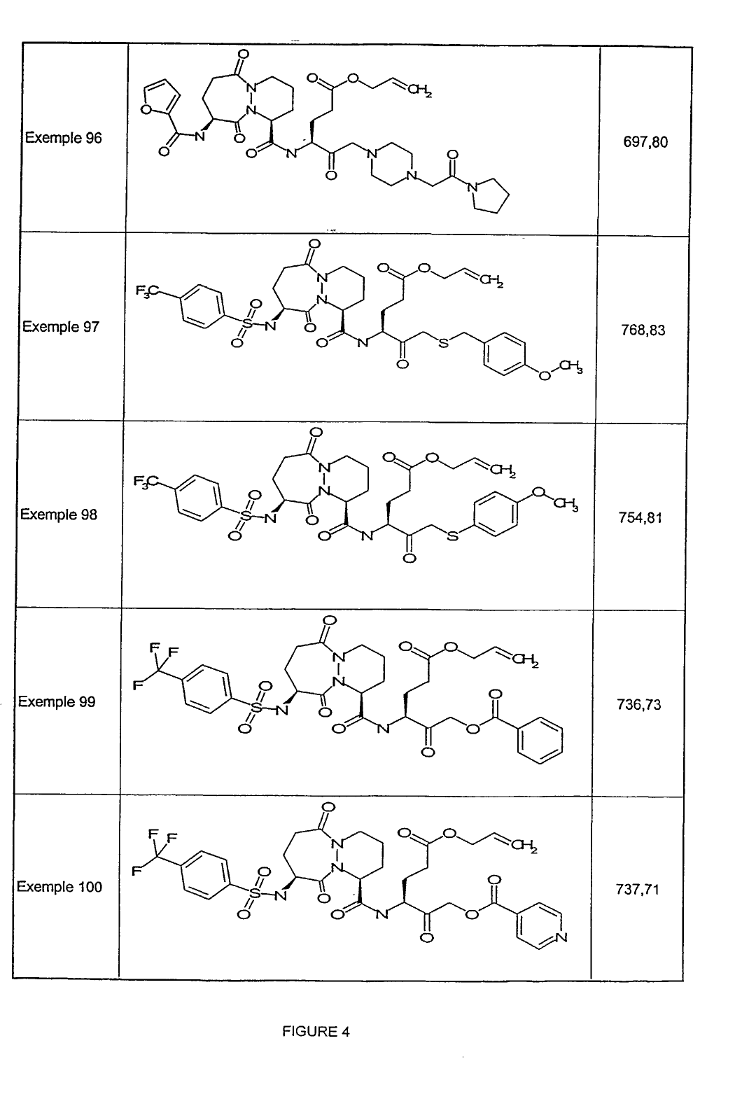

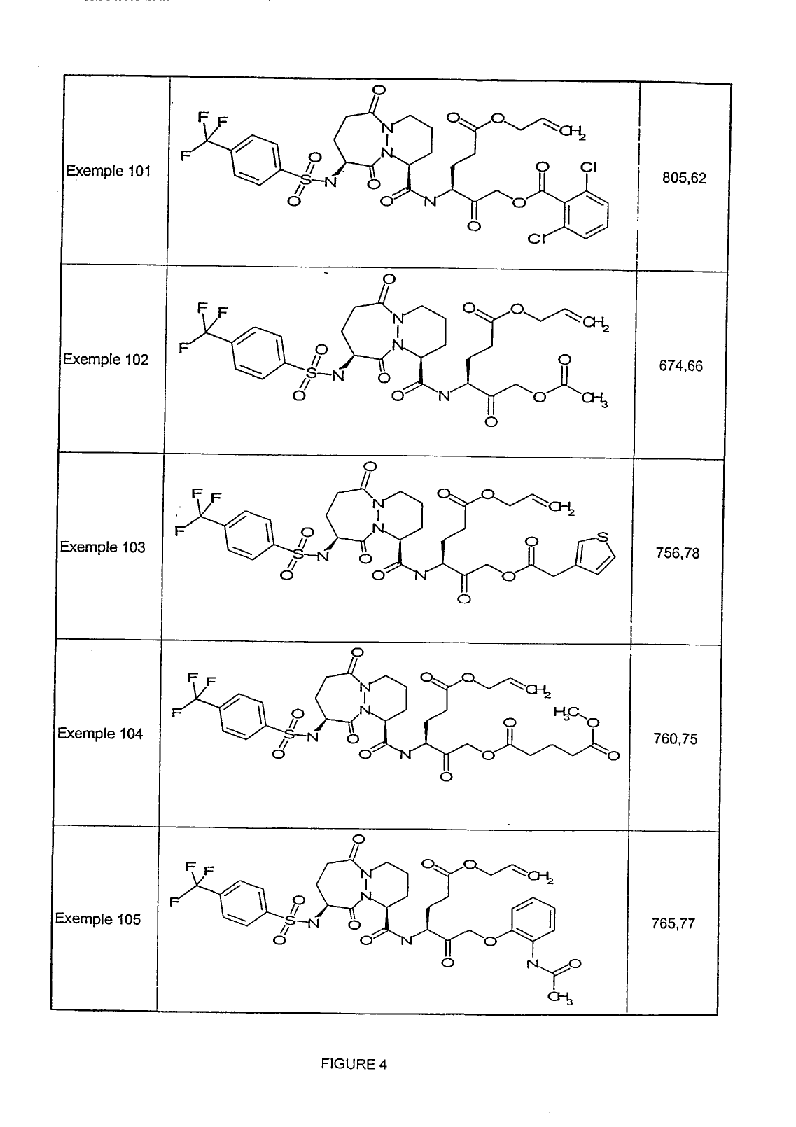

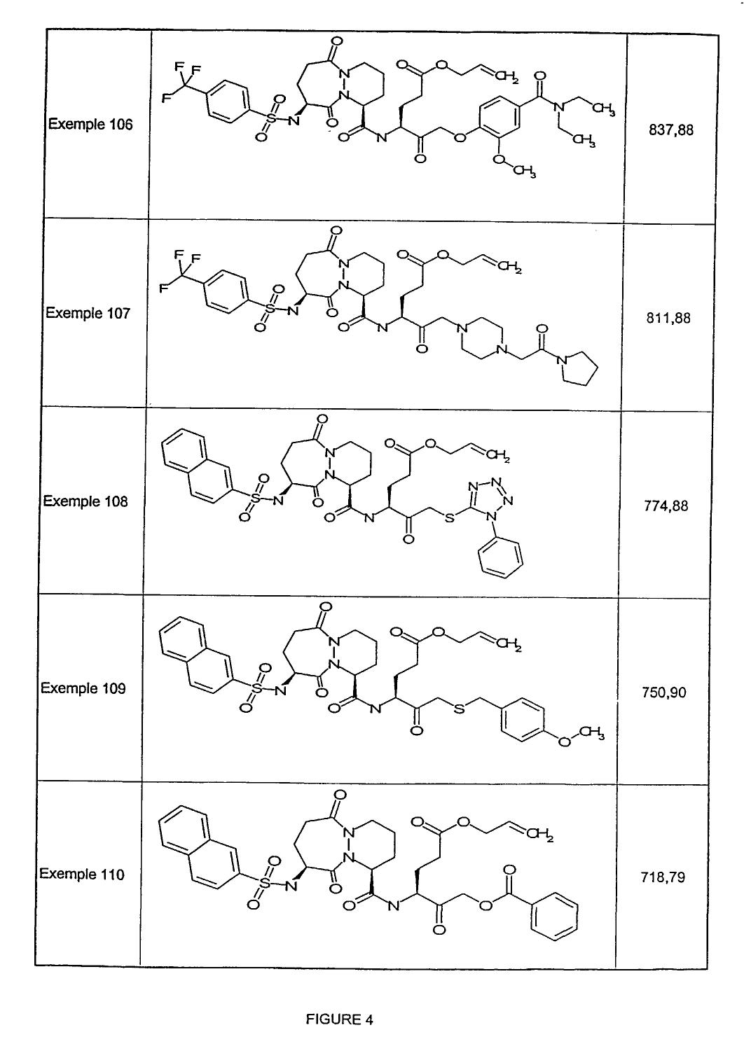

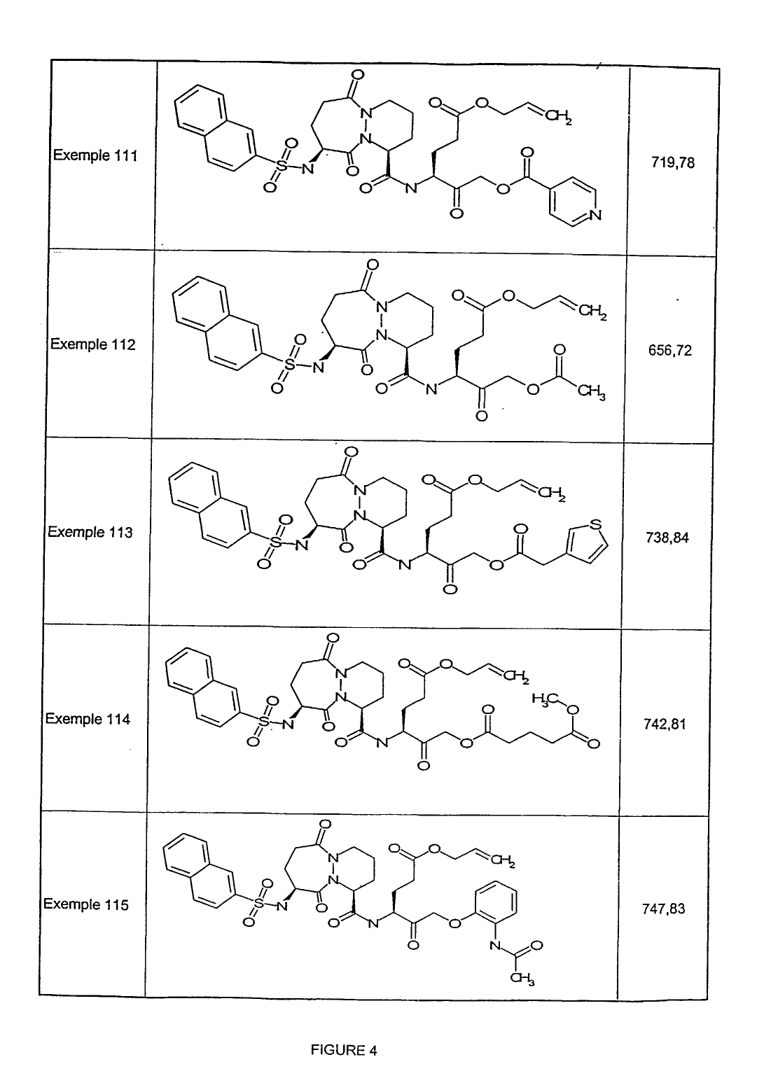

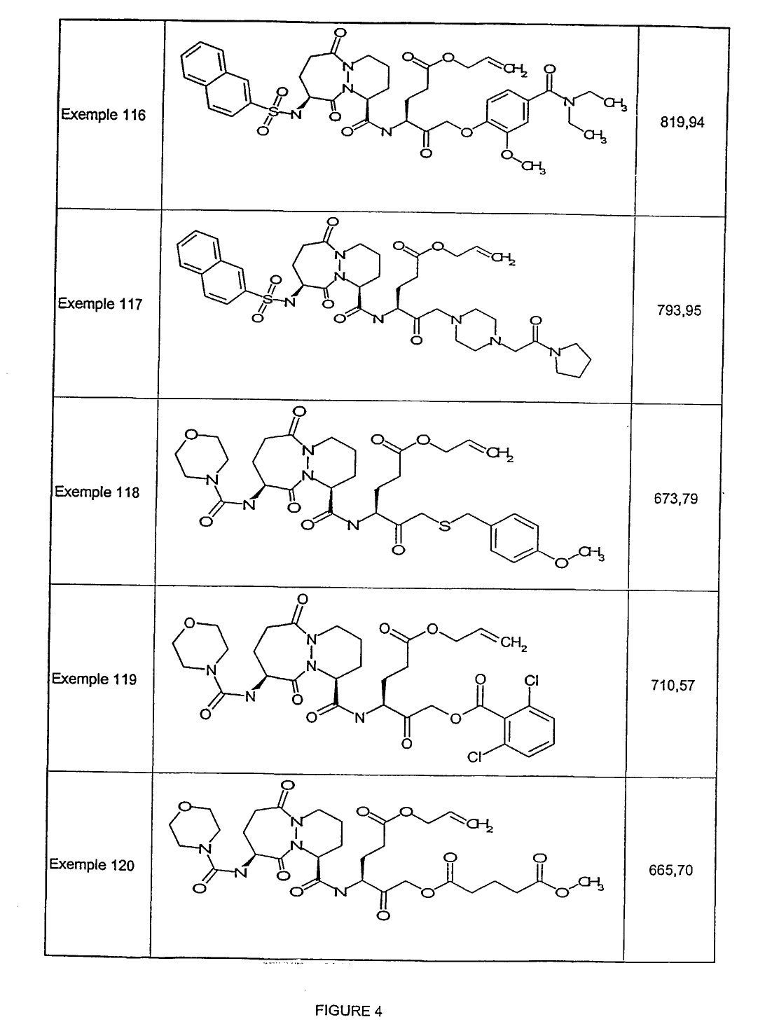

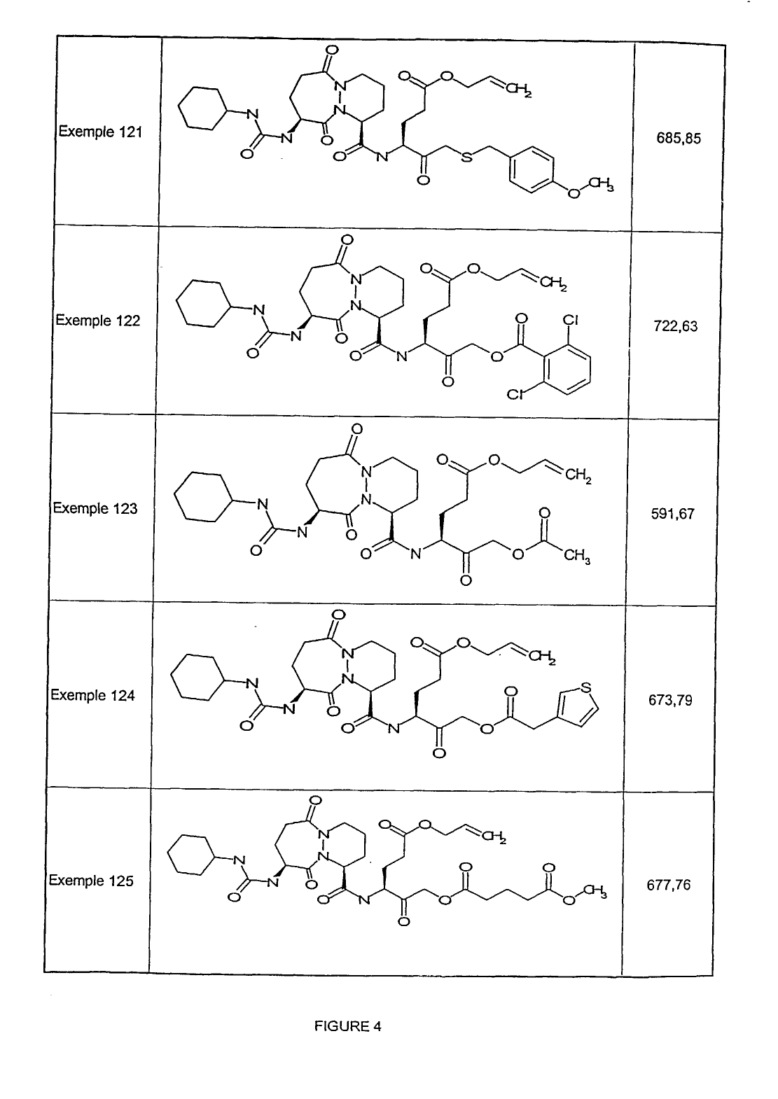

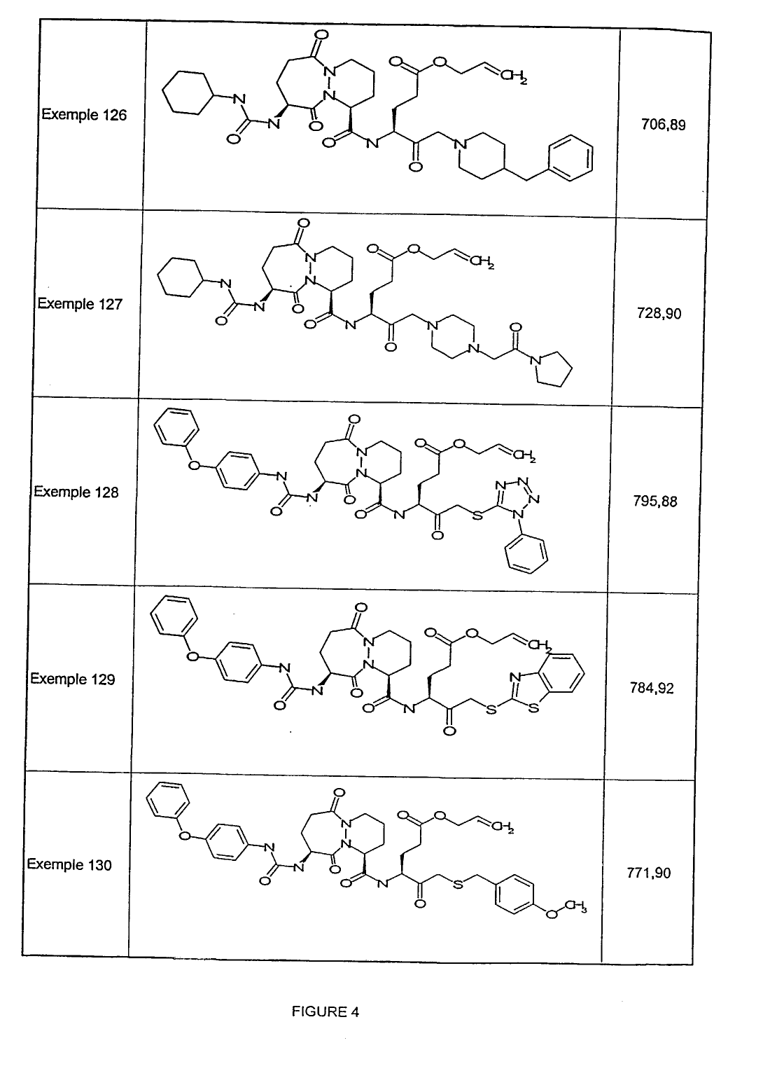

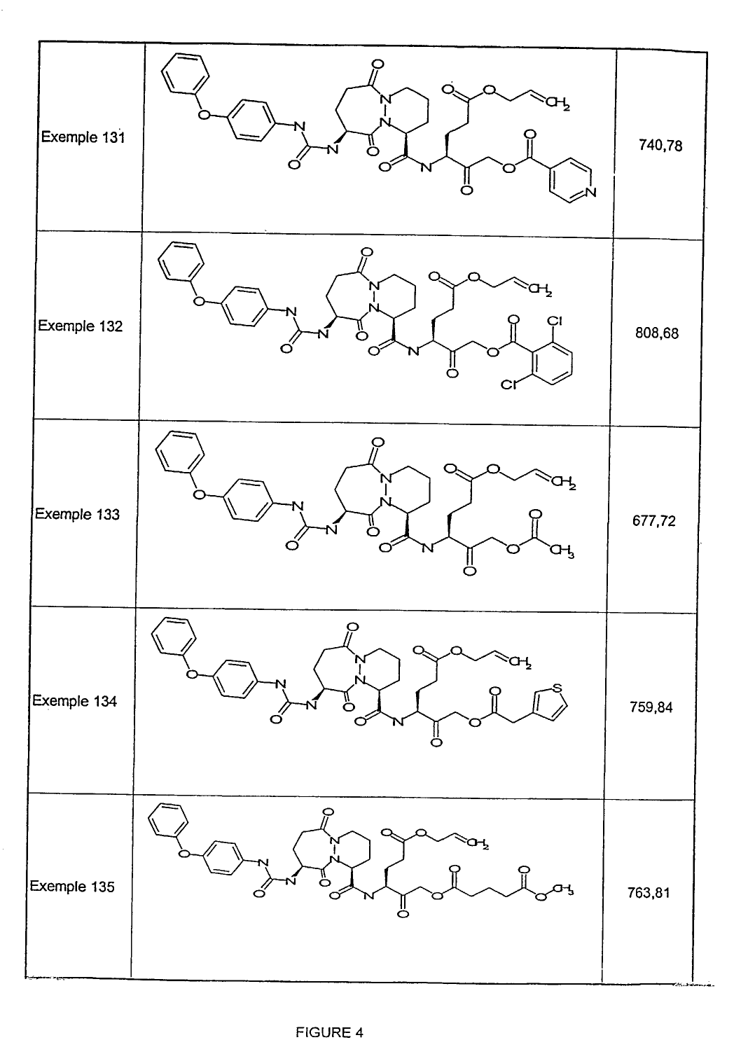

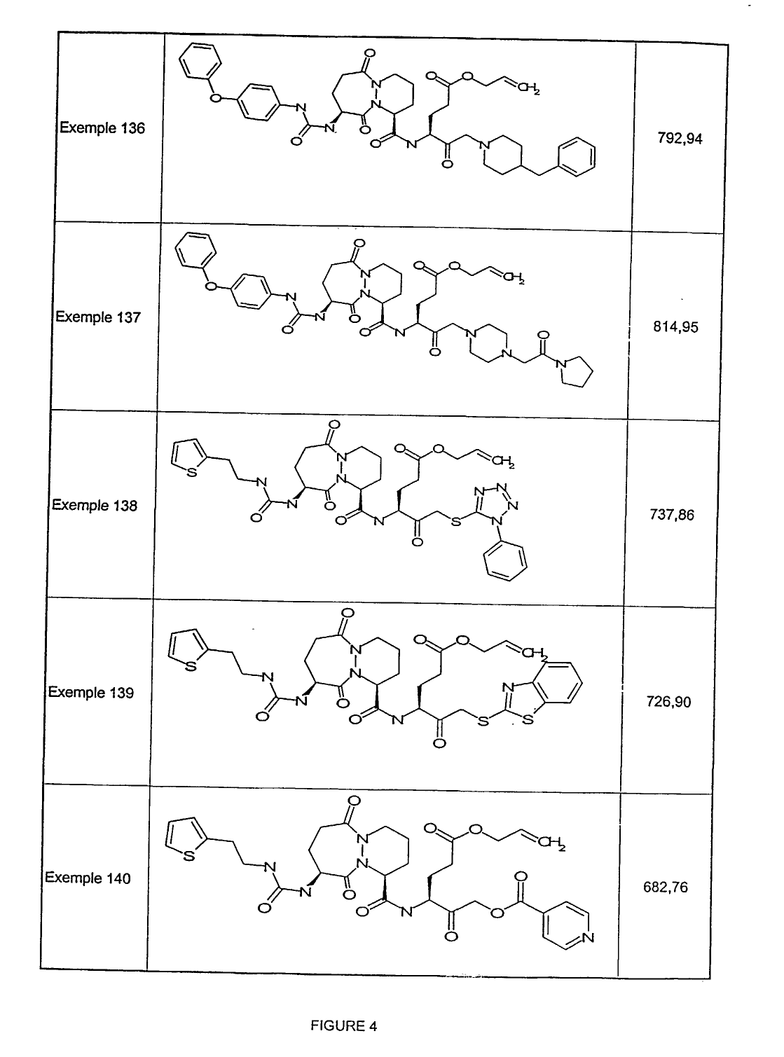

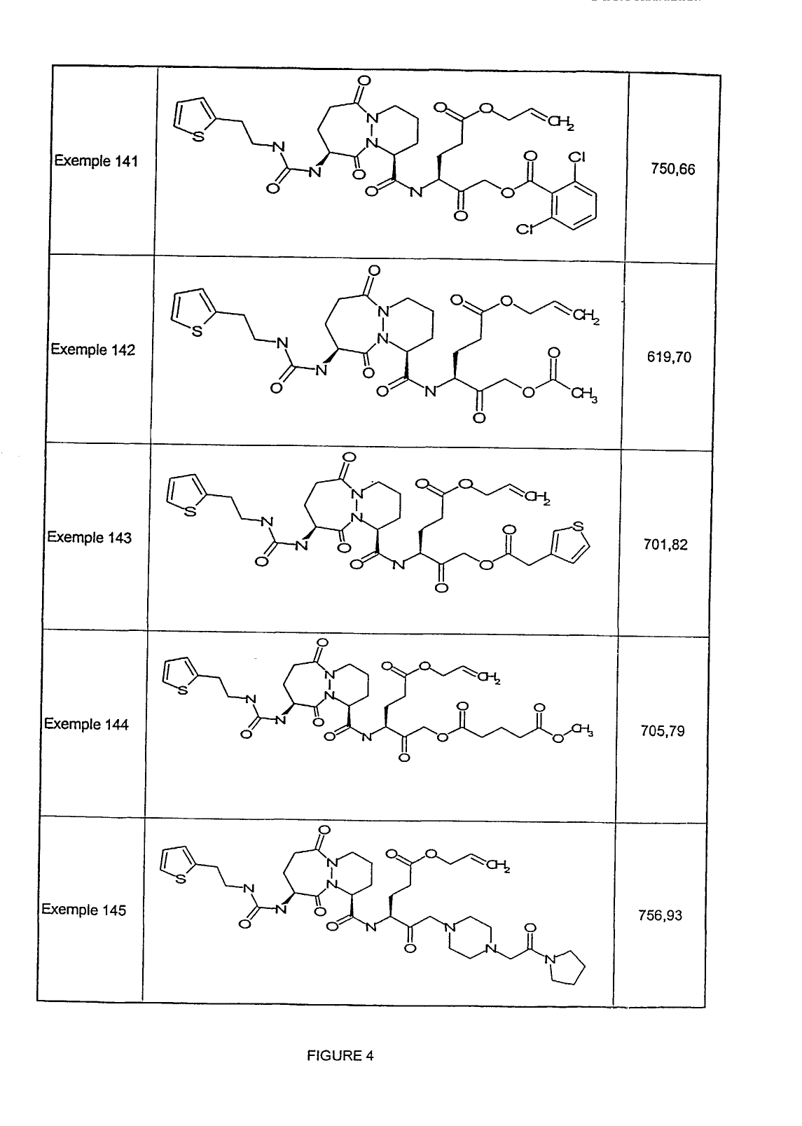

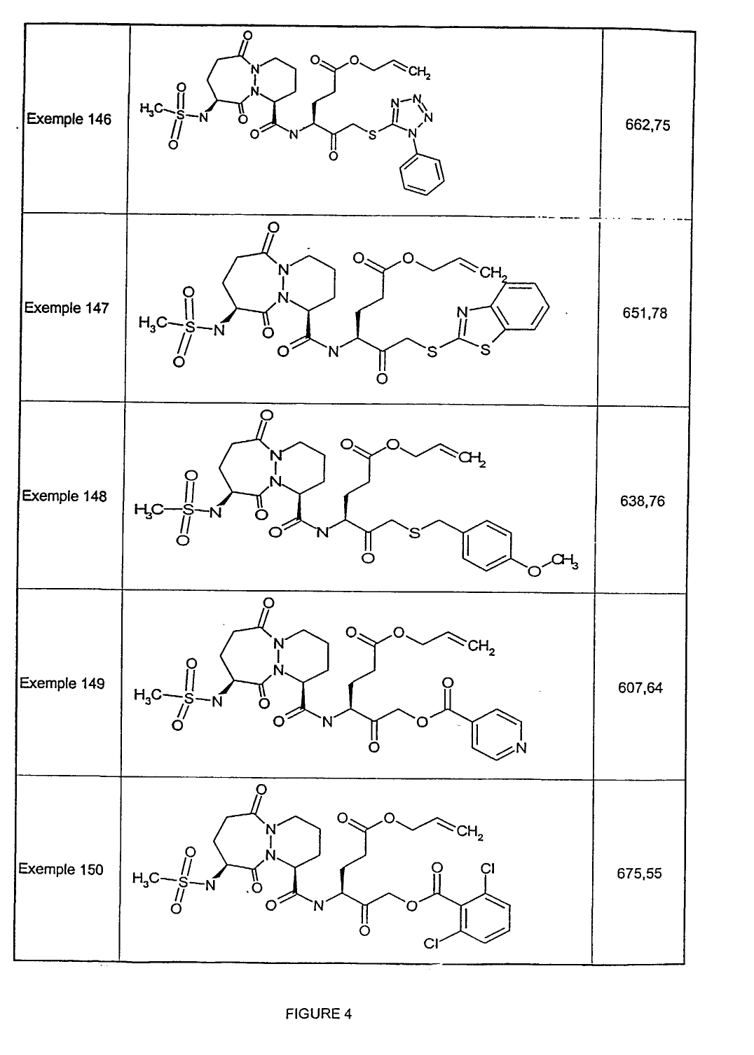

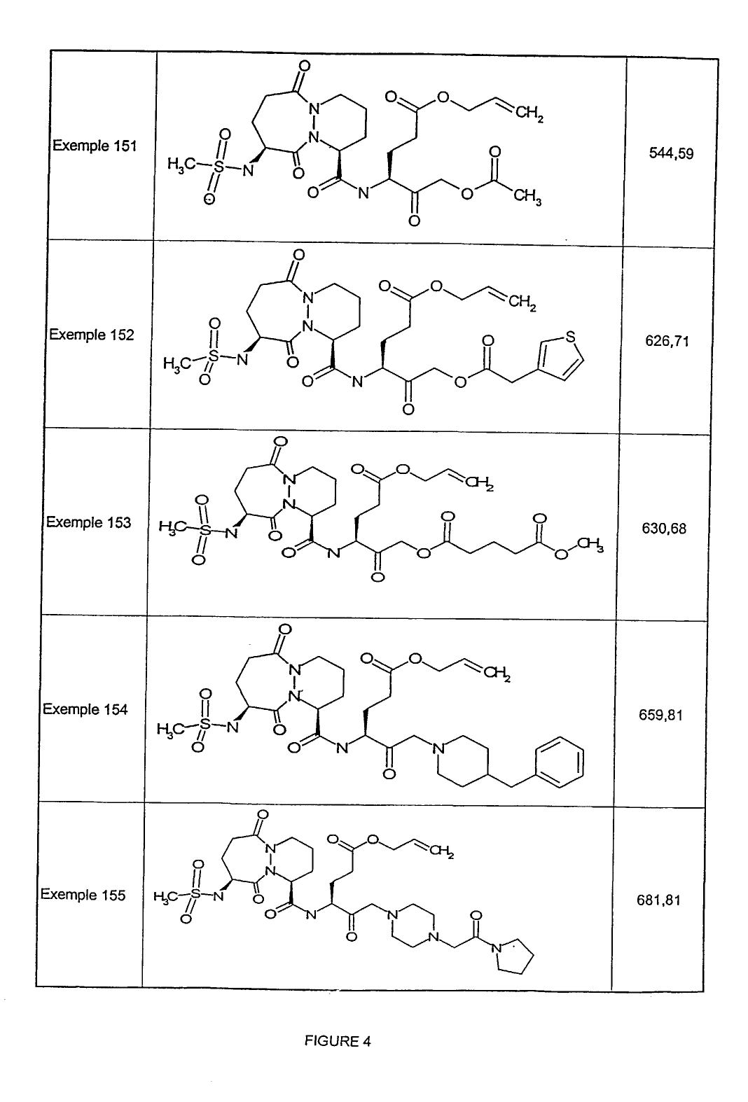

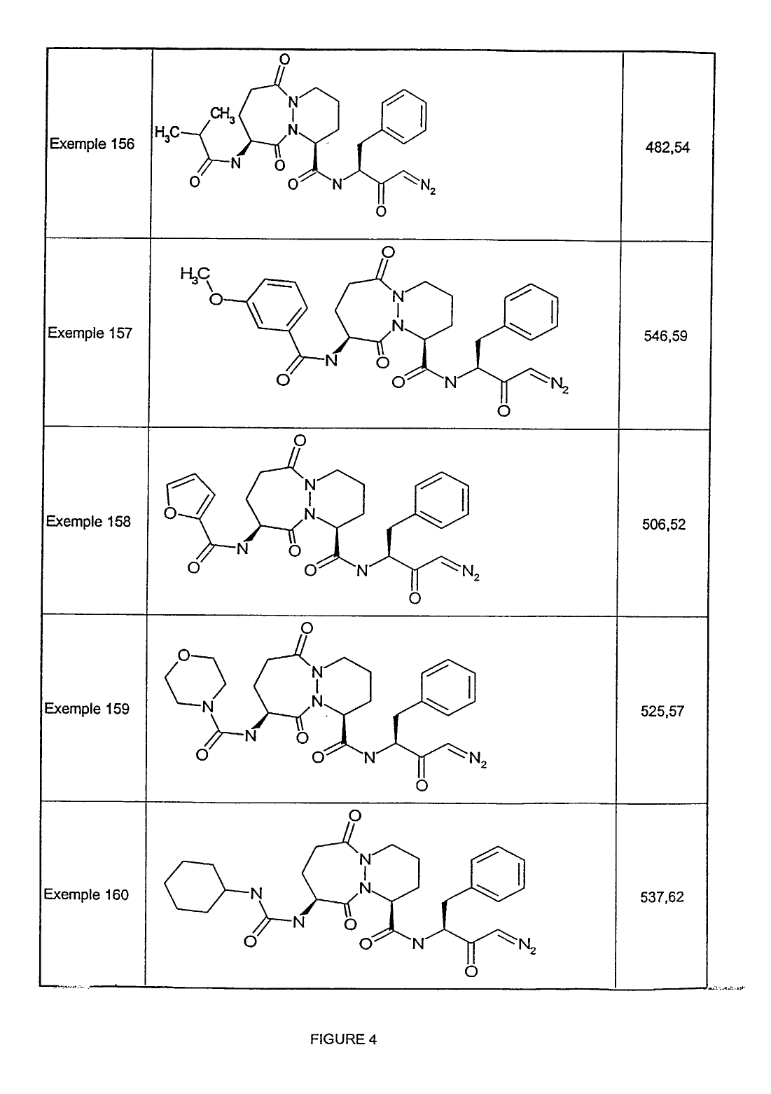

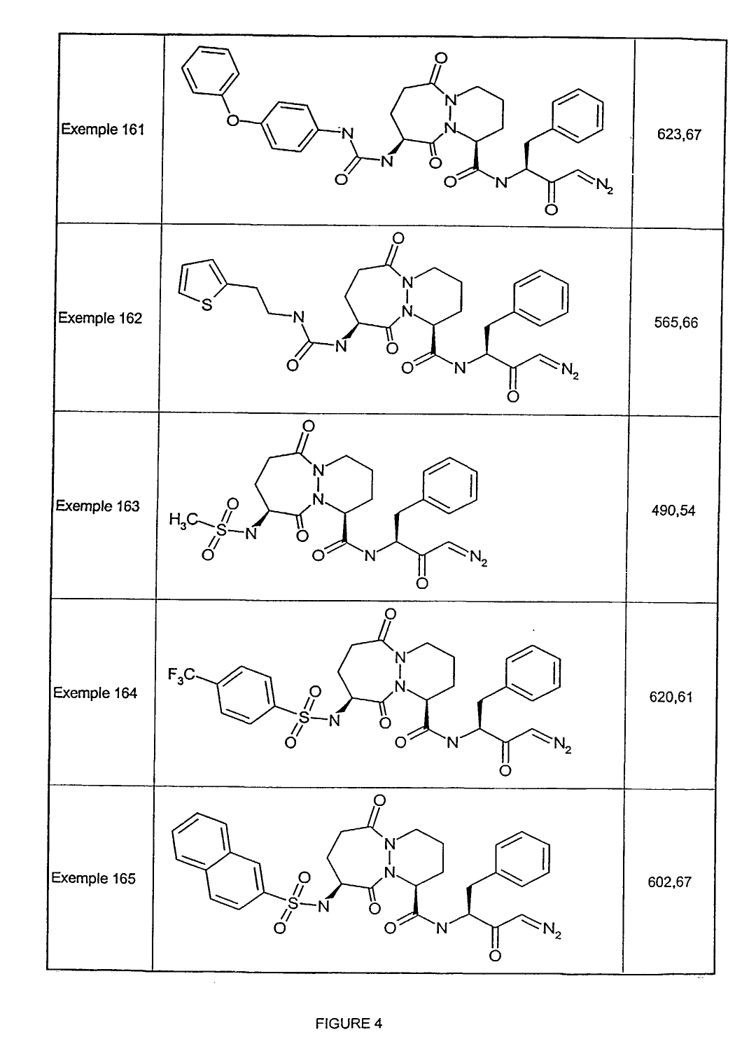

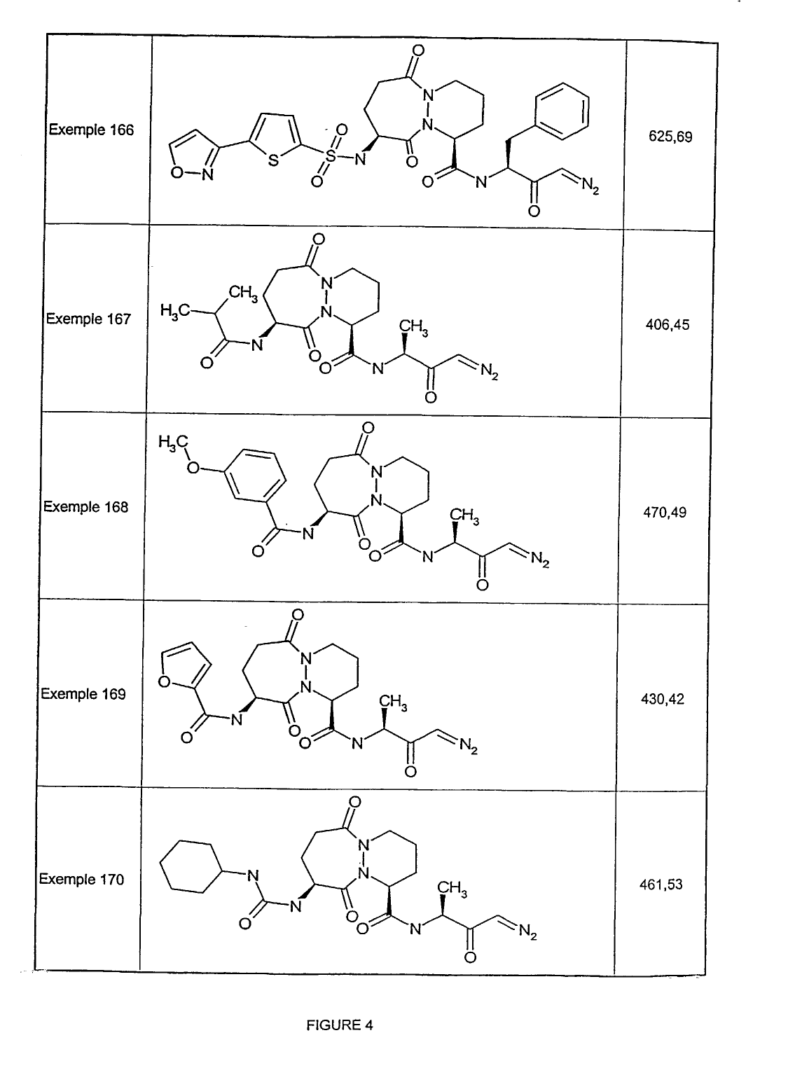

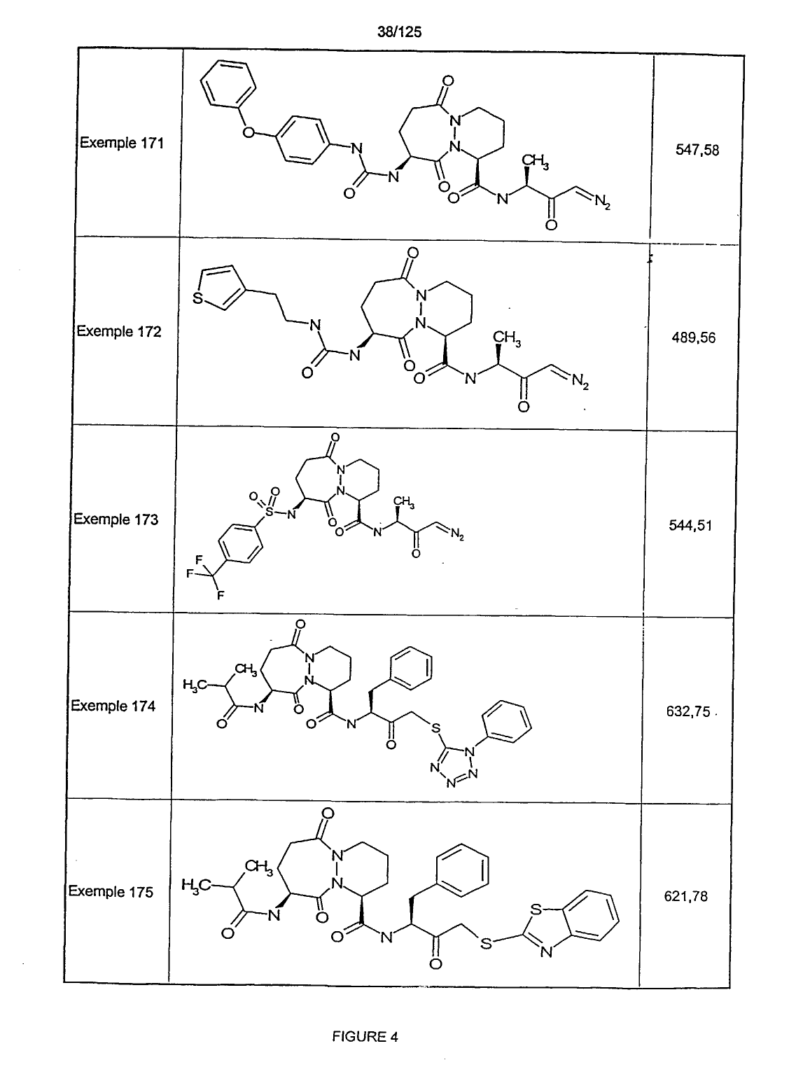

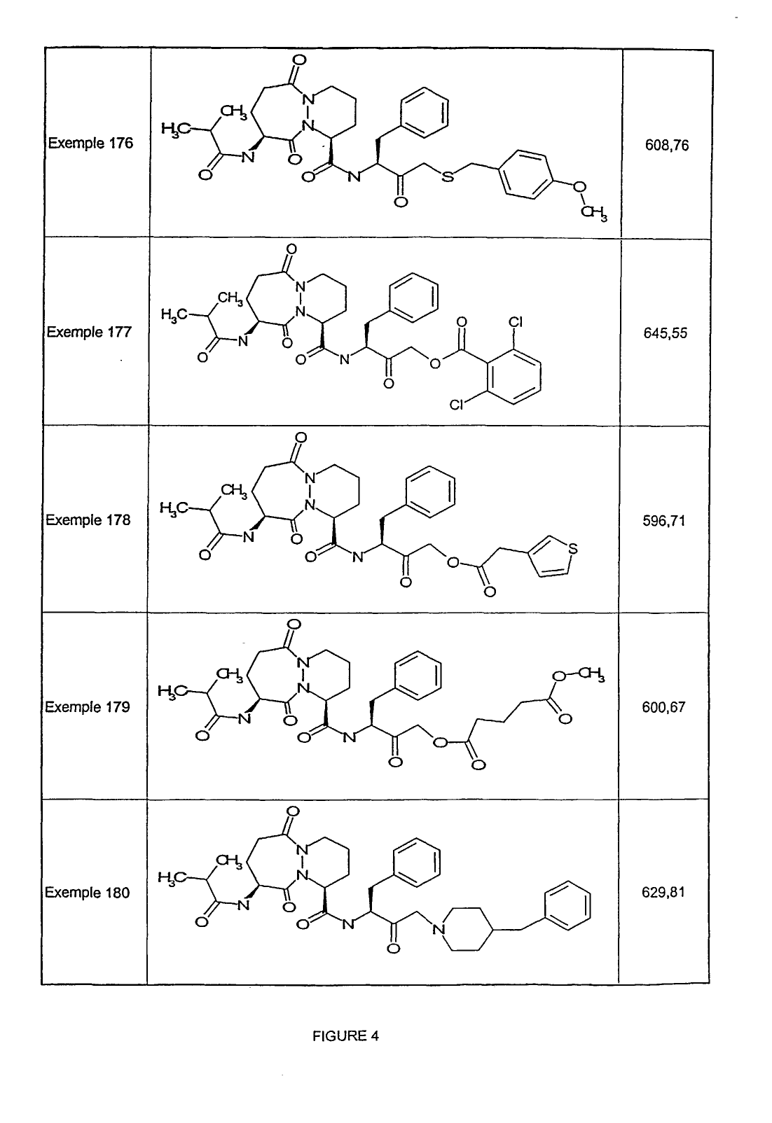

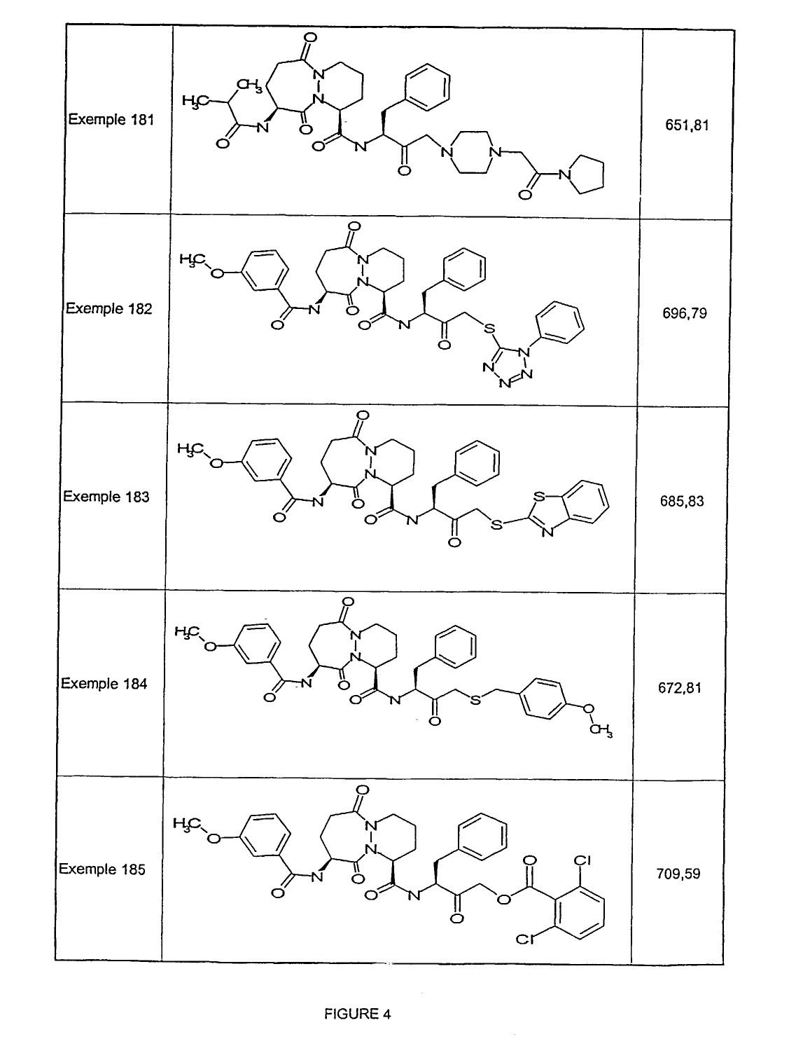

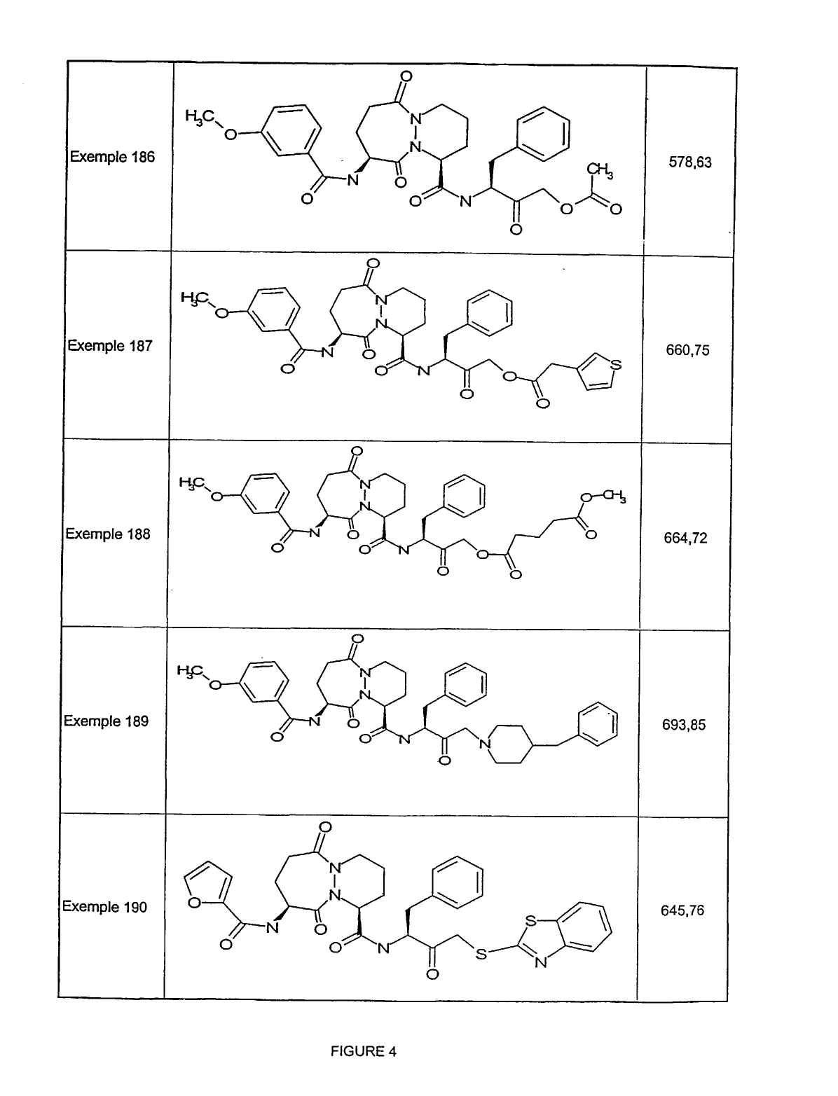

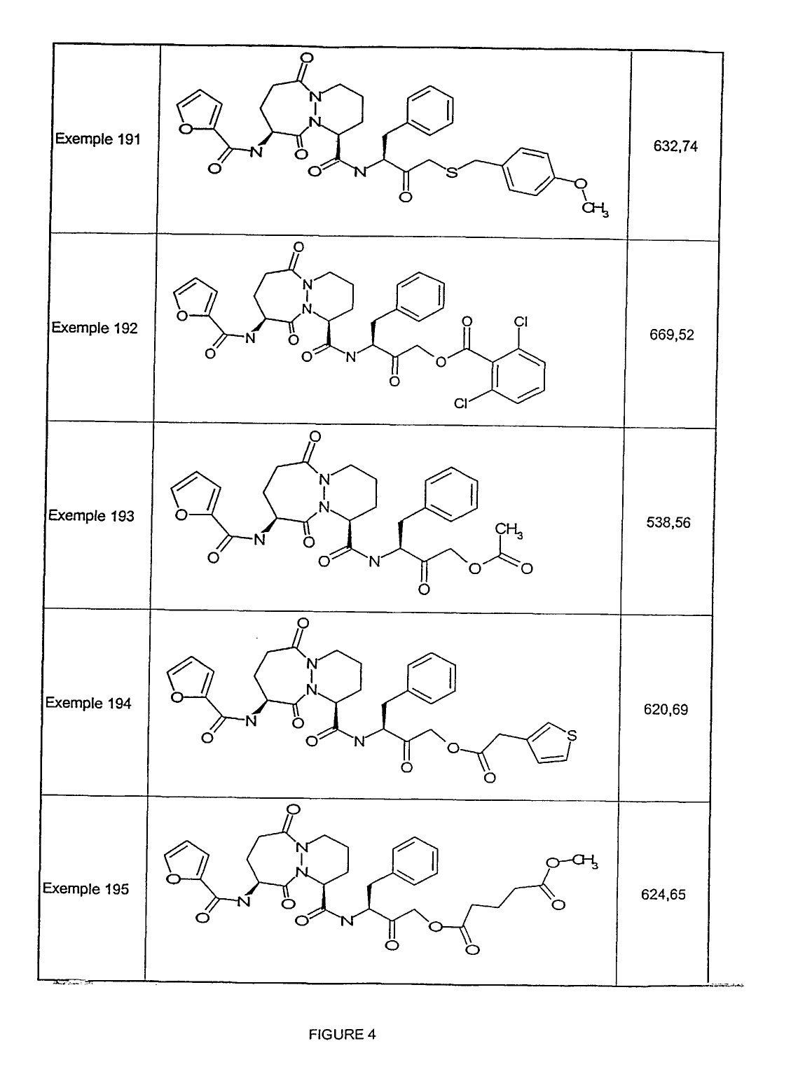

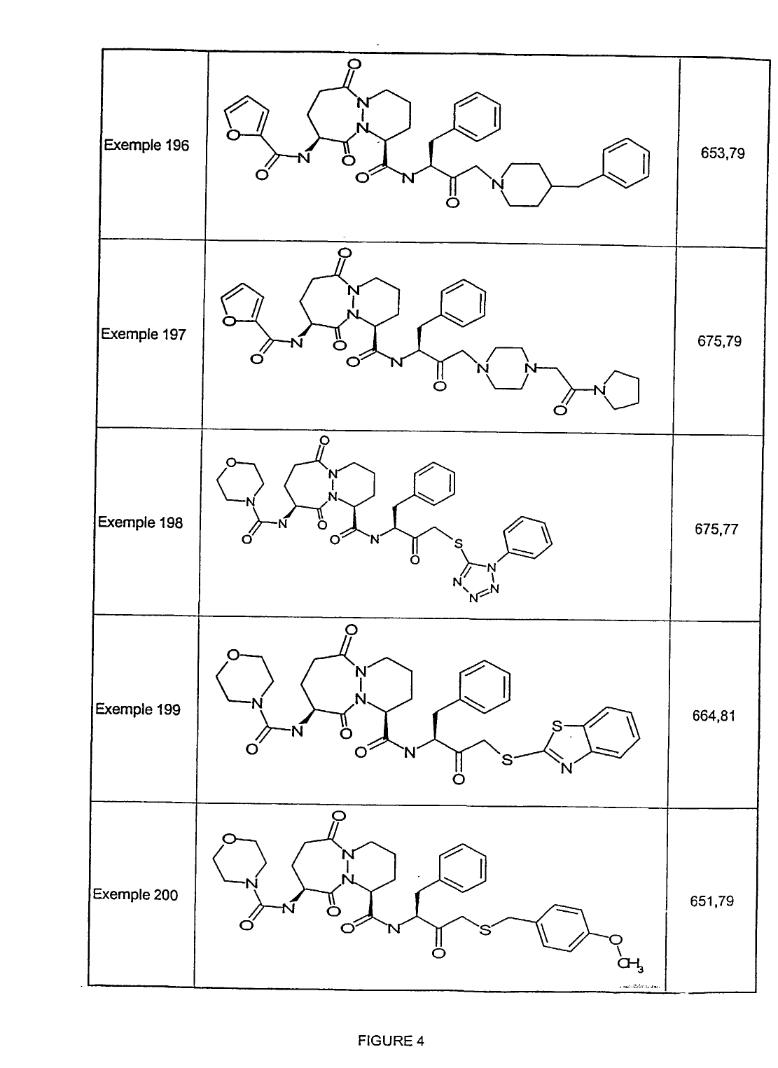

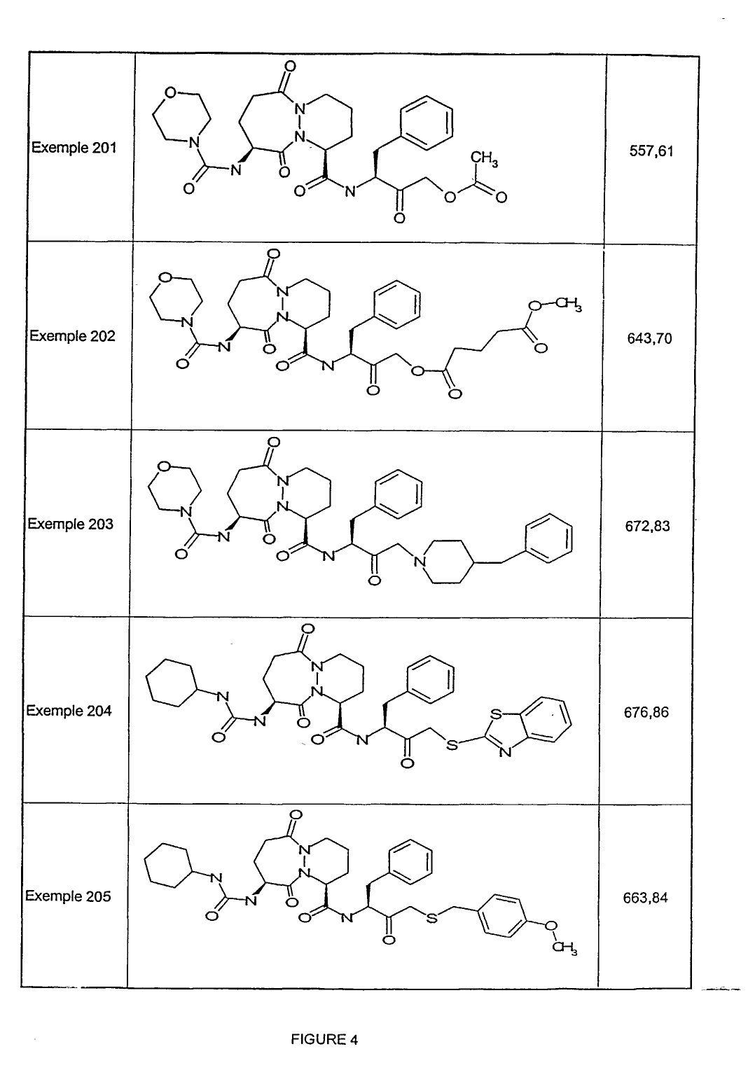

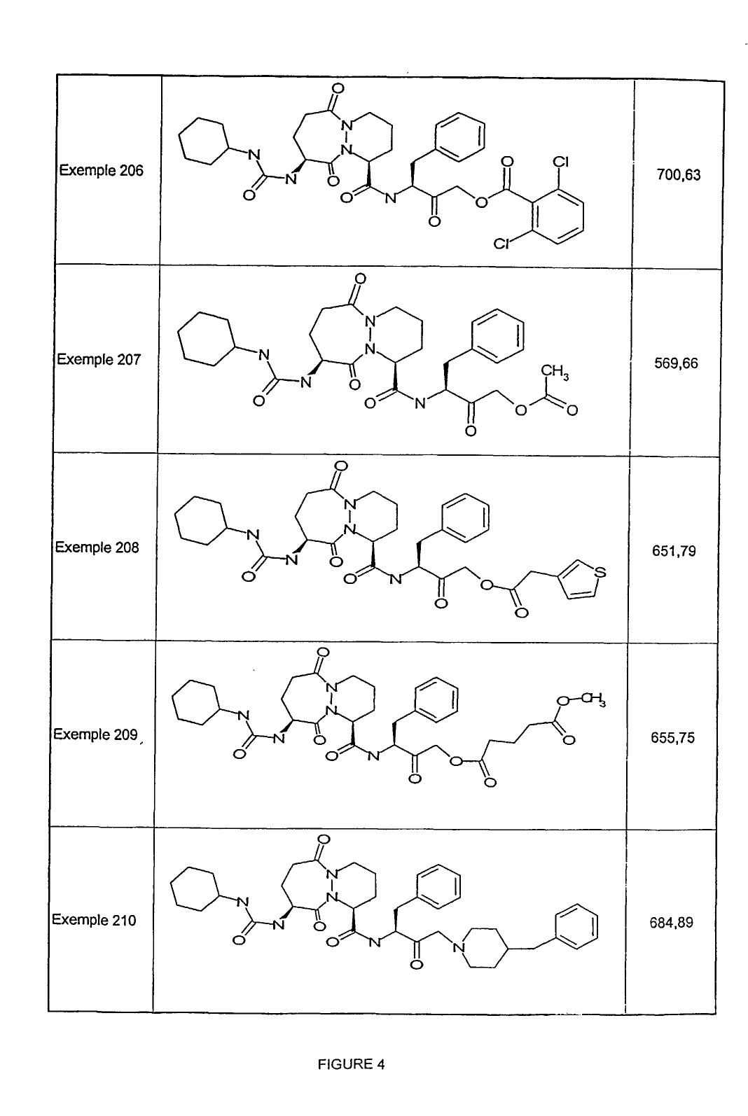

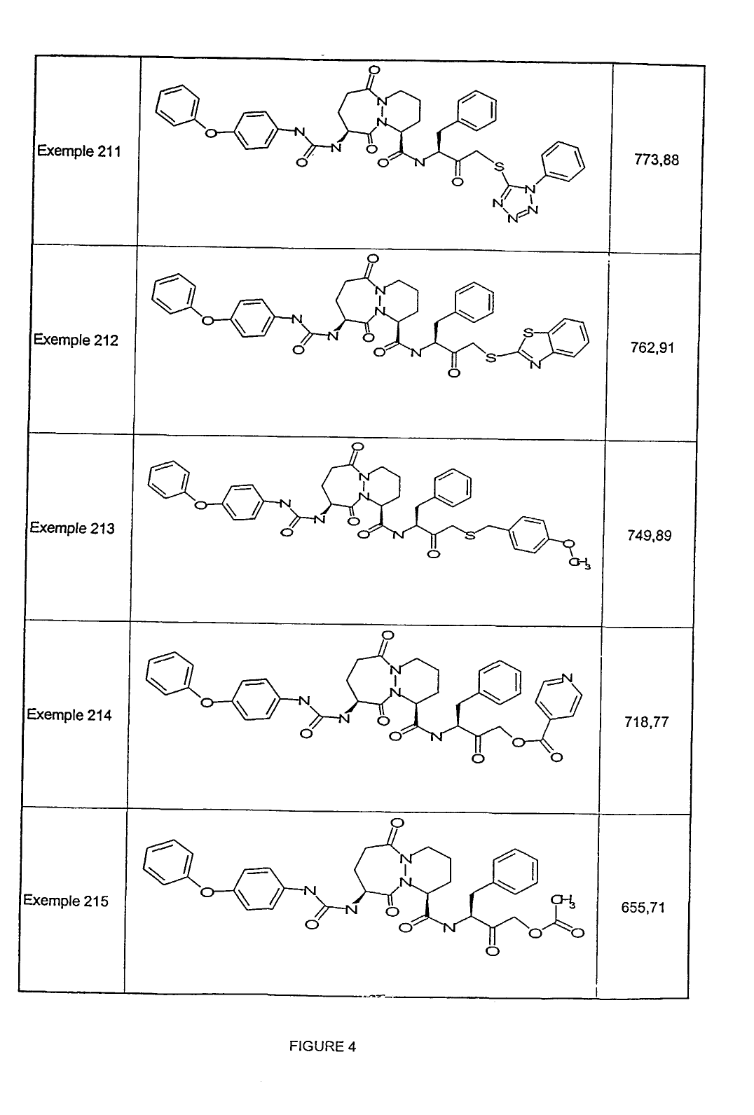

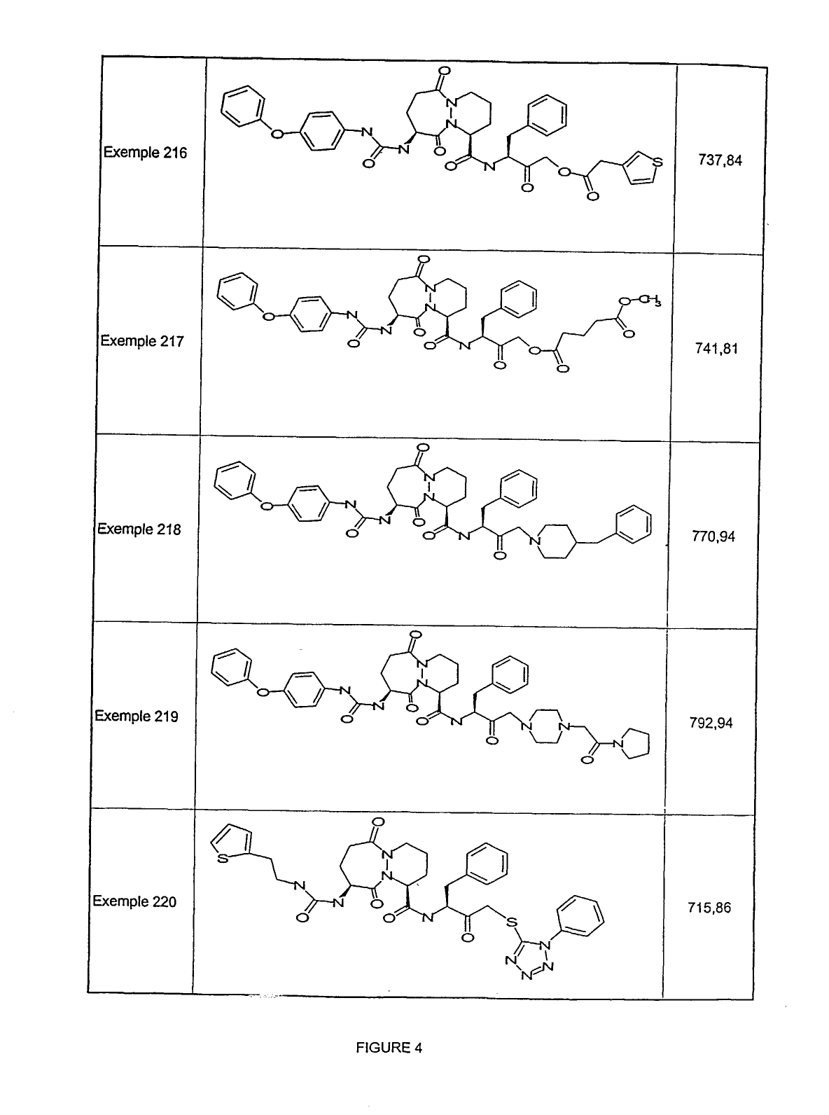

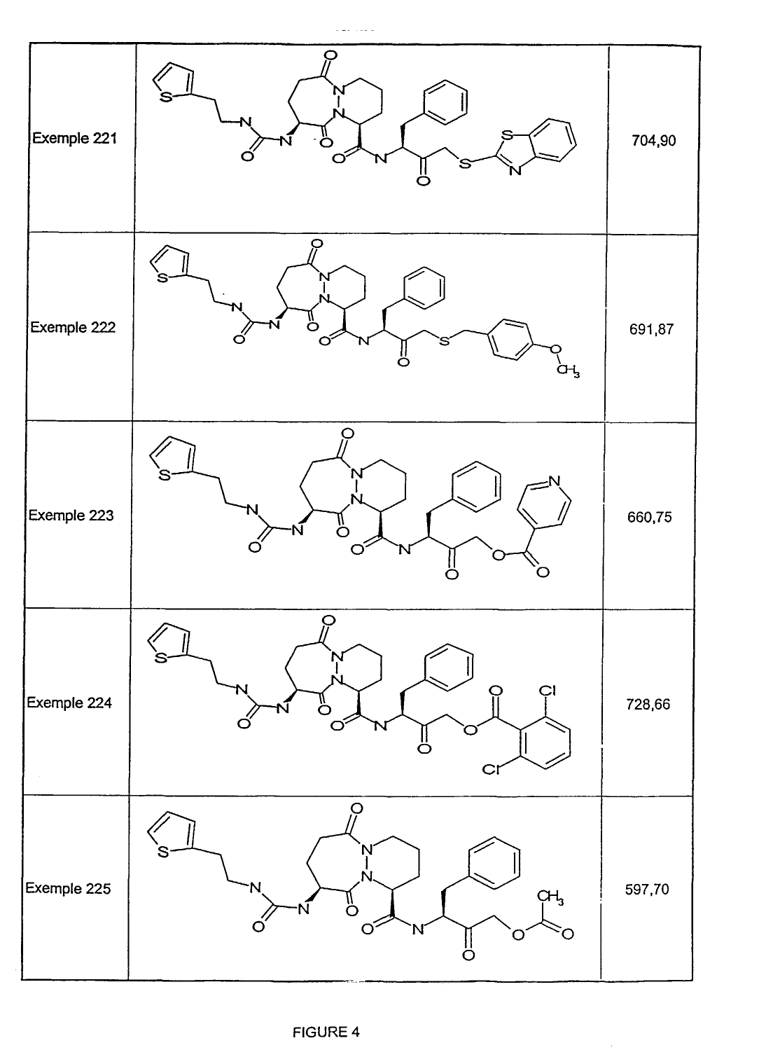

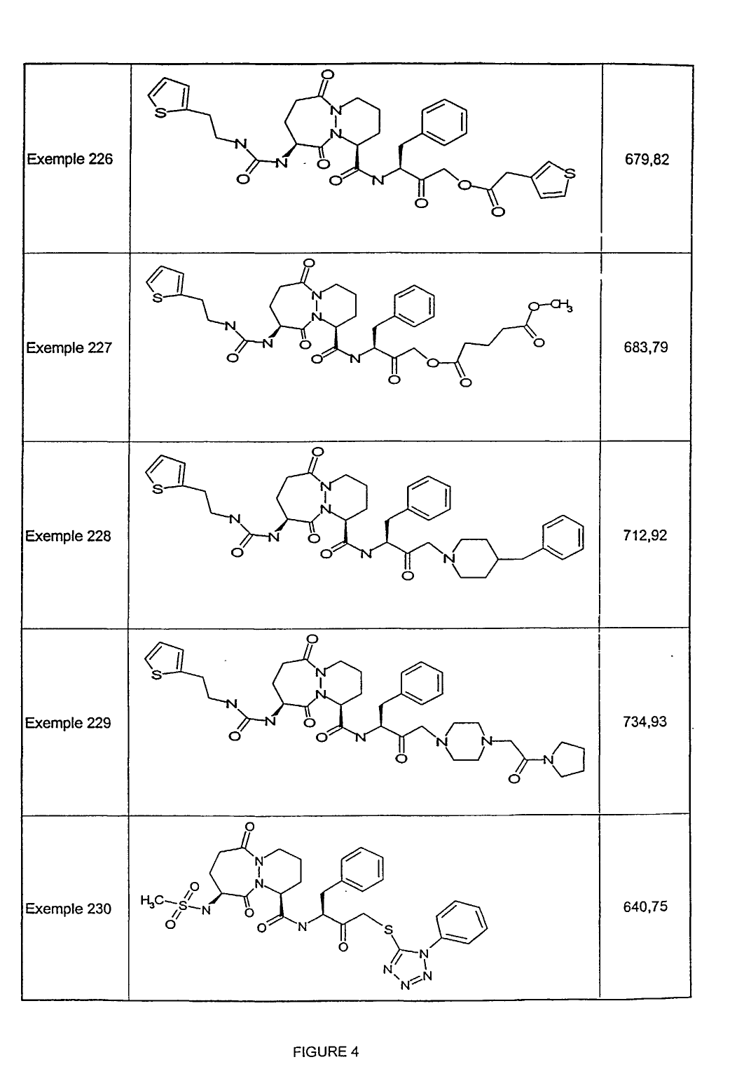

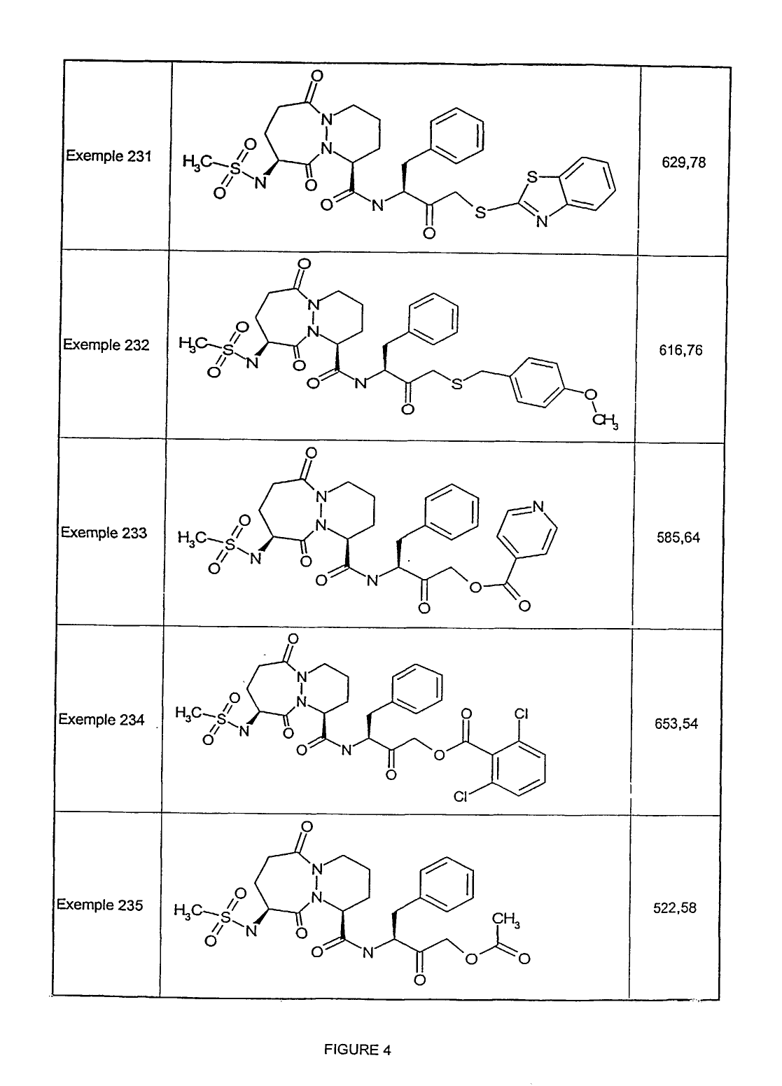

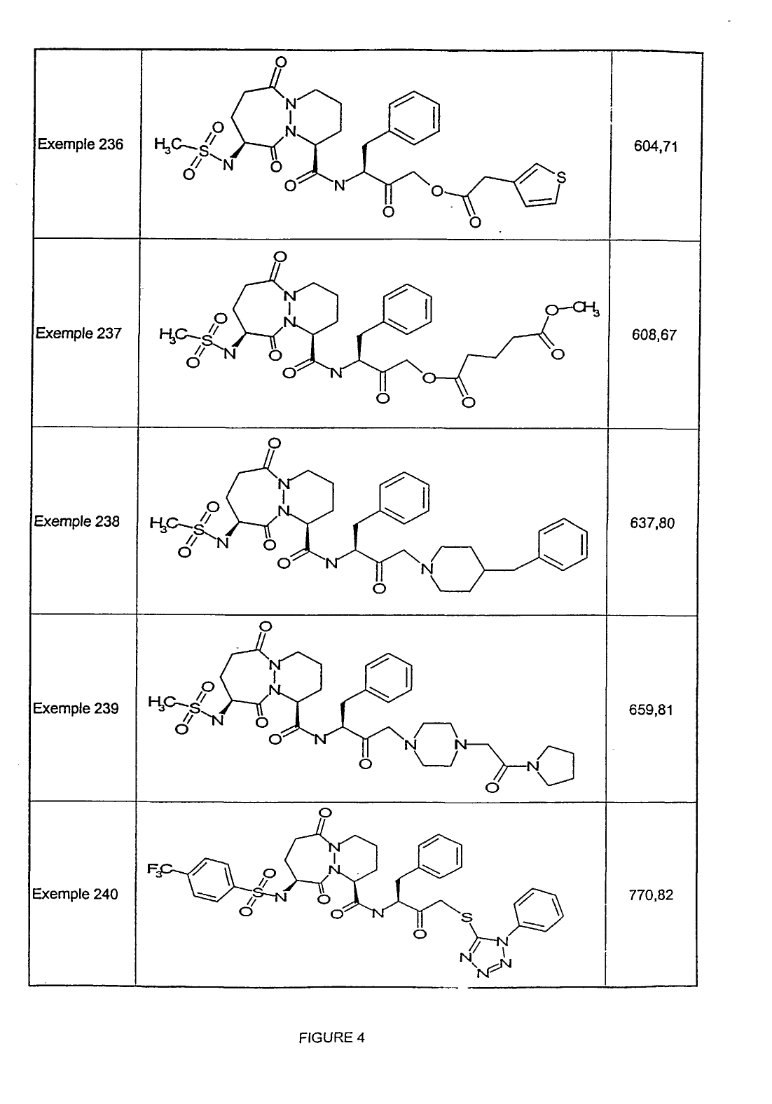

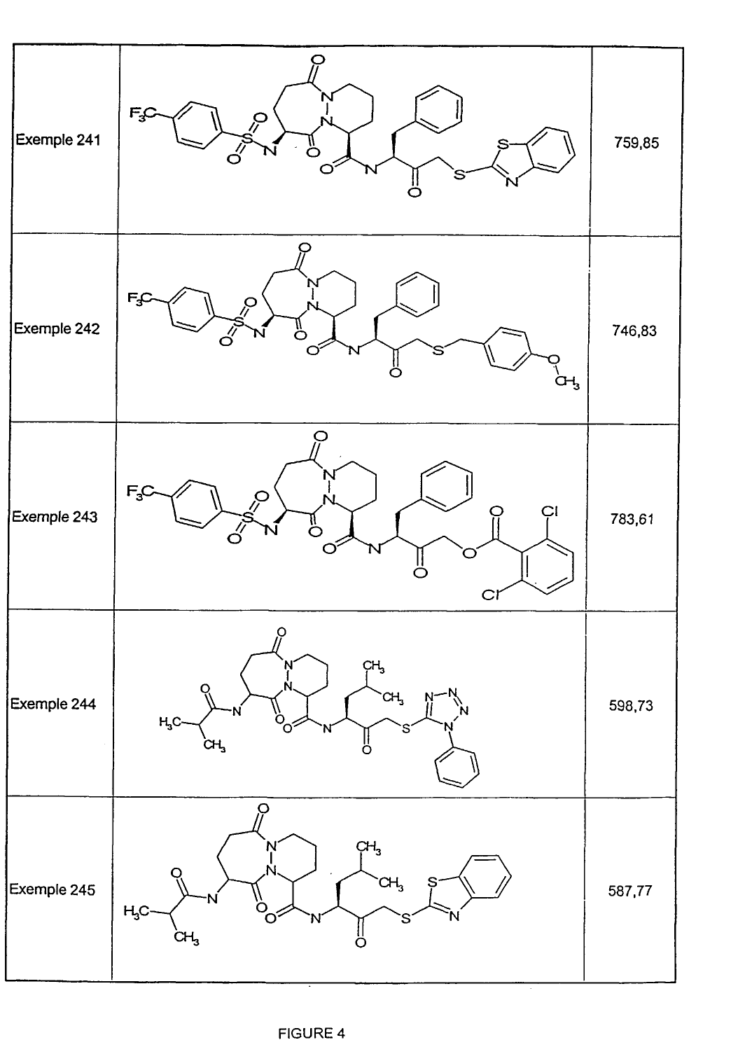

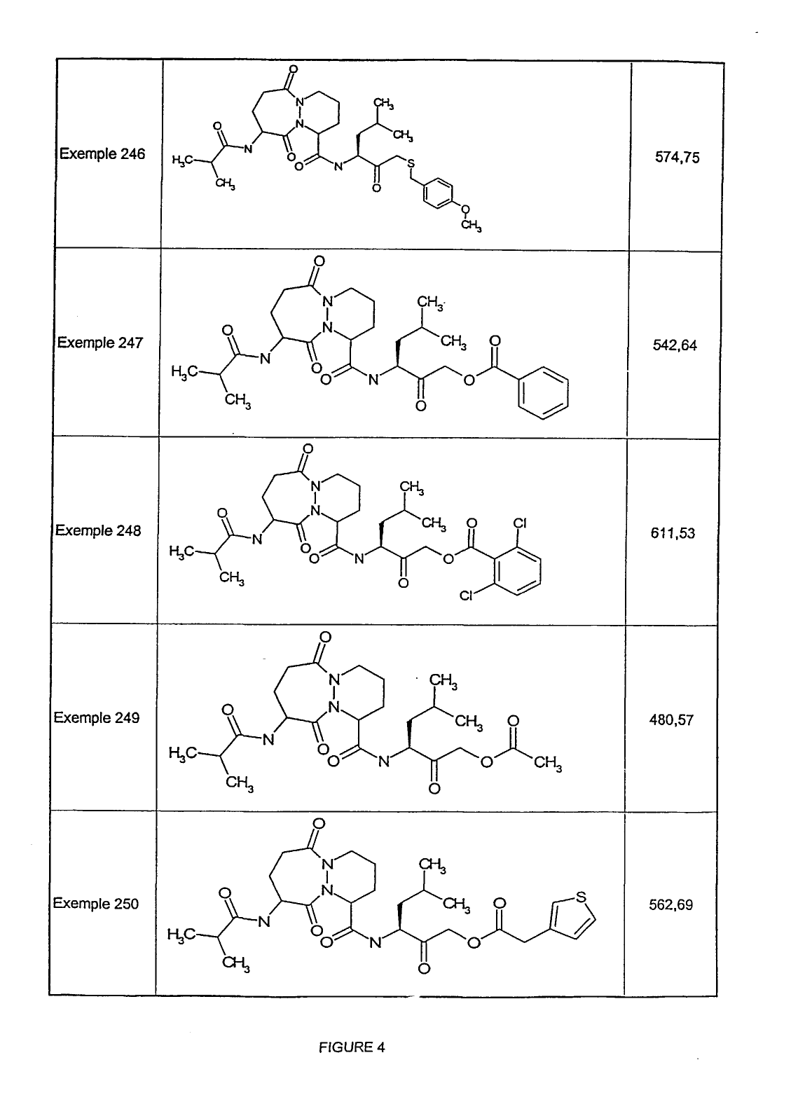

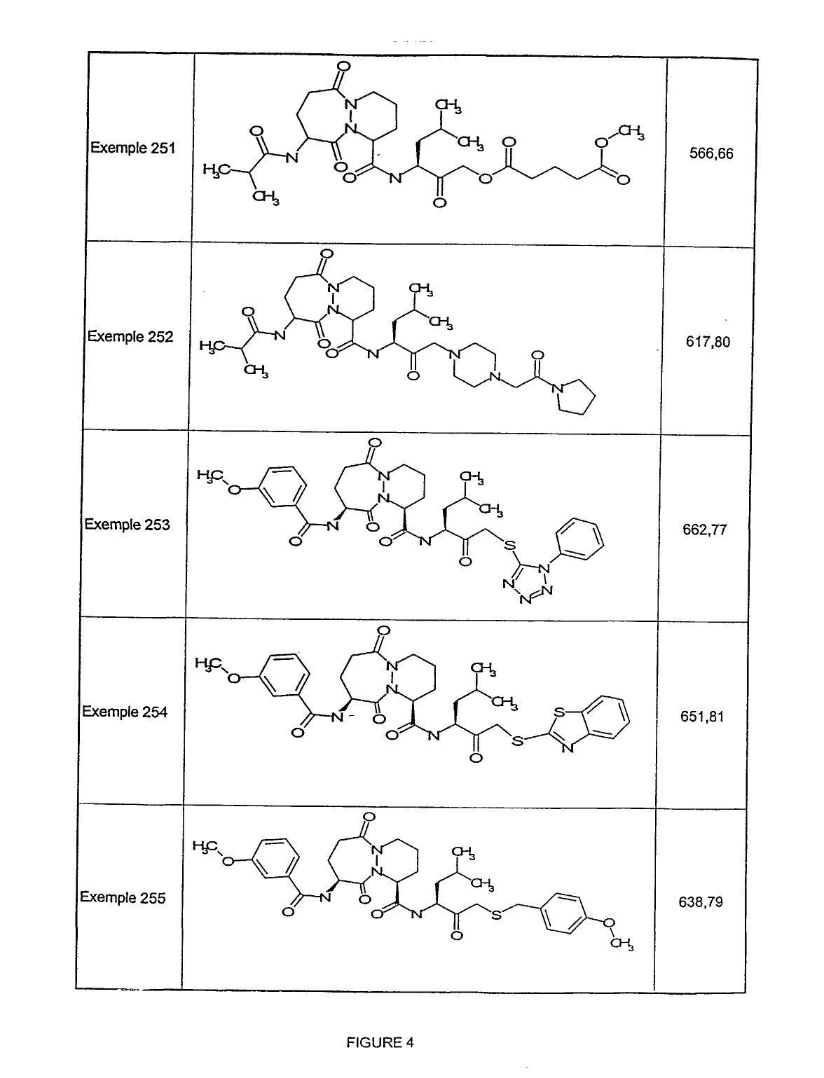

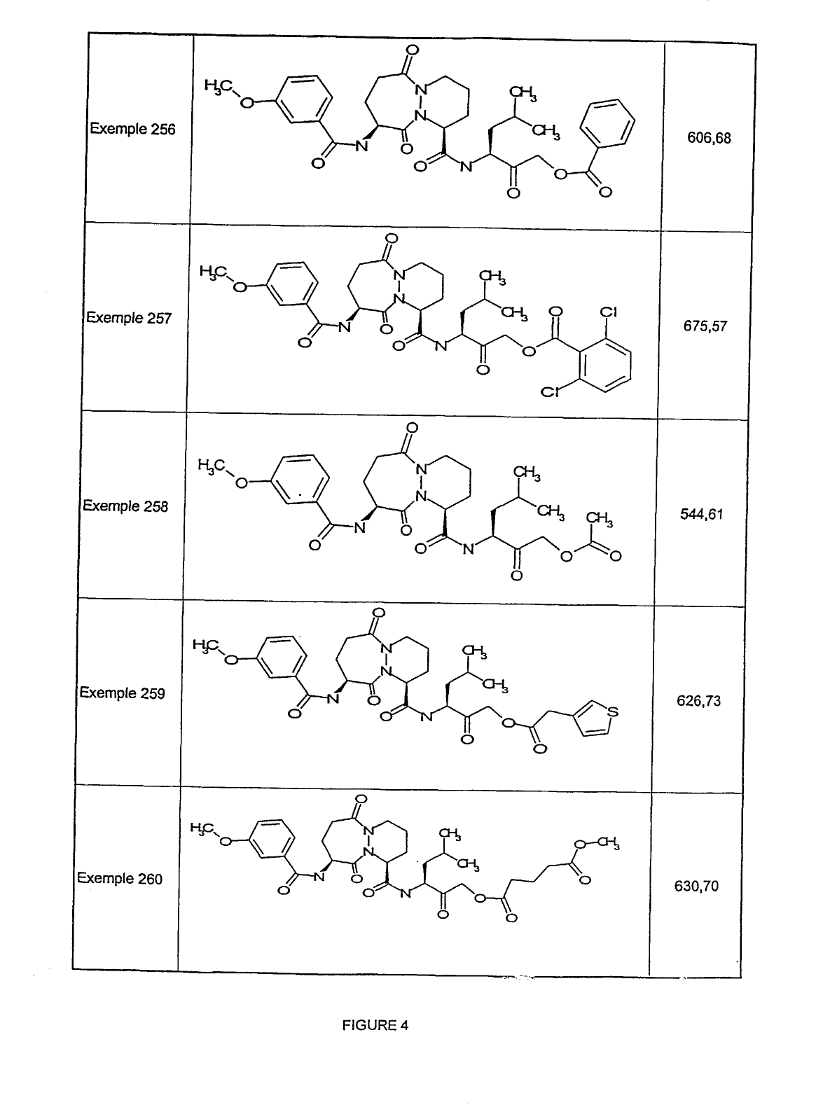

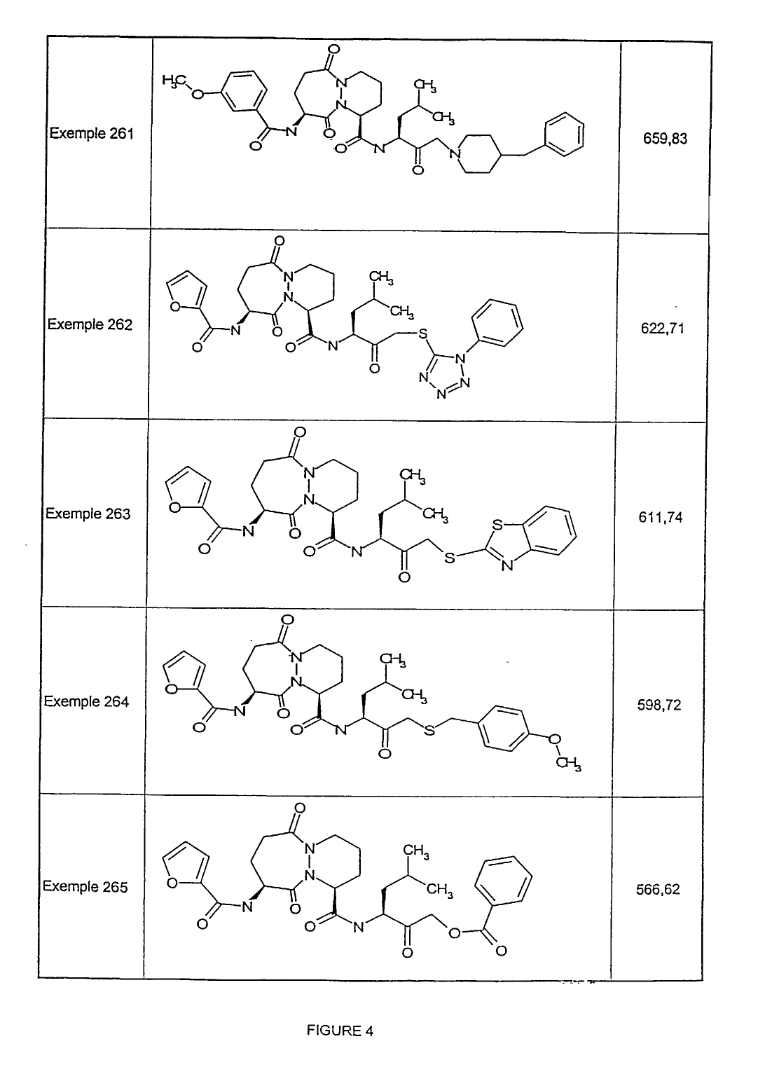

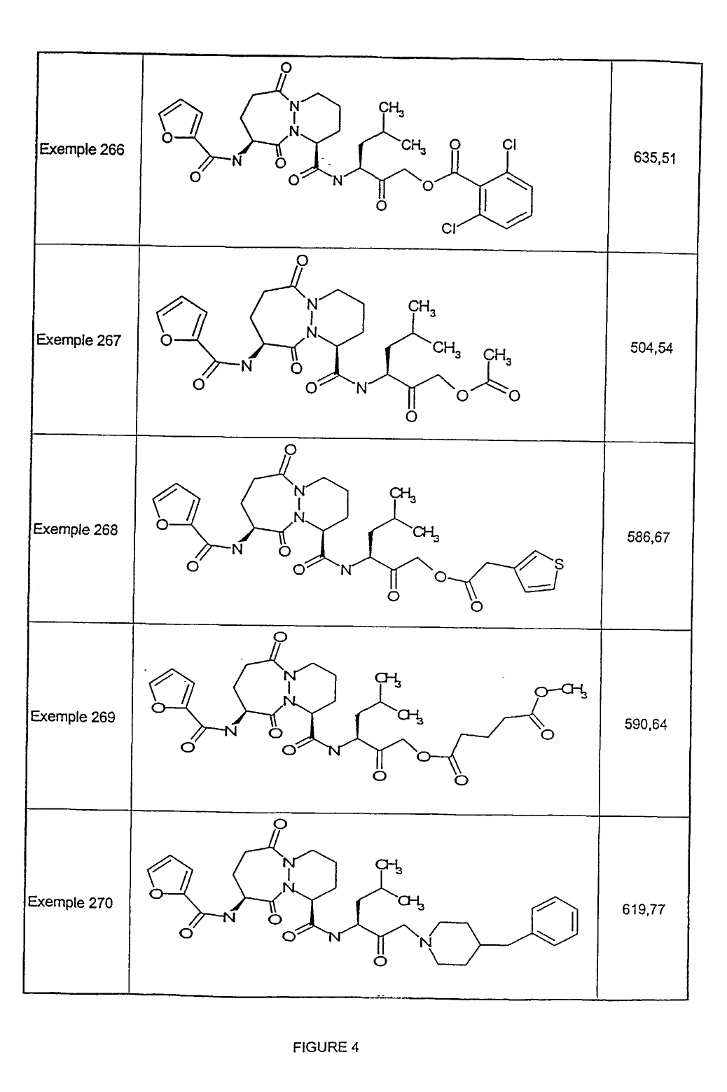

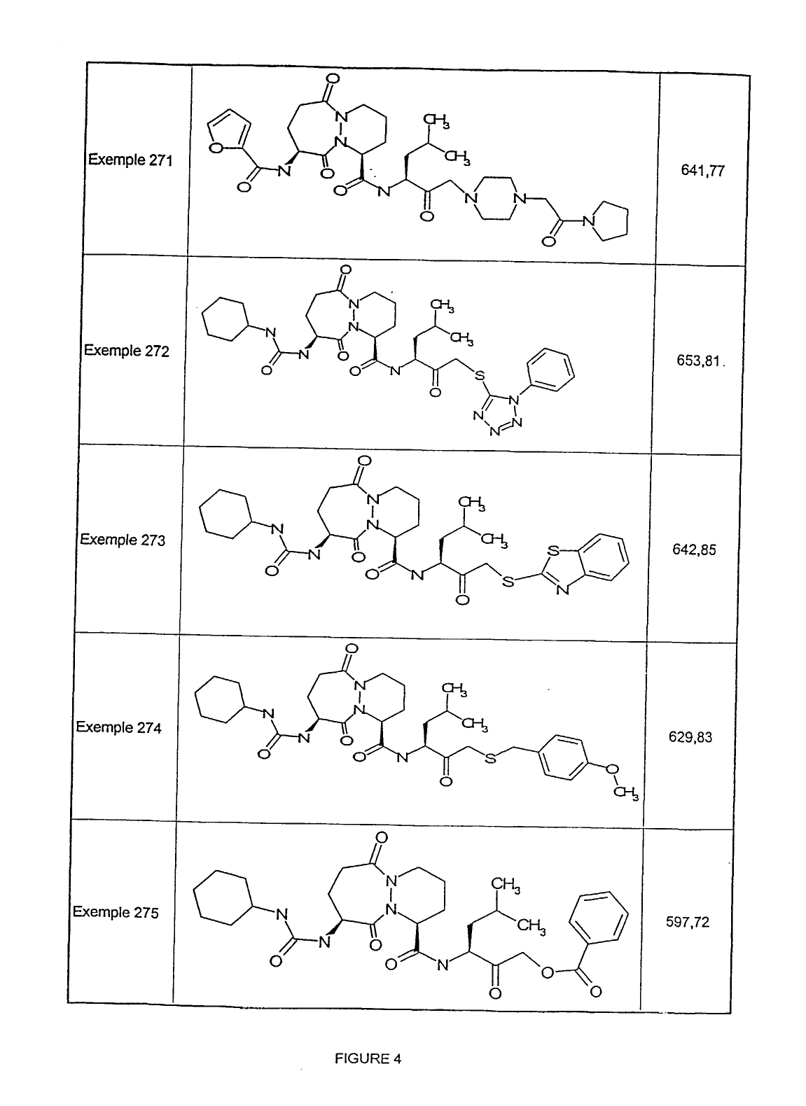

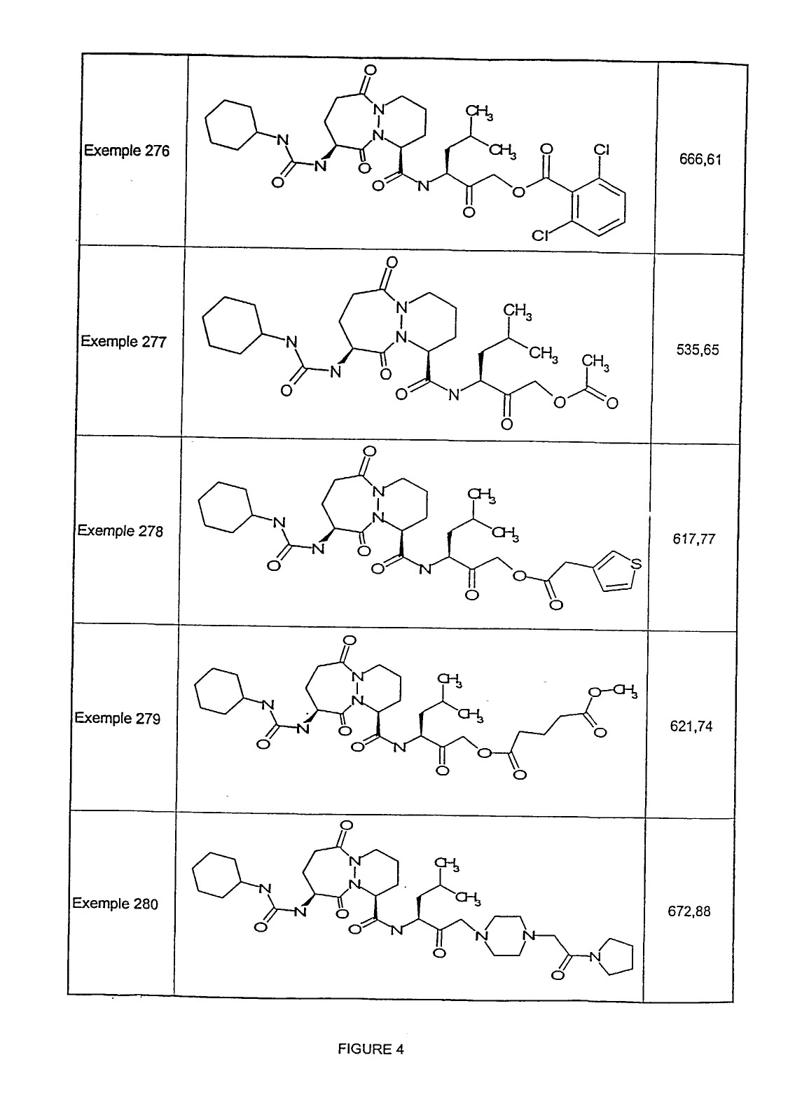

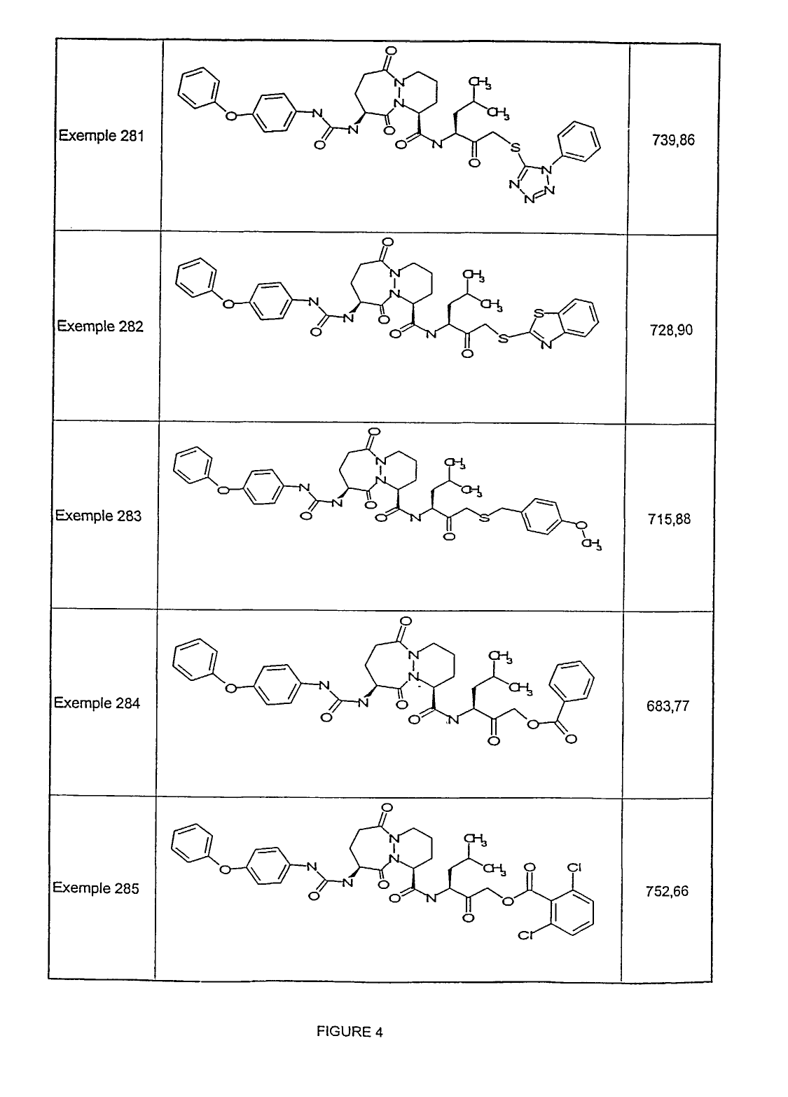

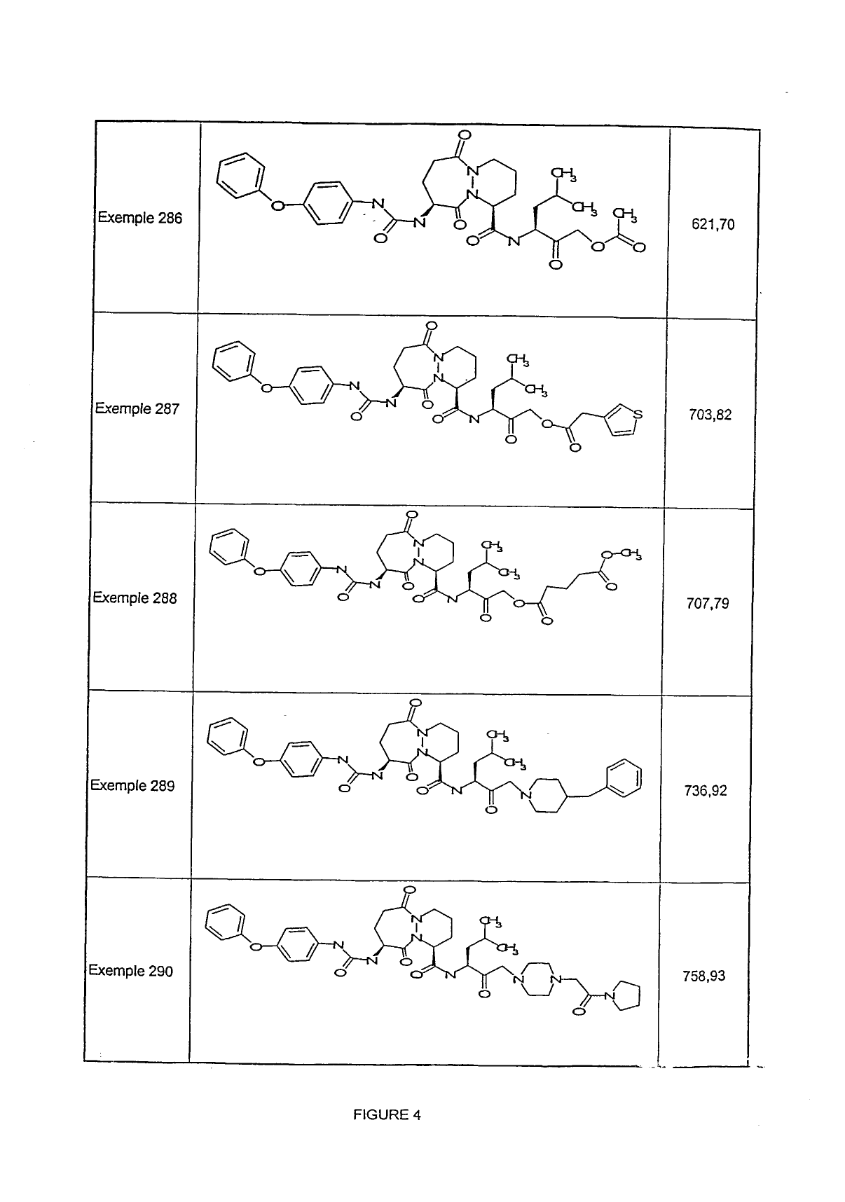

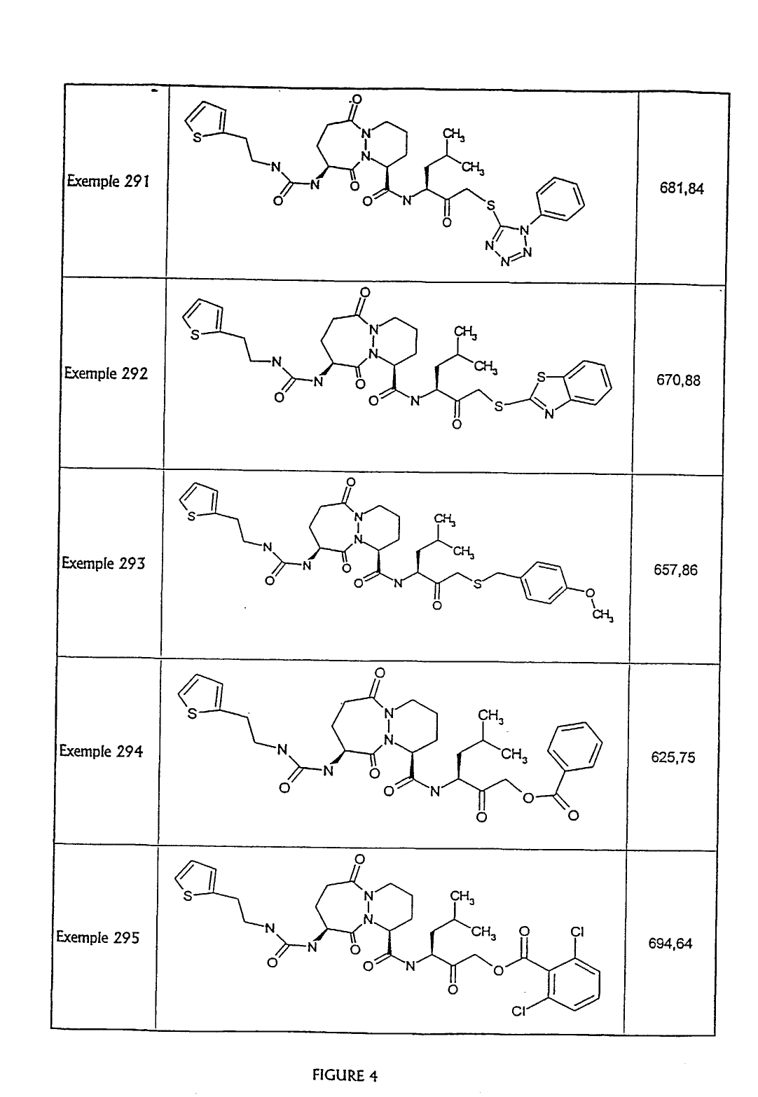

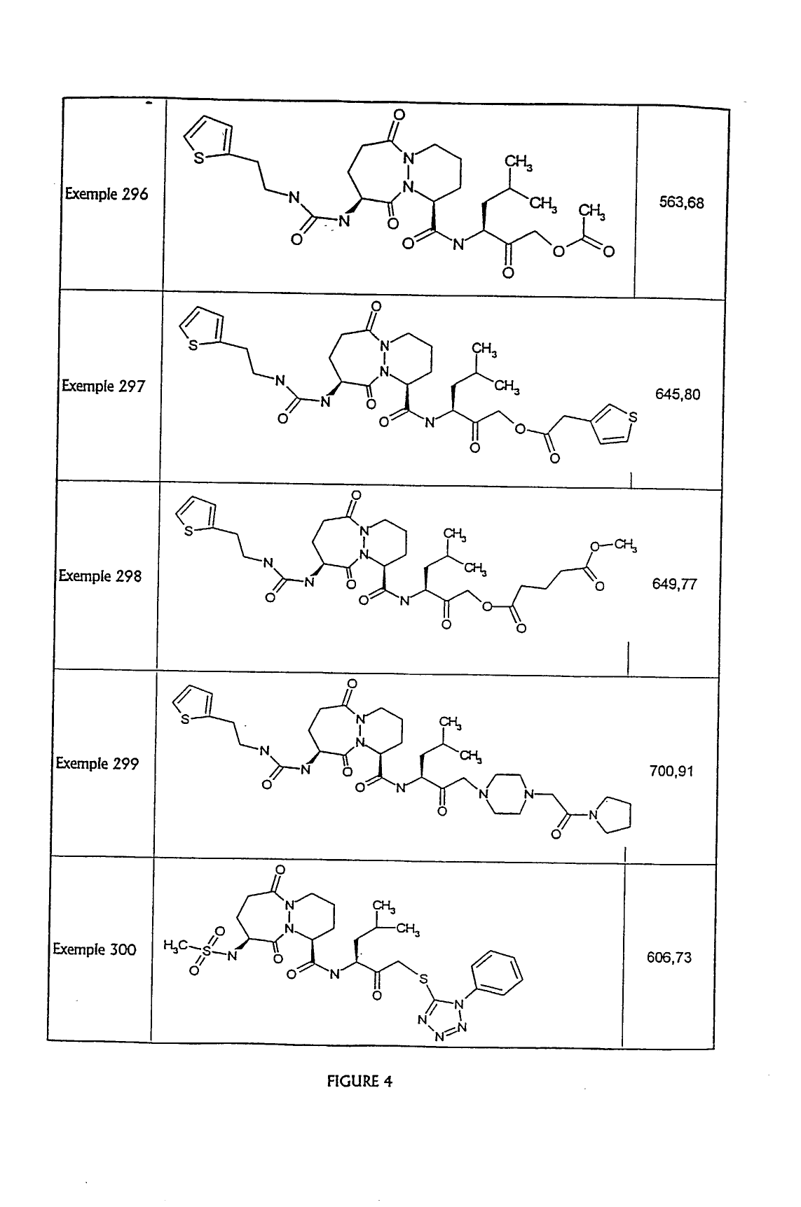

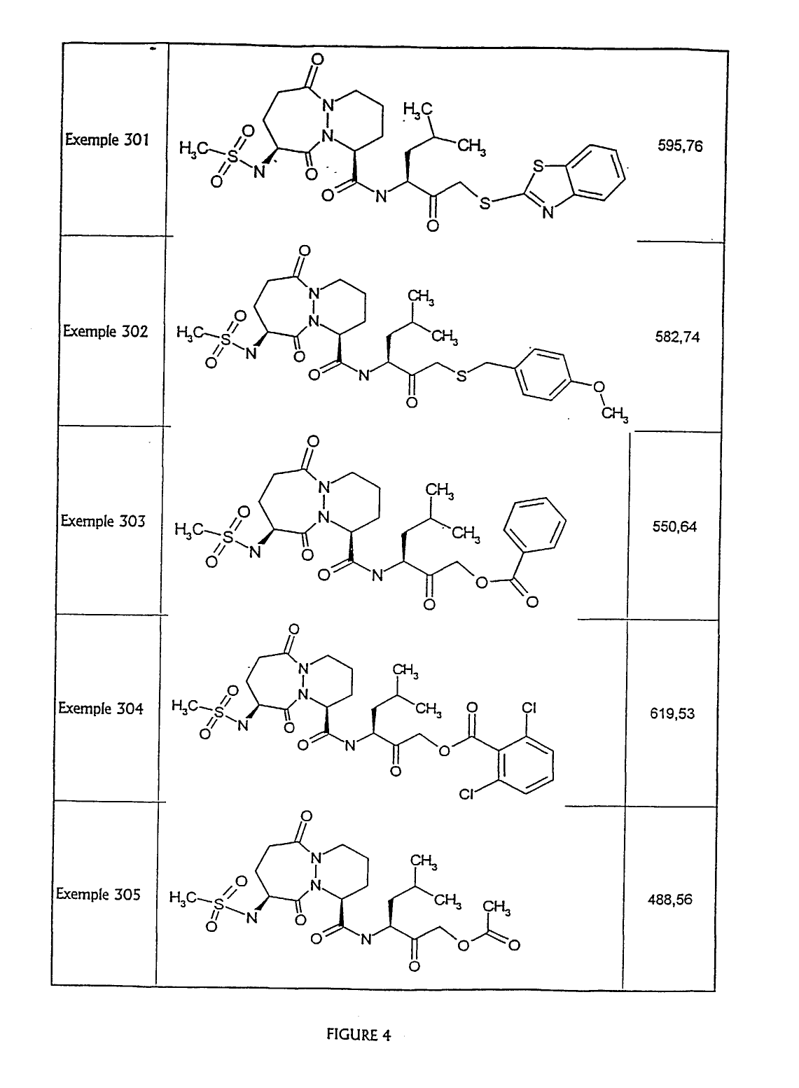

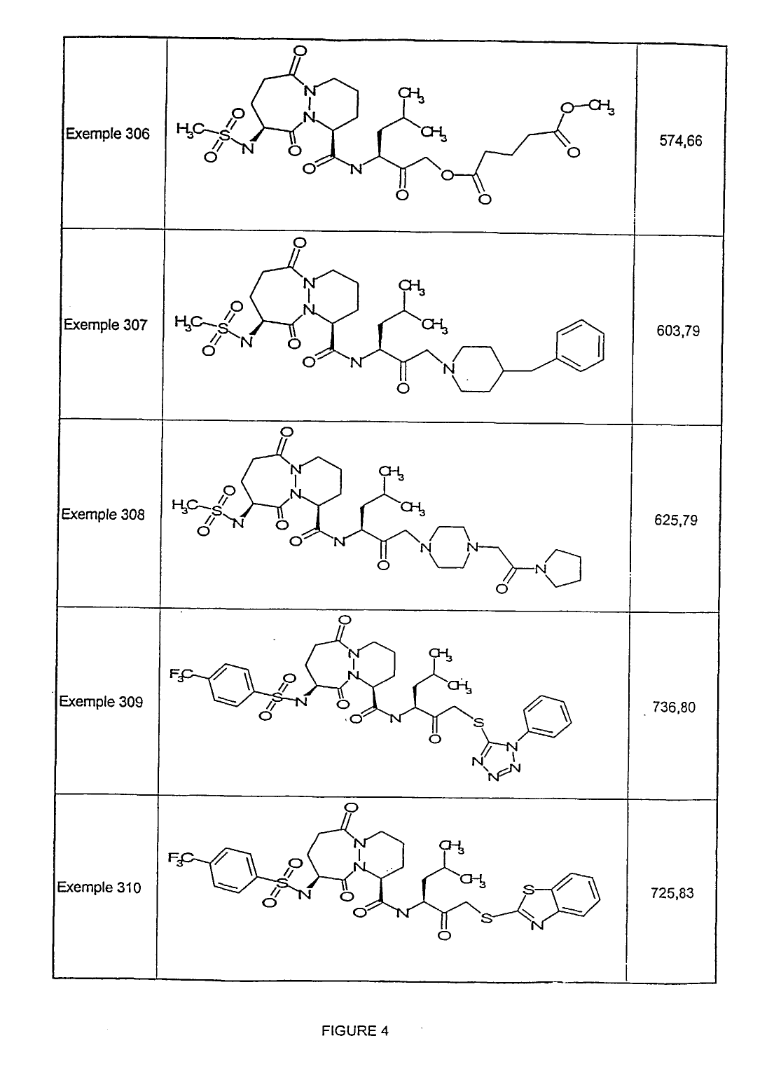

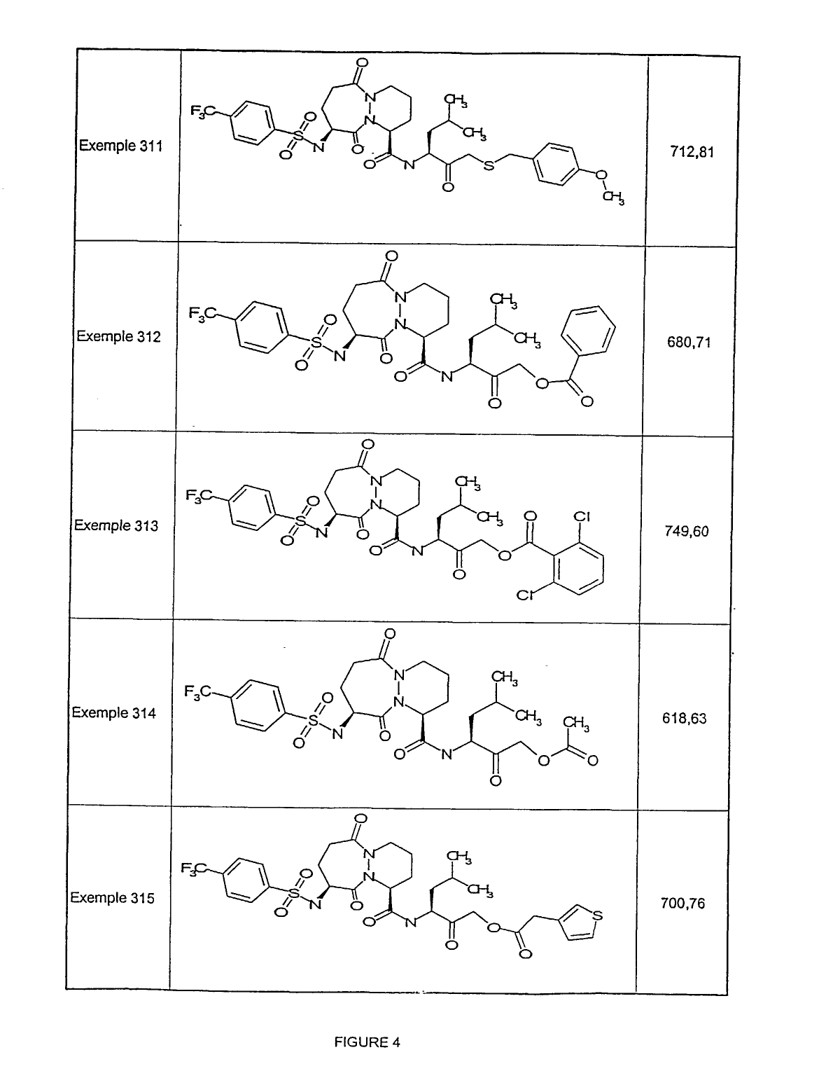

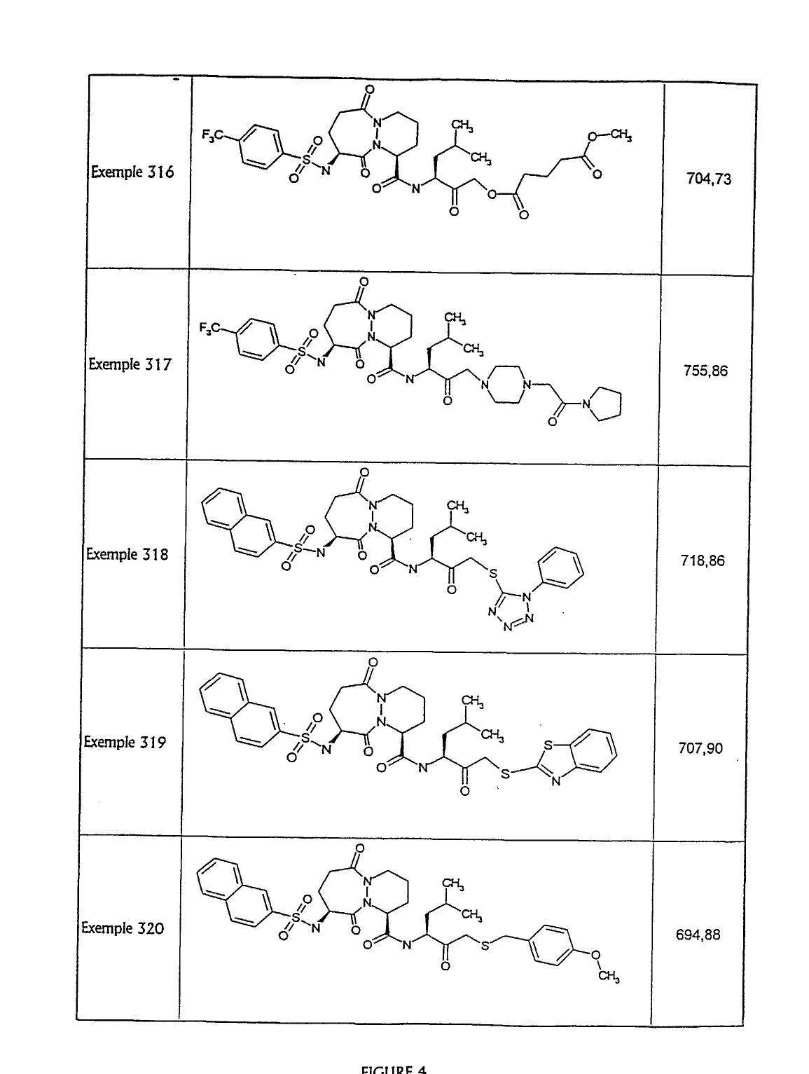

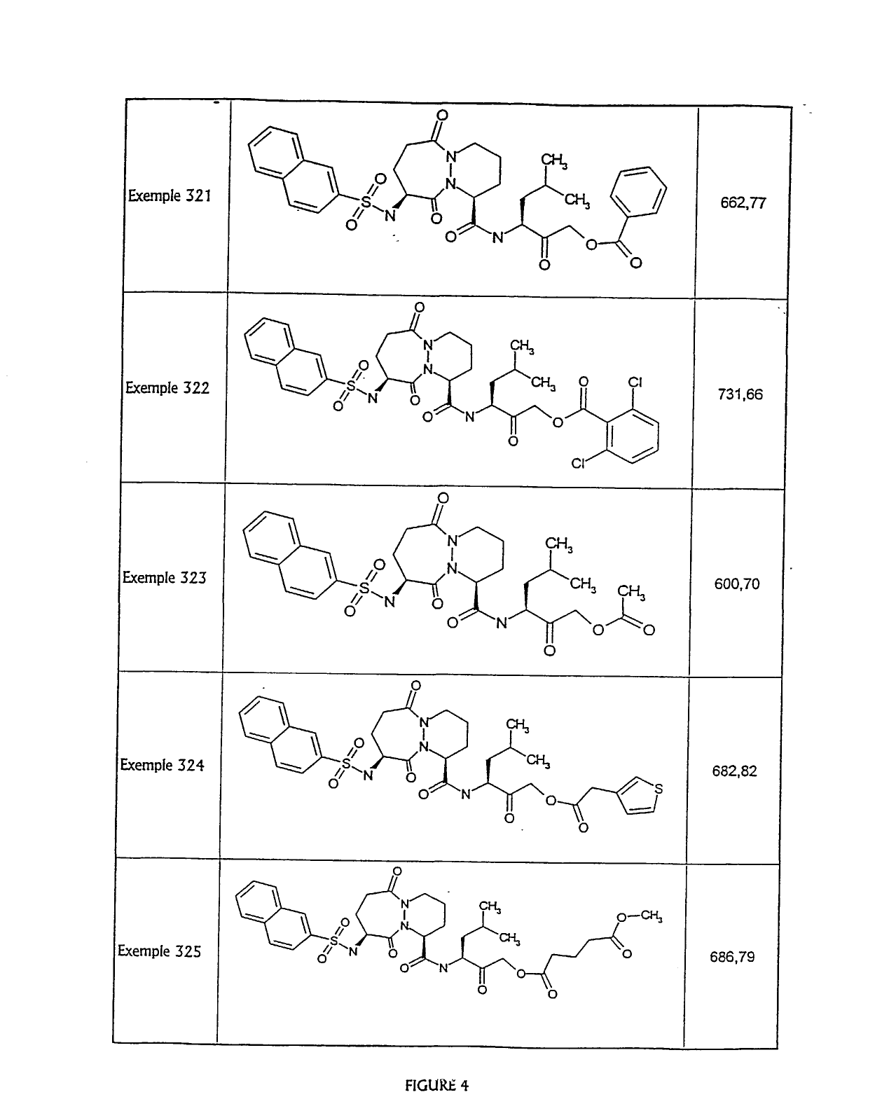

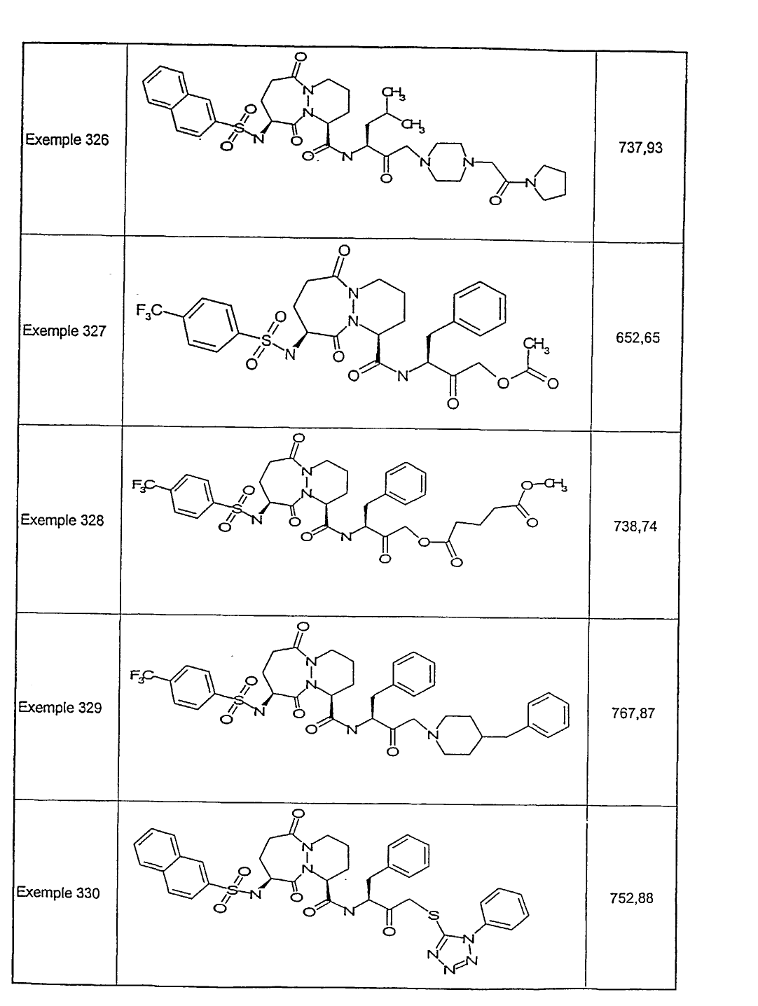

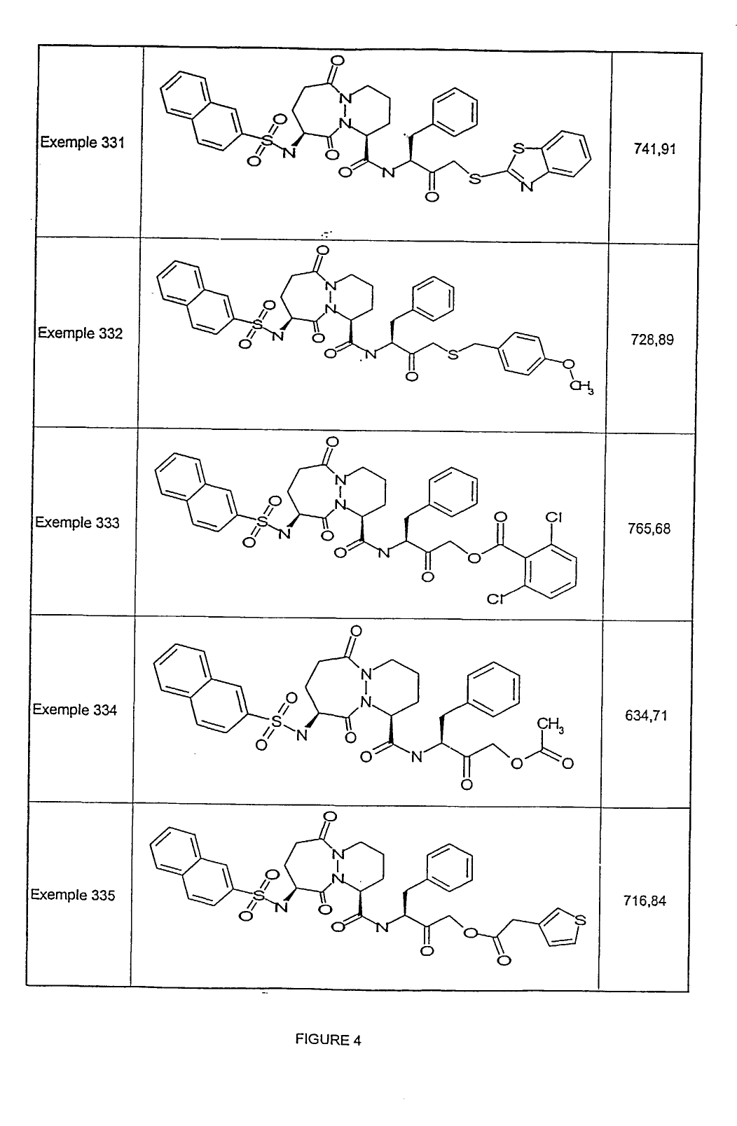

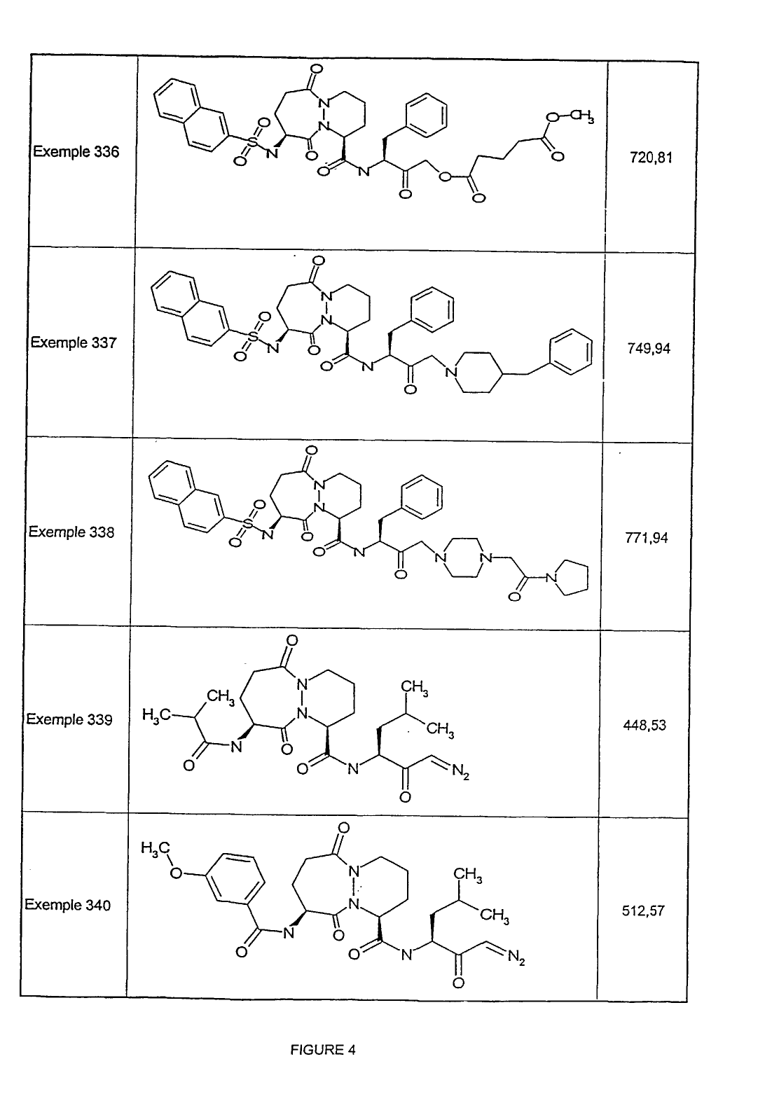

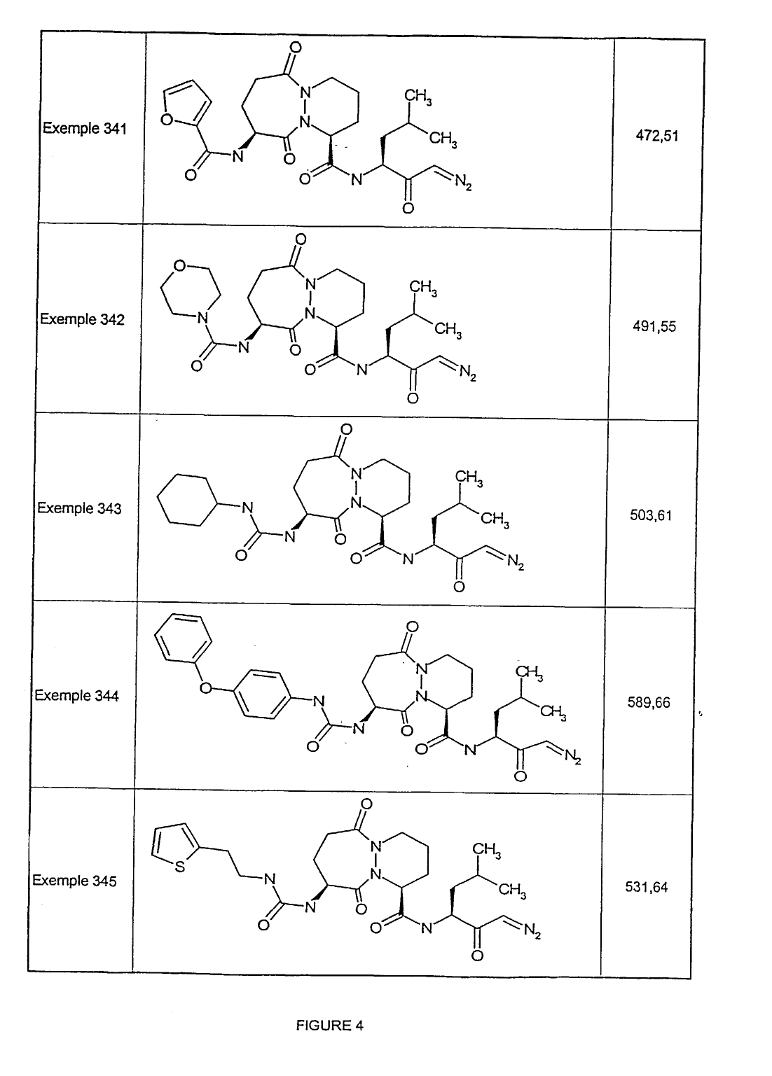

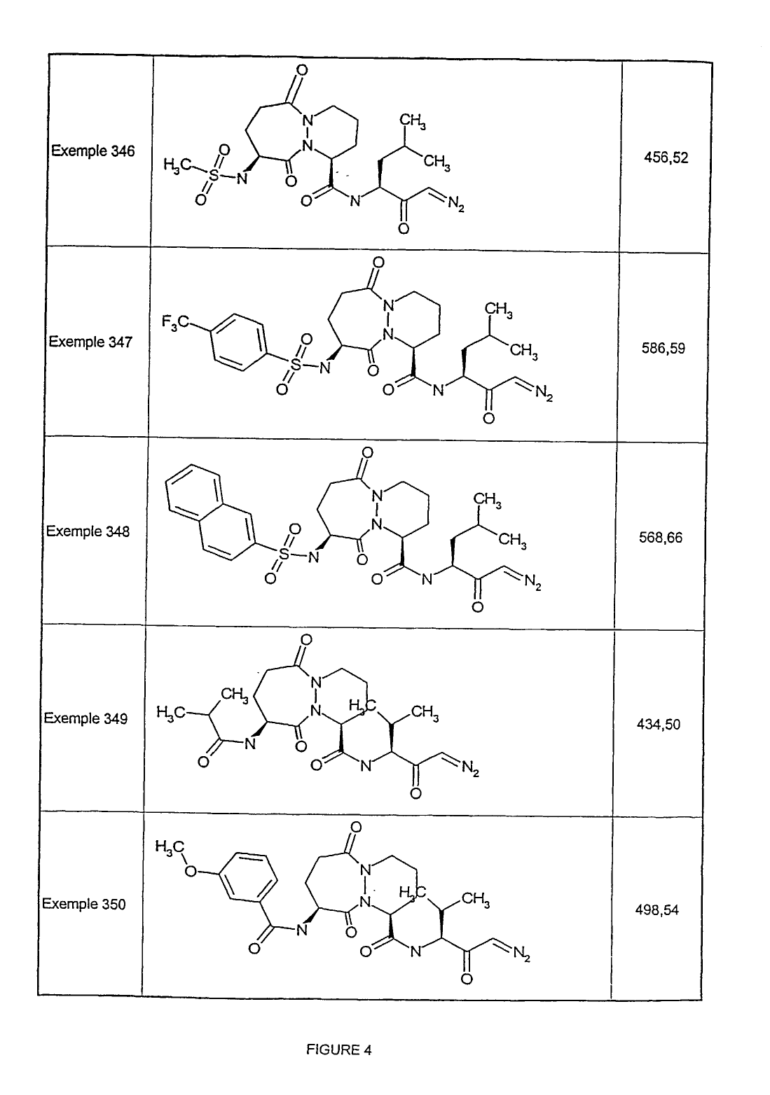

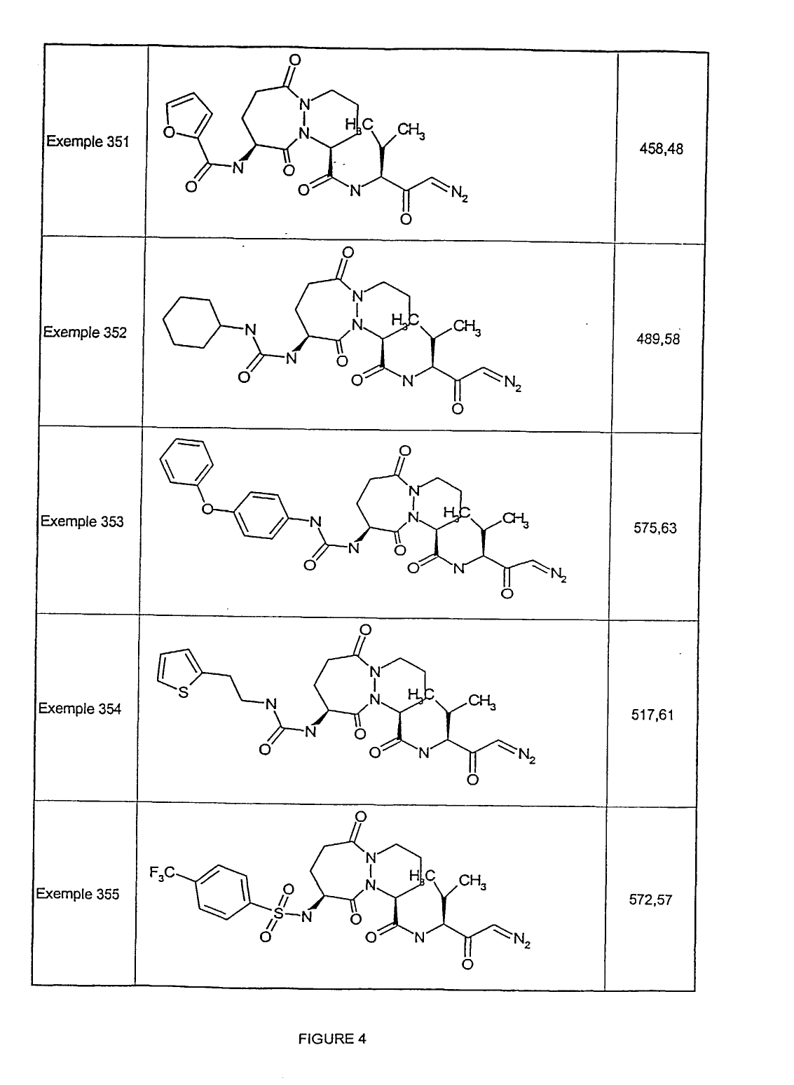

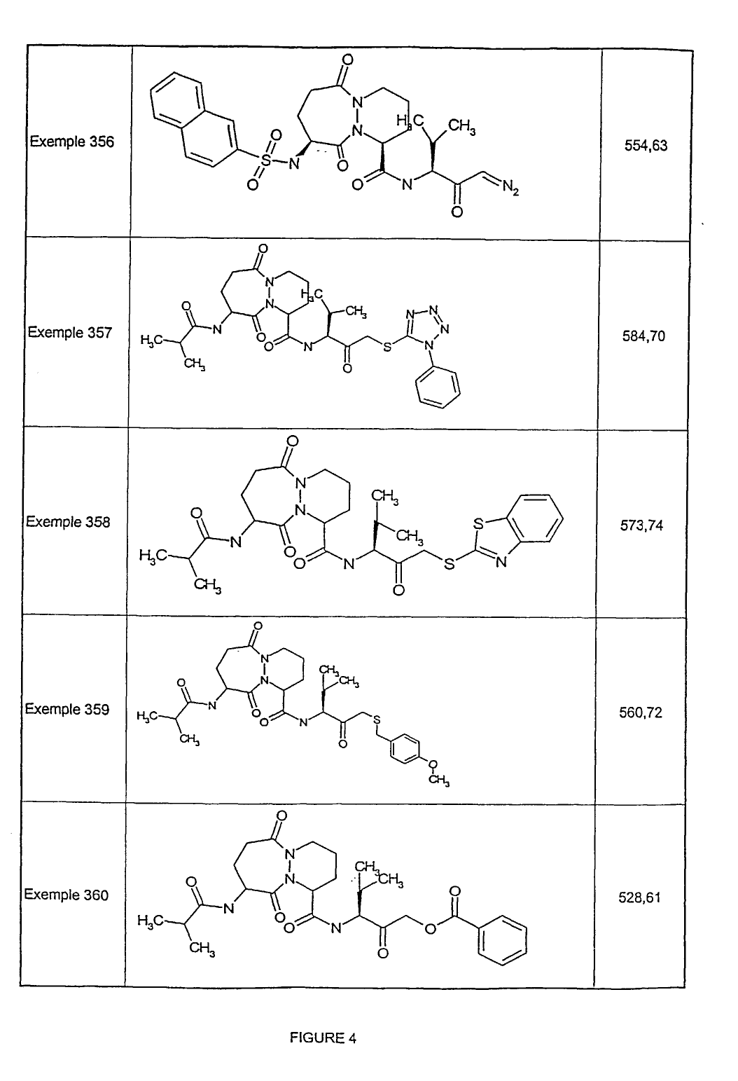

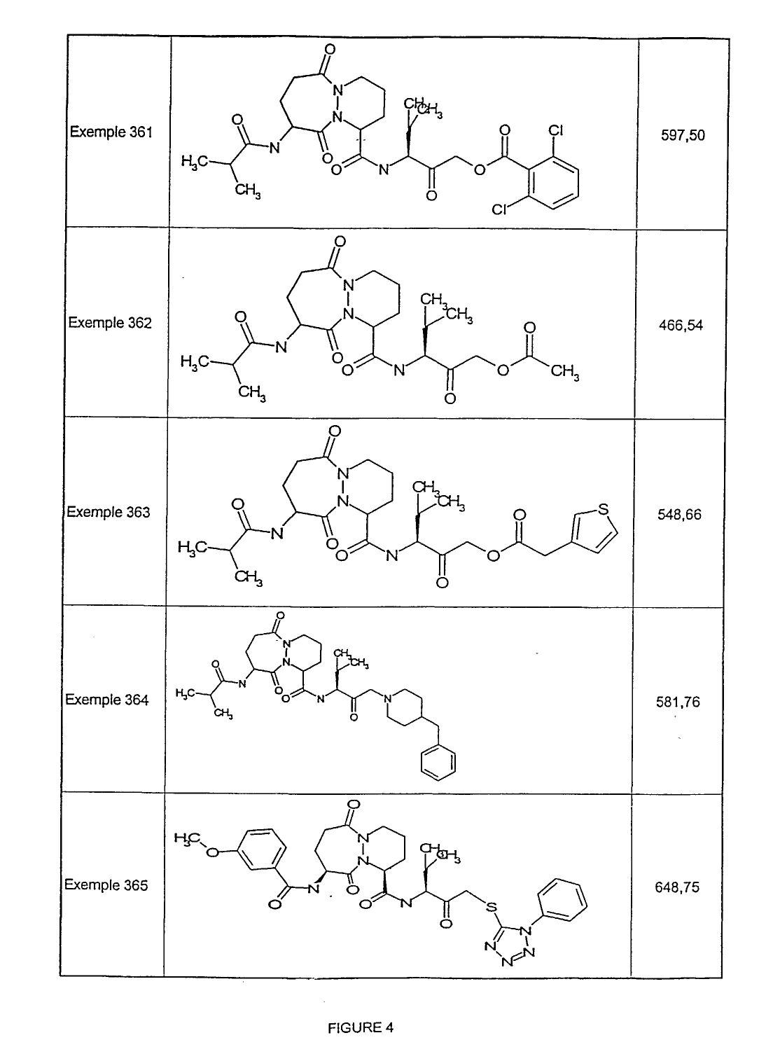

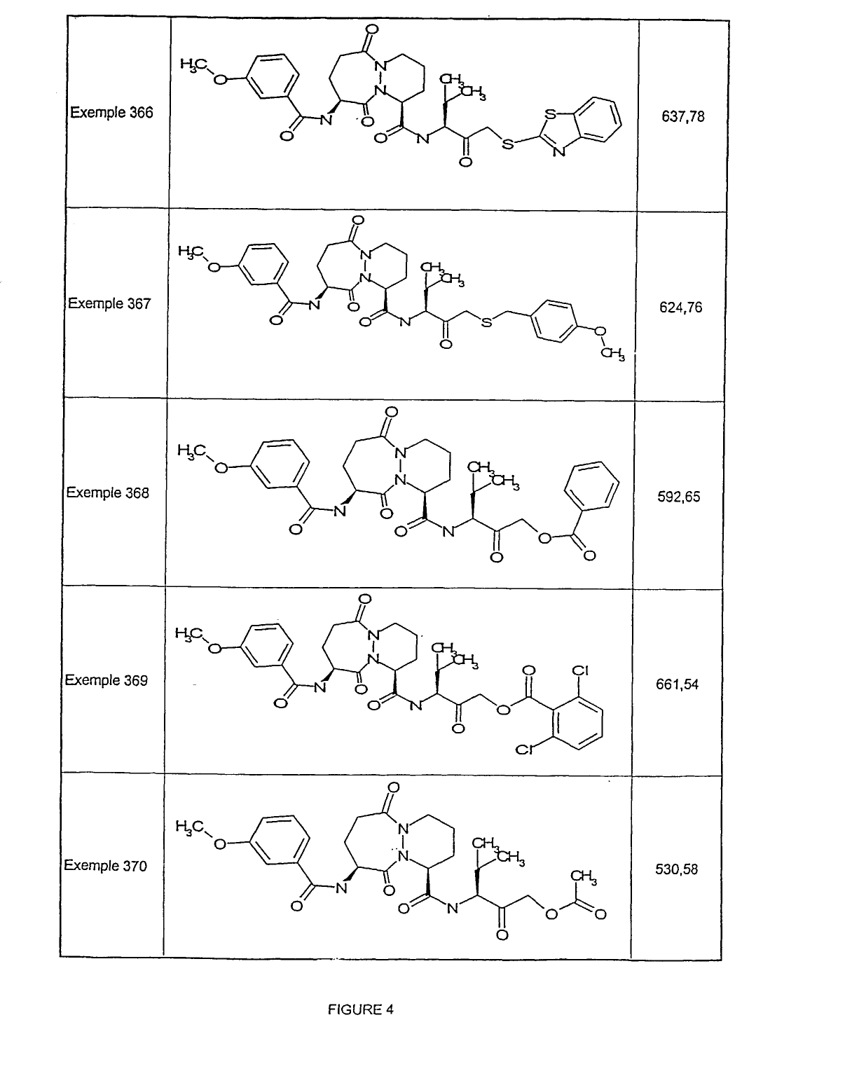

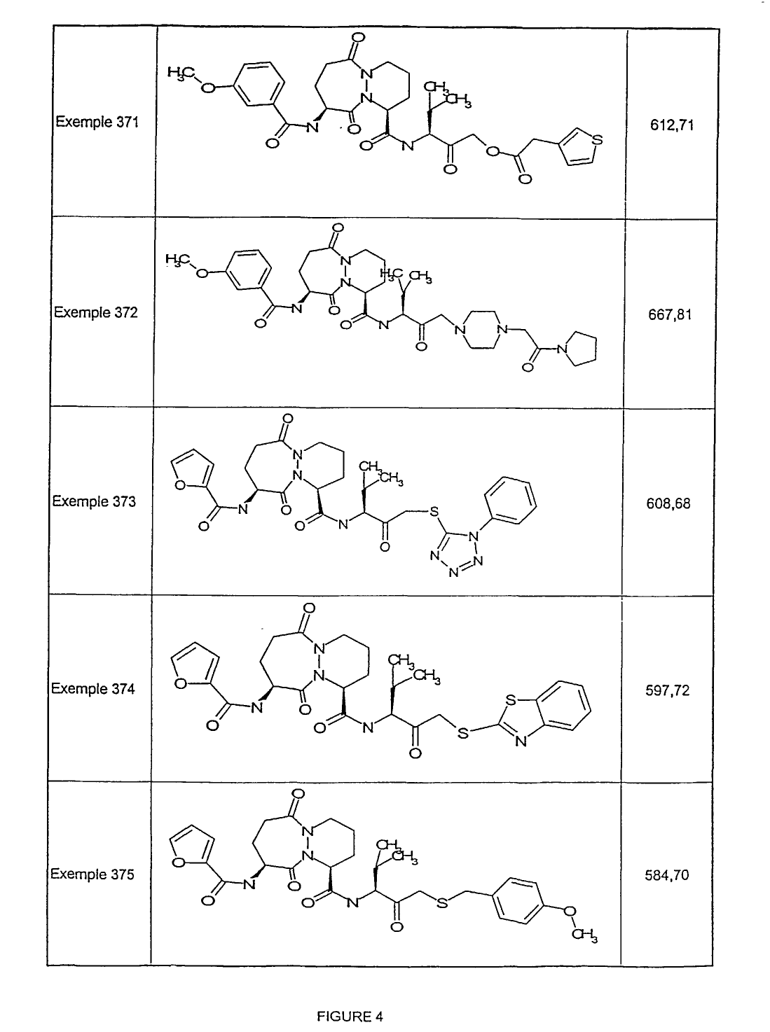

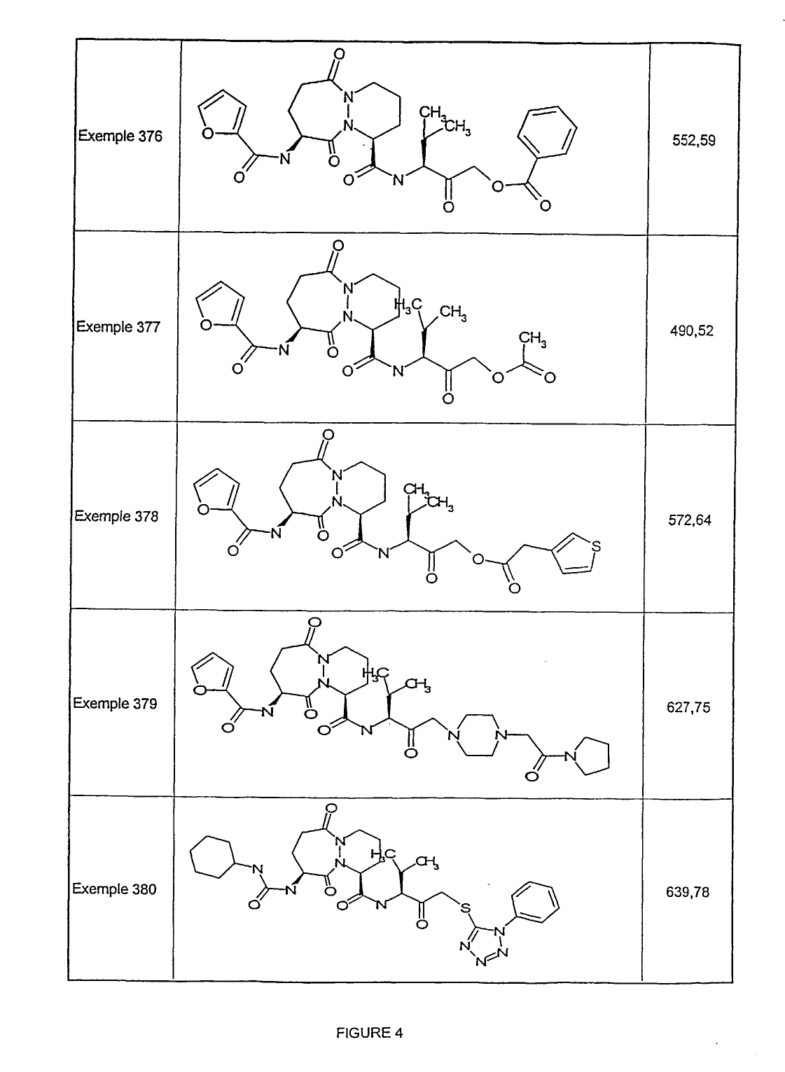

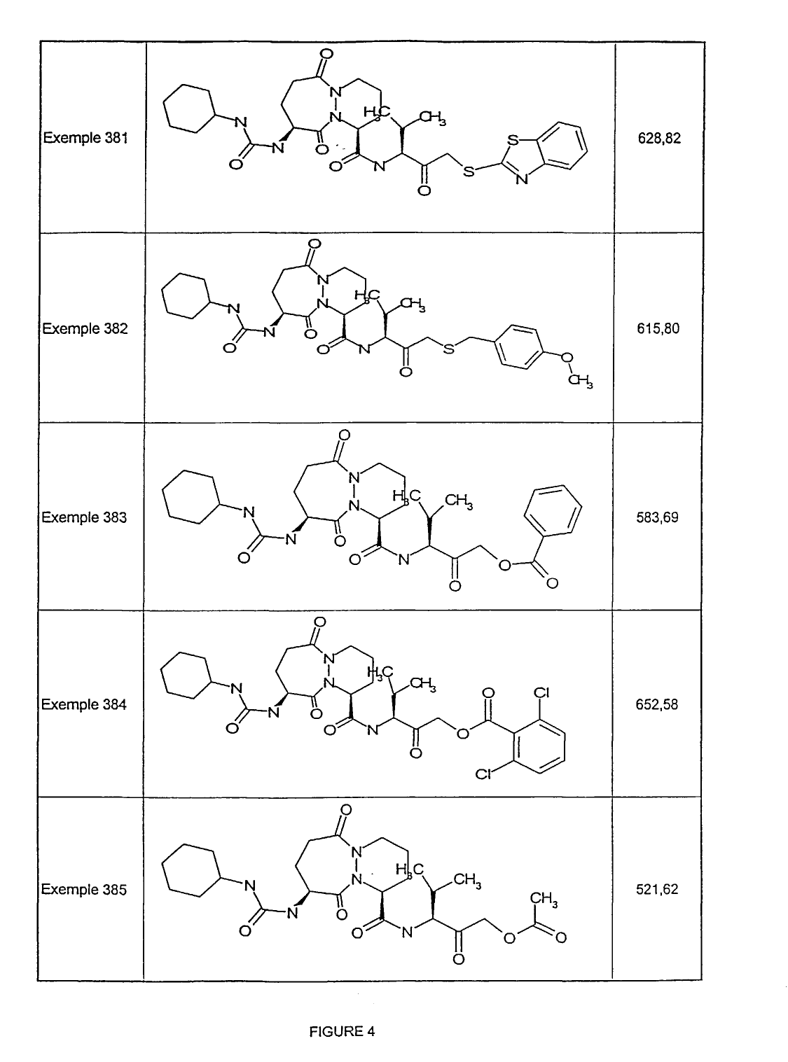

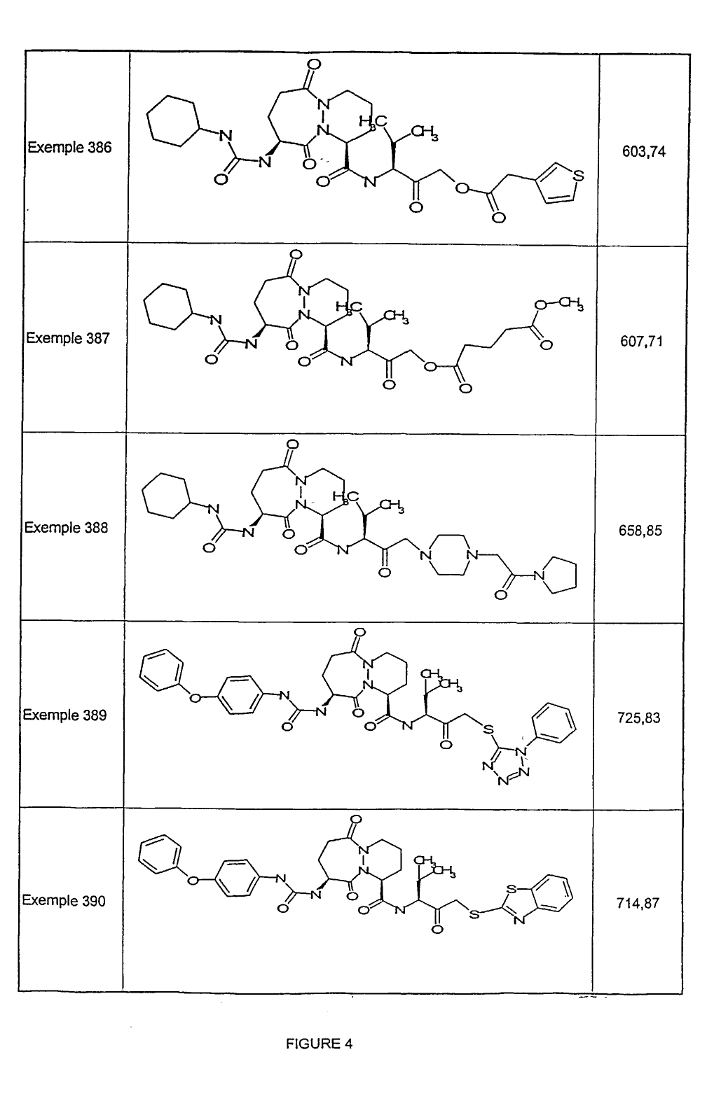

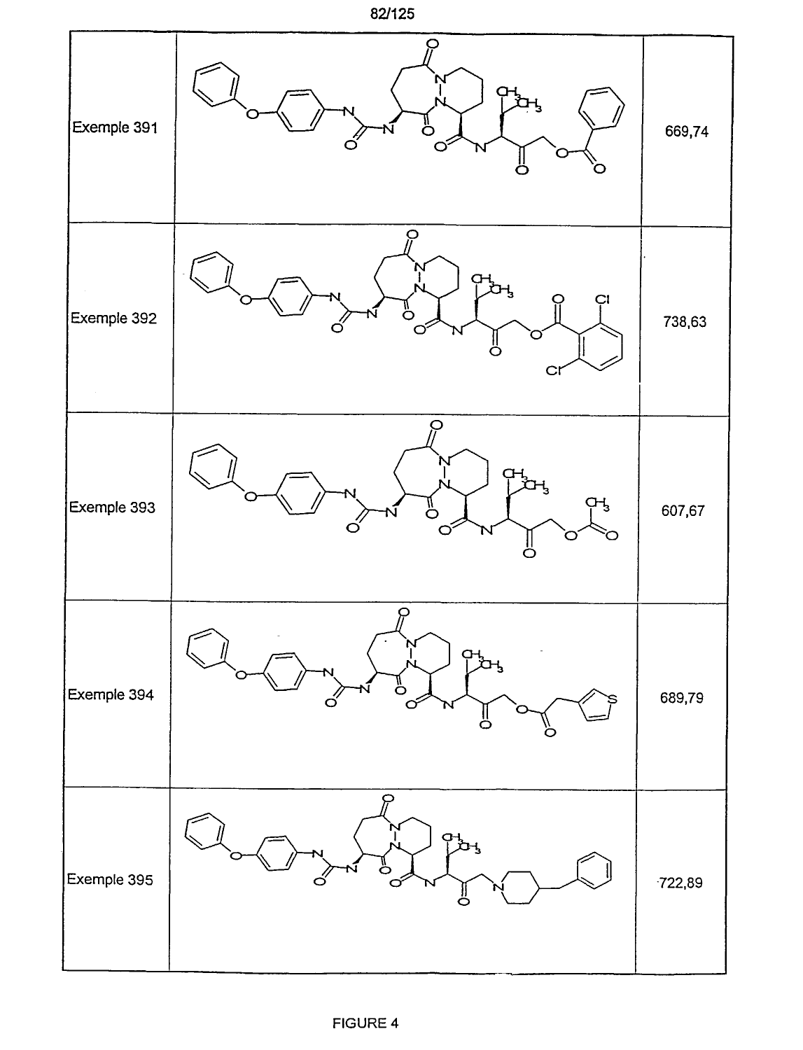

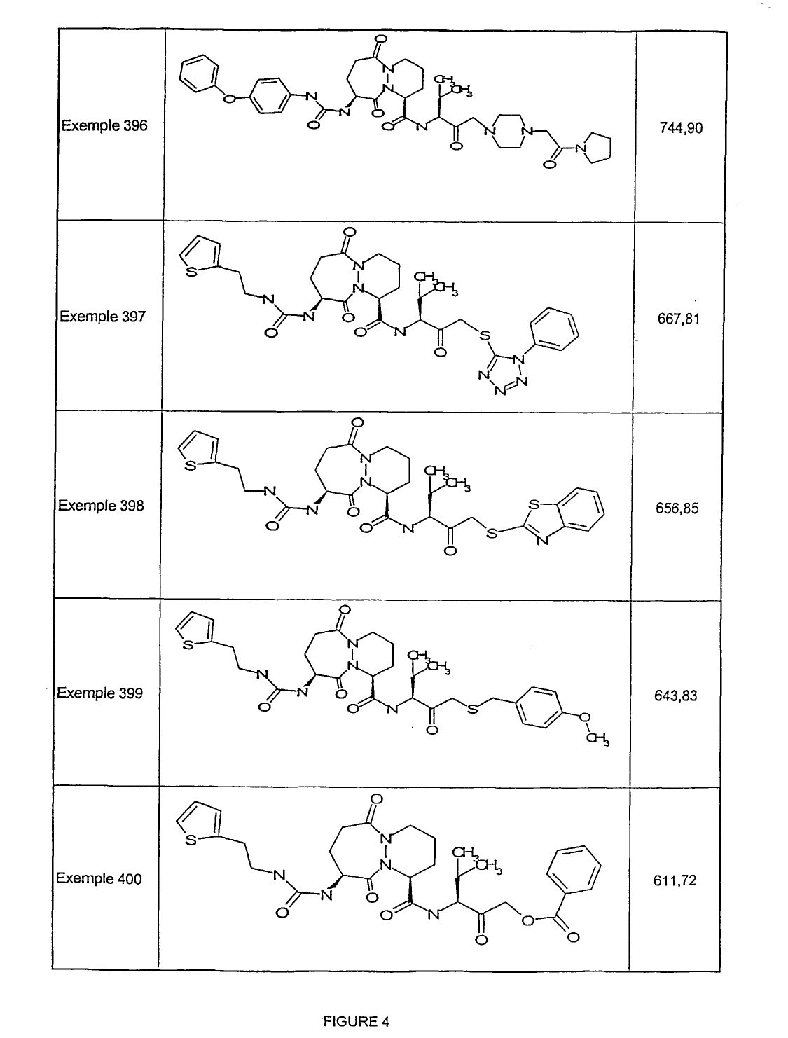

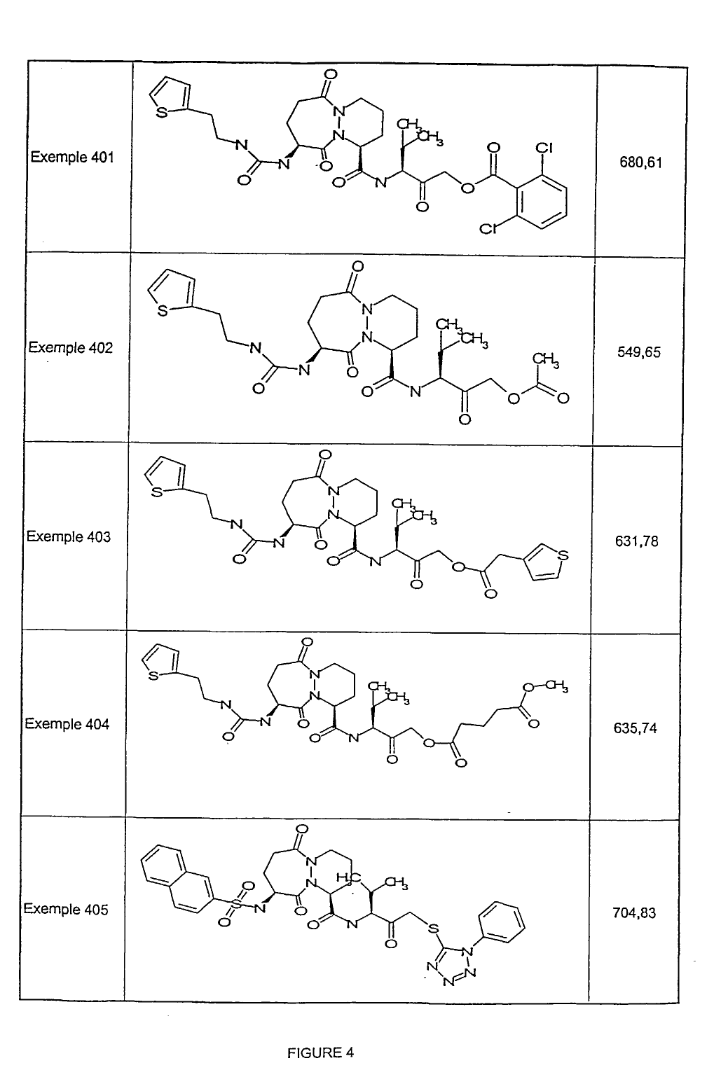

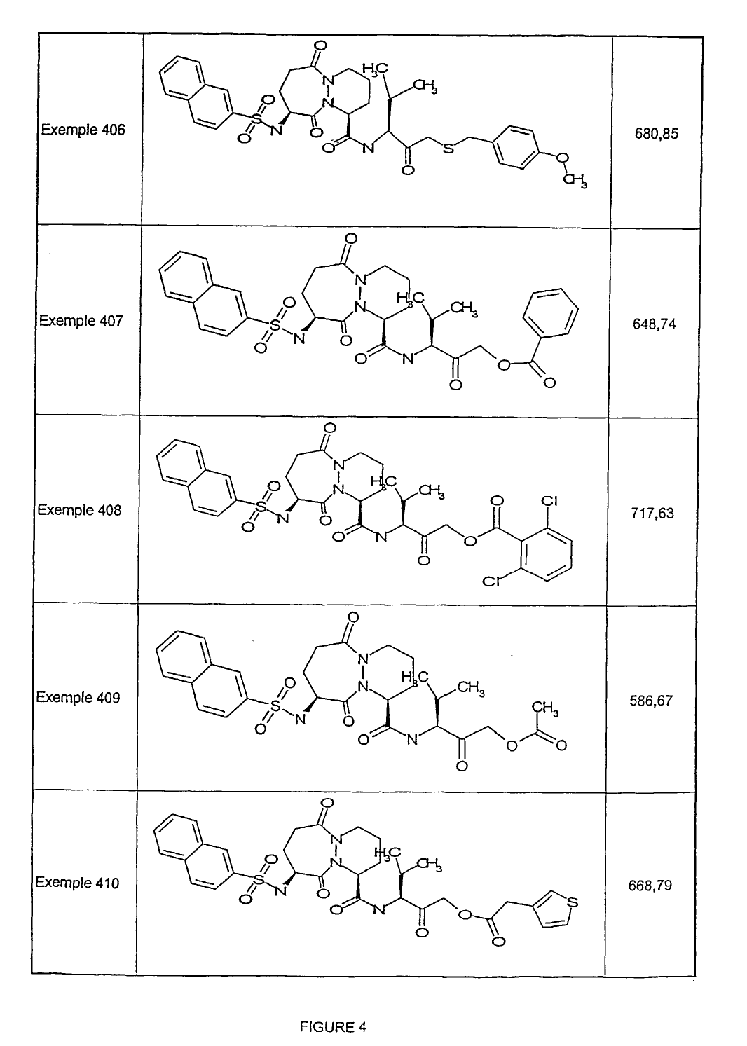

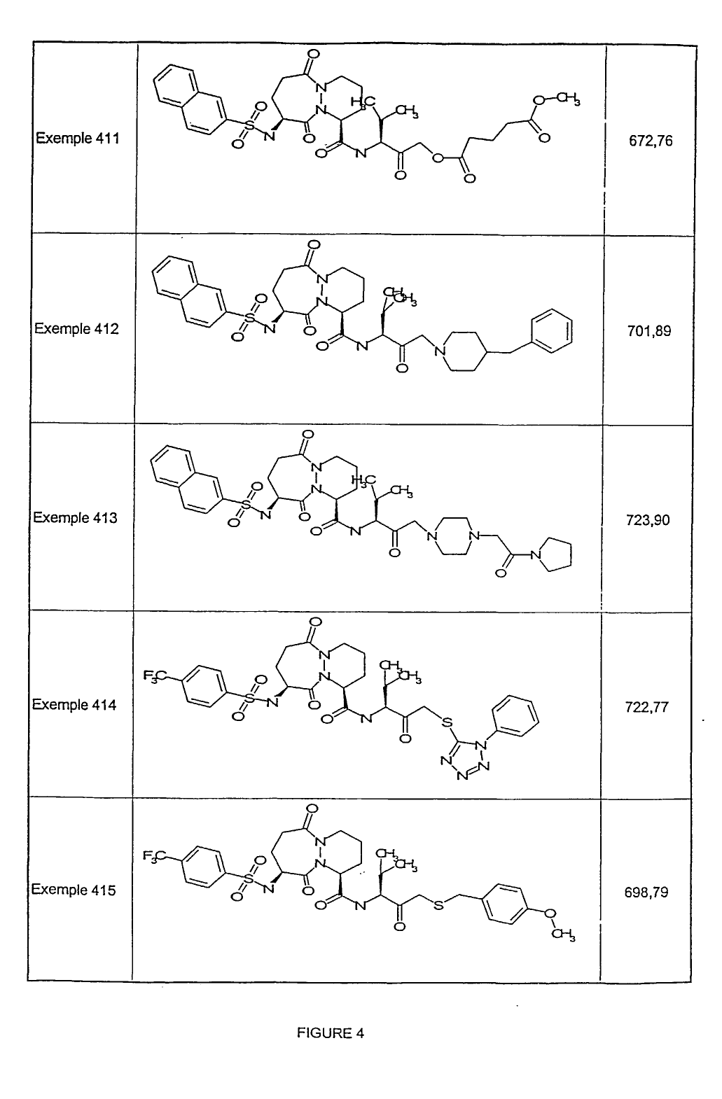

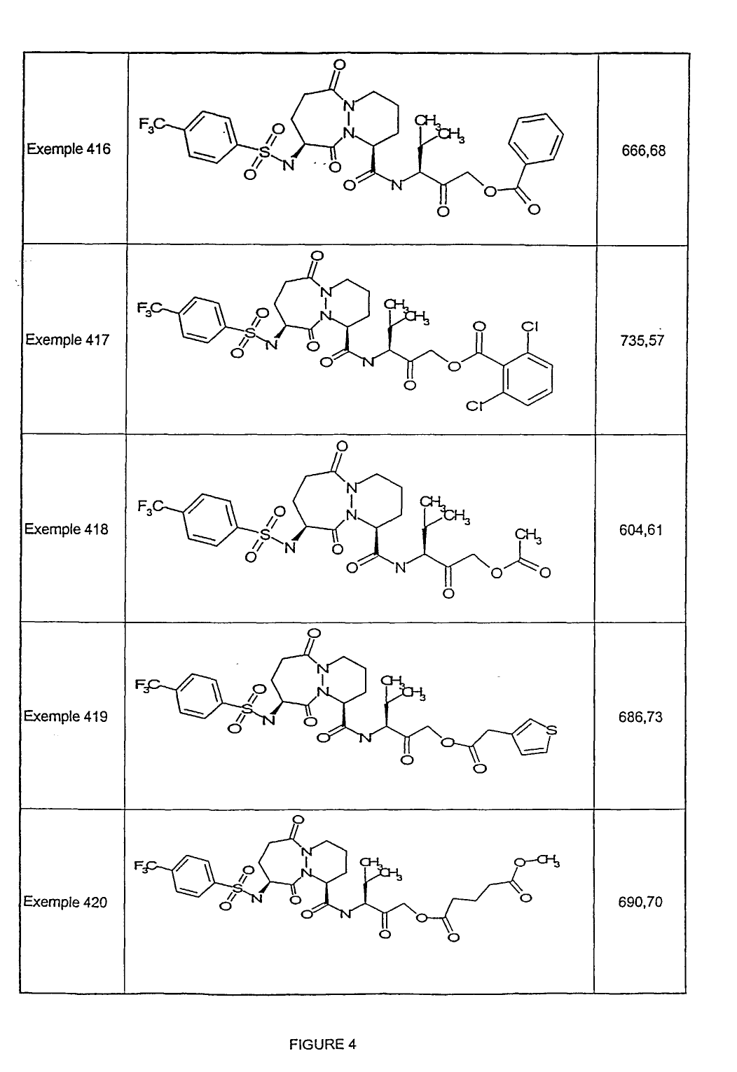

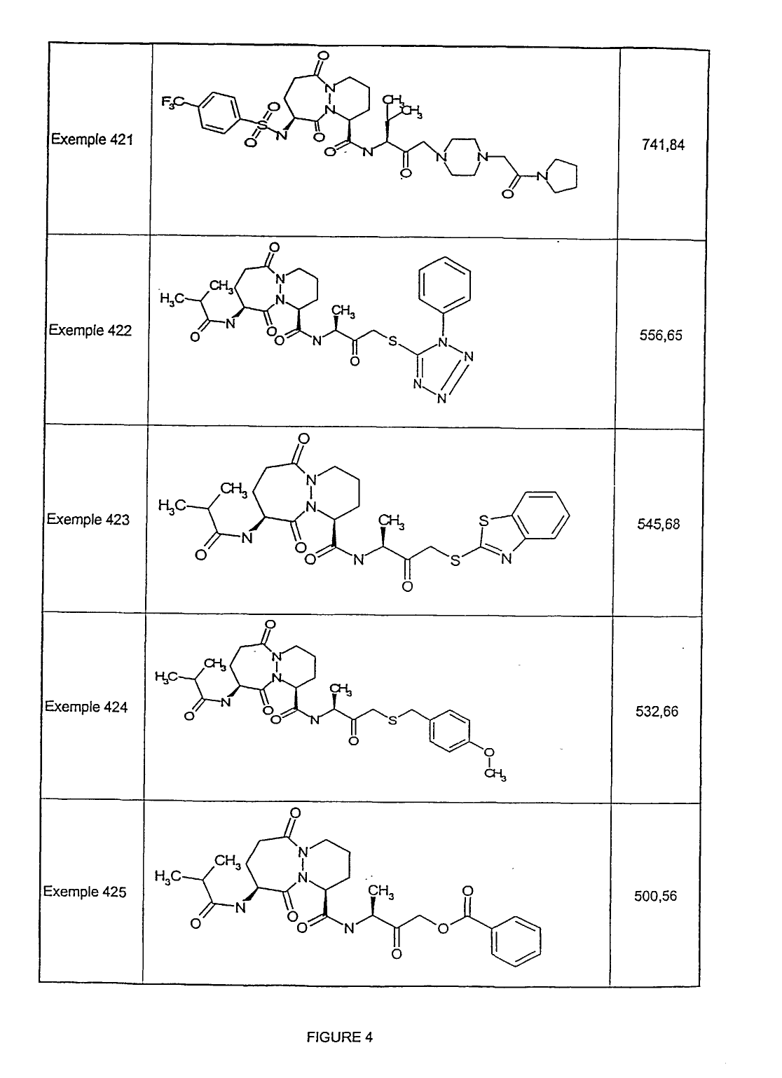

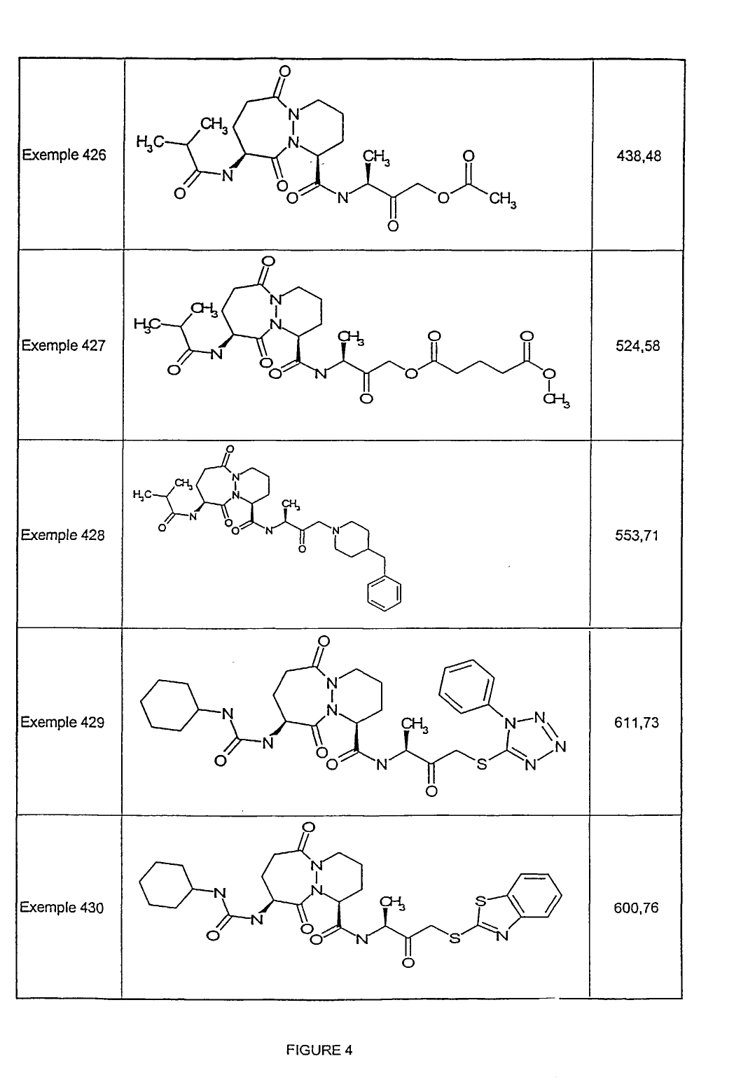

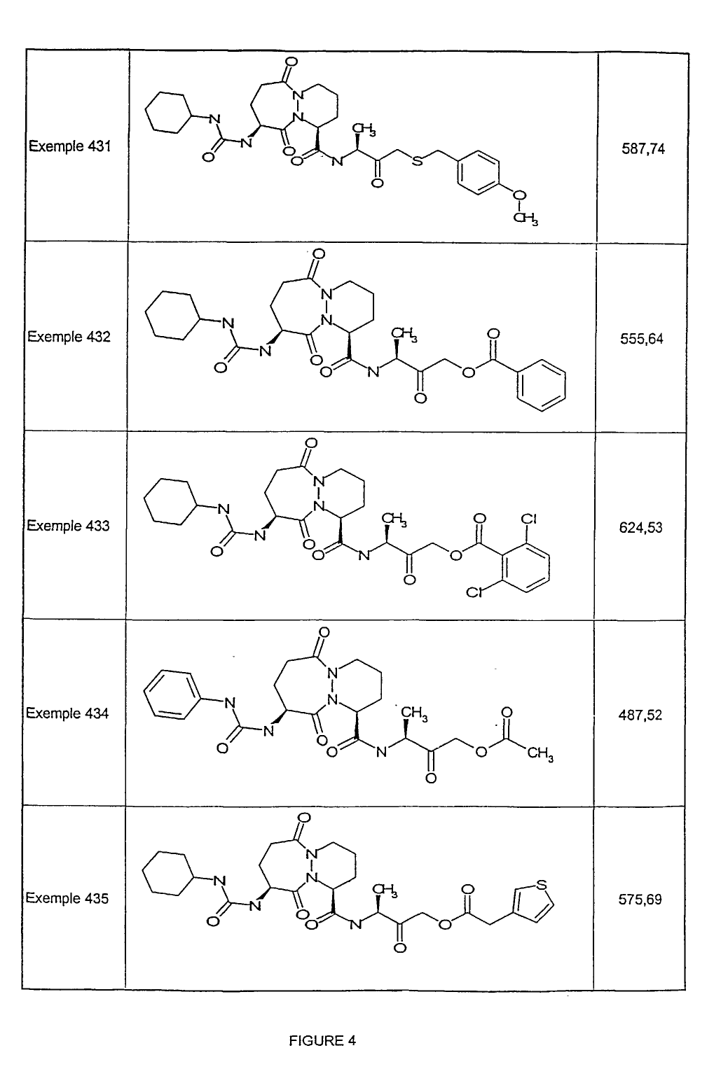

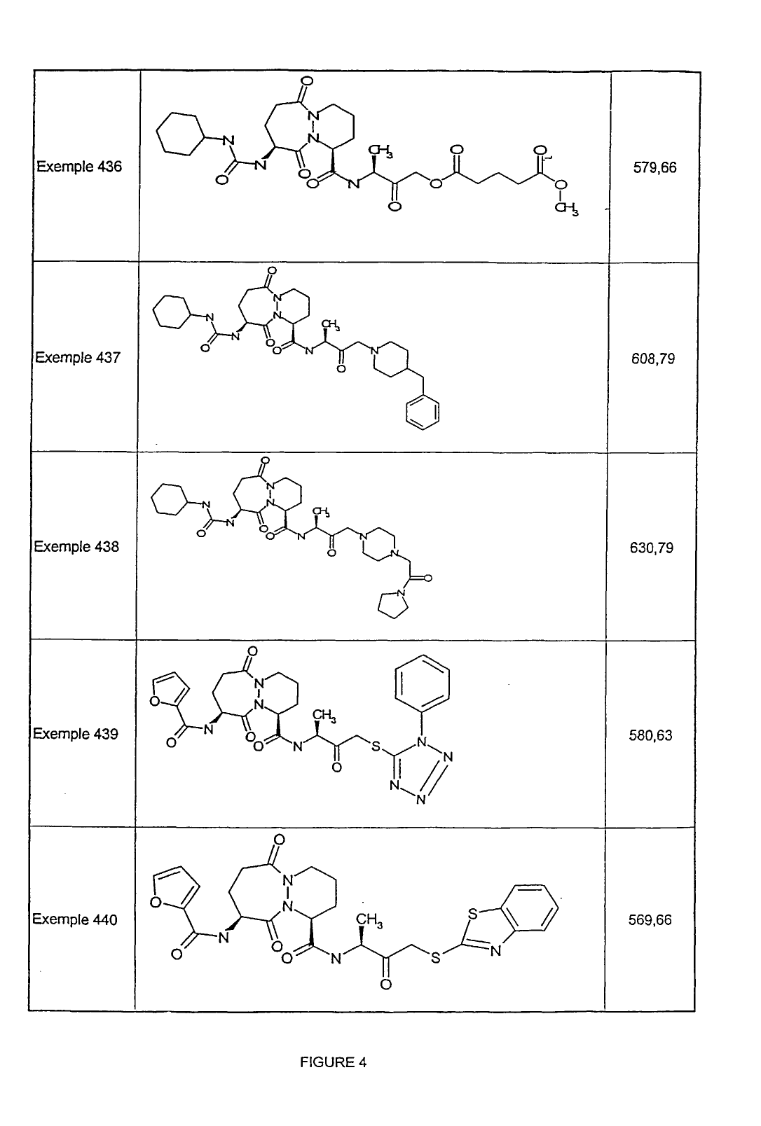

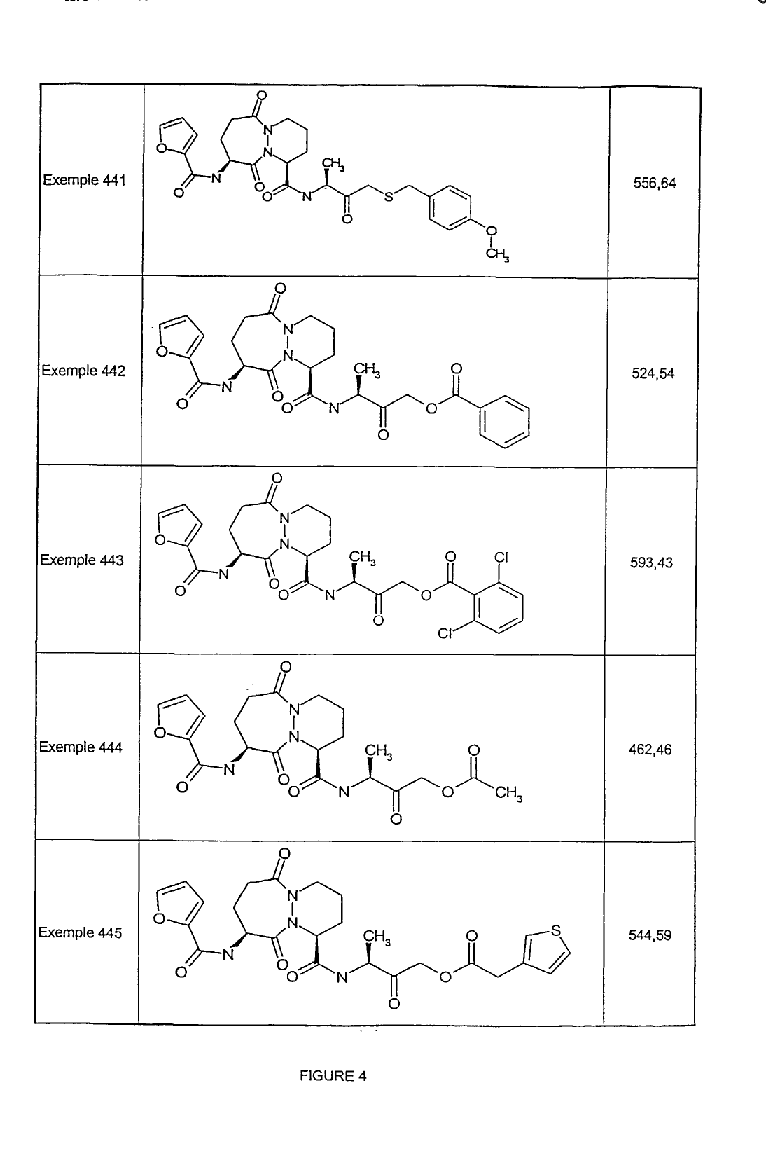

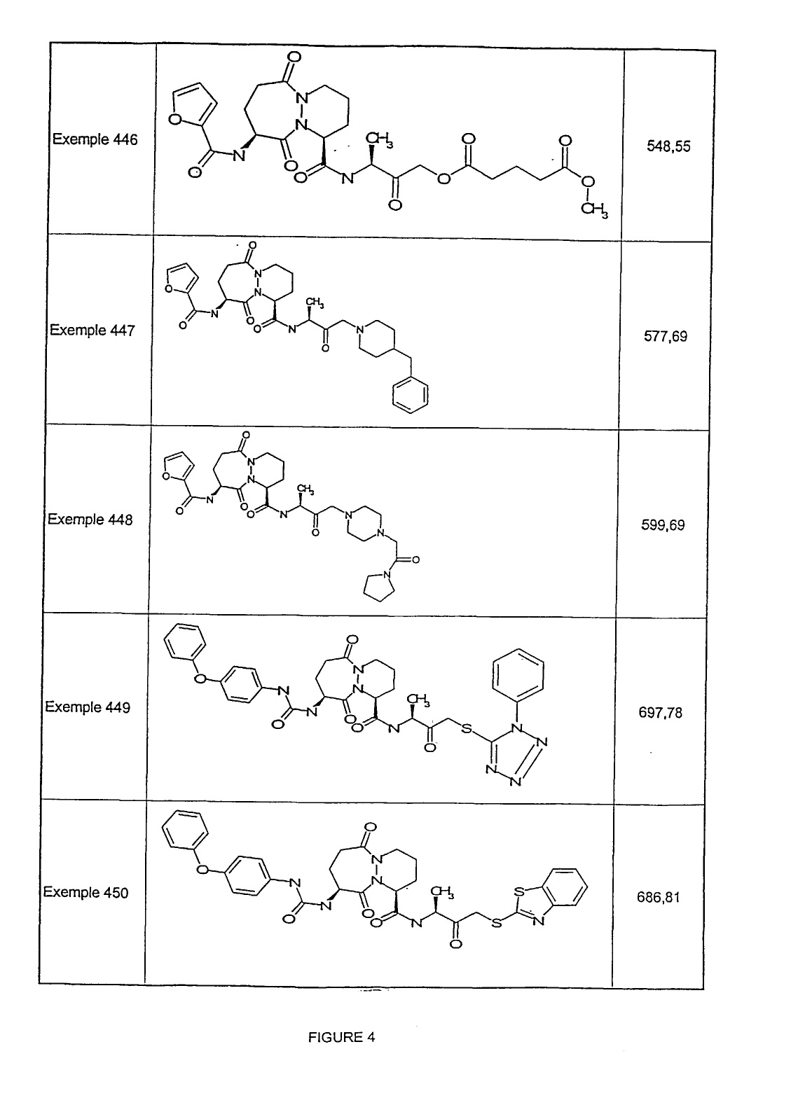

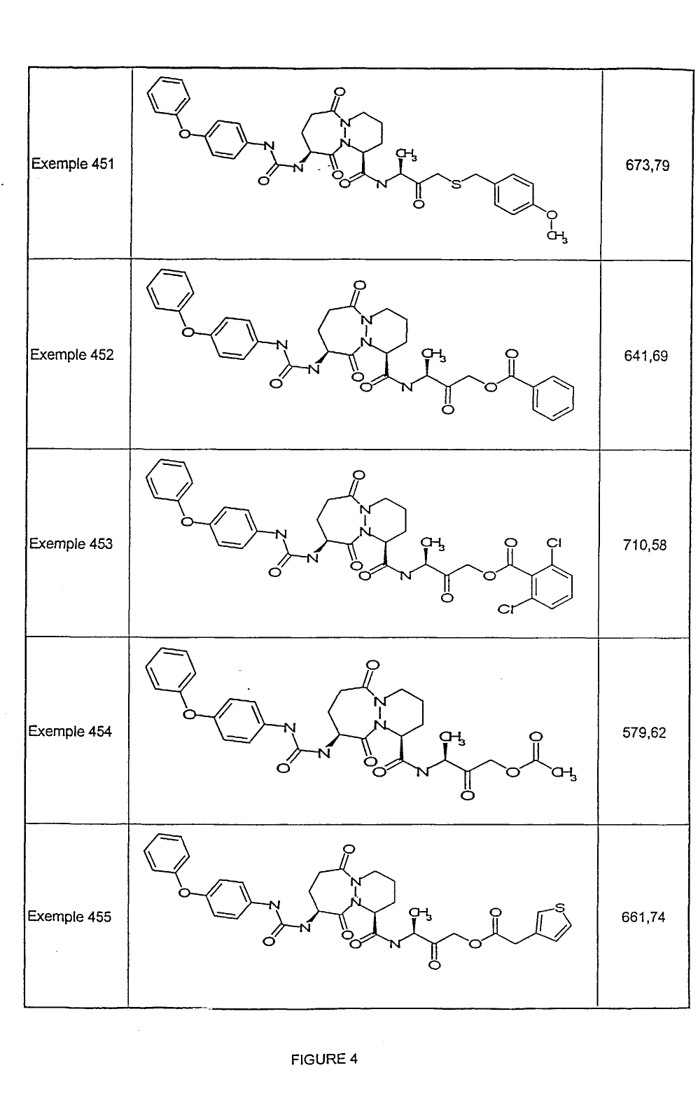

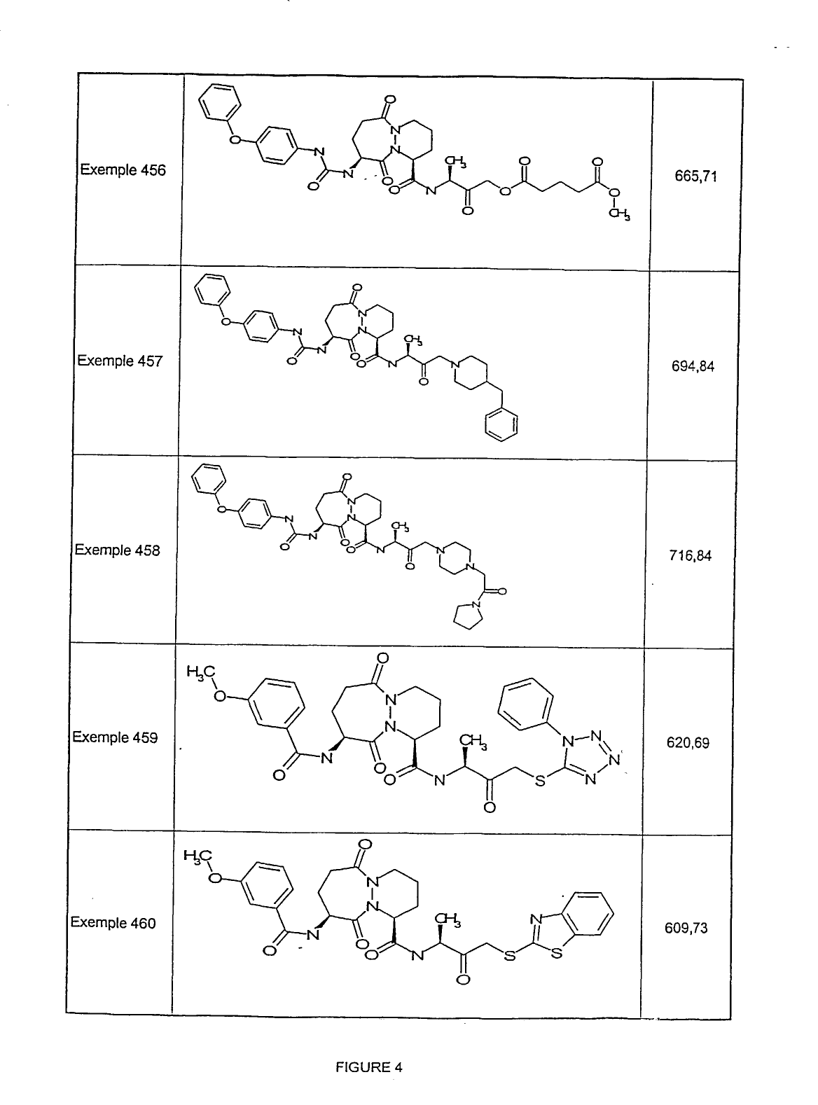

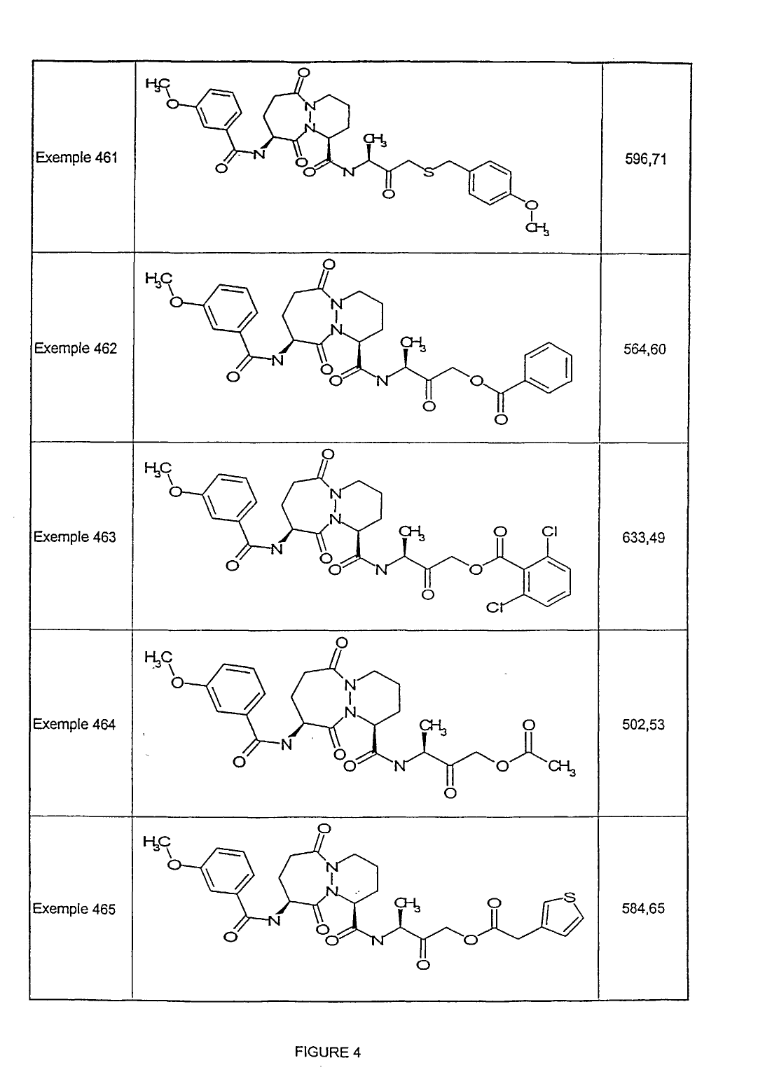

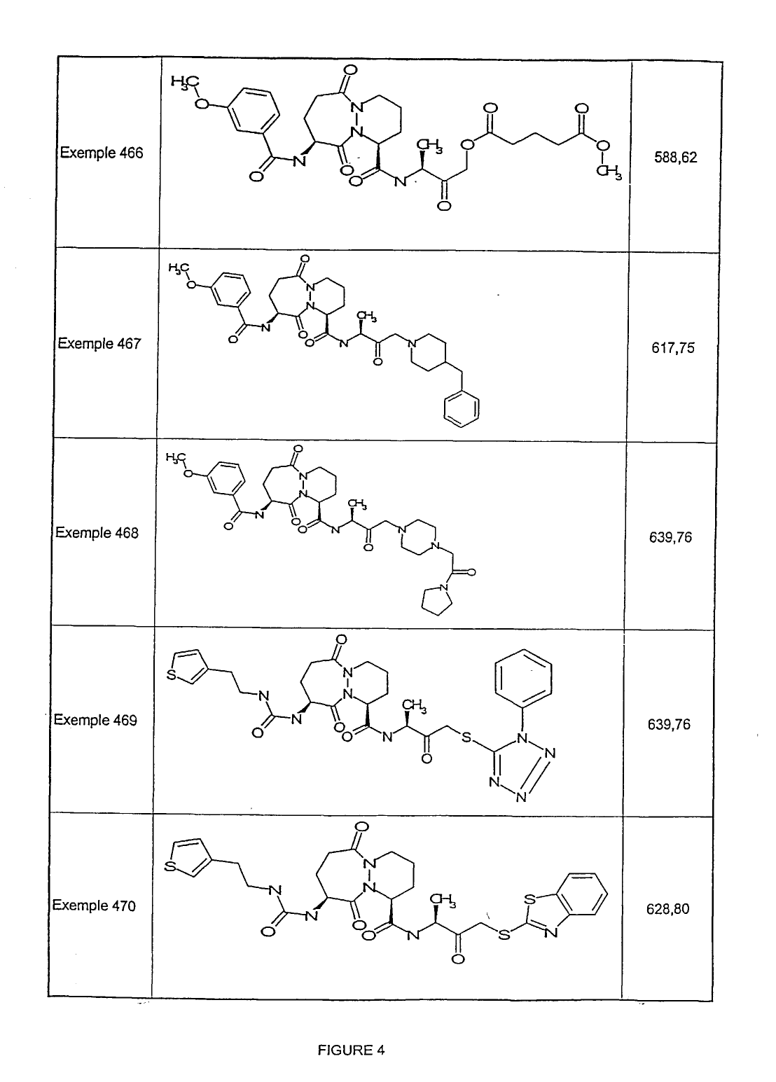

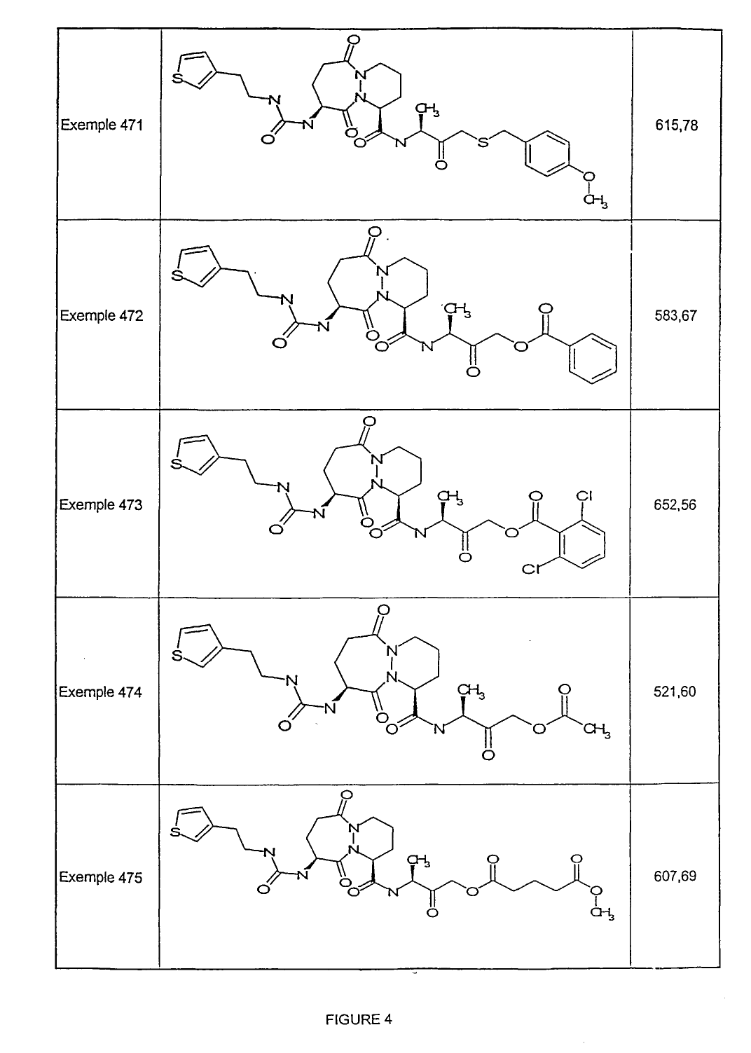

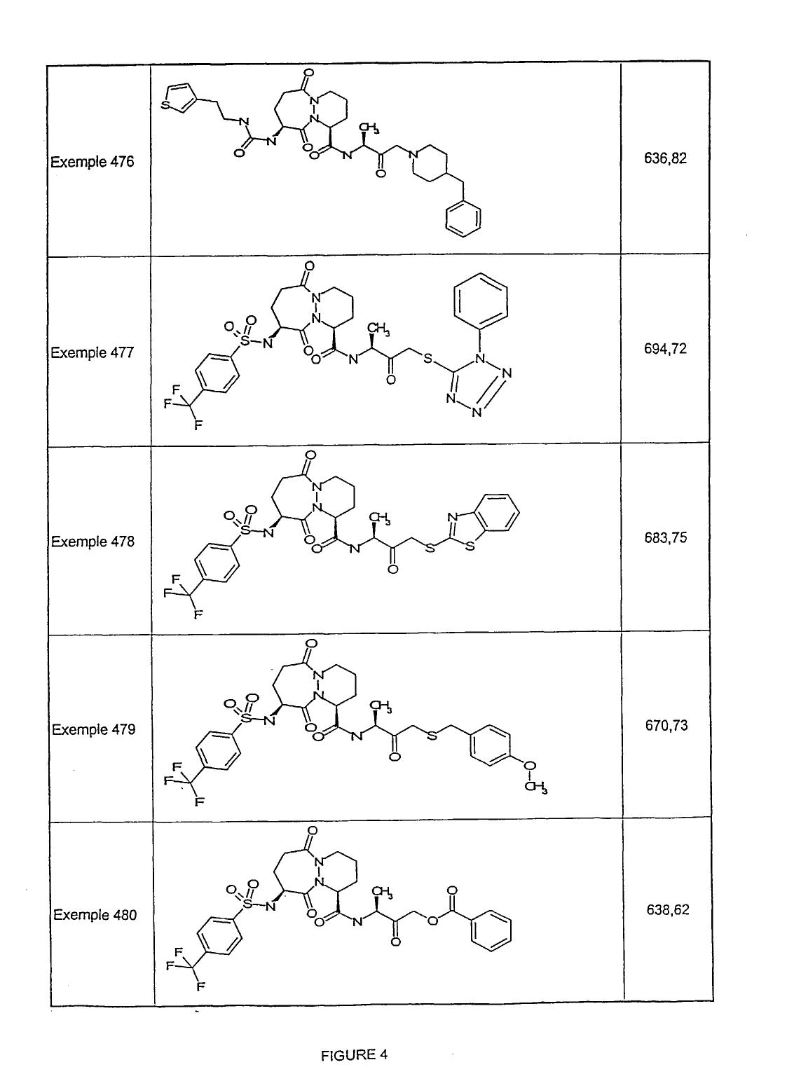

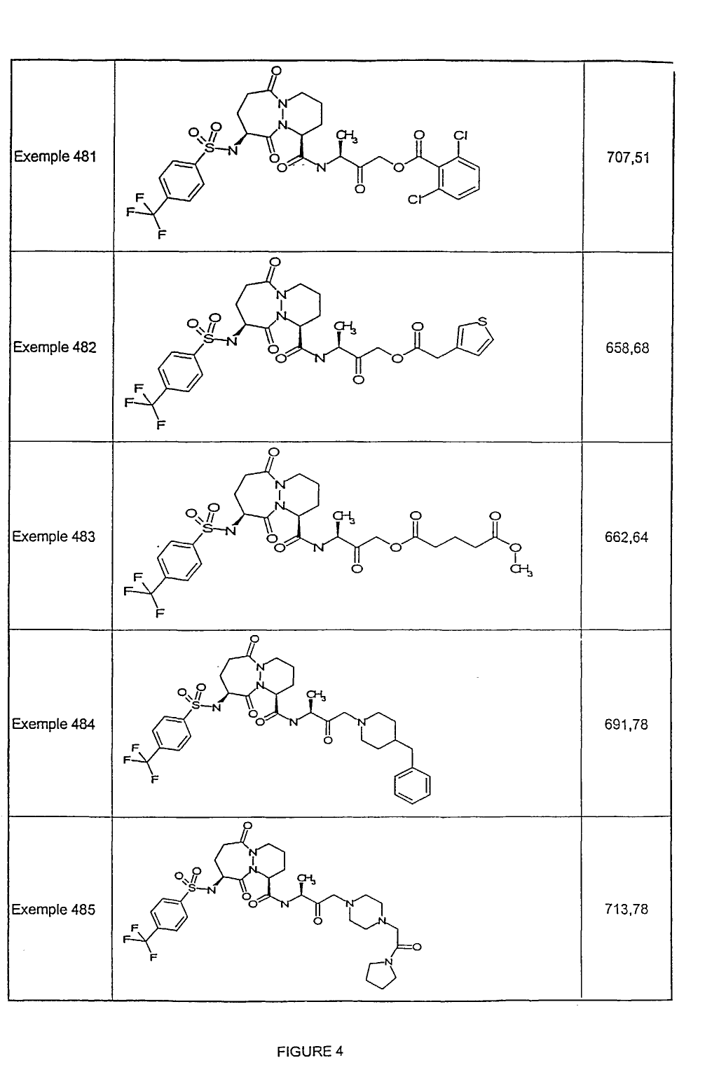

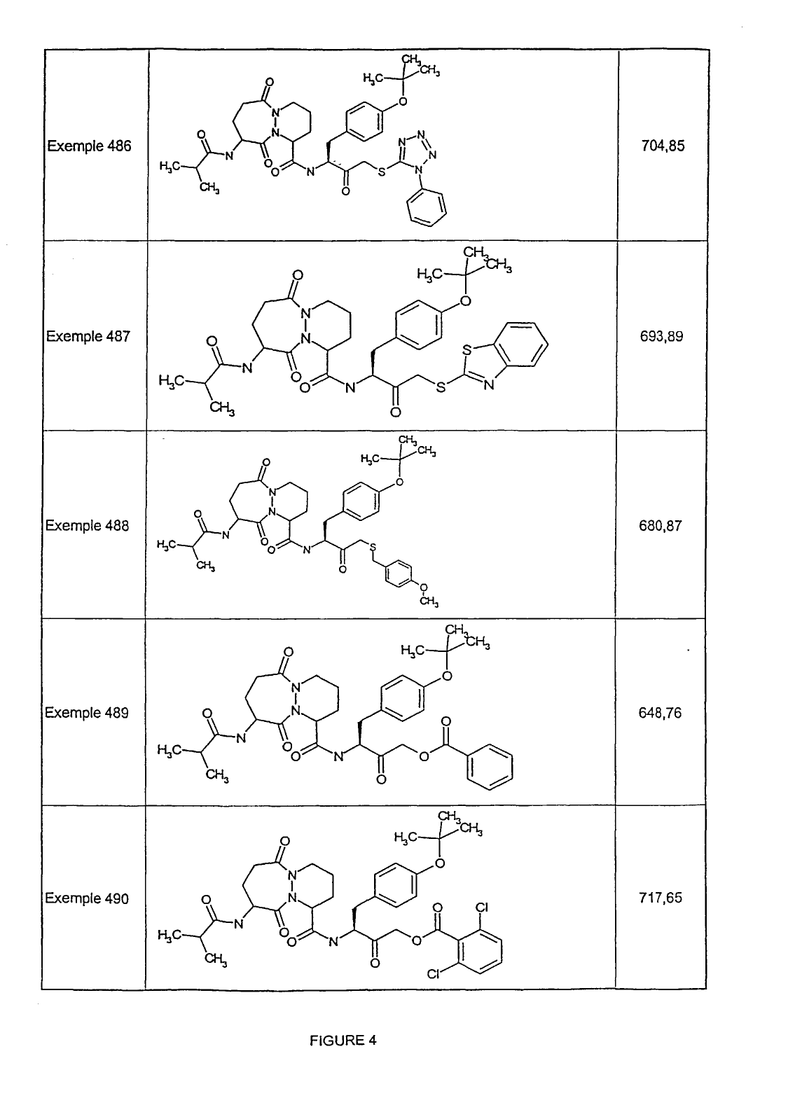

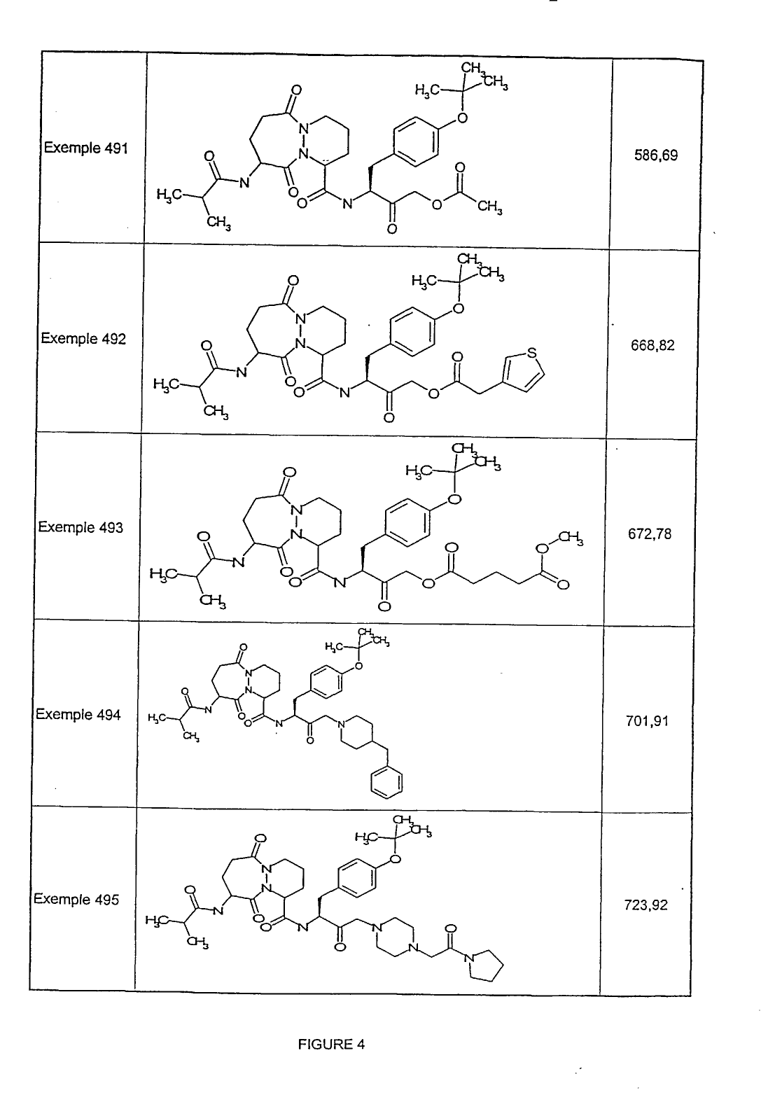

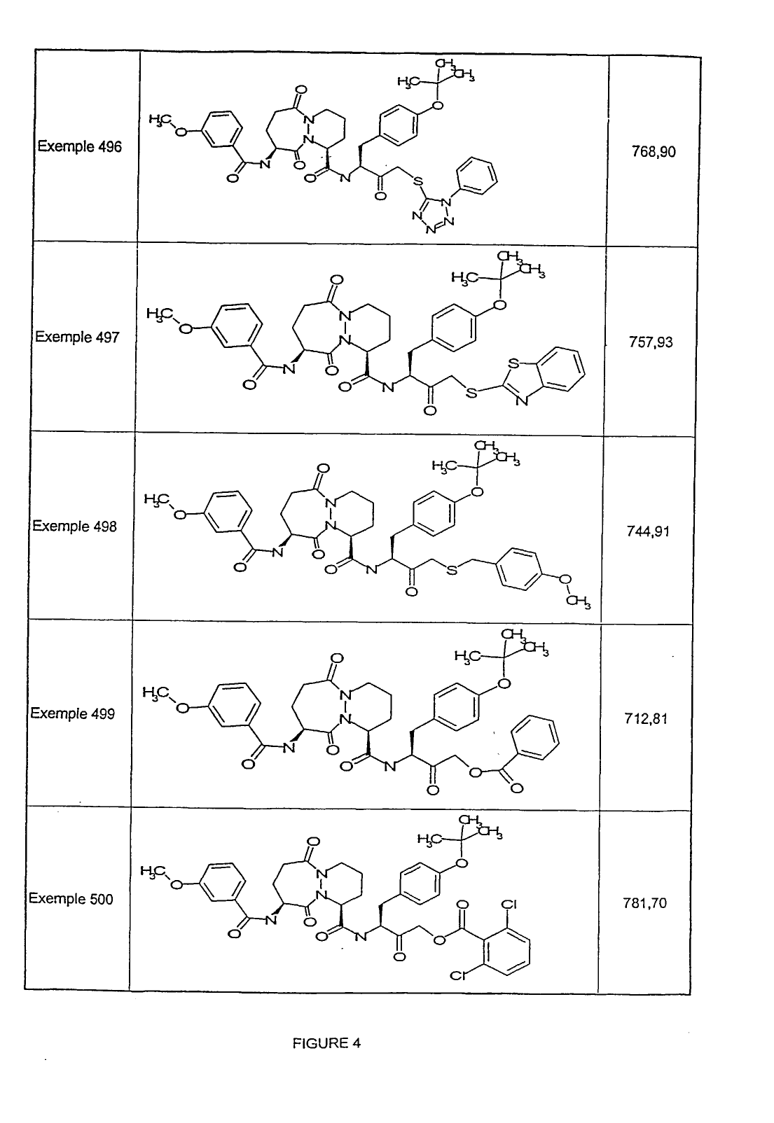

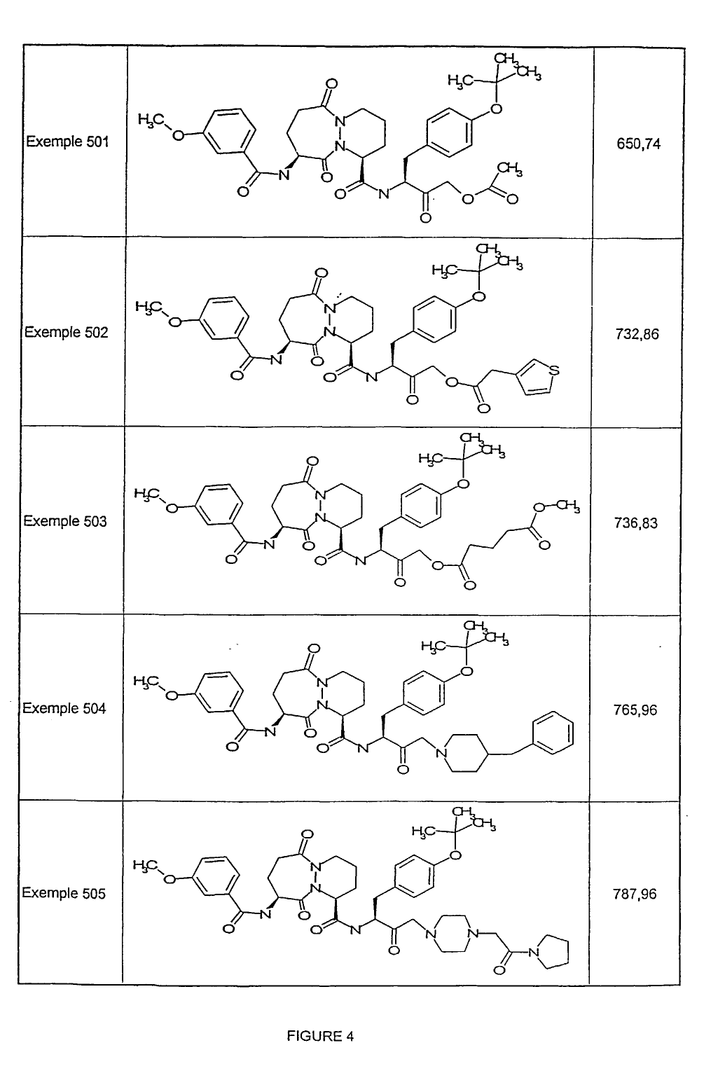

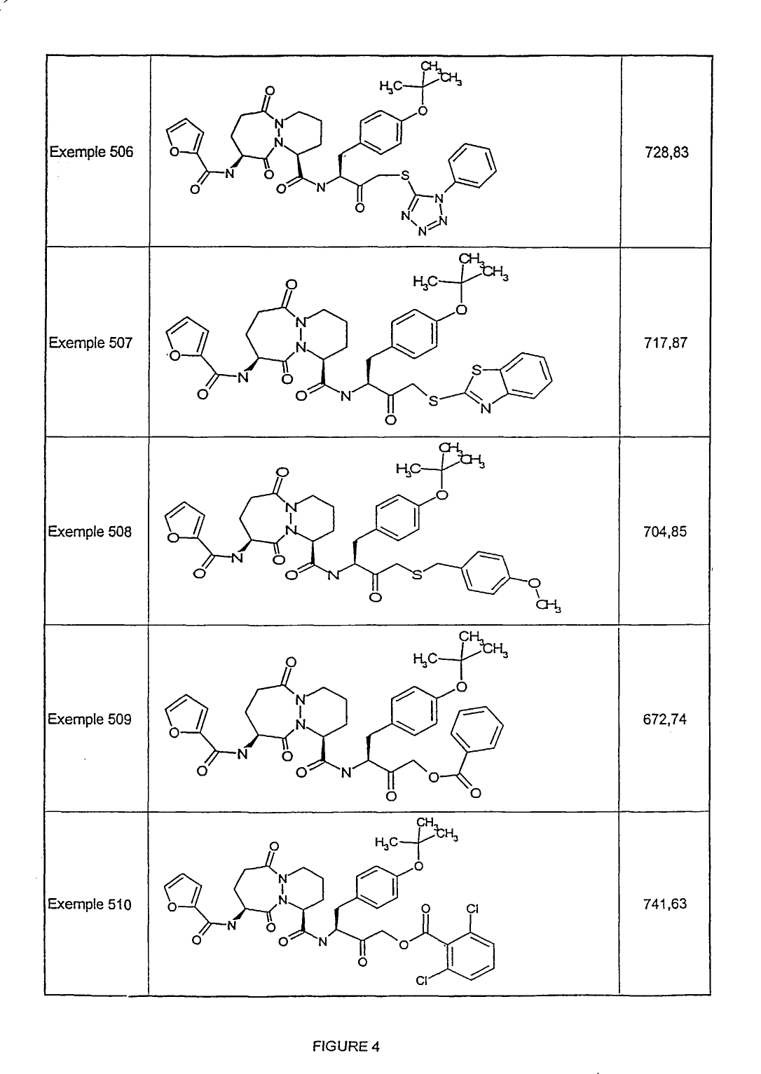

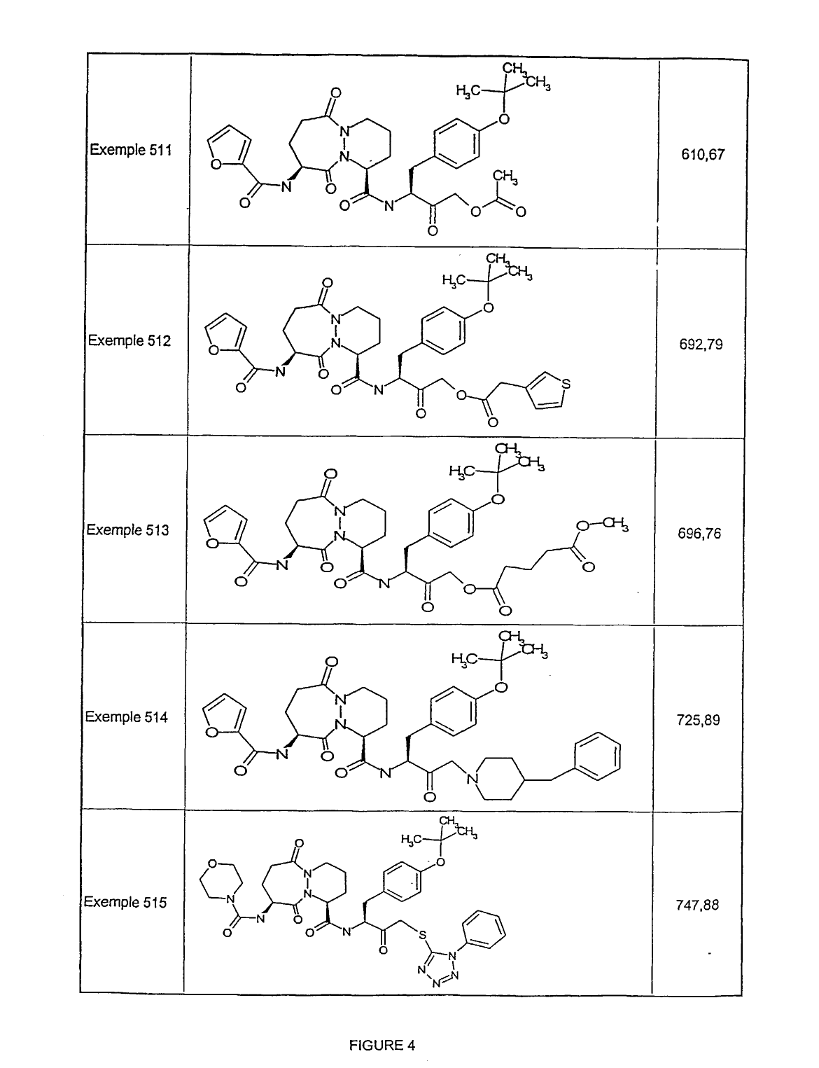

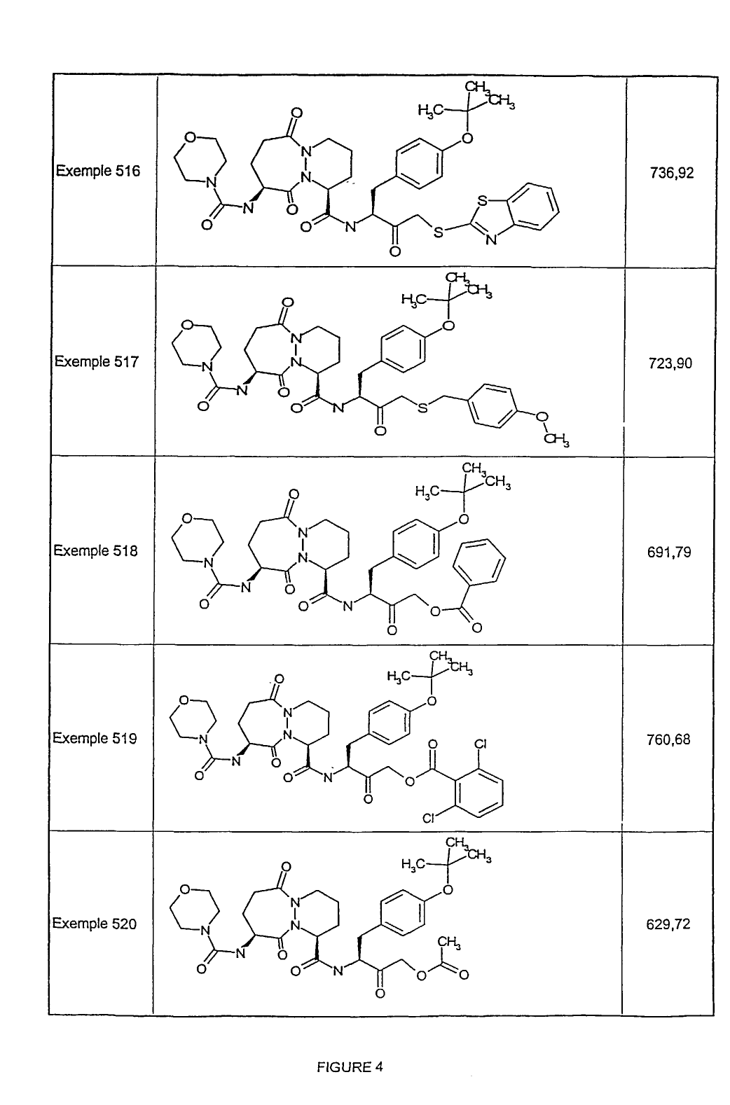

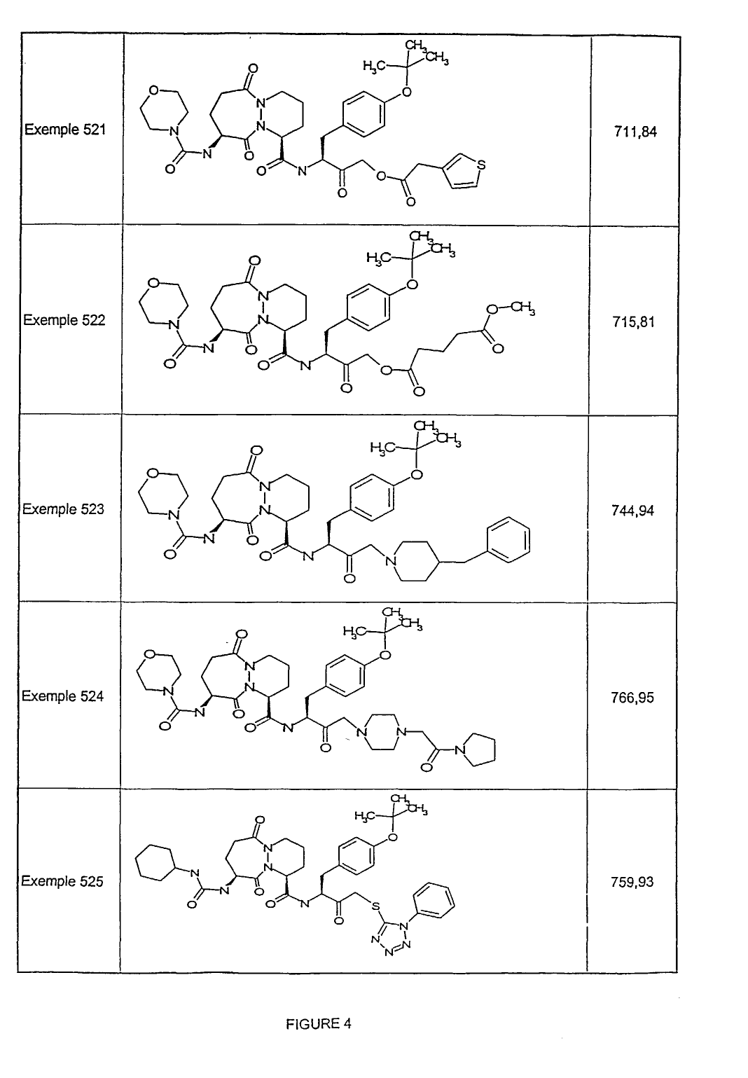

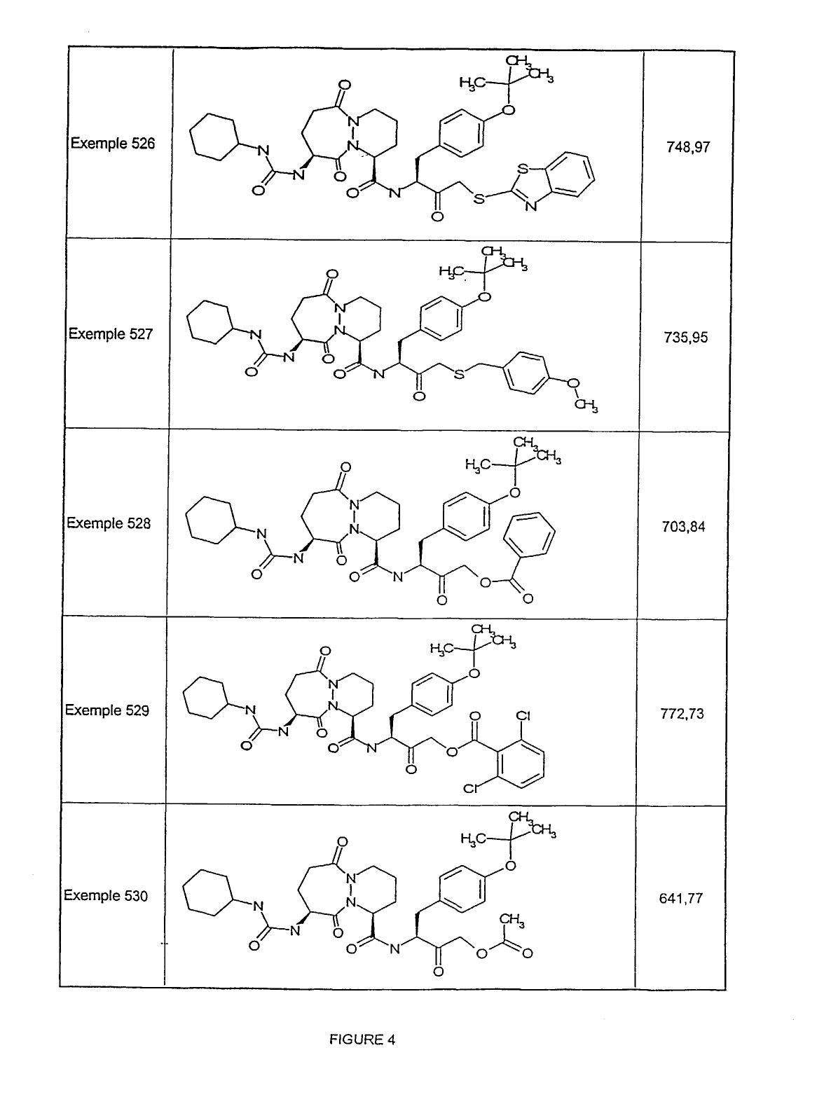

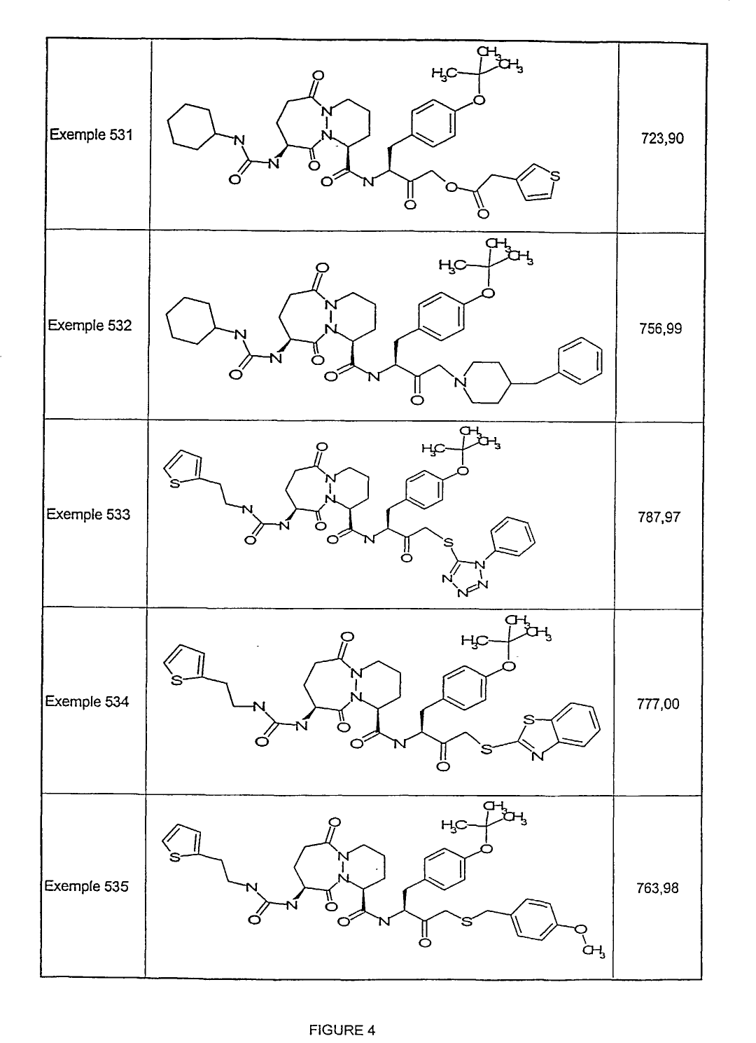

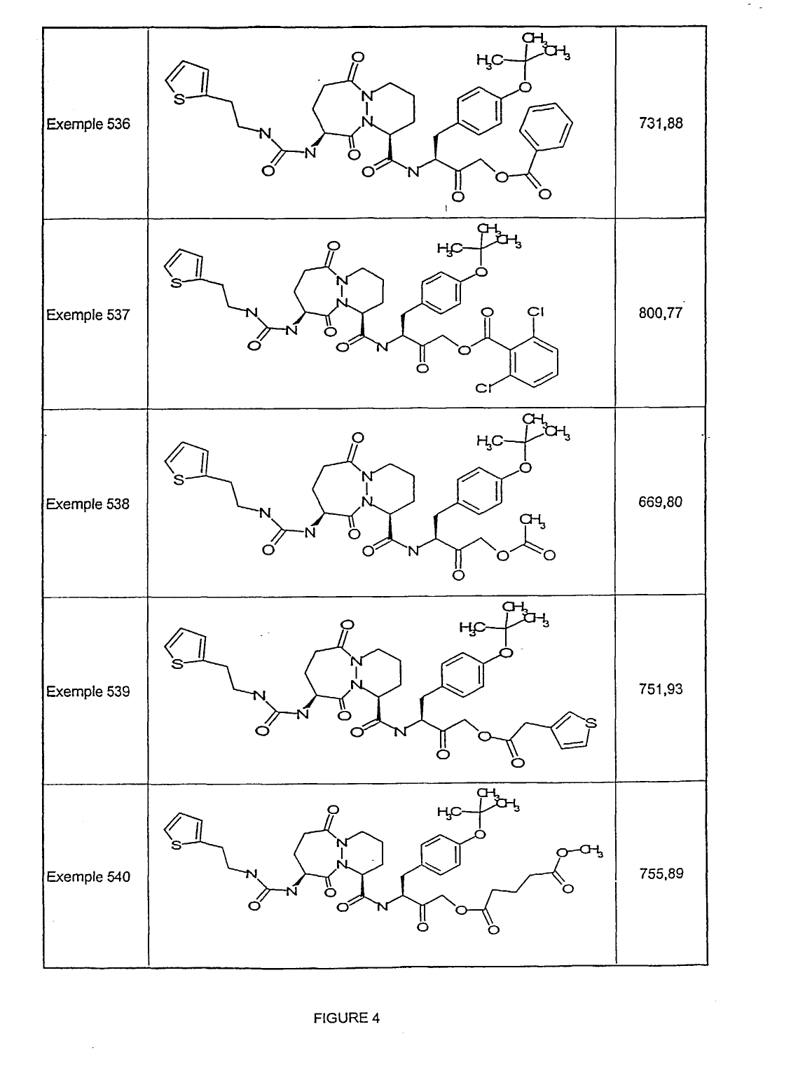

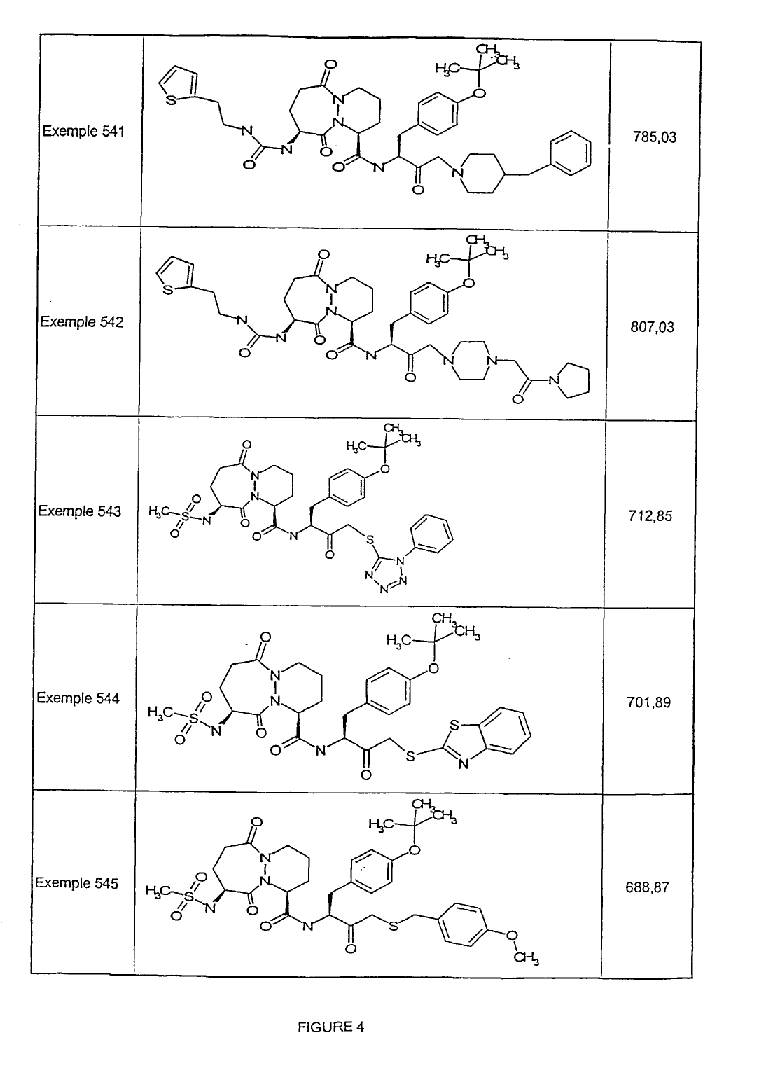

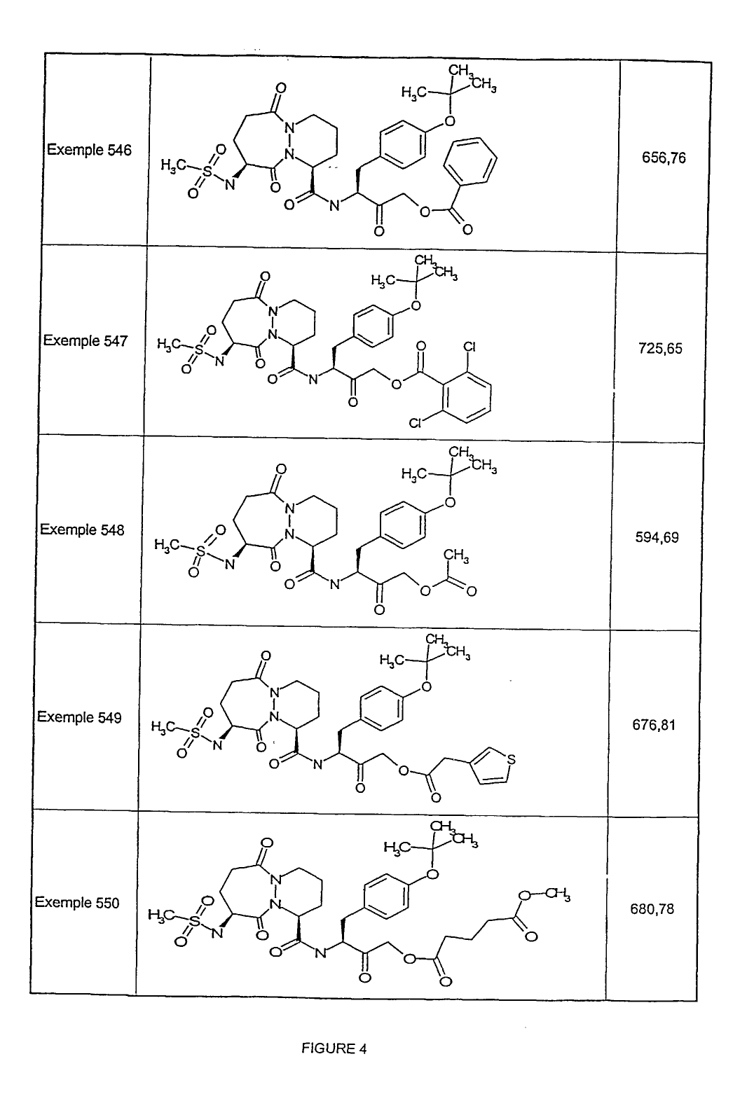

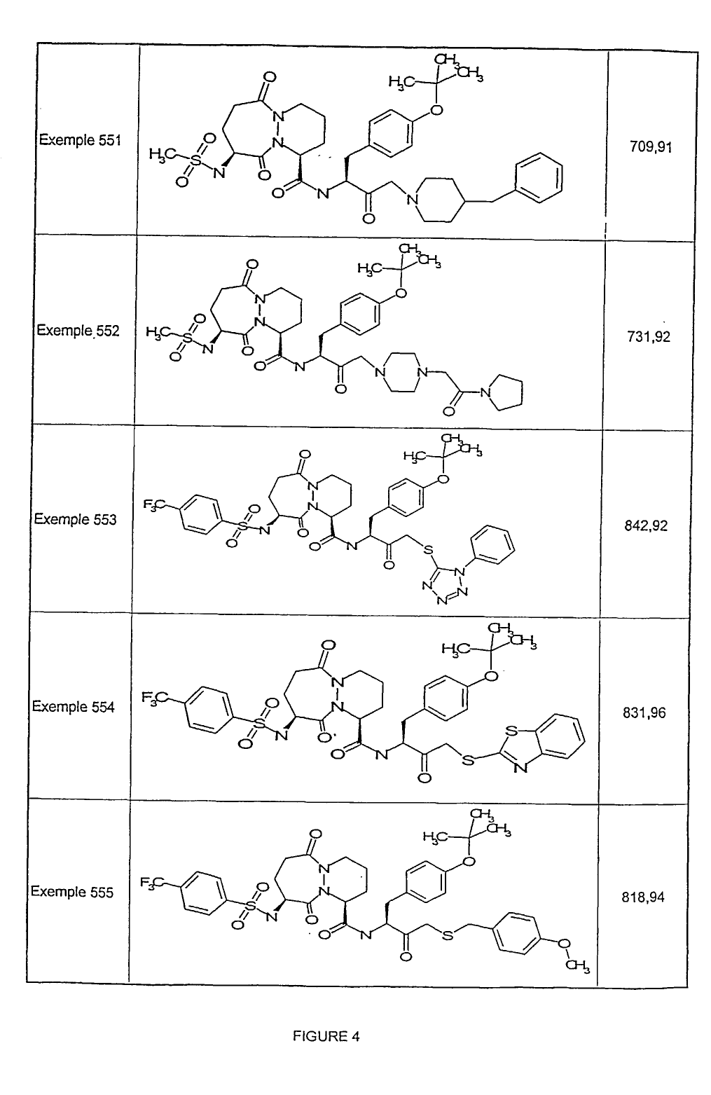

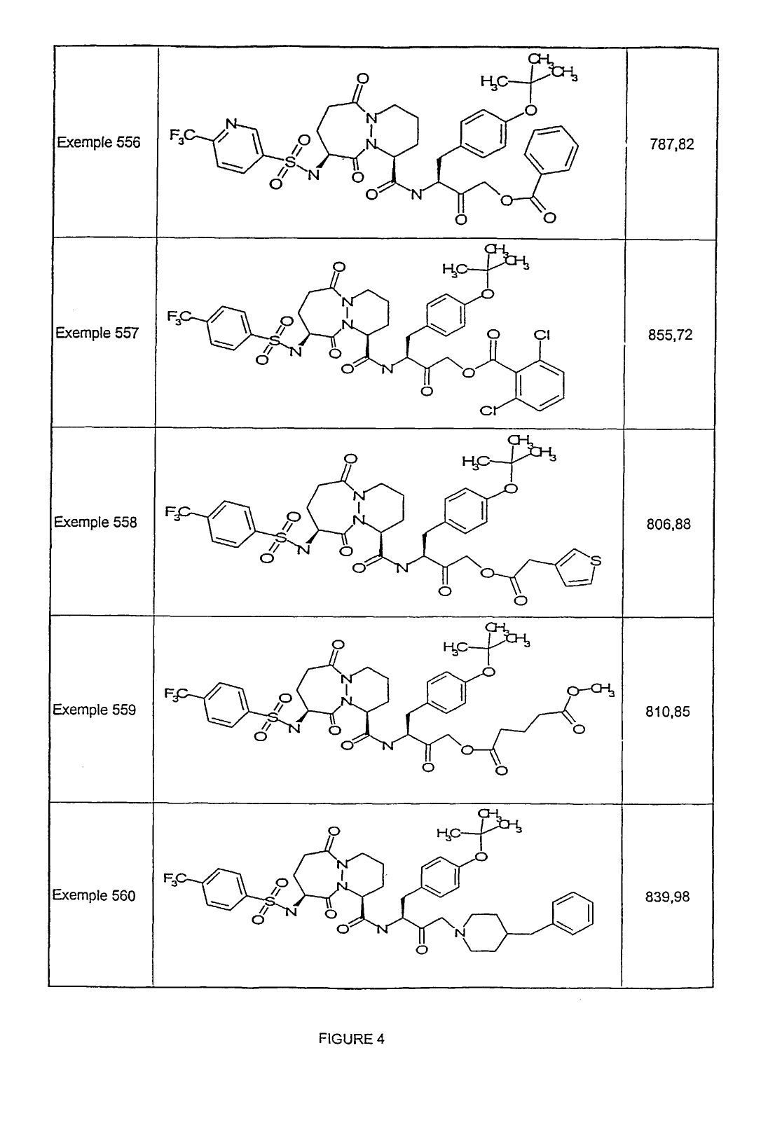

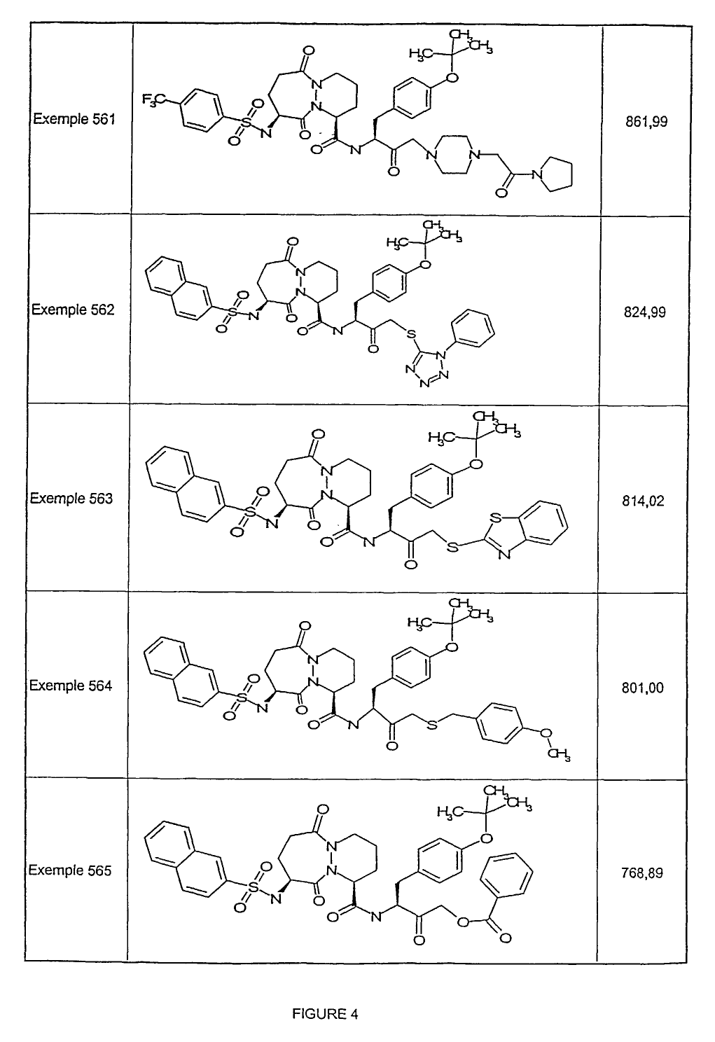

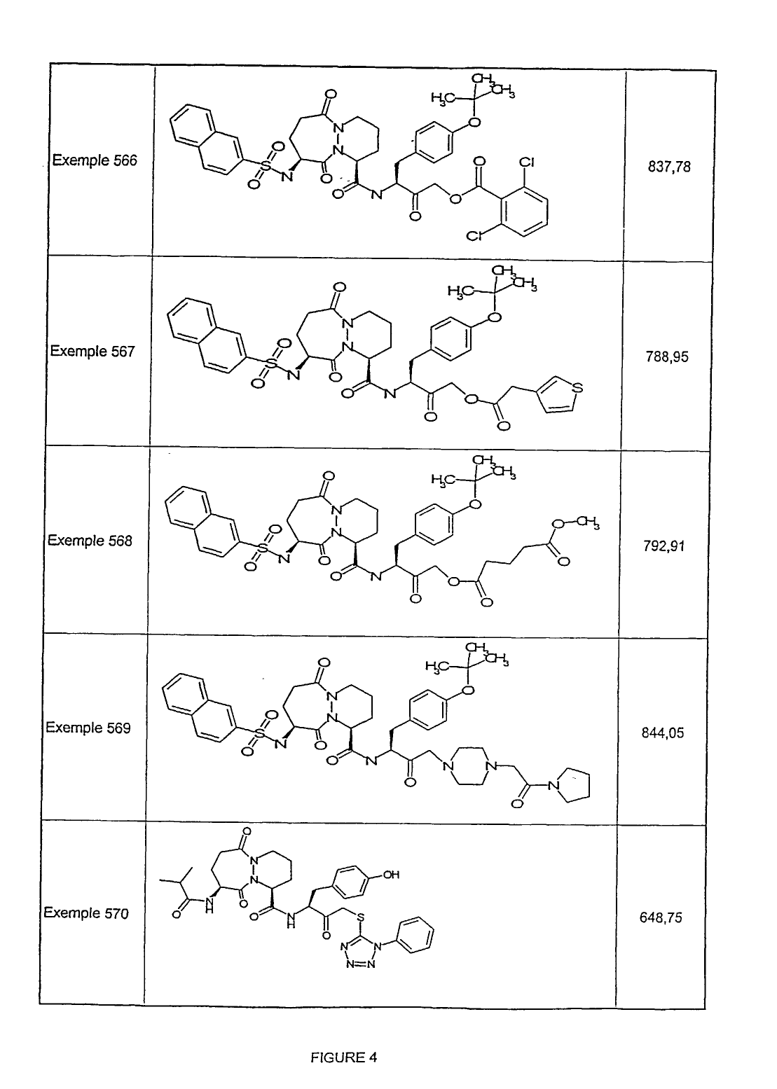

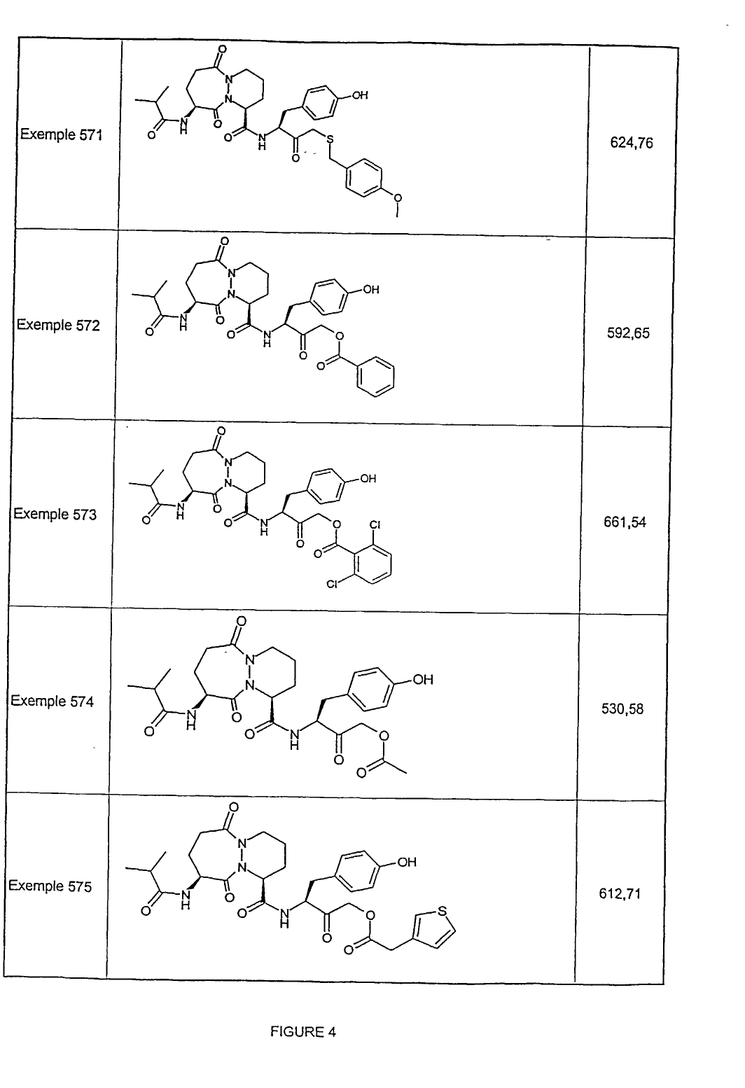

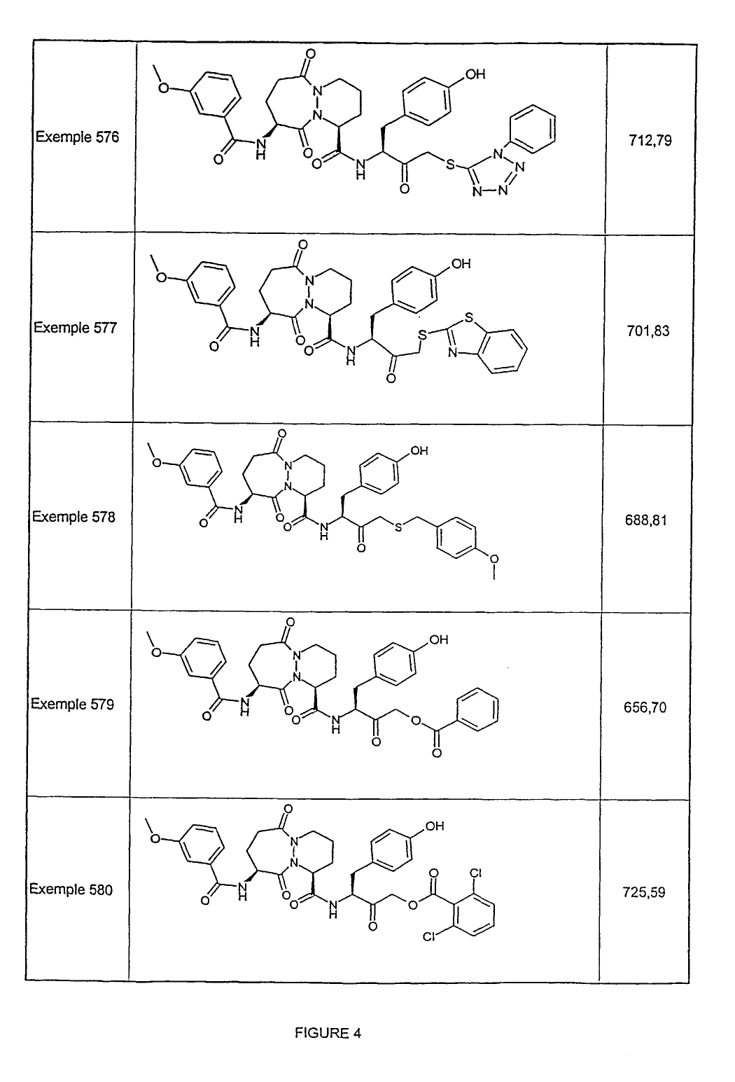

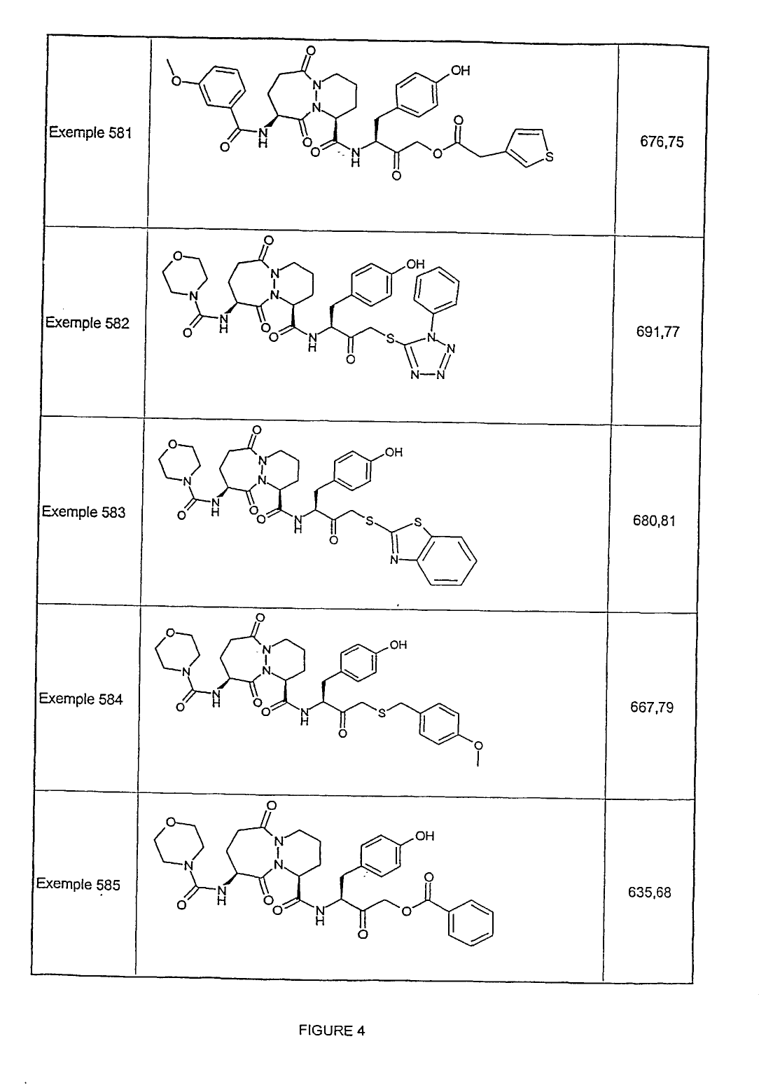

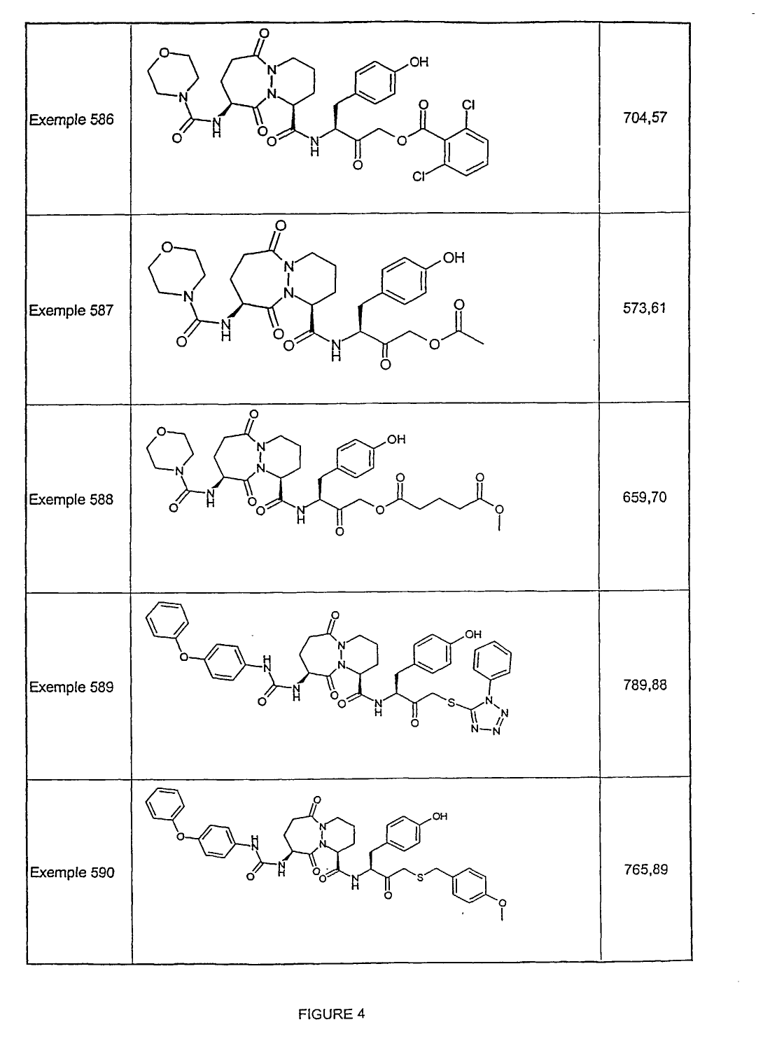

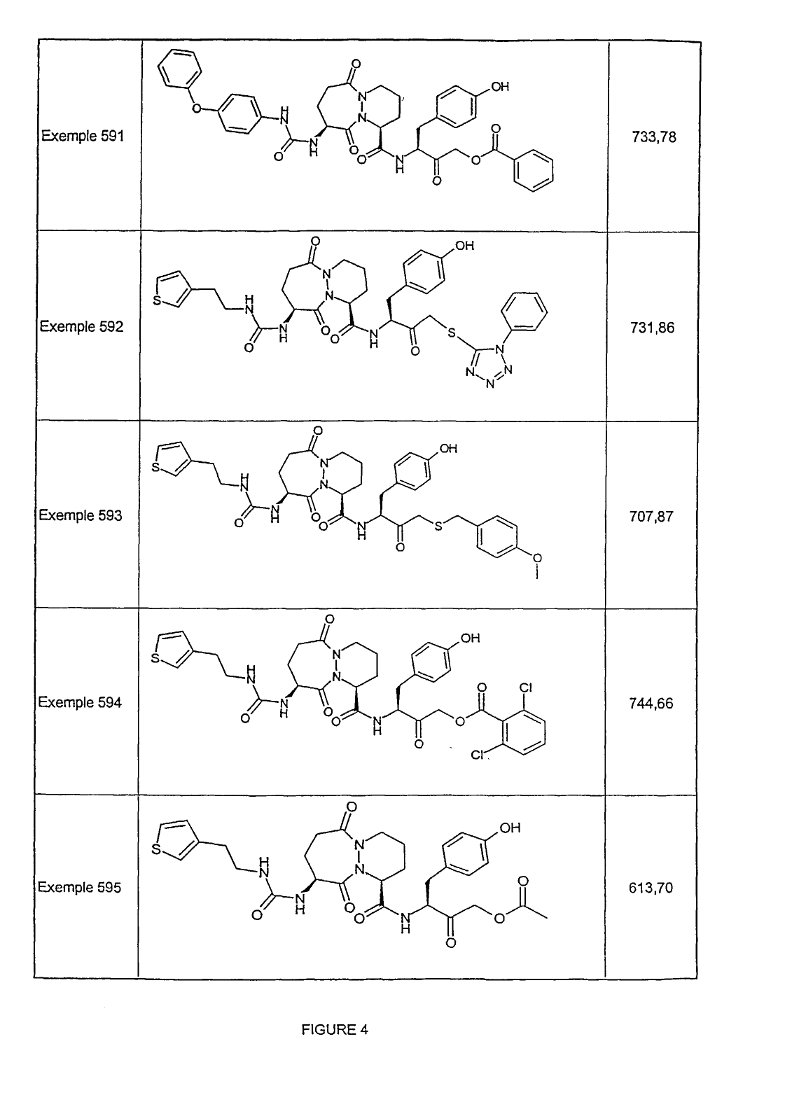

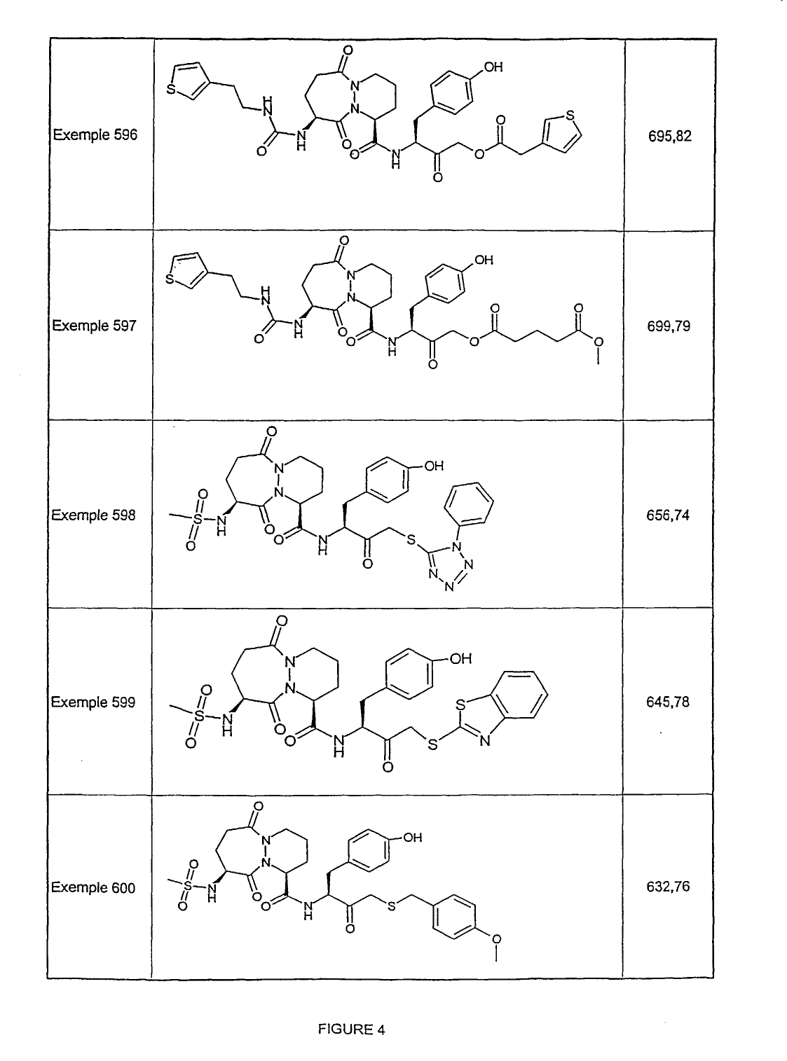

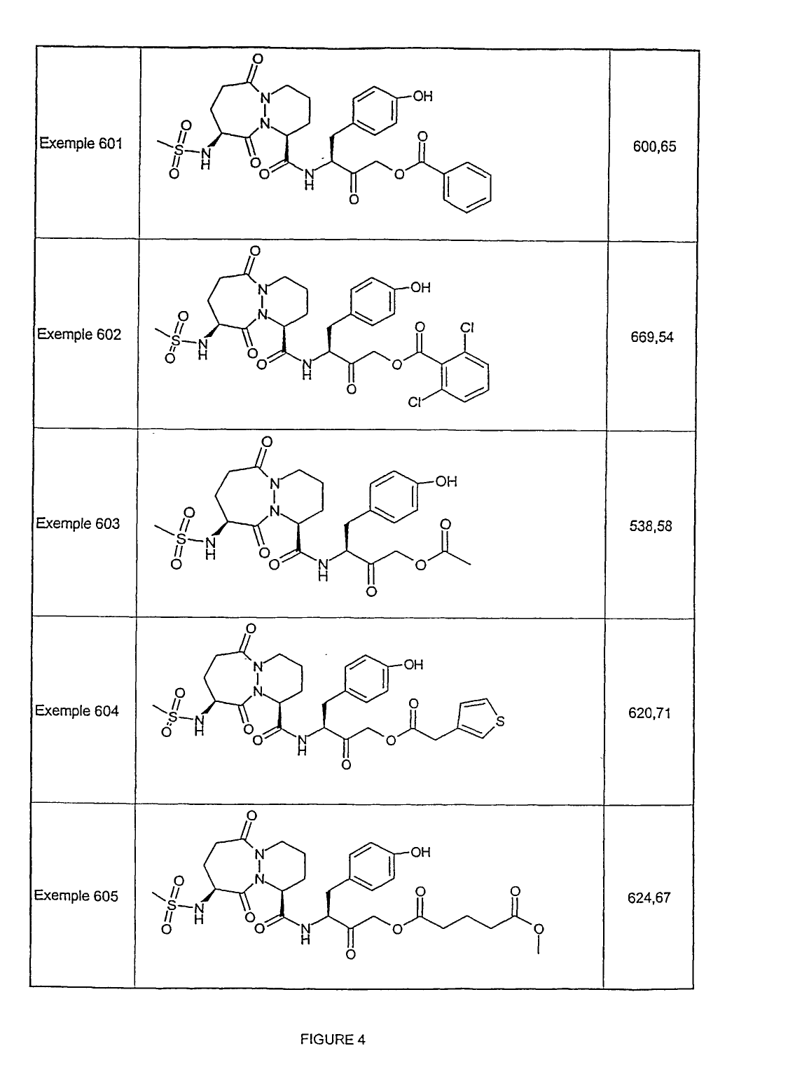

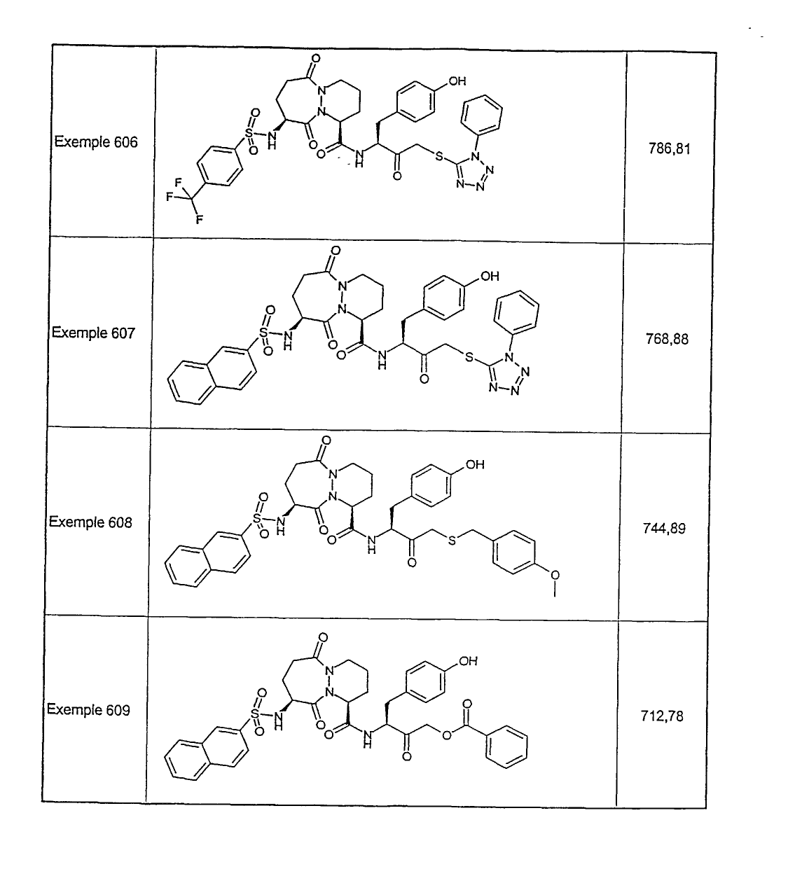

It should be noted that conversion reactions of substituents to other substituents can also be carried out on the starting products as well as on the intermediates as defined above before continuing the synthesis according to the reactions indicated in the process described above. The process described above, as represented in FIG. 1, is the process used in order to obtain all the products of formula (I) of the present Application and in particular those described in Examples 1 to 3 in the experimental part of the present Application as well as those represented in the tables of FIG. 4 described hereafter.

Such a process represented in FIG. 1 is constituted by the 4 following stages:

Stage 1 allows the product of formula (VI) to be obtained from the amino acid of formula (VIa)

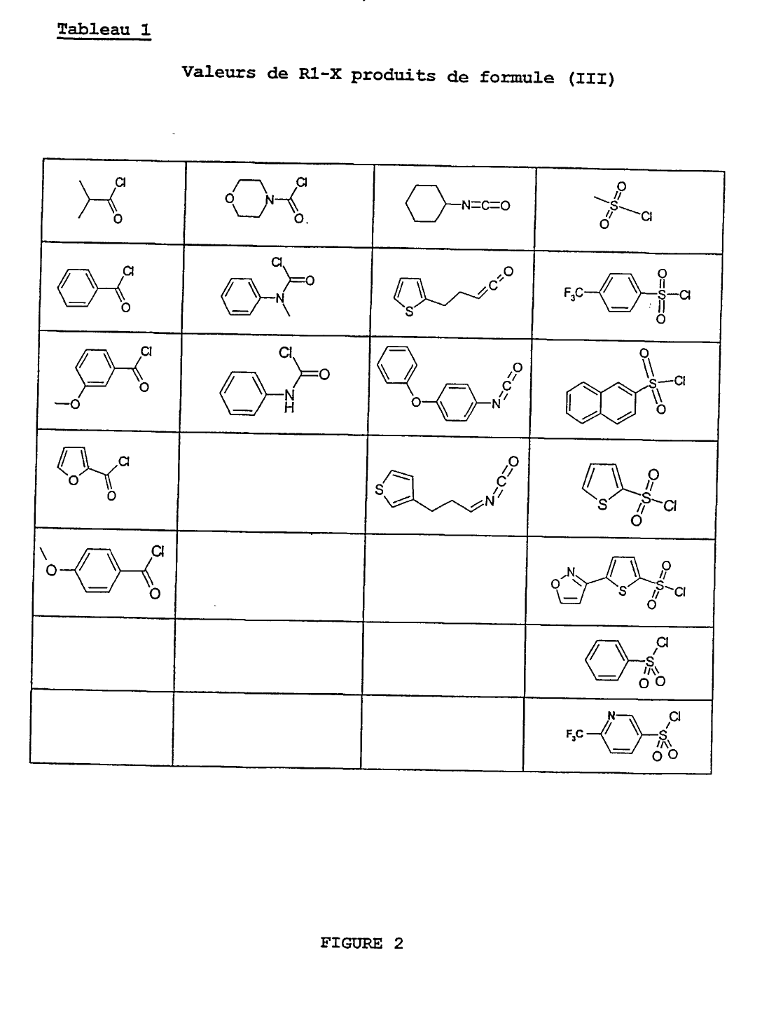

Stage 2 allows the product of formula (V) to be obtained from the starting product of formula (II) by reaction with the product of formula (III) i.e. R1-X chosen in particular from the values indicated in Table 1 of FIG. 2.

Stage 3 allows the product of formula (Ix) to be obtained by peptide coupling of the product of formula (VI) obtained in Stage 1 with the product of formula (V) obtained in Stage 2.

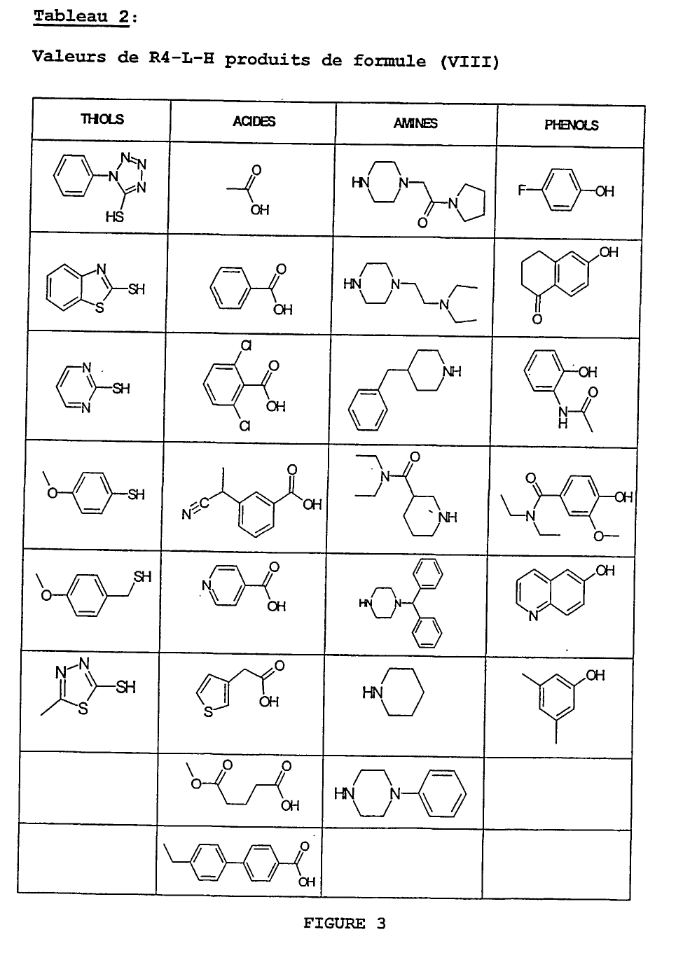

Stage 4 allows the product of formula (Iy) to be obtained from the product of formula (Ix) obtained in Stage 3 by reaction with the compound of formula (VIII) i.e. H—L—R4′ chosen in particular from the values indicated in Table 2 represented in FIG. 3: this Stage 4 can be carried out according to two different methods one called ‘KF/DMF’ (potassium fluoride in dimethylformamide) and the other called ‘Supported amine’.

By ‘supported amine’ is meant an amine bonded onto a polystyrene support that is commercially available such as for example dimethylaminomethylpolystyrene resin, diethylaminomethyl or other commercially available supported tertiary amine.

Under preferred conditions of implementation of the invention, the process of the present invention as represented in FIG. 1 described hereafter and as defined above in 4 stages, can be carried out in the following manner.

Stage 1: Preparation of the Diazoted Amino Acid of Formula (VI)

This stage can be broken down into 3 stages a1), b1) and c1) a1) obtaining the product (VIb) from an amino acid (VIa)

Addition of fmoc on the N-terminal amine function of the starting amino acid: the conditions for this reaction are described in particular in the article referred to above: ‘Mueller, A.; Vogt, C.; Sewald, N.; Synthesis (1998),(6),837-841’. As regards the Fmoc-amino acids, the document: ‘Methods Enzymol. (1997), 289 (Solid-phase peptide synthesis), 44-67 can also be mentioned.

Such reaction conditions can be applied to any so-called natural or non-natural amino acid.

b1) obtaining the diazoted fmoc product of formula (VIc) starting from the product of formula (VIb) obtained above in a) in two reactions bi) and bii) described hereafter:

bi) preparation of the mixed anhydride

A 1.5 equivalent of 4-Methyl morpholine followed dropwise by a 1.03 equivalent of isobutyl chloroformate are added successively under nitrogen and at −10° C. to a suspension of the product of formula (VIb) obtained in Stage a) above, in dichloromethane (3 ml/mmole). The reaction is maintained for 1 hour at −10° C. and the mixture is filtered rapidly.

bii) Diazotation

The filtrate is cooled down to −10° C., then under nitrogen, 2 equivalents of diazomethane in solution in dichloromethane (0.3M) are added dropwise. The reaction medium is brought progressively to ambient temperature then left under agitation for 1 hour. The solution is poured into a saturated sodium bicarbonate solution (10 ml/mmole). The organic phase is washed with water (10 ml/mmole), dried over magnesium sulphate and concentrated under vacuum. The crude product obtained is chromatographed on silica with a CH2Cl2/AcOEt mixture 95/5.

c1) obtaining the diazoted amino acid of formula (VI) starting from the product of formula (VIc) obtained above in b) by deprotection of the fmoc-amino diazoketones

A solution of the Fmoc-amino diazoketone product of formula (VIc) obtained in Stage b2) above in dichloromethane (3.5 ml/mmole) is treated at 0° C. under nitrogen with 2 equivalents of diazabicyclo-undecene (DBU)(2.1 equi.). The reaction is left for half an hour, then the solution is deposited directly onto a silica column. The amine is rapidly eluted with a solution of CH2Cl2/MeOH 90/10. The crude free amine obtained is directly reacted for the peptide coupling that constitutes Stage 3.

Stage 2: Preparation of the Bicyclic Scaffold of Formula (V)

This stage is broken down into 2 Stages a2) and b2) a2) Functionalization of the amine i.e. obtaining the product of formula (IV) from the product of formula (II) by reaction with a product of formula (III),

The product of formula (II) is the methyl ester of 9(S)-amino-6,10-dioxo-1,2,3,4,7,8,9,10-octahydro-6H-pyridazino[1,2-a][1,2]diazepine-1(S)-carboxylic acid. In order to obtain the product (IV) from the product of formula (II), the product of formula (II) is reacted with a product R1-X of formula (III) as defined above chosen for example from Table 1 described in FIG. 2 and the process is carried out as follows:

1 equivalent of acid chloride, sulphonyl chloride, carbamoyl chloride or isocyanate, followed by 2 equivalents of diisopropylethylamine (DIEA) are successively added at 0° C. and under nitrogen to a solution of the amine of formula (II) in dichloromethane (6 ml/mmole). The reaction medium is poured into iN HCl (3 ml/mmole). The organic solution is washed successively with a saturated solution of NaCl (3 ml/mmole), then a saturated solution of sodium bicarbonate (3 ml/mmole). After drying over magnesium sulphate the solution is concentrated under vacuum. The crude product obtained is used directly for the following stage of saponification of the ester function b2) Obtaining the product of formula (V) from the product of formula (IV) by saponification of the ester function The methyl ester obtained in Stage a) above is treated with a 2M methanolic solution of lithine (2 equi.). After reacting overnight at ambient temperature, the mixture is concentrated to half its volume with a rotary evaporator, then the same volume of water is added. The pH of the solution is adjusted to 1 by the addition of concentrated acid. The mixture is extracted with ethyl acetate (2×5 ml/mmole). The organic phase is washed with a saturated solution of sodium chloride. After drying over magnesium sulphate, the solution is concentrated to dryness. The product obtained is purified if necessary on silica eluting with a CH2Cl2/MeOH/AcOH, mixture 95/5/0.5.

Stage 3: Peptide Coupling

The peptide coupling is carried out by reacting the product of formula (VI) obtained in Stage 1 and the product of formula (V) obtained in Stage 2 in order to obtain a product of formula (Ix) 1.1 equivalent of TBTU (2-(1H-Benzotriazole-1yl)1,1,3,3-tetramethyluronium tetrafluoroborate) and 2 equivalents of DIEA are added successively, under nitrogen and at 0° C. to a solution of the free amine and the acid of formula (V) in CH2Cl2 (3.5 ml/mmole). The reaction is left overnight at ambient temperature. The solution is poured into a saturated solution of NaHCO3 (20 ml/mmole) and extraction is carried out with ethyl acetate (2×10 ml/mmole). The combined organic phases are washed with water (20 ml/mmole), then a saturated solution of KHSO3, dried over MgSO4 and concentrated under vacuum.

Stage 4: Obtaining a Product of Formula (I) i.e. (Iy) from Another Product of Formula (I) i.e. a Product of Formula (Ix) Obtained in Stage 3 Above

This stage is broken down into 2 Stages a4) and b4). a4) bromination of the diazoketone of the product of formula (Ix). A solution of diazoketone in CH2Cl2 (6 ml/mmole) is treated at 0° C. under nitrogen with a solution of HBr (37%)/acetic acid 30/70 (0.6 ml/mmole). At the end of the reaction the mixture is poured into a saturated solution of NaHCO3 (50 ml/mmole). The mixture is extracted with ethyl acetate (2×30 ml/mmole). The organic solution is washed with water (230 ml/mmole), dried over MgSO4 and concentrated to dryness. If necessary the crude product is purified on silica by eluting with CH2Cl2/MeOH 95/5.

b4) Substitution by two possible methods b4i) or b4ii)

b4i) Method with KF

2 equivalents of potassium fluoride (KF) are added to a solution of bromoketone in DMF (6 ml/mmole) followed by 1.2 equivalent of nucleophile of formula (VIII) i.e. R4′—L—H which can in particular be in the form of acid, thiol or hydroxyl as indicated in Table 2 of FIG. 3 described hereafter. The reaction is left overnight under agitation then the mixture is poured into a saturated solution of NaHCO3 (100 ml/mmole) and extraction is carried out with ethyl acetate (2×50 ml/mmole). The combined organic phases are washed with water, dried over MgSO4 and concentrated to dryness.

If necessary chromatography on silica is carried out eluting with CH2Cl2/MeOH.

b4ii) Method using dimethylaminomethylpolystyrene resin.

2 equivalents of dimethylaminomethylpolystyrene resin (Fluka 3-4 mmole of base/g) is added to a solution of bromoketone (1 equivalent) and the nucleophile of formula (VIII) (1.1 equivalent) in DMF (15 ml/mmole). The reaction is left overnight under gentle agitation. The reaction mixture is filtered then evaporated under vacuum. If necessary the products obtained are purified on silica eluting with a Methanol/CH2Cl2 mixture.

By way of example, the preparation of the products of Examples 1, 2 and 3 of the present application is described hereafter in the experimental part of the present application.

It should be noted that Stage 1 described hereafter for Example 1 is that of all the products of formula (I) of the present application in which R2 represents a remainder of the amino acid Leucine and therefore for which the starting amino acid of formula (VIa) is leucine (Leu): the same method can be applied in the case where the amino acid is different i.e. in particular when the starting amino acid of formula (VIa) represents GLU-alloc, GLY, ALA, VAL, PHE or TYR(tBu). Such amino acids and also the GLU-alloc compound are commercially available.

AA amino acids in the form of compound fmoc-AA of formula (VIb) are in particular commercially available.

When the starting amino acid of formula (VIa) is tyrosine TYR, the process comprises one additional stage as indicated in the preparation of Example 3 hereafter.

The reaction of the product of formula (II) with the product of formula (III) in order to produce a product of formula (IV) can be carried out in particular in the presence of DIEA or also in a solvent such as CH2-Cl2 or also (DMF).

In the product of formula (III), the R1′ radical represents the values indicated above for R1 in protected form if necessary and X represents a halogen atom such as chlorine or bromine.

The product of formula (IV) thus obtained is subjected, in order to produce the product of formula (V), to a saponification reaction under conditions known to a person skilled in the art such as in particular in the presence of LiOH in an alcohol such as methanol or also ethanol, dioxane or dimethoxyethane, in the presence of soda or potash.

The product of formula (VIb) is obtained by fixing the fmoc radical on the terminal amine function of the amino acid of formula (VIa): such an amino acid of formula (VIa) can be any so-called natural or non-natural amino acid as indicated above.

The product of formula (VIb) is subjected in two stages as indicated above to a diazotation reaction in order to produce the product of formula (VIc).

The product of formula (VIc) thus obtained is then released from the fmoc radical in order to produce the product of formula (VI).

The coupling reaction of the product of formula (V) with the product of formula (VI) in order to produce the product of formula (Ix) is carried out for example in TBTU/DIEA in a solvent such as DMF or also dichloromethane.

The products of formula (Ix) represent the products of formula (I) in which the reactive functions are optionally protected and in which R3 represents the CH═N2 radical.

In order to obtain the products of formula (I) in which R3 represents the —CH2—L—R4 radical, the products of formula (Ix) are subjected firstly to bromination under the conditions as defined above in order to produce the products of formula (VII). In order to produce the products of formula (Iy) as defined above, the products of formula (VII) are then subjected to the action of the products of formula (VIII) H—L—R4′: such a reaction can be carried out in particular according to two different reaction methods as indicated above.

According to the values of R′1, R2′, R4′ and R7′, the products of formulae (Ix) and (Iy) constitute or do not constitute the products of formula (I) and can produce the products of formula (I), or be converted into other products of formula (I) by being subjected to one or more of the reactions a) to k) indicated above.

Therefore the various reactive functions can be carried by certain compounds of the reactions defined above can, if necessary, be protected: for example it concerns the hydroxyl, acyl, free carboxy or also amino and monoalkylamino radicals which can be protected by the appropriate protective groups.

The following, non-exhaustive list of examples of protecting reactive functions can be mentioned:

the hydroxyl groups can be protected for example by alkyl radicals such as tert-butyl, trimethylsilyl, tert-butyldimethylsilyl, methoxymethyl, tetrahydropyrannyl, benzyl or acetyl,

the amino groups can be protected for example by the acetyl, trityl, benzyl, tert-butoxycarbonyl, benzyloxycarbonyl, phthalimido radicals or other radicals known in peptide chemistry,

the acyl groups such as the formyl group can be protected for example in the form of ketals or thioketals cyclic or non-cyclic such as dimethyl or diethylketal or ethylene dioxyketal, or diethylthioketal or ethylenedithioketal,

the acid functions of the products described above can be, if desired, amidified by a primary or secondary amine for example in methylene chloride in the presence, for example, of 1-ethyl-3-(dimethylamino-propyl)carbodiimide hydrochloride at ambient temperature:

the acid functions can be protected for example in the form of esters formed with easily cleavable esters such as the benzylic or terbutylic esters or esters known in peptide chemistry.

The reactions to which the products of formulae (Ix) and(Iy) as that defined above can be subjected, if desired or if necessary, can be carried out, for example, as indicated hereafter.

a) The products described above can, if desired, be the subject, on the optional carboxy functions, of esterification reactions which can be carried out according to the usual methods known to a person skilled in the art.

b) The optional conversions of ester functions to acid functions of the products described above can be, if desired, carried out under the usual conditions known to a person skilled in the art in particular by acid or alkaline hydrolysis for example by soda or potash in an alcoholic medium such as, for example, in methanol or also by hydrochloric or sulphuric acid.

c) The optional alkylthio groups of the products described above can be, if desired, converted to the corresponding sulphoxide or sulphone functions under the usual conditions known to a person skilled in the art such as for example by peracids such as for example peracetic acid or metachloroperbenzoic acid or also by ozone, oxone, sodium periodate in a solvent such as for example methylene chloride or dioxane at ambient temperature.

Obtaining the sulphoxide function can be encouraged by an equimolar mixture of the product containing an alkylthio group and a reagent such as in particular a peracid.

Obtaining the sulphone function can be encouraged by a mixture of the product containing an alkylthio group with an excess of reagent such as in particular a peracid.

d) The conversion reaction of the ketone function to an oxime function can be carried out under usual conditions known to a person skilled in the art, such as in particular reaction in the presence of an optionally O-substituted hydroxylamine in an alcohol such as for example ethanol, at ambient temperature or while heating.

e) The optional free or esterified carboxy functions of the products described above can be, if desired, be reduced to an alcohol function by the methods known to a person skilled in the art: the optional esterified carboxy functions can be, if desired reduced to an alcohol function by the methods known to a person skilled in the art and in particular by lithium aluminium hydride in a solvent such as for example tetrahydrofuran or also dioxane or ethyl ether. The optional free carboxy functions of the products described above can be, if desired, reduced to an alcohol function in particular by boron hydride.

f) The optional alkoxy functions such as in particular methoxy of the products described above can be, if desired, converted to a hydroxyl function under the usual conditions known to a person skilled in the art for example by boron tribromide in a solvent such as for example methylene chloride, by pyridine hydrobromide or hydrochloride or also by hydrobromic or hydrochloric acid in water or trifluoroacetic acid under reflux.

g) The optional alcohol functions of the products described above can be, if desired, converted to an aldehyde or acid function by oxidation under the usual conditions known to a person skilled in the art such as for example by the action of manganese oxide in order to obtain aldehydes or Jones reagent in order to access acids.

h) The optional nitrile functions of the products described above can be, if desired, converted to tetrazolyl under the usual conditions known to a person skilled in the art such as for example by cycloaddition of a metallic azide such as for example sodium azide or an trialkyltin azide on the nitrile function as indicated in the method described in the article with the following reference:

J. Organometallic Chemistry., 33, 337 (1971) KOZIMA S.& coll. It should be noted that the conversion reaction of a carbamate to urea and in particular of a sulphonylcarbamate to sulphonylurea, can be carried out for example under reflux of a solvent such as for example toluene in the presence of the suitable amine.

It is understood that the reactions described above can be carried out as indicated or also, if appropriate, according to other usual methods known to a person skilled in the art.

i) The elimination of protective groups such as for example those indicated above can be carried out under the usual conditions known to a person skilled in the art in particular by acid hydrolysis carried out with an acid such as hydrochloric, benzene sulphonic or para-toluene sulphonic, formic or trifluoroacetic acid or also by catalytic hydrogenation.

The phthalimido group can be eliminated by hydrazine.

A list of different protective groups which can be used can be found for example in the Patent BF 2 499 995.

j) The products described above can, if desired, be the subject of salification reactions for example by a mineral or organic acid or by a mineral or organic base according to the usual methods known to a person skilled in the art. The salification reaction can for example be carried out in the presence of hydrochloric acid for example or also tartaric, citric or methane sulphonic acid, in an alcohol such as for example ethanol or methanol.

k) The optional optically active forms of the products described above can be prepared by resolution of the racemics according to the usual methods known to a person skilled in the art.

Illustrations of the reactions defined above are given in the preparation of the examples described hereafter.

The products of formula (I) as defined above as well as their addition salts with acids have useful pharmacological properties.

The products of the present invention can therefore be endowed with properties inhibiting one or more metabolic enzymes as defined above in particular kinases or proteases. Certain products of formula (I) of the present invention as defined above, can therefore in particular have properties inhibiting certain protein kinases or proteases.

Several kinase inhibitors have been described such as butyrolactone, flavopiridol and 2(2-hydroxyethylamino)-6-benzylamino-9-methylpurine called olomoucine.

Several protease inhibitors have been also described, in particular in the ‘Journal of Medicinal Chemistry, vol. 43 -No. 3 (D. Leung, G. Abbenante and D. P. Fairlie). According to a non-exhaustive list, argatroban, napsagatran, inogatran, efegatran, CVS-1123 as well as melagatran can for example be mentioned as thrombin inhibitors.

As powerful inhibitors of factor Xa, DX-9065a, YM-60828 and ZK 807191 or also FX 2212 can also be mentioned.

As elastase inhibitors ICI-200800, MR 889, L-658.758, MDL 101,146, ZD 8321 or also different heterocyclic derivatives such as penicillins, penems, β-lactames, isocoumarins, benzisothiazolones, alkylazetidinones can be mentioned.

As tryptase inhibitors, APC-366 can be mentioned, as “complement convertase” inhibitors, sepimostat mesylate (FUT-187) or nafamostat mesylate (FUT 175) can be mentioned.

As a powerful inhibitor of cathepsin K, Cbz-Leu-Leu-Leu-CHO, 1,3-bis(acylamino)-2-propanone or 1,5-diacylcarbohydrazide derivatives can be mentioned; as caspase inhibitors, 5-aminopyrimidine-6-one derivatives, phenyl ketomethyl ether or amino methylene ketone derivatives can be mentioned; as inhibitors of calpaines, peptide aldehydes such as Cbz-Val-Phe-CHO and calpeptine can be mentioned.

The levels, regulation and activity of a certain number of protein kinases or proteases play a role in several human pathologies. The activity of a protein kinase or protease can in particular be associated with receptors possessing transmembrane domains or intracellular proteins.

Certain kinases or proteases can play a role in the initiation, the development and completion of the events of the cell cycle and therefore, inhibitory molecules of such kinases or proteases are capable of limiting undesirable cellular proliferation such as that observed in cancer, psoriasis, fungal growth, parasites (animal, protist): such inhibitory molecules of these kinases or proteases are also therefore capable of playing a role in the regulation of neurodegenerative diseases such as Alzheimer's disease.

Certain products of formula (I) of the present invention can therefore be endowed with antimitotic properties.

Certain products of formula (I) as defined above can as kinase or protease inhibitors have in particular the property of inhibiting the bone resorption mediated by the osteoclasts. They can therefore be useful for the therapeutic or prophylactic treatment of diseases which are caused at least in part by an undesirable increase in bone resorption, for example osteoporosis.

Certain products of formula (I) of the present invention can therefore for example inhibit the adhesion of the osteoclasts on the surface of the bone and therefore bone resorption by the osteoclasts.

Bone diseases, the treatment or the prevention of which requires the use of the compounds of formula (I) or their prodrugs, are in particular osteoporosis, hypercalcemia, osteopenia, for example caused by bony metastases, dental disorders for example parodontitis, hyperparathyroidism, periarticular erosions in rhumatoid arthritis, Paget's disease, osteopenia induced by immobilisation. Moreover the compounds of formula (I) can be used to relieve, prevent or treat bone disorders which are caused by treatments by glucocorticoids, therapies linked to taking steroids or corticosteroids or male or female sexual hormone deficiencies.

All of these disorders are characterized by bone loss, which is caused by a fault in the balance between bone formation and bone destruction and which can be favourably influenced by the inhibition of bone resorption by the osteoclasts.

Certain products of formula (I) of the present invention can possess in addition to their specific inhibitory properties of kinases or proteases, useful cellular effects such as antiproliferative properties and in particular effects on apoptosis.

It is known from work described in the literature such as WO 97/20842, that relationships exist between the cell cycle and apoptosis. Among the routes leading to apoptosis, some are dependant on kinases or proteases.

The products of the present invention are in particular useful for tumour therapy.

The products of the invention can also therefore increase the therapeutic effects of currently used anti-tumoral agents. The products of formula (I) of the present invention therefore more particularly have antimitotic and anti-neurodegenerative properties.

Certain products of the present invention can be inhibitors of vasoconstrictive and hypertensive effects and therefore produce an anti-ischemic effect, or also combat stimulant effects at the level of certain cellular types in particular the smooth muscle cells, fibroblasts, neuronal cells and bone cells.

The products according to the present invention can therefore be used in the treatment of diseases such as proliferative diseases, cancer, the recurrence of stenosis, inflammation; allergies, cardiovascular diseases or certain infectious diseases.

The products of the present invention can also be used in the treatment of certain gastrointestinal, gynecological disorders and in particular for a relaxing effect at the level of the uterus.

The products of formula (I) of the present application can therefore have useful pharmacological properties justifying their use in therapeutics.

A particular subject of the invention is therefore as medicaments, the products of formula (I)as defined above, said products of formula (I) being in all possible racemic, enatiomeric and diastereoisomeric isomer forms, as well as the addition salts with pharmaceutically acceptable mineral and organic acids or mineral and organic bases of said products of formula (I).

A more particular subject of the invention is therefore as medicaments, the products as defined by formula (Ia) or (Ib), said products of formula (Ia) or (Ib) being in all possible racemic, enatiomeric and diastereoisomeric isomer forms, as well as the addition salts with pharmaceutically acceptable mineral and organic acids or mineral and organic bases of said products of formula (Ia) or (Ib).

A quite particular subject of the invention is, as medicaments, the products described hereafter in the examples and in particular the products of formula (I) as defined above, corresponding to the following formulae:

3-[9(S)-benzoylamino-6,10-dioxo-1,2,3,4,7,8,9,10-octahydro-6H-pyridazino[1,2-a][1,2]diazepine-1(S)-carboxamide]-5-methyl-1-benzoyloxy-hexane-2-one

3-[9(S)-(2-furanoyl)amino-6,10-dioxo-1,2,3,4,7,8,9,10-octahydro-6H-pyridazino[1,2-a][1,2]diazepine-1(S)-carboxamide]-5-methyl-1-(2,6-dichlorobenzoyloxy-hexane-2-one

3(S)-[9(S)-(2-furanoyl)amino-6,10-dioxo-1,2,3,4,7,8,9,10-octahydro-6H-pyridazino[1,2-a][1,2]diazepine-1(S)-carboxamide]-4-(4-hydroxyphenyl)-1-(2,6-dichlorobenzoyloxy)-butane-2-one as well as the products of formula (I) as defined above corresponding to the following formulae:

(2-propenyl)(4S)6-diazo-4-[[[(1S,9S)-9-[(2-methyl-1-oxo-propyl)amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[(3-methoxybenzoyl)amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[(2-furanyl)carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[(cyclohexylamino)carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[[2-(p-2-yl)-ethyl]amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[(methylsulphonyl)amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[(2-naphthalenyl)sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-diazo-4-[[[(1S,9S)-9-[[[5-(isoxazol-3-yl)-2-thienyl]sulphonyl]amino]-octahydro-6,10-dioxo-6H-pyridazino[1,2-a][1,2]diazepine-1-yl]carbonyl]amino]-5-oxo-hexanoate

9-[(9S)-[[(4-phenoxyphenyl)amino]carbonyl]amino]-N-[(2S)-4-diazo-1-[4-[(1,1-dimethyl)ethoxy]phenyl]-3-oxo-2-butyl]-6H-(1S)-pyridazino[1,2-a][1,2]diazepine-1-carboxamide

(2-propenyl)(4S)6-[(2,6-dichlorobenzoyl)oxy]-4-[[[(1S,9S)-9-[[(2-furanyl)carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-[4-[[2-oxo-2-(1-pyrrolidinyl)ethyl]piperazin-1-yl]-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-[4-[[2-oxo-2-(1-pyrrolidinyl)ethyl]piperazin-1-yl]-4-[[[(1S,9S)-9-[[(2-naphthalenyl)sulphonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[(cyclohexylamino)carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(1-phenyl-1H-tetrazol-5-yl)thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(benzothiazol-2-yl)thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[[(4-methoxyphenyl)methyl]thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[[(4-pyridinyl)carbonyl]oxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(2,6-dichlorobenzoyl)oxy]-4-[[[(1S,9S)-9-[[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)((4S)6-acetyloxy-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[1-oxo-2-(thien-3-yl)ethoxyl-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate

(2-propenyl)(4S)6-[(1-phenyl-1H-tetrazol-5-yl)thio]-4-[[[(1S,9S)-9-[[[[2(thien-2yl)-ethyl]amino]carbonyl]amino]-octahydro-6,10dioxo-6H-pyridazino[1,2a][1,2]diazepine-1-yl]carbonyl]amino]-5oxo-hexanoate

(2propenyl)(4S)6[(2,6dichlorobenzoyl)oxy]-4-[[[(1S,9S)-9[[[[2(thien-2yl)-ethyl]amino]carbonyl]amino]-octahydro-6,10dioxo-6H-pyridazino[1,2a][1,2]diazepine-1-yl]carbonyl]amino]-5oxo-hexanoate

(2propenyl)(4S)6[(2,6dichlorobenzoyl)oxy]-4-[[[(1S,9S)-9[(methylsulphonyl)amino]-octahydro-6,10-dioxo-6H-pyridazino[1,2a][1,2]diazepine-1yl]carbonyl]amino]-5-oxo-hexanoate

9[(9S)[[(4phenoxyphenyl)amino]carbonyl]amino]-N-((2S)-4-diazo-1phenyl-3oxo-2butyl)-octahydro-6,10dioxo-6H-1S)-pyridazino[1,2a][1,2]diazepine-1-carboxamide

9[(9S)-[[[2(thien-2yl)-ethyl]amino]carbonyl]amino]-N-((2S)-4diazo-1phenyl-3oxo-2butyl)-octahydro-6,10-dioxo-6H-(1S)-pyridazino[1,2a][1,2]diazepine-1-carboxamide

9[(9S)-[(2naphthalenyl)sulphonyl]amino ]-N-((2S)-4-diazo-1phenyl-3oxo-2butyl)-octahydro-6,10dioxo-6H-(1S)-pyridazino[1,2a][1,2]diazepine-1carboxamide

9[(9S)-[[5(isoxazol-3yl)-2thienyl]sulphonyl]amino]-N-((2S)-4diazo-1phenyl-3oxo-2butyl)-octahydro-6,10-dioxo-6H-(1S)-pyridazino[1,2a][1,2]diazepine-1-carboxamide

9[(9S)-[[(4phenoxyphenyl)amino]carbonyl]amino]-N-((2S)-4-diazo-3oxo-2butyl)-octahydro-6,10dioxo-6H-(1S)-pyridazino[1,2a][1,2]diazepine-1-carboxamide

9[(9S)-[[[3(thien-2yl)-ethyl]amino]carbonyl]amino]-N-((2S)-4diazo-3oxo-2butyl)-octahydro-6,10dioxo-6H-(1S)-pyridazino[1,2a][1,2]diazepine-1-carboxamide

9[(9S)-(3methoxybenzoyl)amino]-N-((2S)-4[(2,6-dichlorobenzoyl)oxy]-1phenyl-3oxo-2butyl]-octahydro-6,10dioxo-6H-(1S)-pyridazino[1,2a][1,2]diazepine-1-carboxamide

9[(9S)-[(2furanyl)carbonyl]amino]-N-[(2S)-4[(2,6-dichlorobenzoyl)oxy]-1phenyl-3oxo-2butyl]-octahydro-6,10dioxo-6H-(1S)-pyridazino[1,2a][1,2]diazepine-1-carboxamide

9[(9S)-[(cyclohexylamino)carbonyl]amino]-N-[(2S)-4[(2,6-dichlorobenzoyl)oxy]-1phenyl-3oxo-2butyl]-octahydro-6,10dioxo-6H-(1S)-pyridazino[1,2a][1,2]diazepine-1-carboxamide

9[(9S)-[[(4phenoxyphenyl)amino]carbonyl]amino]-N-[(2S)-1-phenyl-4[(1phenyl-1H-tetrazol-5yl)thio]-3oxo-2butyl]-octahydro-6,10dioxo-6H-(1S)-pyridazino[1,2a][1,2]diazepine-1-carboxamide

9[(9S)-[[(4phenoxyphenyl)amino]carbonyl]amino]-N-[(2S)-1-phenyl-4[1oxo-2(3thienyl)ethoxy]-3oxo-2butyl]-octahydro-6,10dioxo-6H-(1S)-pyridazino[1,2a][1,2]diazepine-1-carboxamide

(methyl)[2oxo-4phenyl-3[(3S)-[[(4phenoxyphenyl)amino]carbonyl]amino]-octahydro-6,10dioxo-6H-(1S)-pyridazino[1,2-a][1,2]diazepin-1yl]carbonyl]amino]butyl]pentanedioate

9[(9S)-[[(4phenoxyphenyl)amino]carbonyl]amino]-N-[(2S)-4-[(4phenylmethyl)-1piperidinyl]-1phenyl-3oxo-2butyl]-octahydro-6,10dioxo-6H-(1S)-pyridazino[1,2a][1,2]diazepine-1-carboxamide

9[(9S)-[[[2(thien-2yl)-ethyl]amino]carbonyl]amino]-N-[(2S)-1phenyl-4[(1phenyl-1H-tetrazol-5yl)thio]-3oxo-2-butyl]-octahydro-6,10dioxo-6H-(1S)-pyridazino [1,2-a][1,2]diazepine-1-carboxamide

9[(9S)-[[[2(thien-2yl)-ethyl]amino]carbonyl]amino]-N-[(2S)-1phenyl-4[(2benzothiazolyl)thio]-3oxo-2butyl]-octahydro-6,10dioxo-6H-(1S)-pyridazino[1,2a][1,2]diazepine-1-carboxamide

9[(9S)-[[[2(thien-2yl)-ethyl]amino]carbonyl]amino]-N-[(2S)-1phenyl-4[(-2,6dichlorobenzoyl)oxy]-3oxo-2butyl]-octahydro-6,10dioxo-6H-(1S)-pyridazino[1,2a][1,2]diazepine-1-carboxamide

9[(9S)-[[[2(thien-2yl)-ethyl]amino]carbonyl]amino]-N-[(2S)-1phenyl-4[4[2(1pyrrolidinyl)-2-oxo-ethyl]piperazin-1yl]]-3oxo-2butyl]-octahydro-6,10-dioxo-6H-(1S)-pyridazino[1,2a][1,2]diazepine-1-carboxamide

9[(9S)-(methylsulphonyl)amino]-N-[(2S)-1phenyl-4[(-2,6-dichlorobenzoyl)oxy]-3oxo-2butyl]-octahydro-6,10-dioxo-6H-(1S)-pyridazino[1,2a][1,2]diazepine-1-carboxamide

9[(9S)-[[[4(trifluoromethyl)phenyl]sulphonyl]amino]-N-[(2S)-1phenyl-4[(1phenyl-1H-tetrazol-5yl)thio]-3oxo-2-butyl]-octahydro-6,10dioxo-6H-(1S)-pyridazino [1,2-a][1,2]diazepine-1-carboxamide

9[(9S)-[[[4(trifluoromethyl)phenyl]sulphonyl]amino]-N-[(2S)-1phenyl-4(2benzothiazolyl)thio]-3oxo-2butyl]-octahydro-6,10dioxo-6H-(1S)-pyridazino[1,2a][1,2]diazepine-1-carboxamide

9[(9S)-(methylsulphonyl)amino]-N-[(3S)-5methyl-1[(2,6-dichlorobenzoyl)oxy]-2oxo-3hexyl]-octahydro-6,10-dioxo-6H-(1S)-pyridazino[1,2a][1,2]diazepine-1-carboxamide

9[(9S)-[[[4(trifluoromethyl)phenyl]sulphonyl]amino]]-N-[(3S)-5methyl-1[(2,6dichlorobenzoyl)oxy]-2oxo-3hexyl]-octahydro-6,10dioxo-6H-(1S)-pyridazino[1,2a][1,2]diazepine-1-carboxamide

9[(9S)-[(2naphthalenyl)sulphonyl]amino ]-N-[(3S)-5-methyl-1[(2,6dichlorobenzoyl)oxy]-2oxo-3hexyl]-octahydro-6,10dioxo-6H-(1S)-pyridazino[1,2a][1,2]diazepine-1-carboxamide

9[(9S)-(2methyl-1oxo-propyl)amino]-N-((3S)-1diazo-5-methyl-2oxo-3hexyl)-octahydro-6,10dioxo-6H-(1S)-pyridazino[1,2a][1,2]diazepine-1-carboxamide

9[(9S)-(3methoxybenzoyl)amino]-N-((3S)-1diazo-5-methyl-2oxo-3hexyl)-octahydro-6,10dioxo-6H-(1S)-pyridazino[1,2-a][1,2]diazepine-1-carboxamide

9[(9S)-[(2furanyl)carbonyl]amino]-N-((3S)-1diazo-5-methyl-2oxo-3hexyl)-octahydro-6,10dioxo-6H-(1S)-pyridazino[1,2a][1,2]diazepine-1-carboxamide

9[(9S)-[(cyclohexylamino)carbonyl]amino]-N-((3S)-1-diazo-5methyl-2oxo-3hexyl)-octahydro-6,10dioxo-6H-(1S)-pyridazino[1,2a][1,2]diazepine-1-carboxamide

9[(9S)-[[(4phenoxyphenyl)amino]carbonyl]amino]-N-((3S)-1-diazo-5methyl-2oxo-3hexyl)-octahydro-6,10dioxo-6H-(1S)-pyridazino[1,2a][1,2]diazepine-1-carboxamide

9[(9S)-[[[2(thien-2yl)-ethyl]amino]carbonyl]amino]-N-((3S)-1diazo-5methyl-2oxo-3hexyl)-octahydro-6,10-dioxo-6H-(1S)-pyridazino[1,2a][1,2]diazepine-1-carboxamide

9[(9S)-(methylsulphonyl)amino]-N-((3S)-1diazo-5methyl-2-oxo-3hexyl)-octahydro-6,10dioxo-6H-(1S)-pyridazino[1,2-a][1,2]diazepine-1-carboxamide

9[(9S)-[[4(trifluoromethyl)phenyl]sulphonyl]amino]-N-((3S)-1diazo-5methyl-2oxo-3hexyl)-octahydro-6,10-dioxo-6H-(1S)-pyridazino[1,2a][1,2]diazepine-1-carboxamide

9[(9S)-[(2naphthalenyl)sulphonyl]amino]-N-((3S)-1-diazo-5methyl-2oxo-3hexyl)-octahydro-6,10dioxo-6H-(1S)-pyridazino[1,2a][1,2]diazepine-1-carboxamide

9[(9S)-(3methoxybenzoyl)amino]-N-[(3S)-1[(benzothiazol-2yl)thio]-4methyl-2oxo-3pentyl]-octahydro-6,10-dioxo-6H-(1S)-pyridazino[1,2-a][1,2]diazepine-1-carboxamide

9-[(9S)-[(2-furanyl)carbonyl]amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide

9-[(9S)-[(cyclohexylamino)carbonyl]amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide

9-[(9S)-[[(4-phenoxyphenyl)amino]carbonyl]amino]-N-[(3S)-1-[(1-phenyl-1H-tetrazol-5-yl)thio]-4-methyl-2-oxo-3-pentyl]-octahydro-6,10-dioxo-6H-(1S)-pyridazino [1,2-a][1,2]diazepine-1-carboxamide

9-[(9S)-[[(4-phenoxyphenyl)amino]carbonyl]amino]-N-[3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide

9-[(9S)-[[[2-(thien-2-yl)-ethyl]amino]carbonyl]amino]-N-[(3S)-1-[(1-phenyl-1H-tetrazol-5-yl)thio]-4-methyl-2-oxo-3-pentyl]-octahydro-6,10-dioxo-6H-(1S)-pyridazino[1,2-a][1,2]diazepine-1-carboxamide

9-[(9S)-[[[2-(thien-2-yl)-ethyl]amino]carbonyl]amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide

9-[(9S)-[(cyclohexylamino)carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide

9-[(9S)-[(2-furanyl)carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide.

The products of formula (I) of the present invention can therefore in particular be used in the form of medicaments to inhibit an undesirable mechanism provoking a pathology under the effect of one or more protease(s) or kinase(s): the method can therefore consist of the administration to a patient whose treatment requires the inhibition of the effect of such a protease or kinase, of an inhibitory quantity of such a medicament according to the present invention.

The medicaments which are a subject of the invention can therefore be used in the treatment and prevention of cardiovascular illnesses associated with an alteration of vasomotoricity or of volaemia, myocardial infarction and its consequences, cardiac insufficiency, renal insufficiency, angina pectoris, hyperaldosteronism, arterial hypertension and its consequences, the treatment and prevention of complications in particular cardiac and vascular hypertrophies as well as the development of fibroses at the level of the target organs, prevention and treatment of hypertension, metabolic, endocrinological and neurological disorders, endotoxic shocks, “subarachnoid” haemorrhages, arrhythmia, asthma, acute renal failure, preeclampsia and diabetes.

The medicaments which are a subject of the invention can therefore be used in the treatment of vascular spasms, in treatment of the consequences of a cerebral haemorrhage, in the treatment of coronary spasms, peripheral vascular spasms. These medicaments can in particular be used in the treatment of congestive cardiac insufficiency, in the prevention of the post-angioplasty recurrence of stenosis, the prevention of cardiac and vascular fibroses, in the treatment of atherosclerosis and of certain forms of hypertension such as pulmonary hypertension as well as in the treatment of asthma.

These medicaments which are a subject of the invention can also be used for the treatment of glaucoma and different types of visceral spasms, as well as neuronal protecting substances or also in the prevention of the post-angioplasty recurrence of stenosis.

In particular the medicaments which are a subject of the invention can be used for their anti-hypertrophic and anti-fibrotic effects at cardiac and vascular levels. More particularly, they can be used for the treatment and prevention of the cardiovascular disorders associated with diabetes.

The medicaments which are a subject of the present invention can also be of use in the treatment of osteoporosis and as neuronal protectors.

The medicaments which are a subject of the invention can also be used in the treatment of memory disorders and disorders of the cognitive functions as well as anxiety.

The medicaments, which are a subject of the invention, can also be of use, as antimitotics, in the chemotherapy of cancers, or also in the treatment of psoriasis, parasitoses such as that those caused by protists or by fungi or also in the treatment of Alzheimer's disease or in the treatment of neuronal apoptosis.

A quite particular subject of the invention is the pharmaceutical compositions containing as active ingredient at least one of the medicaments as defined above.

A quite particular subject of the invention is pharmaceutical compositions as defined above characterised in that that they are used as medicaments as defined above.

The pharmaceutical compositions of the present invention as defined above can be administered by buccal route, by parenteral route or by local route as a topical application on the skin and the mucous membranes or by injection by intravenous or intramuscular route.

These compositions can be solids or liquids and be presented in all the pharmaceutical forms commonly used in human medicine such as, for example, plain or sugar-coated tablets, pills, tablets, gelatin capsules, drops, granules, injectable preparations, ointments, creams or gels; they are prepared according to usual methods. The active ingredient can be incorporated with excipients usually used in these pharmaceutical compositions, such as talc, gum arabic, lactose, starch, magnesium stearate, cocoa butter, aqueous or non-aqueous vehicles, fatty substances of animal or vegetable origin, paraffin derivatives, glycols, various wetting, dispersing or emulsifying agents, preservatives.

The usual dose, which is variable according to the product used, the subject treated and the illness in question, can be, for example, from 0.05 to 5 g per day in adults, or preferably from 0,1 to 2 g per day.

A particular subject of the invention is the pharmaceutical compositions as defined above characterised in that that they are used as antimitotic medicaments, in particular for the chemotherapy of cancers or also for the treatment of psoriasis, of parasitoses such as that those caused by fungi or protists or Alzheimer's disease.

A quite particular subject of the invention is also the pharmaceutical compositions as defined above characterised in that that they are used as antineurodegenerative medicaments in particular neuronal anti-apoptosis medicaments.

A particular subject of the invention is the use of the products of formula (1) as defined above for the preparation of pharmaceutical compositions intended for the treatment or the prevention of illnesses requiring an inhibition of one or more metabolic enzymes as defined above.

A particular subject of the invention is therefore the use of the products of formula (I) as defined above for the preparation of pharmaceutical compositions intended for the treatment of diseases resulting from anomalies at the level of the regulation or the activity of a certain number of metabolic enzymes. Such anomalies play a role in certain human pathologies which are particularly concerned by the present invention.

Therefore, the present invention relates in particular to the use of the products of formula (I) of the present invention as protease or kinase inhibitors. The present invention relates therefore to the inhibition of some of these protease or kinase enzymes and claims the use of products of the present invention as inhibitors in the case where the inhibition of such a protease or kinase is indicated.

A quite particular subject of the invention is therefore the use of the products of formula (I) as defined above or of pharmaceutically acceptable salts of said products of formula (I) for the preparation of medicaments intended for the prevention or treatment of medicaments intended for the prevention or treatment of diseases in which metabolic enzymes such as proteases or kinases are involved.

A more particular subject of the invention is the use of the products of formula (I) as defined above or of pharmaceutically acceptable salts of said products of formula (I) characterized in that the diseases to be prevented or treated are chosen from the following group of diseases: cardiovascular diseases, cancers, diseases of the central nervous system, inflammatory diseases, infectious diseases or also bone diseases.

A quite particular subject of the invention is the use of the products of formula (I) as defined above or of pharmaceutically acceptable salts of said products of formula (I) characterized in that the diseases to be prevented or treated are diseases of the central nervous system or bone diseases such as in particular osteoporosis.

A subject of the present invention is therefore the use of the products of formula (I) as defined above or of pharmaceutically acceptable salts of said products of formula (I) for the preparation of medicaments intended in particular for the prevention and treatment of diseases of bone metabolism.

A quite particular subject of the invention is therefore the use of the products of formula (I) as defined above for the preparation of pharmaceutical compositions intended for the treatment of arterial hypertension, cardiac insufficiency, the post-angioplasty recurrence of stenosis, vascular spasms, the consequences of a cerebral haemorrhage and renal insufficiencies.

A quite particular subject of the invention is therefore the use of the products of formula (1) as defined above for the preparation of pharmaceutical compositions intended for the treatment of myocardial infarction, for the prevention of the post-angioplasty recurrence of stenosis and for the prevention of cardiac and vascular fibroses.

A quite particular subject of the invention is therefore the use of the products of formula (1) as defined above for the preparation of pharmaceutical compositions intended for the treatment of renal insufficiency.

A quite particular subject of the invention is therefore the use of the products of formula (1) as defined above for the preparation of pharmaceutical compositions intended for the treatment and for the prevention of cardiovascular disorders associated with diabetes.

A particular subject of the invention is the use of the products of formula (I) as defined above for the preparation of medicaments intended for the chemotherapy of cancers, the treatment of psoriasis, parasitoses such as those caused by fungi or protists, the treatment of Alzheimer's disease or the treatment of neurodegenerative illnesses in particular neuronal apoptosis.

The starting products of formula (VIa) which are therefore natural or non-natural amino acids are commercially available or can be prepared according to the usual methods known to a person skilled in the art.

The following products of formula (VIa) can in particular be mentioned: Glu, Gly, Ala, Val, Phe or Tyr, these amino acids being optionally in a protected form for example by an alloc or terbutyl group.

The products of formula (VIb) can be prepared from so-called natural or non-natural amino acids of formula (VIa), as indicated above, by fixing the Fmoc radical on the N-terminal amine function of the amino acid concerned: such a reaction is in particular described in the article with the following reference: ‘Mueller, A.; Vogt, C.; Sewald, N.; Synthesis (1998),(6),837-841’.

A large number of products of formula (VIb)are also commercially available which are therefore amino acids in fmoc protected form and which can then constitute starting products for the process of the present application described in FIG. 1.

The following products of formula (VIb) can in particular be mentioned: Fmoc-Leu, Fmoc-L-Glu(Alloc); Fmoc-Gly; Fmoc-Ala; Fmoc-Val; Fmoc-Phe or also Fmoc-Tyr(OtBu)

The product of formula (II) is the methyl ester of 9(S)-amino-6,10-dioxo-1,2,3,4,7,8,9,10-octahydro-6H-pyridazino[1,2-a][1,2]diazepine-1(S)-carboxylic acid. Such a starting product of formula (II) can be prepared according to the usual methods known to a person skilled in the art or also as indicated in Patent Application EP 94095 or in the following publications:

Attwood M. and al, J. Chem. Soc., Perkin trans, 1, (1986), (6), 1011-19

Thomas, W and al, J. Chem. Soc., Perkin trans, 2, (1986), (5), 747-55

The starting product of formula (III) i.e. R1X in which X represents a halogen atom can in particular be chosen from Table 1 described in FIG. 2 of the present invention: in such a table, X represents a chlorine atom but can also represent a bromine atom. Such a product of formula (III) is commercially available or can be prepared according to the usual methods known to a person skilled in the art.

The following products of formula (III) can for example be mentioned: benzoyl chloride, isopropanoic acid chloride, cyclohexylisocyanate, methanesulphonyl chloride or also benzenesulphonyl chloride.

The starting product of formula (VIII) i.e. R4′—L—H in which R4′ and L have the meanings indicated above can in particular be chosen from Table 2 described in FIG. 3 of the present invention. Such a product of formula (VIII) is commercially available or can be prepared according to the usual methods known to a person skilled in the art.

The following products of formula (VIII) can in particular be mentioned: mercaptobenzimidazol, benzoic acid, 1-phenylpiperazine, 4-fluorophenol, piperidine.

The experimental part hereafter gives examples of such starting products.

Finally, a subject of the present invention is as new industrial products, the compounds of formula (VII) as defined in the process of the present invention.

The tables of FIG. 4 represented hereafter on pages 69 to 182 represent products which have been obtained by the process described above according to the present invention: these products constitute examples of products of formula (I) of the present invention and are characterized in the tables below by their molecular weights represented by MW in these tables. Such MW values indicated in these tables were determined as follows.

The products represented in the tables of FIG. 4 were prepared and characterized by their HPLC-MS spectra i.e. by high pressure chromatography coupled with a mass spectrograph: such chromatography was carried out using an ODS-AQ type column and an acetonitrile/water eluent mixture in variable proportions as a function of the compounds.

Electrospray in positive mode was used for the determination of molecular weights. The weight of each product was determined by the weight of the H+ molecular ion or in the form of sodium or acetonitrile adduct.

It should be noted that the starting amino acid for the products of Examples 1 and 2 is Leucine: in fact the corresponding product of formula (VIb) i.e. Fmoc-Leu is commercially available and constitutes the starting point of the synthesis of the product of Example 1.

For the other examples of the present application indicated in the tables of FIG. 4 in which the amino acid concerned is also Leu, Stage 1 of the synthesis of these products is carried out in the same way as for Example 1 described hereafter in the experimental part.

For the other examples of the present application indicated in the tables of FIG. 4 for which the amino acid concerned is an amino acid other than Leu, Stage 1 of the synthesis of these products is also carried out in the same way as for Example 1 described hereafter in the experimental part-using instead of Fmoc-Leu, the compound of formula (VIb) corresponding to the amino acid concerned i.e. the following Fmoc amino acids: Fmoc-L-Glu(Alloc); Fmoc-Gly; Fmoc-Ala; Fmoc-Val; Fmoc-Phe and Fmoc-Tyr(OtBu): final products in which the amino acid remainder is a Glu or Glu(Alloc), Gly, Ala, Val, Phe or Tyr(OtBu) remainder are obtained respectively in this way.

For the other examples of the present application indicated in the tables of FIG. 4 for which the amino acid concerned is tyrosine, the preparation of Example 3 is carried out as indicated hereafter, using the product of formula (VI) Fmoc-Tyr(OtBu) in order to obtain the corresponding final product of formula (I) in which the hydroxyl radical is in terbutoxy form: the hydroxyl function is then released on the product of formula (I) obtained in this way, in order to obtain a new product of formula (I) in which the amino acid remainder is then a tyrosine remainder. The deprotection of such formula (I) derivatives where R2 represents (tBuO)PhCH2 can be carried out for example by the standard method with trifluoroacetic acid in a 50/50 mixture with dichloromethane. The diagram of such a reaction is indicated hereafter.

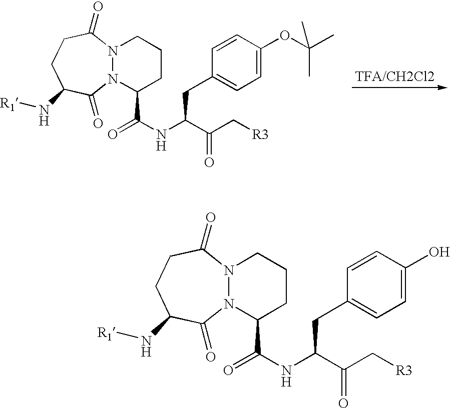

The examples given in the tables of FIG. 4 described hereafter illustrate the process described above but in no way limit the present invention.

The examples described in the experimental part of the present application which are therefore Examples 1 to 3 of the tables of FIG. 4, give examples of the implementation of the present invention which illustrate the invention without however limiting it.

### EXPERIMENTAL PART

### Example 1

### 3-[9(S)-benzoylamino-6,10-dioxo-1,2,3,4,7,8,9,-10-octahydro-6H-pyridazino[1,2-a][1,2]diazepine-1(S)-carboxamide]-5-methyl-1-benzoyloxy-hexane-2-one

Stage 1: N-Fmoc-3-amino-1-diazo-5-methyl-hexane-2-one

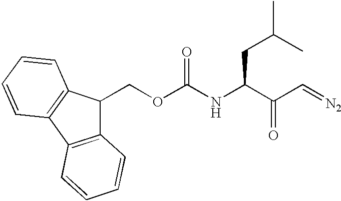

1.3 ml of 4-methyl-morpholine (11.9 mmoles; 1.05 equ.) and dropwise, 1.5 ml of isobutyl chloroformate (11.6 mmoles; 1.03 equ.) are successively added under nitrogen and at −10° C. to a suspension of 3.52 g of Fmoc-Leucine (11.3 mmoles) in 35 ml of dichloromethane. The reaction mixture becomes clear, then a precipitate of 4-methyl-morpholine hydrochloride appears. After agitation for 1 hour at −10° C., the precipitate is filtered.

The resulting solution is cooled down to −10° C., then, under nitrogen, 75 ml (19.1 mmoles, 2 equ.) of 0.3M solution of diazomethane in CH2Cl2 is added.

The temperature is taken progressively to ambient temperature. After reaction for 1 hour at ambient temperature the mixture is poured into 100 ml of a saturated solution of sodium bicarbonate. The organic phase is washed with water (100 ml), dried over magnesium sulphate, then concentrated to dryness. The crude product is directly chromatographed on 500 g of silica using CH2Cl2/AcOEt 95/5.

2.6 g (yield=65%) of a yellowish solid) is isolated. MS: MH⁺ 378

NMR H¹ (CDCl3) (250 MHz) 0.95 (6H; d; 2CH3), 1.40 to 1.85(3H; 2 m; 1CH and 1CH2) 4.18(1H, t, CH Fmoc),4.4 to 4.55(2H; m;1CH and 1CHN₂), 5.28(1H, bd, NHCO), 4.42(2H; bd; CH2 Fmoc), 5.12(1H; bs; CHN2), 6.90 to 7.45(9H; m; aromatic H's), 7.55(2H; bt; aromatic H's), 7.77(2H; bd; aromatic H's)

Stage 2

a) methyl ester of 9(S)-benzoylamino-6,10-dioxo-1,2,3,4,7,8,9,10-octahydro-6H-pyridazino[1,2-a][1,2]diazepine-1(S)-carboxylic acid

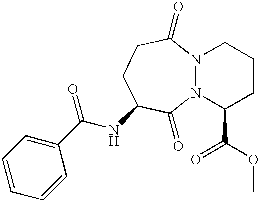

0.68 ml of diisopropylethylamine (2 mmoles; 2 equi.) followed by 0.26 ml of benzoyl chloride (2.16 mmoles, 1.1 equi.) are added successively at 0° C. under nitrogen, to a solution of 500 mg of amine (1) (1.96 mmoles) in 12 ml of CH2Cl2. The temperature is taken to ambient temperature. After agitation for 1 hour, 12 ml of 1N HCl is added. The mixture is decanted and the organic phase washed successively with 12 ml of a saturated solution of NaCl and 12 ml of a saturated solution of sodium bicarbonate. The organic solution is dried over magnesium sulphate then concentrated to dryness. 0.424 mg (yield=54%) of methyl benzoylamino-pyrazepate is isolated.

MS: [MH]⁺ 358; MH⁺ 360; MNa⁺ 382

b) 9(S)-benzoylamino-6,10-dioxo-1,2,3,4,7,8,9,10-octahydro-6H-pyridazino[1,2-a][1,2]diazepine-1(S)-carboxylic acid

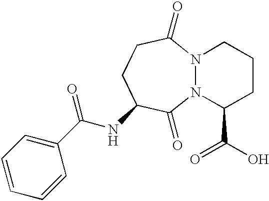

99 mg of lithium hydroxide (2.36 mmoles; 2 equi.) at 0° C. is added to a solution of 424 mg (1.18 mmole) of 9(S)-benzoylamino-octahydro-6,10-dioxo-6H-pyridazino[1,2-a][1,2]diazepine-1(S)-carboxylic methyl ester in 10 ml of methanol. The temperature is raised to ambient temperature then the reaction is maintained overnight. The solution is concentrated with a rotary evaporator to 5 ml. 5 ml of water is added and the pH of the solution is adjusted to 1 by adding a few drops of concentrated HCl. The mixture is extracted with 2×5 ml of ethyl acetate. The organic phase is washed with 5 ml of a saturated solution of NaCl, dried over MgSO4 and concentrated to dryness. 305 mg of 9(S)-benzoylamino-octahydro-6,10-dioxo-6H-pyridazino[1,2-a][1,2]diazepine-1(S)-carboxylic acid is obtained (yield=75%).

MS: [MH]⁻ 344, MH⁺ 346, MNa⁺ 368

NMR-H¹ (CDCl3) (300 MHz): 1.76(2H; m; CH 2) , 1.96 and 2.35(2H; 2 m; CH2), 1.97 and 2.84(2H; 2m; CH2) , 2.41 and 3.48(2H; 2m; CH2), 4.96 and 4.68(2H; m and bd; CH2), 5.17(1H; m; HN), 5.47(1H; dd; CHCO2H), 7.26(1H; d; CONH), 7.38 to 7.53(3H; m; aromatic H's), 7.81(2H; bd; aromatic 2H's)

Stage 3: 3-[9(S)-benzoylamino-6,10-dioxo-1,2,3,4,7,8,9,10-octahydro-6H-pyridazino[1,2-a][1,2]diazepine-1(S)-carboxamide]-5-methyl-1-diazo-hexane-2-one

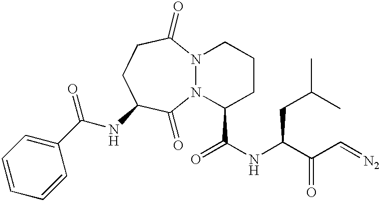

378 mg (1 mmole) of N-Fmoc-3-amino-1-diazo-5-methyl-hexane-2-one in solution in 3.5 ml of CH2Cl2 is treated with 0.31 ml of DBU(2.1 mmole; 2.1 equi.) at 0° C. under nitrogen. After reacting for one hour the solution is placed on a silica column (40 g) and eluted rapidly with a CH2Cl2/methanol solution 90/10. The yellow oil obtained (198 mg, yield=100%) is solubilized in 3.5 ml of CH2Cl2 then 0.35 g (1 mmole) of acid obtained in Stage 2 b) is added. 625 mg of TBTU(1.1 mmole; 1.1 equi.) followed by 0.35 ml of DIEA(2 mmoles; 2 equi) are added successively under nitrogen at 0° C. The reaction is maintained overnight at ambient temperature then the reaction medium is poured into 20 ml of a saturated solution of NaHCO3 and extraction is carried out with 2×10 ml of ethyl acetate. The combined organic phases are washed with water (20 ml) then with 20 ml of a saturated solution of KHSO3. The solution is dried over MgSO4 and concentrated to dryness. 244 mg of a foam is isolated (yield=50%).

MS: [M+Na]⁺ 505; MH⁺ —N2 455; MH⁺ 483

NMR-H¹ (CDCl3) (300 MHz): 0.95(6H; dd; 2CH3), 1.56(2H m; CH2), 1.69(1H; m; CH), 4.53(2H; m; CH2; 6.40(1H; bd; NH), 1.66(2H; m; CH2), 1.92(2H; m; CH2), 1.90 and 2.90(2H; 2m, CH2); 2.39 and 3.24(2H; 1dd and 1dt; CH2), 3.01 and 4.66(2H; 1dt and 1m; CH2), 4.66(1H; td; CH), 5.17(1H; m; CH) , 7.02(1H, d; NH), 5.40(1H; bs; CHN₂), 7.46(2H; dd; aromatic 2H's in meta position), 7.51(1H; m; aromatic H's in para position), 7.81(2H; dd; aromatic H's in ortho position).

Stage 4: 3-[9(S)-benzoylamino-6,10-dioxo-1,2,3,4,7,8,9,10-octahydro-6H-pyridazino[1,2-a][1,2]diazepine-1(S) -carboxamide]-5-methyl-1-benzoyloxy-hexane-2-one a) 3-[9(S)-benzoylamino-6,10-dioxo-1,2,3,4,7,8,9,10-octahydro-6H-pyridazino[1,2-a][1,2]diazepine-1(S) -carboxamide]-5-methyl-1-bromo-hexane-2-one

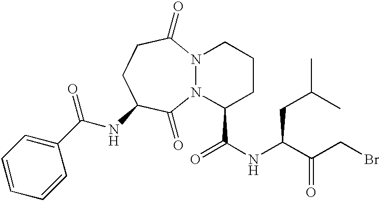

219 mg (0.38 mmole) in solution in 2 ml of CH2Cl2 are treated at 0° C. under nitrogen with 0.2 ml of solution of HBr(37%)/AcOH 30/70. After reaction for half an hour at 0° C. the mixture is poured into 20 ml of a saturated solution of NaHCO3. The mixture is extracted with 2×10 ml of AcOEt. The organic phases are washed with water (20 ml), dried over MgSO4 and concentrated under vacuum. 203 mg (yield=94%) of bromoketone is isolated in the form of a yellow foam.

MS: M+H⁺ 535

NMR H¹ (CDCl3) (300 MHz): 0.97(6H; d; 2CH3),1.67(1H; m; CH), 1.51-1.69(2H, 2m; 2H), 1.66(2H; m; CH2), 1.94(2H; m; CH2), 1.88 to 1.98 and 2.12 to 2.90(2H; 2m; CH2), 2.40 and 3.16 to 3.47(2H; dd and m; CH2), 3.01-4.61(2H; 2m; CH2), 4.57(1H; t; CH), 5.16(1H; m; CH); 7.00(1H; d; NH), 4.05(2H; AB; CH2), 7.47(2H; td; aromatic 2H's in meta position), 7.53(1H; m; aromatic H in para position), 7.82(2H; dd; aromatic 2H's in para position).

b): 3-[9(S)-benzoylamino-6,10-dioxo-1,2,3,4,7,8,9,10-octahydro -6H-pyridazino[1,2-a][1,2]diazepine-1(S) -carboxamide]-5-methyl-1-benzoyloxy-hexane-2-one

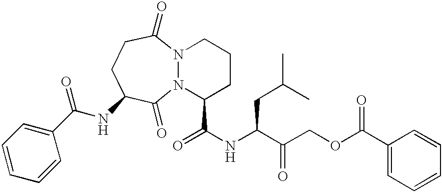

11 mg of KF (0.186 mmole; 2 equi.) followed by 13.7 mg of benzoic acid are added to a solution of 50 mg of bromoketone (0.093 mmole) obtained previously in 0.6 ml of DMF. The reaction is left overnight under agitation at ambient temperature. The reaction mixture is poured into 10 ml of a saturated solution of NaHCO3 and extraction is carried out with 2×5 ml ethyl acetate. The combined organic phases are washed with water (10 ml), dried over MgSO4 and concentrated under vacuum. 42 mg of pale yellow resin is obtained (yield=78%)

MS: MH⁺577; MNa⁺599

NMR-H¹: 0.97(6H; d; 2CH3), 1.71(1H; m; CH), 1,52-1.75(2H; 2m; CH2), 4.82(1H; m; CHNH), 6.27(1H; d; CHNH), 2,40-3.19(2H; 2m; CH2), 3.02-4.60(2H; 2m; CH2),1.50 to 2.20(2H; m; CH2), 5.03(2H; AB J=17Hz;COCH2O), 5.17(2H; m; 2CH), 7.00(1H; d; CONHCH), 7.40 to 7.65(6H; m; aromatic 6H's), 7.81 and 8.09(4H; dd; aromatic 2×2H's in ortho position of CO)

### Example 2

### 3-[9(S)-(2-furanoyl)amino-6,10-dioxo-1,2,3,4,7,8,9,10-octahydro-6H-pyridazino[1,2-a][1,2]diazepine-1(S)-carboxamide]-5-methyl-1-(2,6-dichlorobenzoyloxy-hexane-2-one

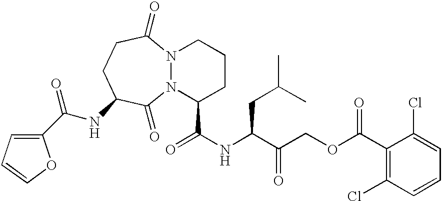

The operation is carried out starting from the product obtained in Stage b) of Stage 3 of Example 1 as follows: 11 mg of dimethylaminomethylpolystyrene resin (0.044 mmole; 2 equi.) is added to 11.6 mg (0.022 mmole) of 3(S)-[9(S)-(2-furanoyl)amino-6,10-dioxo-1,2,3,4,7,8,9,10-octahydro-6H -pyridazino[1,2-a][1,2]diazepine-1(S)-carboxamide]-5-methyl-1-bromo-hexane-2-one in solution in 0.35 ml of DMF. The reaction is left overnight under agitation. The mixture is filtered on sintered glass and the resin is washed twice with 2 ml of CH2Cl2. After intense evaporation under vacuum 9 mg of expected product is isolated (yield=82%). LC-MS (purity >80%) MH⁺ 636

### Example 3

### 3(S)-[9(S)-(2-furanoyl)amino-6,10-dioxo -1,2,3,4,7,8,9,10-octahydro-6H-pyridazino[1,2-a][1,2]diazepine-1(S)-carboxamide]-4-(4-hydroxyphenyl)-1-(2,6-dichlorobenzoyloxy)-butane-2-one

Stage 1: 3(S)-[9(S)-(2-furanoyl)amino-6,10-dioxo -1,2,3,4,7,8,9,10-octahydro-6H-pyridazino[1,2-a][1,2]diazepine-1(S)-carboxamide]-4-(4-terbutyloxyphenyl)1-(2,6-dichlorobenzoyloxy)-butane-2-one

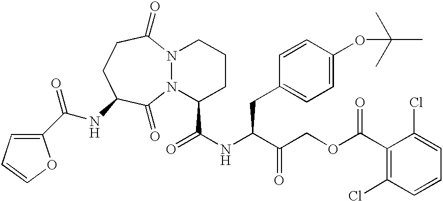

The process is carried out as in Stage 1 of Example 1 using another compound of formula (VI): fmoc-TYR(OtBu) instead of the compound of formula (VI): Leu-fmoc in the same quantity. Then the operation is carried out under the same reaction conditions as for Stages 2 to 4 of Example 1 and in this way the expected product is obtained.

Stage 2: 3(S)-[9(S)-(2-furanoyl)amino -6,10-dioxo-1,2,3,4,7,8,9,10-octahydro-6H-pyridazino[1,2-a][1,2]diazepine-1(S)-carboxamide]-4-(4-hydroxyphenyl)-1-(2,6-dichlorobenzoyloxy)-butane-2-one

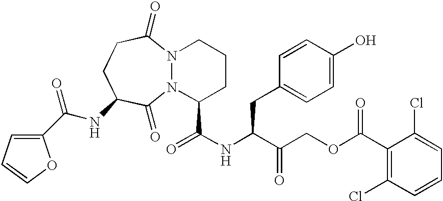

16 mg (0.022 mmole) of the product obtained above in Stage 1 in solution in 0.25 ml of CH2Cl2 is treated with 0.25 ml of trifluoroacetic acid The reaction is left under agitation for one hour at ambient temperature. The solvent is evaporated to dryness. 14.4 mg of expected product is isolated (yield=95%) LC-MS (purity >80%) MH⁺ 686

By operating as indicated above, the products of Examples 4 to 609 were prepared, the structures of which are indicated in the tables of FIG. 4.

### Example 610

### Pharmaceutical Composition

Tablets were prepared corresponding to the following formula:

Product of Example 1 . . . 0,2 g

Excipient for a tablet completed at . . . 1 g

(detail of the excipient: lactose, talc, starch, magnesium stearate).

Pharmacological Study

Study of Cathepsin K Inhibition:

The products to be tested (10 mM) are diluted to 1 MW in DMSO and distributed into Nunc polystyrene 96 well plates at a rate of 2 μl per well. Column 12 of the plate is reserved for the controls and therefore receives 1 μl of DMSO (without products) per well. The plates are stored at −80° C. and thawed on the day of the experiment.

The products are diluted to 50 μM by adding 38 μl of reaction buffer: sodium acetate 100 mM, EDTA 5 mM, DTT 1 MW pH 5.5. The addition as well as all the operations by pipette are carried out with a CybiWell 96 tip pipette. After mixing the solutions, each product is transferred into 2 wells (duplicates) of a Greiner black 384 well plate at a rate of 10 μl per well. Two 96-well plates can therefore be tested in a 384-well plate.

A solution of substrate at 50 μM, Z-Val-arg-AMC (Calbiochem), is prepared in the reaction buffer. The substrate, is then distributed into all the wells of the 384-well plate (20 μl per well).

A solution of Cathepsin K at 12.5 ng/ml is prepared in the reaction buffer and distributed into all the wells of the 384-well plate (20 μl per well) except for the 16 wells serving as 100% inhibition controls (column 23 and 24, lines I to P) which receive 20 μl of enzyme-free buffer. The 100% inhibition controls are carried out in columns 23 and 24, lines A to H which do not contain products.

The plates are then incubated 2H at ambient temperature, then read on Fluoroskan (Labsystems): excitation 390 nm; emission 460 nm

The final concentrations of each of the reagents are: products 10 μM, substrate 20 μM, enzyme 5 ng/ml.

The % inhibition for each of the products is calculated by using points at 0 and 100% inhibition of each plate as references. The products presenting a significant inhibition are then retested on a range of concentrations from 50 to 0.5 μM to determine an IC50.

Study of Cathepsin B Inhibition:

Protocol identical to that of Cathepsin K except for:

Z-arg-arg-AMC substrate (Calbiochem), solution at 50 μM, final concentration 20 μM

Cathepsin B (Calbiochem), solution at 40 ng/ml, final concentration 16 ng/ml

Incubation for 1 hour at ambient temperature

Study of Papain Inhibition:

Protocol identical to that of Cathepsin K except for:

Papain (Sigma), solution at 50 ng/ml, final concentration 20 ng/ml

Incubation for 30 minutes at ambient temperature

Results

The IC50's found for the products of the examples are given in table I hereafter, in micromoles

TABLE I

| | Cathepsin K | Cathepsin B | Papain |
|---|---|---|---|
| Example | IC₅₀ | IC₅₀ | IC₅₀ |
| No. | μM | μM | μM |
| 38 | 5 | 20 (30%) | 20 (25%) |
| 39 | 3 | 20 | 20 (30%) |
| 40 | 1.8 | 20 (40%) | 20 (30%) |
| 42 | 4 | 20 (38%) | 20 (25%) |
| 43 | 1.5 | 15 | 20 (25%) |
| 44 | 2 | 20 (40%) | 20 |
| 45 | 0.9 | 20 (35%) | 0 |
| 46 | 6 | 20 (30%) | 20 (30%) |
| 47 | 5 | 20 (40%) | 20 (40%) |
| 48 | 3 | 7 | 20 (20%) |
| 54 | 0 | 20 (30%) | 15 |
| 91 | 6 | <0.2 | 0 |
| 101 | 0 | 6 | 20 (20%) |
| 107 | 20 | 0 | 0 |
| 117 | 7 | 20 (25%) | 0 |
| 122 | 18 | 0.7 | 20 (45%) |
| 128 | 20 (30%) | 20 (40%) | 6 |
| 129 | 0 | 20 (30%) | 4 |
| 130 | 20 (35%) | 20 (30%) | 6 |
| 131 | 7 | 0 | 9 |
| 132 | 20 (40%) | 2 | 6 |
| 133 | 20 (25%) | 0 | 20 |
| 134 | 20 (25%) | 20 (30%) | 7 |
| 135 | 20 (35%) | 0 | 18%) |
| 136 | 20 | 20 (25%) | 20 (20%) |
| 138 | 29 (20%) | 0 | 8 |
| 141 | 9 | 0.7 | 20 |
| 150 | 6 | 0.6 | 0 |
| 161 | 0 | 9 | 19 |
| 162 | 20 (30%) | 20 (20%) | 20 |
| 165 | 20 (40%) | 20 (30%) | 20 |
| 166 | 20 (20%) | 20 (20%) | 2 |
| 171 | 8 | 20 (30%) | 20 (20%) |
| 172 | 20 | 20 (20%) | 20 (10%) |
| 185 | 0 | 7 | 0 |
| 192 | 20 (15%) | 18 | 0 |
| 206 | 0 | 20 | 20 |
| 211 | 20 (20%) | 20 (30%) | 6 |
| 216 | 0 | 20 (40%) | 6 |
| 217 | 20 (30%) | 0 | 7 |
| 218 | 20 (25%) | 20 | 20 |
| 220 | 20 (20%) | 20 (30%) | 6 |
| 221 | 20 (25%) | 20 (30%) | 7 |
| 224 | 20 (20%) | 9 | 8 |
| 229 | 20 (40%) | 18 | 20 (15%) |
| 234 | 20 (30%) | 18 | 20 (15%) |
| 240 | 0 | 0 | 20 |
| 241 | 0 | 20 (20%) | 7 |
| 304 | 12 | 1.5 | 0 |
| 313 | 0 | 5 | 0 |
| 322 | 20 (20%) | 6 | 20 (20%) |
| 339 | 10 | 0 | 0 |
| 340 | 7 | 20 (0%) | 0 |
| 341 | 8 | 20 (25%) | 0 |
| 343 | 14 | 20 (20%) | 0 |
| 344 | 5 | 0 | 20 (40%) |
| 345 | 5 | 20 (25%) | 0 |
| 346 | 6 | 0 | 0 |
| 347 | 12 | 20 (25%) | 0 |
| 348 | 10 | 20 (35%) | 20 (20%) |
| 366 | 0 | 0 | 17 |
| 374 | 20 (20%) | 0 | 7 |
| 382 | 20 (20%) | 0 | 9 |
| 389 | 20 (20%) | 20 (30%) | 6 |
| 390 | 20 (20%) | 19 | 6 |
| 397 | 20 (20%) | 0 | 18 |
| 398 | 0 | 20 (20%) | 9 |
| 433 | 20 (20%) | 4 | 0 |
| 443 | 0 | 5 | 0 |
| 450 | 20 (25%) | 20 (20%) | 18 |
| 453 | 20 (30%) | 9 | 20 (40%) |
| 463 | 0 | 1.5 | 0 |
| 473 | 20 (25%) | 1.5 | 0 |
| 481 | 0 | 6 | 0 |
| 526 | 0 | 0 | 18 |
| 534 | 20 (20%) | 0 | 7 |
| 562 | 20 (20%) | 20 (20%) | 19 |
| 563 | 0 | 20 (20%) | 18 |

1) Products of formula (I): 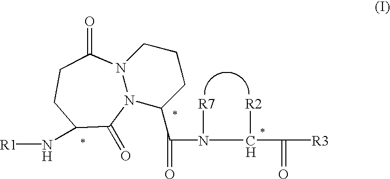
in which:
R₁ represents the —C(O)—R5, —SO2—R5 or —C(O)—NR6R5 radical
in which R6 represents a hydrogen atom or a linear or branched alkyl radical containing at most 4 carbon atoms and R5 represents a linear or branched alkyl radical containing at most 6 carbon atoms, cycloalkyl radical containing at most 6 carbon atoms, phenyl, naphthyl or a monocyclic heterocyclic radical saturated or unsaturated with 5 or 6 members containing one or more identical or different heteroatoms chosen from O, N or S,
the alkyl, phenyl and heterocyclic radicals as defined above for R1, being optionally substituted by one or more radicals chosen from the halogen atoms and the linear or branched alkyl or alkoxy radicals containing at most 4 carbon atoms, hydroxyl, acyl radicals containing at most 7 carbon atoms, trifluoromethyl, cyano, thienyl, phenyl, phenoxy and isoxazolyl radicals;
R2 and R7 are such that
either R7 represents a hydrogen atom and
R2 is such that the 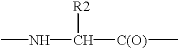
group in the products of formula (I) represents a so-called natural or non-natural amino acid remainder of 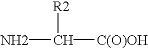
structure with the exception of aspartic acid,
or R2 and R7 together with the nitrogen and carbon atoms to which they are linked form a ring in such a way that the group thus formed in the products of formula (I): 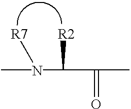
represents so-called natural or non-natural amino acid remainder of: 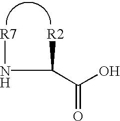
structure
R3 represents the —CH═N2 radical or the —CH2—L—R4 radical in which L represents a single bond or a divalent radical chosen from —O—, —O—C(O)—, —NH— and —S—(CH2)n-, with n representing an integer from 0 to 6,
and R4 represents a linear or branched alkyl radical containing at most 6 carbon atoms or a monocyclic or bicyclic carbocyclic or heterocyclic radical containing from 5 to 10 members, saturated or unsaturated, containing one or more identical or different heteroatoms chosen from O, N, NH or S and able to contain a —C(O) member,
the alkyl, carbocyclic and heterocyclic radicals as defined above for R4, being optionally substituted by one or more radicals chosen from the halogen atoms; the hydroxyl; free, salified or esterified carboxy; —C(O)—NH2, —C(O)—NH(alkyl), —C(O)—N(alkyl)(alkyl), —NH—C(O)-(alkyl), —N(alkyl)-C(O)—(alkyl); thienyl; phenyl; alkylphenyl; alkyl and alkoxy radicals themselves optionally substituted by one or more radicals chosen from the acyl radicals containing at most 7 carbon atoms, cyano, —NH2, —NH(alkyl), —N(alkyl)(alkyl) and phenyl radicals, it being understood that in the above radicals, the alkyl and alkoxy radicals are linear or branched and contain at most 4 carbon atoms,
the * sign indicating the carbon atoms which can be in S or R configuration,
said products of formula (I) being in all possible racemic, enatiomeric and diastereoisomeric isomer forms, as well as the addition salts with mineral and organic acids or mineral and organic bases of said products of formula (I).

2) Products of formula (1) as defined in claim 1 in which R1 and R3 have the meanings indicated in claim 1 and R2 represents a so-called natural amino acid remainder with the exception of aspartic acid,
said products of formula (I) being in all possible racemic, enatiomeric and diastereoisomeric isomer forms, as well as the addition salts with mineral and organic acids or mineral and organic bases of said products of formula (I).

3) Products of formula (I) as defined in any one of claims 1 and 2 in which R1 and R3 have the meanings indicated in claim 1 or 2 and
R2 and R7 are such that:
either R7 represents a hydrogen atom and R2 represents a hydrogen atom or a linear or branched alkyl radical containing at most 6 carbon atoms optionally substituted by one or more radicals chosen from the amino; acylamino; NH═C(NH2)—NH—; mercapto; linear or branched alkylthio radical containing at most 4 carbon atoms; hydroxyl; linear or branched alkoxy radical containing at most 4 carbon atoms; imidazolyl; indolyl; phenyl radical itself optionally substituted by one or more radicals chosen from the halogen atoms, the hydroxyl radical, a linear or branched alkoxy radical containing at most 4 carbon atoms and the phenoxy radical itself optionally substituted by one or more radicals chosen from the iodine atoms and the hydroxyl radical; and the —C(O)—O—R8 radical in which R8 represents a hydrogen atom or a linear or branched alkyl or alkenyl radical containing at most 6 carbon atoms,
or R2 and R7 together with the nitrogen and carbon atoms to which they are linked form a ring comprising 5 to 7 members optionally substituted by one or more hydroxyl or linear or branched alkoxy radicals containing at most 4 carbon atoms,
said products of formula (I) being in all possible racemic, enatiomeric and diastereoisomeric isomer forms, as well as the addition salts with mineral and organic acids or mineral and organic bases of said products of formula (I).

4) Products of formula (I) as defined in any one of claims 1 to 3 in which R1 and R3 have the meanings indicated in any one of claims 1 to 3,
R2 and R7 are such that:
either R7 represents a hydrogen atom and R2 has the meaning indicated in claim 3
or R2 and R7 together with the nitrogen and carbon atoms form a pyrrolidinyl ring optionally substituted by one or more hydroxyl or linear or branched alkoxy radicals containing at most 4 carbon atoms,
said products of formula (I) being in all possible racemic, enatiomeric and diastereoisomeric isomer forms, as well as the addition salts with mineral and organic acids or mineral and organic bases of said products of formula (I).

5) Products of formula (I) as defined in any one of claims 1 to 3 corresponding to formula (Ia): 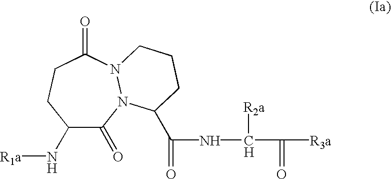
in which
R₁ₐ represents the —C(O)—R5a, —SO2-R5a or —C(O)—NR6aR5a radical in which R6a represents a hydrogen atom or a linear or branched alkyl radical containing at most 4 carbon atoms and R5a represents a radical chosen from the linear or branched alkyl radicals containing at most 6 carbon atoms; cycloalkyl radical containing at most 6 carbon atoms; phenyl; naphthyl; furyl; morpholinyl; thienyl; pyridyl, radicals
the alkyl, phenyl, thienyl and pyridyl radicals being optionally substituted by one or more radicals chosen from the halogen atoms and the linear or branched alkyl or alkoxy radicals containing at most 4 carbon atoms, hydroxyl, acyl radicals containing at most 7 carbon atoms, phenoxy, trifluoromethyl, cyano, thienyl, phenyl and isoxazolyl radicals;
R2a has the meaning indicated in any one of claims 1 to 4 when R7 represents a hydrogen atom,
R3a represents the —CH═N2 radical or the —CH2-La-R4a radical in which La represents a single bond or a divalent radical chosen from —O—, —O—C(O)— and —S—(CH2)na-, with na representing an integer from 0 to 3,
and R4a represents a radical chosen from the linear or branched alkyl radicals containing at most 6 carbon atoms; phenyl; tetrazolyl; piperazinyl; piperidyl; pyridyl; benzothiazolyl; quinolyl; thiadiazolyl; pyrimidinyl; tetralone radicals;
the alkyl, phenyl, tetrazolyl, piperazinyl and piperidyl radicals as defined above for R4a, being optionally substituted by one or more radicals chosen from the halogen atoms; the hydroxyl; free, salified or esterified carboxy radicals;
—C(O)—NH2; —C(O)—NH(alkyl); —C(O)—N(alkyl)(alkyl); —NH—C(O)-(alkyl); —N(alkyl)-C(O)-(alkyl); thienyl; phenyl; alkylphenyl; alkyl and alkoxy radicals themselves optionally substituted by one or more radicals chosen from the acyl radicals containing at most 7 carbon atoms, cyano, —NH2, —NH(alkyl), —N(alkyl)(alkyl) and phenyl radicals,
it being understood that in the above radicals, the alkyl and alkoxy radicals are linear or branched and contain at most 4 carbon atoms,
said products of formula (Ia) being in all possible racemic, enatiomeric and diastereoisomeric isomer forms, as well as the addition salts with mineral and organic acids or mineral and organic bases of said products of formula (Ia).

6) Products of formula (I) as defined in any one of claims 1 to 5 corresponding to formula (Ib): 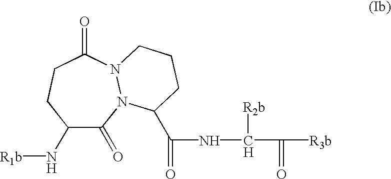
in which:
R_{1b} represents the —C(O) —R5b, —SO2—R5b or —C(O) —NR6bR5b radical
in which R6b represents a hydrogen atom or a methyl radical and R5b represents a radical chosen from the linear or branched alkyl radicals containing at most 4 carbon atoms optionally substituted by a thienyl; cyclohexyl; phenyl radical optionally substituted by a linear or branched alkoxy radical containing at most 4 carbon atoms, a phenoxy or trifluoromethyl; naphthyl; furyl; morpholinyl; thienyl radical optionally substituted by an isoxazolyl radical; pyridyl radical optionally substituted by a trifluoromethyl radical
R2b represents a hydrogen atom or a linear or branched alkyl radical containing at most 6 carbon atoms optionally substituted either by the phenyl radical itself optionally substituted by the hydroxyl or linear or branched alkoxy radicals containing at most 4 carbon atoms, or by the —C(O)—O—R8b radical in which R8b represents a linear or branched alkyl or alkenyl radical containing at most 6 carbon atoms,
R3b represents the —CH═N2 radical or the —CH2—Lb—R4b radical in which Lb represents a single bond or a divalent radical chosen from —O—, —O—C(O)— and —S—(CH2)nb-, with nb representing the integer 0 or 1,
and R4b represents a radical chosen from the pyridyl; benzothiazolyl; quinolyl; thiadiazolyl; pyrimidinyl; tetralone; linear or branched alkyl radicals containing at most 4 carbon atoms optionally substituted by a free, salified or esterified carboxy or thienyl radical; phenyl optionally substituted by one or more radicals chosen from the halogen atoms, the free, salified or esterified carboxy radicals, the linear or branched alkyl and alkoxy radicals containing at most 4 carbon atoms, cyanoalkyl, alkylphenyl, —NH—C(O)—CH3, —C(O)—N(alkyl)(alkyl); tetrazolyl radicals optionally substituted by a phenyl radical; piperazinyl optionally substituted on the second nitrogen atom by a phenyl or linear or branched alkyl radical containing at most 4 carbon atoms themselves optionally substituted by an acyl (pyrrolidinylcarbonyl) radical, one or two phenyl radicals or an —NH2, —NH(alkyl) or —N(alkyl)(alkyl) radical; piperidyl optionally substituted by a benzyl or —C(O)—N(alkyl)(alkyl) radical, it being understood that in the above radicals, the alkyl and alkoxy radicals are linear or branched and contain at most 4 carbon atoms,
said products of formula (Ib) being in all possible racemic, enatiomeric and diastereoisomeric isomer forms, as well as the addition salts with mineral and organic acids or mineral and organic bases of said products of formula (Ib).

7) Products of formula (I) as defined in any one of claims 1 to 6 in which R1 represents a radical chosen from the following radicals: 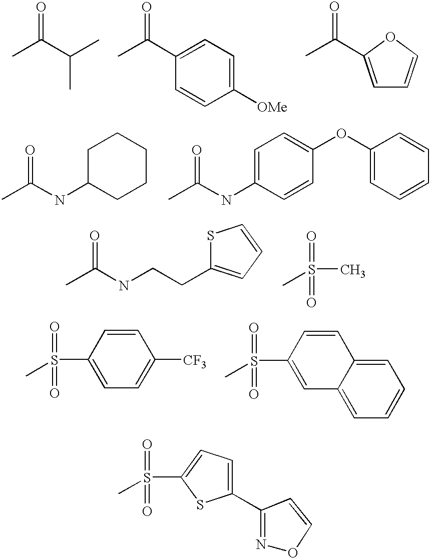
and R2, R3 and R7 have the values indicated in claim 1.

8) Products of formula (I) as defined in any one of claims 1 to 7 in which R7 represents a hydrogen atom and R2 represents a radical chosen from the following radicals: 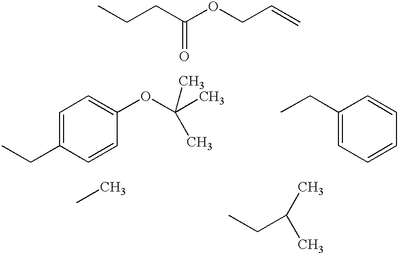
and R1 and R3 have the values indicated in claim 1.

9) Products of formula (I) as defined in any one of claims 1 to 8 in which R3 represents a radical chosen from the following radicals: 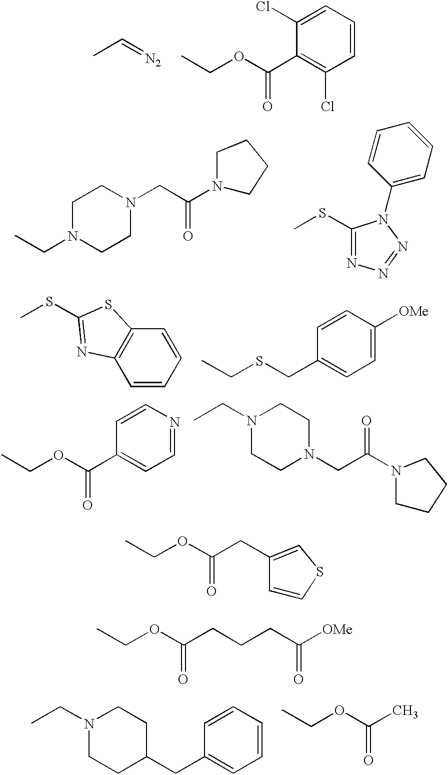
and R1, R2 and R7 have the values indicated in claim 1.

10) Products of formula (I) the names of which follow:
3-[9(S)-benzoylamino-6,10-dioxo-1,2,3,4,7,8,9,10-octahydro-6H-pyridazino[1,2-a][1,2]diazepine-1(S) -carboxamide]-5-methyl-1-benzoyloxy-hexane-2-one
3-[9(S)-(2-furanoyl)amino-6,10-dioxo-1,2,3,4,7,8,9,10-octahydro-6H-pyridazino[1,2-a][1,2]diazepine-1(S)-carboxamide]-5-methyl-1-(2,6-dichlorobenzoyloxy-hexane-2-one
3(S)-[9(S)-(2-furanoyl)amino-6,10-dioxo-1,2,3,4,7,8,9,10-octahydro-6H-pyridazino[1,2-a][1,2]diazepine-1(S)-carboxamide]-4-(4-hydroxyphenyl)-1-(2,6-dichlorobenzoyloxy)-butane-2-one

11) Products of formula (I) the names of which follow:
(2-propenyl)(4S)6-diazo-4-[[[(1S,9S)-9-[(2-methyl-1-oxo-propyl)amino]-octahydro-6,10-dioxo-6H-pyridazino[1,2-a][1,2]diazepine-1-yl]carbonyl]amino]-5-oxo-hexanoate
(2-propenyl)(4S)6-diazo-4-[[[(1S,9S)-9-[(3-methoxybenzoyl)amino]-octahydro-6,10-dioxo-6H-pyridazino[1,2-a][1,2]diazepine-1-yl]carbonyl]amino]-5-oxo-hexanoate
(2-propenyl)(4S)6-diazo-4-[[[(1S,9S)-9-[[(2-furanyl)carbonyl]amino]-octahydro-6,10-dioxo-6H -pyridazino[1,2-a][1,2]diazepine-1-yl]carbonyl]amino]-5-oxo -hexanoate
(2-propenyl)(4S)6-diazo-4-[[[(1S,9S)-9-[[(cyclohexylamino)carbonyl]amino]-octahydro-6,10-dioxo-6H -pyridazino[1,2-a][1,2]diazepine-1-yl]carbonyl]amino]-5-oxo -hexanoate
(2-propenyl)(4S)6-diazo-4-[[[(1S,9S)-9-[[[(4-phenoxyphenyl)amino]carbonyl]amino]-octahydro-6,10-dioxo-6H -pyridazino[1,2-a][1,2]diazepine-1-yl]carbonyl]amino]-5-oxo -hexanoate
(2-propenyl)(4S)6-diazo-4-[[[(1S,9S)-9-[[[[2-(p-2-yl) -ethyl]amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-diazo-4-[[[(1S,9S)-9-[(methylsulphonyl)amino]-octahydro-6,10-dioxo-6H-pyridazino[1,2-a][1,2]diazepine-1-yl]carbonyl]amino]-5-oxo-hexanoate
(2-propenyl)(4S)6-diazo-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate
(2-propenyl)(4S)6-diazo-4-[[[(1S,9S)-9-[[(2-naphthalenyl)sulphonyl]amino]-octahydro-6,10-dioxo-6H-pyridazino[1,2-a][1,2]diazepine-1-yl]carbonyl]amino]-5-oxo-hexanoate
(2-propenyl)(4S)6-diazo-4-[[[(1S,9S)-9-[[[5-(isoxazol-3-yl)-2-thienyl]sulphonyl]amino]-octahydro-6,10-dioxo-6H-pyridazino[1,2-a][1,2]diazepine-1-yl]carbonyl]amino]-5-oxo-hexanoate
9-[(9S)-[[(4-phenoxyphenyl)amino]carbonyl]amino]-N-[(2S)-4diazo-1-[4-[(1,1-dimethyl)ethoxy]phenyl]-3-oxo-2-butyl]-6H-(1S)-pyridazino[1,2-a][1,2]diazepine-1-carboxamide
(2-propenyl)(4S)6-[(2,6-dichlorobenzoyl)oxy]-4-[[[(1S,9S)-9-[[(2-furanyl)carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(2,6-dichlorobenzoyl)oxy]-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate
(2-propenyl)(4S)6-[4-[[2-oxo-2-(1-pyrrolidinyl)ethyl]piperazin-1-yl]-4-[[[(1S,9S)-9-[[[4-(trifluoromethyl)phenyl]sulphonyl]amino]-octahydro-6,10-dioxo-6H-pyridazino[1,2-a][1,2]diazepine-1-yl]carbonyl]amino]-5-oxo-hexanoate
(2-propenyl)(4S)6-[4-[[2-oxo-2-(1-pyrrolidinyl)ethyl]piperazin-1-yl]-4-[[[(1S,9S)-9-[[(2-naphthalenyl)sulphonyl]amino]-octahydro-6,10-dioxo-6H-pyridazino[1,2-a][1,2]diazepine-1-yl]carbonyl]amino]-5-oxo-hexanoate
(2-propenyl)(4S)6-[(2,6-dichlorobenzoyl)oxy]-4-[[[(1S,9S)-9-[[(cyclohexylamino)carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(1-phenyl-1H-tetrazol-5-yl)thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(benzothiazol-2-yl)thio]-4-[[[(1S,9S) -9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[[(4-methoxyphenyl)methyl]thio]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[[(4-pyridinyl)carbonyl]oxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(2,6-dichlorobenzoyl)oxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)((4S)6-acetyloxy-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[1-oxo-2-(thien-3-yl)ethoxy]-4-[[[(1S,9S)-9-[[[(4-phenoxyphenyl)amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(1-phenyl-1H-tetrazol-5-yl)thio]-4-[[[(1S,9S)-9-[[[[2-(thien-2-yl)-ethyl]amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(2,6-dichlorobenzoyl)oxy]-4-[[[(1S,9S)-9-[[[[2-(thien-2-yl)-ethyl]amino]carbonyl]amino]-octahydro-6,10-dioxo-6H-pyridazino[1,2-a][1,2]diazepine-1-yl]carbonyl]amino]-5-oxo-hexanoate
(2-propenyl)(4S)6-[(2,6-dichlorobenzoyl)oxy]-4-[[[(1S,9S)-9-[(methylsulphonyl)amino]-octahydro-6,10-dioxo-6H-pyridazino[1,2-a][1,2]diazepine-1-yl]carbonyl]amino]-5-oxo-hexanoate
9-[(9S)[[(4-phenoxyphenyl)amino]carbonyl]amino]-N-((2S)-4-diazo-1-phenyl-3-oxo-2-butyl)-octahydro-6,10-dioxo -6H-(1S)-pyridazino[1,2-a][1,2]diazepine-1-carboxamide
9-[(9S)-[[[2-(thien-2-yl)-ethyl]amino]carbonyl]amino]-N-((2S)-4-diazo-1-phenyl-3-oxo-2-butyl)-octahydro-6,10-dioxo-6H-(1S)-pyridazino[1,2-a][1,2]diazepine-1-carboxamide
9-[(9S)-[(2-naphthalenyl)sulphonyl]amino ]-N-((2S)-4-diazo-1-phenyl-3-oxo-2-butyl)-octahydro-6,10-dioxo-6H-(1S)-pyridazino[1,2-a][1,2]diazepine-1-carboxamide
9-[(9S)-[[5-(isoxazol-3-yl)-2-thienyl]sulphonyl]amino]-N-((2S)-4-diazo-1-phenyl-3-oxo-2-butyl)-octahydro-6,10-dioxo-6H-(1S)-pyridazino[1,2-a][1,2]diazepine-1-carboxamide -9-[(9S)-[[(4-phenoxyphenyl)amino]carbonyl]amino]-N-((2S)-4-diazo-3-oxo-2-butyl)-octahydro-6,10-dioxo -6H-(1S)-pyridazino[1,2-a][1,2]diazepine-1-carboxamide
9-[(9S)-[[[3-(thien-2-yl)-ethyl]amino]carbonyl]amino]-N-((2S)-4-diazo-3-oxo-2-butyl)-octahydro-6,10-dioxo -6H-(1S) -pyridazino[1,2-a][1,2]diazepine-1-carboxamide
9-[(9S)-(3-methoxybenzoyl)amino]-N-((2S)-4-[(2,6-dichlorobenzoyl)oxy]-1-phenyl-3-oxo-2-butyl]-octahydro-6,10-dioxo-6H-(1S)-pyridazino[1,2-a][1,2]diazepine-1-carboxamide
9-[(9S)-[(2-furanyl)carbonyl]amino]-N-[(2S)-4-[(2,6-dichlorobenzoyl)oxy]-1-phenyl-3-oxo-2-butyl]-octahydro-6,10-dioxo-6H-(1S)-pyridazino[1,2-a][1,2]diazepine-1-carboxamide
9-[(9S)-[(cyclohexylamino)carbonyl]amino]-N-[(2S)-4-[(2,6dichlorobenzoyl)oxy]-1-phenyl-3-oxo-2-butyl]-octahydro-6,10-dioxo-6H-(1S)-pyridazino[1,2-a][1,2]diazepine-1-carboxamide
9-[(9S)-[[(4-phenoxyphenyl)amino]carbonyl]amino]-N-[(2S)-1-phenyl-4-[(1-phenyl-1H-tetrazol-5-yl)thio]-3-oxo-2-butyl]-octahydro-6,10-dioxo-6H-(1S)-pyridazino[1,2-a][1,2]diazepine-1-carboxamide
9-[(9S)-[[(4-phenoxyphenyl)amino]carbonyl]amino]-N-[(2S)-1-phenyl-4-[1-oxo-2-(3-thienyl)ethoxy]-3-oxo-2-butyl]-octahydro-6,10-dioxo-6H-(1S)-pyridazino[1,2-a][1,2]diazepine-1-carboxamide
(methyl)[2-oxo-4-phenyl-3[(3S)-[[(4-phenoxyphenyl)amino]carbonyl]amino]-octahydro-6,10-dioxo-6H-(1S)-pyridazino[1,2-a][1,2]diazepin-1-yl]carbonyl]amino]butyl]pentanedioate
9-[(9S)-[[(4-phenoxyphenyl)amino]carbonyl]amino]-N-[(2S)-4-[(4-phenylmethyl)-1-piperidinyl ]-1-phenyl-3-oxo-2-butyl]-octahydro-6,10-dioxo-6H-(1S)-pyridazino[1,2-a][1,2]diazepine-1-carboxamide
9-[(9S)-[[[2-(thien-2-yl)-ethyl]amino]carbonyl]amino]-N-[(2S)-1-phenyl-4-[(1-phenyl-1H-tetrazol-5-yl)thio]-3-oxo-2-butyl]-octahydro-6,10-dioxo-6H-(1S)-pyridazino[1,2-a][1,2]diazepine-1-carboxamide
9-[(9S)-[[[2-(thien-2-yl)-ethyl]amino]carbonyl]amino]-N-[(2S)-1-phenyl-4-[(2-benzothiazolyl)thio]-3-oxo-2-butyl]-octahydro-6,10-dioxo-6H-(1S)-pyridazino[1,2-a][1,2]diazepine-1-carboxamide
9-[(9S)-[[[2-(thien-2-yl)-ethyl]amino]carbonyl]amino]-N-[(2S)-1-phenyl-4-[(-2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[[[2-(thien-2-yl)-ethyl]amino]carbonyl]amino]-N-[(2S)-1-phenyl-4-[4-[2-(1-pyrrolidinyl)-2-oxo-ethyl]piperazin-1-yl]]-3-oxo-2-butyl]-octahydro-6,10-dioxo -6H-(1S)-pyridazino[1,2-a][1,2]diazepine-1-carboxamide -9-[(9S)-(methylsulphonyl)amino]-N-[(2S)-1-phenyl-4-[(-2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[[[4-(trifluoromethyl)phenyl]sulphonyl]amino]-N-[(2S)-1-phenyl-4-[(1-phenyl-1H-tetrazol-5-yl)thio]-3-oxo-2-butyl]-octahydro-6,10-dioxo-6H-(1S)-pyridazino [1,2-a][1,2]diazepine-1-carboxamide
9-[(9S)-[[[4-(trifluoromethyl)phenyl]sulphonyl]amino]-N-[(2S)-1-phenyl-4-(2-benzothiazolyl)thio]-3-oxo-2-butyl]-octahydro-6,10-dioxo-6H-(1S)-pyridazino[1,2-a][1,2]diazepine-1-carboxamide
9-[(9S)-(methylsulphonyl)amino]-N-[(3S)-5-methyl-1-[(2,6-dichlorobenzoyl)oxy]-2-oxo-3-hexyl]-octahydro-6,10-dioxo-6H-(1S)-pyridazino[1,2-a][1,2]diazepine-1-carboxamide
9-[(9S)-[[[4-(trifluoromethyl)phenyl]sulphonyl]amino]]-N-[(3S)-5-methyl-1-[(2,6-dichlorobenzoyl)oxy]-2-oxo-3-hexyl]-octahydro-6,10-dioxo-6H-(1S)-pyridazino[1,2-a][1,2]diazepine-1-carboxamide
9-[(9S)-[(2-naphthalenyl)sulphonyl]amino ]-N-[(3S)-5-methyl-1-[(2,6-dichlorobenzoyl)oxy]-2-oxo-3-hexyl]-octahydro-6,10-dioxo-6H-(1S)-pyridazino[1,2-a][1,2]diazepine-1-carboxamide
9-[(9S)-(2-methyl-1-oxo-propyl)amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-(3-methoxybenzoyl)amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-[(2-furanyl)carbonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-[(cyclohexylamino)carbonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-[[(4-phenoxyphenyl)amino]carbonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-[[[2-(thien-2-yl)-ethyl]amino]carbonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide -9-[(9S)-(methylsulphonyl)amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-[[4-(trifluoromethyl)phenyl]sulphonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-[(2-naphthalenyl)sulphonyl]amino]-N-((3S)-1-diazo-5-methyl-2-oxo-3-hexyl)-octahydro-6,10-dioxo-6H-(1S)-pyridazino[1,2-a][1,2]diazepine-1-carboxamide
9-[(9S)-(3-methoxybenzoyl)amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide
9-[(9S)-[(2-furanyl)carbonyl]amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide
9-[(9S)-[(cyclohexylamino)carbonyl]amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide
9-[(9S)-[[(4-phenoxyphenyl)amino]carbonyl]amino]-N-[(3S)-1-[(1-phenyl-1H-tetrazol-5-yl)thio]-4-methyl-2-oxo-3-pentyl]-octahydro-6,10-dioxo-6H-(1S)-pyridazino [1,2-a][1,2]diazepine-1-carboxamide
9-[(9S)-[[(4-phenoxyphenyl)amino]carbonyl]amino]-N-[3S)-1-[(benzothiazol-2-yl)thio]-4-methyl-2-oxo-3-pentyl]-octahydro-6,10-dioxo-6H-(1S)-pyridazino[1,2-a][1,2-a]diazepine-1-carboxamide
9-[(9S)-[[[2-(thien-2-yl)-ethyl]amino]carbonyl]amino]-N-[(3S)-1-[(1-phenyl-1H-tetrazol-5-yl)thiol-4-methyl-2-oxo-3-pentyl]-octahydro-6,10-dioxo-6H-(1S)-pyridazino [1,2-a][1,2]diazepine-1-carboxamide
9-[(9S)-[[[2-(thien-2-yl)-ethyl]amino]carbonyl]amino]-N-[(3S)-1-[(benzothiazol-2-yl)thio]-4-methyl-2-oxo-3-pentyl]-octahydro-6,10-dioxo-6H-(1S)-pyridazino[1,2-a][1,2]diazepine-1-carboxamide
9-[(9S)-[(cyclohexylamino)carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[(2-furanyl)carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[[(4-phenoxyphenyl)amino]carbonyl]amino]-N-[(2S)-4-[(benzothiazol-2-yl)thio]-3-oxo-2-butyl]-octahydro-6,10-dioxo-6H-(1S)-pyridazino[1,2-a][1,2]diazepine-1-carboxamide
9-[(9S)-[[(4-phenoxyphenyl)amino]carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-(3-methoxybenzoyl)amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[[[2-(thien-2-yl)-ethyl]amino]carbonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[[4-(trifluoromethyl)phenyl]sulphonyl]amino]-N-[(2S)-4-[(2,6-dichlorobenzoyl)oxy]-3-oxo-2-butyl]-octahydro-6,10-dioxo-6H-(1S)-pyridazino[1,2-a][1,2]diazepine-1-carboxamide
9-[(9S)-[(cyclohexylamino)carbonyl]amino]-N-[(2S)-4-[(benzothiazol-2-yl)thio]-1-[4-[(1,1-dimethyl)ethoxy]phenyl]-3-oxo-2-butyl]-6H-(1S)-pyridazino[1,2-a][1,2]diazepine-1-carboxamide

12) Process for the preparation of the products of formula (I), as defined in claim 1, characterized in that the compound of formula (II): 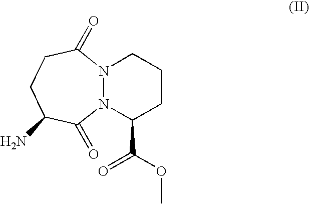
is subjected to a reaction with a compound of formula (III):
R1′—X (III)
in which X represents a halogen atom and R1′ has the meaning indicated in claim 1 for R1, in which the optional reactive functions are optionally protected by protective groups, in order to obtain the product of formula (IV): 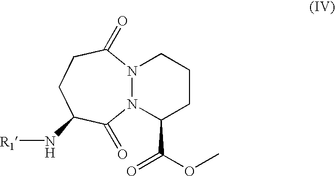
in which R1′ has the meaning indicated above, which product of formula (IV) is subjected to a saponification reaction in order to obtain the product of formula (V): 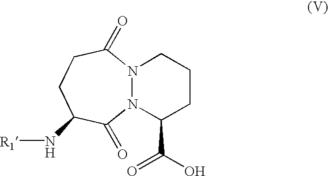
in which R1′ has the meaning indicated above, which product of formula (V) is subjected to a reaction with a product of formula (VI): 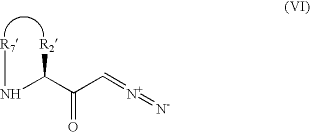
in which R2′ and R7′ have the meanings indicated in claim 1 for R2 and R7 respectively, in which the optional reactive functions are optionally protected by protective groups, in order to obtain a product of formula (Ix): 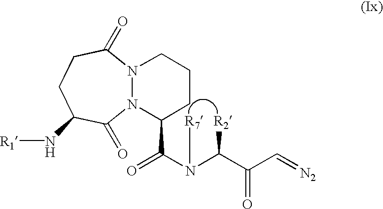
in which R1′, R2′ and R7′ have the meanings indicated above, which product of formula (Ix) can be subjected to a bromination reaction in order to obtain a product of formula (VII): 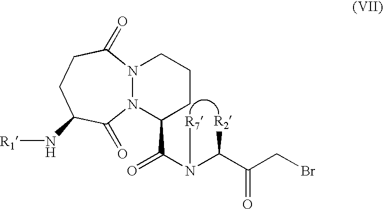
in which R1′, R2′ and R7′ have the meanings indicated above, which product of formula (VII) is subjected to a reaction with a product of formula (VIII):
H′—L—R4′ (VIII)
in which L has the meaning indicated in claim 1 and R4′ has the meaning indicated in claim 1 for R4, in which the optional reactive functions are optionally protected by protective groups, in order to obtain a product of formula (Iy): 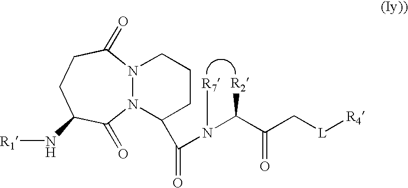
in which R1′, R2′, R4′ and R7′ have the meanings indicated above,
which products of formulae (Ix) and (Iy) can be products of formula (I) and which, in order to obtain products or other products of formula (I), can be subjected, if desired and if necessary, to one or more of the following conversion reactions, in any order:
a) an esterification reaction of the acid function,
b) a saponification reaction of the ester function to an acid function,
c) an oxidation reaction of the alkylthio group to a corresponding sulphoxide or sulphone,
d) a conversion reaction of the ketone function to an oxime function,
e) a reduction reaction of the free or esterified carboxy function to an alcohol function,
f) a conversion reaction of the alkoxy function to a hydroxyl function, or also of the hydroxyl function to an alkoxy function,
g) an oxidation reaction of the alcohol function to an aldehyde, acid or ketone function,
h) a conversion reaction of the nitrile radical to a tetrazolyl,
i) an elimination reaction of the protective groups which can be carried by the protected reactive functions,
j) a salification reaction by a mineral or organic acid or by a base in order to obtain the corresponding salt,
k) a resolution reaction of the racemic forms to resolved products, said products of formula (I) obtained in this way being in all possible racemic, enatiomeric and diastereoisomeric isomer forms.

13) As medicaments, the products of formula (I) as defined in claims 1 to 4, as well as the addition salts with pharmaceutically acceptable mineral or organic acids or mineral or organic bases of said products of formula (I).

14) As medicaments, the products of formulae (Ia) and (Ib) as defined in claim 5 or 6, as well as the addition salts with pharmaceutically acceptable mineral or organic acids or mineral or organic bases of said products of formulae (Ia) and (Ib).

15) As medicaments the products of formula (I) as defined in any one of claims 7 to 9 as well as the addition salts with pharmaceutically acceptable mineral or organic acids or mineral or organic bases of said products of formula (I).

16) As medicaments, the products of formula (I) as defined in claim 10 or 11, as well as the addition salts with pharmaceutically acceptable mineral or organic acids or mineral or organic bases of said products of formula (I).

17) Pharmaceutical compositions containing as active ingredient, at least one of the medicaments as defined in claims 13 to 16.

18) Use of the products according to one of claims 1 to 11 or pharmaceutically acceptable salts of said products for the preparation of medicaments intended for the prevention or treatment of diseases in which metabolic enzymes such as proteases or kinases are involved.

19) Use according to claim 18 for the preparation of medicaments intended for the prevention or treatment of diseases in which cathepsin K, cathepsin B or papain are involved.

20) Use according to claim 18 characterized in that the diseases to be prevented or treated are chosen from the following group of diseases: cardiovascular diseases, cancers, diseases of the central nervous system, inflammatory diseases, infectious diseases or also bone diseases.

21) Use according to claim 18 characterized in that the diseases to be prevented or treated are diseases of the central nervous system or bone diseases such as in particular osteoporosis.

22) As industrial products, the compounds of formula (VII).
